(19) United States
(12) Patent Application Publication
(10) Pub. No.: US 2006/0063751 A1
(43) Pub. Date: Mar. 23, 2006

(54) Novel fused heterocycles and uses thereof

(75) Inventors: Brian Aquila, Waltham, MA (US); Michael Howard Block, Waltham, MA (US); Audrey Davies, Waltham, MA (US); Jayachandran Ezhuthachan, Waltham, MA (US); Sandra Filla, Brownsberg, IN (US); Richard William Luke, Macclesfield (GB); Timothy Pontz, Waltham, MA (US); Daniel John Russell, Waltham, MA (US); Maria-Elena Theoclitou, Waltham, MA (US); XiaoLan Zheng, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 11/207,089

(22) Filed: Aug. 18, 2005

Related U.S. Application Data
(63) Continuation-in-part of application No. PCT/SE04/00304, filed on Mar. 4, 2004.
(60) Provisional application No. 60/602,399, filed on Aug. 18, 2004.
(60) Provisional application No. 60/602,366, filed on Aug. 18, 2004.

(30) Foreign Application Priority Data
Mar. 7, 2003 (SE) 0300627-7
Apr. 15, 2003 (SE) 0301138-4
Jun. 10, 2003 (SE) 0301697-9
Oct. 24, 2003 (SE) 0302826-3

(51) Int. Cl. A61K 31/513; C07D 417/02; C07D 413/02; C07D 403/02

(52) U.S. Cl. 514/210.2; 514/269; 544/310

(57) ABSTRACT

This invention relates to novel compounds having the formula (I) 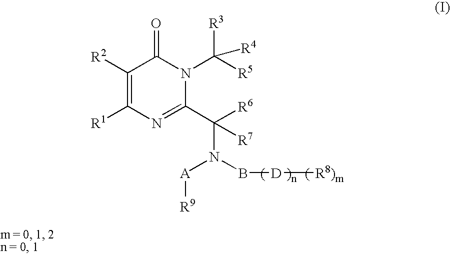
wherein R¹, R², R³, R⁴, R⁵, R⁶, R⁷, R⁸ and R⁹ are as defined in the specification and to their pharmaceutical compositions and to their methods of use. These novel compounds provide a treatment or prophylaxis of cancer.

This application is a continuation in part of International Patent Application No. PCT/SE2004/000304 filed 4 Mar. 2004, which claims priority to SE 0300627-7 filed 7 Mar. 2003, SE 0301138-4 filed 15 Apr. 2003, SE 0301697-9 filed 10 Jun. 2003 and SE 0302826-3 filed 24 Oct. 2003. The contents of PCT/SE2004/000304, SE 0300627-7, SE 0301138-4, SE 0301697-9 and SE 0302826-3 are incorporated herein by reference. This application also claims the benefit of U.S. provisional application U.S. 60/602,399 filed 18 Aug. 2004 and U.S. provisional application U.S. 60/602,366 filed 18 Aug. 2004. The contents of U.S. 60/602,399 and U.S. 60/602,366 are also incorporated herein by reference.

### SECTION 1

### SECTION 1: FIELD OF THE INVENTION

The present invention relates to novel fused heterocycles, their pharmaceutical compositions and methods of use. In addition, the present invention relates to therapeutic methods for the treatment and prevention of cancers.

### SECTION 1: BACKGROUND OF THE INVENTION

One sub-class of anti-cancer drugs now used extensively in the clinic (taxanes, vinca-alkaloids) are directed at microtubules and block the cell division cycle by interfering with normal assembly or disassembly of the mitotic spindle (see Chabner, B. A., Ryan, D. P., Paz-Ares, I., Garcia-Carbonero, R., and Calabresi, P: Antineoplastic agents. In Hardman, J. G., Limbird, L. E., and Gilman, A. G., eds. Goodman and Gilman's The Pharmacological Basis of Therapeutics, 10^{th} edition, 2001, The MacGraw-Hill Companies, Inc). Taxol® (paclitaxel), one of the most effective drugs of this class, is a microtubule stabilizer. It interferes with the normal growth and shrinkage of microtubules thus blocking cells in the metaphase of mitosis. Mitotic block is often followed by slippage into the next cell cycle without having properly divided, and eventually by apoptosis of these abnormal cells (Blagoskionny, M. V. and Fojo, T.: Molecular effects of paclitaxel: myths and reality (a critical review). Int J Cancer 1999, 83:151-156.)

Some of the side effects of treatment with paclitaxel are neutropenia and peripheral neuropathy. Paclitaxel is known to cause abnormal bundling of microtubules in interphase cells. In addition, some tumour types are refractory to treatment with paclitaxel, and other tumours become insensitive during treatment. Paclitaxel is also a substrate for the multi-drug resistance pump, P-glycoprotein ((see Chabner et al., 2001).

Thus, there is a need for effective anti-mitotic agents that are more specific and have fewer side effects than anti-microtubule drugs, and also for agents that are effective against taxane-resistant tumours.

Kinesins are a large family of molecular motor proteins, which use the energy of ATP hydrolysis to move in a stepwise manner along microtubules. For a review, see Sablin, E. P.: Kinesins and microtubules: their structures and motor mechanisms. Curr Opin Cell Biol 2000, 12:35-41 and Schief, W. R. and Howard, J.: Conformational changes during kinesin motility. Curr Opin Cell Biol 2001, 13:19-28.

Some members of this family transport molecular cargo along microtubules to the sites in the cell where they are needed. For example, some kinesins bind to vescicles and transport them for long distances along microtubules in axons. Several family members are mitotic kinesins, as they play roles in the reorganization of microtubules that establishes a bipolar mitotic spindle. The minus ends of the microtubules originate at the centrosomes, or spindle poles, whilst the plus ends bind to the kinetochore at the centromeric region of each chromosome. Thus the mitotic spindle lines up the chromosomes at metaphase of mitosis and coordinates their movement apart and into individual daughter cells at anaphase and telophasei (cytokinesis). See Alberts, B., Bray, D., Lewis, J., Raff, M., Roberts, K., and Watson, J. D., Molecular Biology of the Cell, 3^{rd} edition, Chapter 18, The Mechanics of Cell Division, 1994, Garland Publishing, Inc. New York.

HsEg5 (Accession X85137; see Blangy, A., Lane H. A., d'Heron, P., Harper, M., Kress, M. and Nigg, E. A.: Phosphorylation by p34cdc2 regulates spindle association of human Eg5, a kinesin-related motor essential for bipolar spindle formation in vivo. Cell 1995, 83(7): 1159-1169) or, KSP, is a mitotic kinesin whose homologs in many organisms have been shown to be required for centrosome separation in the prophase of mitosis, and for the assembly of a bipolar mitotic spindle. For a review see Kashina, A. S., Rogers, G. C., and Scholey, J. M.: The bimC family of kinesins: essential bipolar mitotic motors driving centrosome separation. Biochem Biophys Acta 1997, 1357: 257-271. Eg5 forms a tetrameric motor, and it is thought to cross-link microtubules and participate in their bundling (Walczak, C. E., Vemos, I., Mitchison, T. J., Karsenti, E., and Heald, R.: A model for the proposed roles of different microtubule-based motor proteins in establishing spindle bipolarity. Curr Biol 1998, 8:903-913). Several reports have indicated that inhibition of Eg5 function leads to metaphase block in which cells display monastral spindles. Recently an Eg5 inhibitor called monastrol was isolated in a cell-based screen for mitotic blockers (Mayer, T. U., Kapoor, T. M., Haggarty, S. J., King, R. w., Schreiber, S. L., and Mitchison, T. J.: Small molecule inhibitor of mitotic spindle bipolarity identified in a phenotype-based screen. Science 1999, 286: 971-974).

Monastrol treatment was shown to be specific for Eg5 over kinesin heavy chain, another closely related motor with different functions (Mayer et al., 1999). Monastrol blocks the release of ADP from the Eg5 motor (Maliga, Z., Kapoor, T. M., and Mitchison, T. J.: Evidence that monastrol is an allosteric inhibitor of the mitotic kinesin Eg5. Chem & Biol 2002, 9: 989-996 and DeBonis, S., Simorre, J.-P., Crevel, I., Lebeau, L, Skoufias, D. A., Blangy, A., Ebel, C., Gans, P., Cross, R., Hackney, D. D., Wade, R. H., and Kozielski, F.: Interaction of the mitotic inhibitor monastrol with human kinesin Eg5. Biochemistry 2003, 42: 338-349) an important step in the catalytic cycle of kinesin motor proteins (for review, see Sablin, 2000; Schief and Howard, 2001). Treatment with monastrol was also shown to be reversible and to activate the mitotic spindle checkpoint which stops the progress of the cell division cycle until all the DNA is in place for appropriate division to occur (Kapoor, T. M., Mayer, T. U., Coughlin, M. L., and Mitchison, T. J.: Probing spindle assembly mechanisms with monastrol, a small molecule inhibitor of the mitotic kinesin, Eg5. J Cell Biol 2000, 150(5): 975-988). Recent reports also indicate that inhibitors of Eg5 lead to apoptosis of treated cells and are effective against several tumour cell lines and tumour models (Mayer et al., 1999).

Although Eg5 is thought to be necessary for mitosis in all cells, one report indicates that it is over-expressed in tumour cells (International Patent Application WO 01/31335), suggesting that they may be particularly sensitive to its inhibition. Eg5 is not present on the microtubules of interphase cells, and is targeted to microtubules by phosphorylation at an early point in mitosis (Blangy et al., 1995). See also; Sawin, K. E. and Mitchison, T. J.: Mutations in the kinesin-like protein Eg5 disrupting localization to the mitotic spindle. Proc Natl Acad Sci USA 1995, 92(10): 4289-4293, thus monastrol has no detectable effect on microtubule arrays in interphase cells (Mayer et al., 1999). Another report suggests that Eg5 is involved in neuronal development in the mouse, but it disappears from neurons soon after birth, and thus Eg5 inhibition may not produce the peripheral neuropathy associated with treatment with paclitaxel and other anti-microtubule drugs (Ferhat, L., Expression of the mitotic motor protein Eg5 in postmitotic neurons: implications for neuronal development. J Neurosci 1998, 18(19): 7822-7835). Herein we describe the isolation of a class of specific and potent inhibitors of Eg5, expected to be useful in the treatment of neoplastic disease.

### SECTION 1: SUMMARY OF THE INVENTION

In accordance with section 1 of the present invention, the applicants have hereby discovered novel compounds which possess cell-cycle inhibitory activity and are accordingly useful for their anti-cell-proliferation activity (such as anti-cancer) and are therefore useful in methods of treatment of diseases having cell-proliferation activity in human or animal subjects. In addition to novel compounds section 1 of the present invention also includes pharmaceutical compositions containing such compounds and to the use of such compounds in the manufacture of medicaments having an anti-cell proliferation effect in human or animal subjects. Section 1 of the invention also relates to processes for the manufacture of said compounds.

Section 1 of the present invention includes pharmaceutically acceptable salts or prodrugs of such compounds. Also in accordance with section 1 of the present invention applicants provide pharmaceutical compositions and a method to use such compounds in the treatment of cancer.

Such properties are expected to be of value in the treatment of disease states associated with cell cycle and cell proliferation such as cancers (solid tumours and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

### SECTION 1: DETAILED DESCRIPTION OF THE INVENTION

In a first embodiment of section 1, the present invention provides a novel compound having structural formula (I): 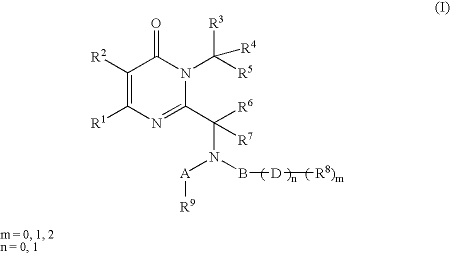
wherein,
A is C═O, CH₂, or SO₂;
B represents optionally substituted alkyl, optionally substituted alkenyl, optionally substituted alkynyl, optionally substituted aryl, optionally substituted cycloalkyl, or optionally substituted heterocycle;
D is O or N wherein 0 is optionally substituted with one R⁸, wherein N is optionally substituted with one or more R⁸, and when n is 0 and m is not 0, R⁸ is attached directly to B;
R¹ and R² in combination form a fused 5-membered heteroaromatic ring that is optionally substituted with 1 or 2 substituents, said ring having at least one nitrogen, oxygen or sulfur atoms, but no more than 2 oxygen atoms or 2 sulfur atoms or 1 oxygen and 1 sulfur atom;
R³ is independently selected from H, optionally substituted alkyl optionally substituted alkenyl, optionally substituted alkynyl, optionally substituted cycloalkyl, optionally substituted cycloalkenyl, optionally substituted cycloalkynyl, optionally substituted aryl or optionally substituted heterocycle;
R⁴ and R⁵ are independently selected from H or optionally substituted alkyl, or R⁴ and R⁵ in combination form a 3-, 4-, 5- or 6-membered ring, which may also be optionally substituted;
R⁶ and R⁷ are independently selected from H, optionally substituted alkyl, optionally substituted alkenyl, optionally substituted alkynyl, optionally substituted cycloalkyl, optionally substituted cycloalkenyl, optionally substituted cycloalkynyl, optionally substituted heterocycle, optionally substituted aryl, or R⁶ and R⁷ in combination form a 3-, 4-, 5- or 6-membered ring, which may also be substituted;
R⁸ is independently selected from H, optionally substituted alkyl, optionally substituted alkenyl, optionally substituted alkynyl, optionally substituted cycloalkyl, optionally substituted cycloalkenyl, optionally substituted cycloalkynyl, optionally substituted aryl, or optionally substituted heterocycle;
R⁹ is independently selected from H, optionally substituted alkyl, optionally substituted alkenyl, optionally substituted alkynyl, optionally substituted cycloalkyl, optionally substituted cycloalkenyl, optionally substituted cycloalkynyl, optionally substituted aryl, or optionally substituted heterocycle.

In a particular embodiment of section 1 of the present invention provides a compound having a structural formula (I) as recited above wherein A is C═O or CH₂.

In a particular embodiment of section 1 of the present invention provides a compound having a structural formula (I) as recited above wherein A is C═O

In a particular embodiment of section 1 of the present invention provides a compound having a structural formula (I) as recited above wherein B is optionally substituted alkyl or optionally substituted heterocycle.

In a particular embodiment of section 1 of the present invention provides a compound having a structural formula (I) as recited above wherein B is optionally substituted C₁₋₄alkyl.

In a particular embodiment of section 1 of the present invention provides a compound having a structural formula (I) as recited above wherein B is an optionally substituted C₁₋₄alkyl wherein such substituent is independently selected from —NH₂, —OH, —NCH₃, —N(CH₃)₂, —N-cyclopropane, —N cyclobutane, azetidine, pyrrolidine, or piperidine.

In a particular embodiment of section 1 of the present invention provides a compound having a structural formula (I) as recited above wherein D is O optionally substituted with one or more R⁸.

In a particular embodiment of section 1 of the present invention provides a compound having a structural formula (1) as recited above wherein D is N optionally substituted with one or more R⁸.

In a particular embodiment of section 1 of the present invention provides a compound having a structural formula (I) as recited above wherein R¹ and R² in combination form a fused 5-membered heteroaromatic ring that is optionally substituted with 1 or 2 substituents, said ring having one nitrogen atom and one sulfur atom, or one nitrogen atom and one oxygen atom.

In a particular embodiment of section 1 of the present invention provides a compound having a structural formula (I) as recited above wherein R¹ and R² in combination form an optionally substituted fused isothiazole, or an optionally substituted fused isoxazole.

In a particular embodiment of section 1 of the present invention provides a compound having a structural formula (I) as recited above wherein R¹ and R² in combination form a fused 5-membered heteroaromatic ring that is optionally substituted with 1 or 2 substituents, said ring having one nitrogen atom and one sulfur atom, or one nitrogen atom and one oxygen atom and wherein said substituent is selected from C₁₋₆alkyl, or halogen.

In a particular embodiment of section 1 of the present invention provides a compound having a structural formula (I) as recited above wherein R³ is optionally substituted aryl.

In a particular embodiment of section 1 of the present invention provides a compound having a structural formula (I) as recited above wherein R³ is optionally substituted C₅₋₇aryl.

In a particular embodiment of section 1 of the present invention provides a compound having a structural formula (I) as recited above wherein R³ is optionally substituted C₅₋₇aryl wherein said substituent is independently selected from C₁₋₆alkyl, F, Cl, Br, or I.

In a particular embodiment of section 1 of the present invention provides a compound having a structural formula (I) as recited above wherein R⁴ and R⁵ are H.

In a particular embodiment of section 1 of the present invention provides a compound having a structural formula (I) as recited above wherein R⁶ and R⁷ are independently selected from H, or optionally substituted alkyl.

In a particular embodiment of section 1 of the present invention provides a compound having a structural formula (I) as recited above wherein R⁶ and R⁷ are independently selected from H, or C₁₋₆alkyl.

In a particular embodiment of section 1 of the present invention provides a compound having a structural formula (I) as recited above wherein R⁸ is independently selected from H, optionally substituted alkyl, or optionally substituted heterocycle.

In a particular embodiment of section 1 of the present invention provides a compound having a structural formula (I) as recited above wherein R⁹ is independently selected from optionally substituted aryl or optionally substituted heterocycle.

In a particular embodiment of section 1 of the present invention provides a compound having a structural formula (I) as recited above wherein R⁹ is independently selected from aryl or heterocycle either of which is optionally substituted with 1 or 2 substituents wherein said substituent is independently selected from —C₁₋₆alkyl, —OC₁₋₆alkyl, F, Cl, Br, I.

In a particular embodiment of section 1 of the present invention provides a compound having a structural formula (I) as recited above wherein R⁹ is C₅₋₇aryl optionally substituted with 1 or 2 substituents wherein said substituent is independently selected from —C₁₋₆alkyl, —OC₁₋₆alkyl, F, Cl, Br, I.

In a particular embodiment of section 1 of the present invention provides a compound having a structural formula (I) as recited above wherein:
n is 0;
A is CO or CH₂;
B is optionally substituted C₁₋₆alkyl;
R¹ and R² in combination form a fused 5 membered heteroaryl;
R³ is optionally substituted C₅₋₇aryl;
R⁴ and R⁵ are H;
R⁶ and R⁷ are independently selected from H or optionally substituted alkyl;
R⁹ is optionally substituted aryl or optionally substituted heterocycle.

In a particular embodiment of section 1 of the present invention provides a compound having a structural formula (I) as recited above wherein:
n is 0;
A is CO or CH₂;
B is C₁₋₆alkyl optionally substituted with one of the following —NH₂, —OH, —NCH₃, —N(CH₃)₂, —N-cyclopropane, —N cyclobutane, azetidine, pyrrolidine, or piperidine;
R¹ and R² in combination form a fused 5-membered heteroaromatic ring that is optionally substituted with 1 or 2 substituents, said ring having one nitrogen atom and one sulfur atom, or one nitrogen atom and one oxygen atom;
R³ is optionally substituted phenyl;
R⁴ and R⁵ are H;
R⁶ and R⁷ are independently selected from H or optionally substituted —C₁₋₆alkyl;
R⁹ is optionally substituted aryl;

In a particular embodiment of section 1 of the present invention provides a compound having a structural formula (I) as recited above wherein:
n is 0;
A is CO;
B is C₁₋₆alkyl optionally substituted with one of the following —NH₂, —OH, —NCH₃, —N(CH₃)₂, —N-cyclopropane, —N cyclobutane, azetidine, pyrrolidine, or piperidine;
R¹ and R² in combination form a fused 5-membered heteroaromatic ring that is optionally substituted with 1 or 2 substituents, said ring having one nitrogen atom and one sulfur atom, or one nitrogen atom and one oxygen atom and wherein said substituent is selected from C₁₋₆alkyl, or halogen;
R³ is optionally substituted C₅₋₇aryl wherein said substituent is independently selected from C₁₋₆alkyl, F, Cl, Br, or I;
R⁴ and R⁵ are H;
R⁶ and R⁷ are independently selected from H or —C₁₋₆alkyl;
R⁹ is C₅₋₇aryl optionally substituted with 1 or 2 substituents wherein said substituent is independently selected from —C₁₋₆alkyl, —OC₁₋₆alkyl, F, Cl, Br, I.

In a particular embodiment of section 1 of the present invention provides a compound having a structural formula (I) as recited above wherein:
n is 1;
A is CO or CH₂;
B is optionally substituted C₁₋₆alkyl;
D is N or O;
R¹ and R² in combination form a fused 5 membered heteroaryl;
R³ is optionally substituted aryl;
R⁴ and R⁵ are H;
R⁶ and R⁷ are H or optionally substituted alkyl;
R⁸ is H or optionally substituted C₁₋₆-alkyl;
R⁹ is optionally substituted aryl.

In a particular embodiment of section 1 of the present invention provides a compound having a structural formula (I) as recited above wherein:
n is 1;
A is CO or CH₂;
B is optionally substituted C₁₋₆alkyl;
D is N or O;
R¹ and R² in combination form a fused isothiazole, isoxazole;
R³ is optionally substituted phenyl;
R⁴ and R⁵ are H;
R⁶ and R⁷ are H or optionally substituted alkyl;
R⁸ is H or optionally substituted C₁₋₆alkyl;
R⁹ is optionally substituted phenyl.

In a particular embodiment of section 1 of the present invention provides a compound having a structural formula (I) selected from:
N-(3-Amino-propyl)-N-{1-[5-(4-fluoro-benzyl)-3-methyl-4-oxo-4,5-dihydro-isothiazolo[5,4-d]pyrimidin-6-yl]-propyl}-4-methyl-benzamide
N-(3-Amino-propyl)-N-[1-(5-benzyl-3-methyl-4-oxo-4,5-dihydro-isothiazolo[5,4-d]pyrimidin-6-yl)-propyl]-4-bromo-benzamide;
N-(3-Amino-propyl)-N-[1-(5-benzyl-3-methyl-4-oxo-4,5-dihydro-isothiazolo[5,4-d]pyrimidin-6-yl)-propyl]-4-chloro-benzamide;
N-(3-Amino-propyl)-N-[1-(5-benzyl-3-methyl-4-oxo-4,5-dihydro-isothiazolo[5,4-d]pyrimidin-6-yl)-propyl]-3-fluoro-4-methyl-benzamide;
N-(3-Amino-propyl)-N-[1-(5-benzyl-3-methyl-4-oxo-4,5-dihydro-isothiazolo[5,4-d]pyrimidin-6-yl)-propyl]-2,3-dichloro-benzamide;
Naphthalene-2-carboxylic acid (3-amino-propyl)-[1-(5-benzyl-3-methyl-4-oxo-4,5-dihydro-isothiazolo[5,4-d]pyrimidin-6-yl)-propyl]-amide;
Benzo[b]thiophene-2-carboxylic acid (3-amino-propyl)-[1-(5-benzyl-3-methyl-4-oxo-4,5-dihydro-isothiazolo[5,4-d]pyrimidin-6-yl)-propyl]-amide;
N-Azetidin-3-ylmethyl-N-[1-(5-benzyl-3-methyl-4-oxo-4,5-dihydro-isothiazolo[5,4-d]pyrimidin-6-yl)-propyl]-4-methyl-benzamide;
N-[1-(5-Benzyl-3-methyl-4-oxo-4,5-dihydro-isothiazolo[5,4-d]pyrimidin-6-yl)-propyl]-4-methyl-N-piperidin-3-ylmethyl-benzamide;
N-(2-Amino-ethyl)-N-[1-(5-benzyl-3-methyl-4-oxo-4,5-dihydro-isothiazolo[5,4-d]pyrimidin-6-yl)-propyl]-4-methyl-benzamide;
N-[1-(5-Benzyl-3-methyl-4-oxo-4,5-dihydro-isothiazolo[5,4-d]pyrimidin-6-yl)-propyl]-N-(2-dimethylamino-ethyl)-4-methyl-benzamide;
N-[1-(5-Benzyl-3-methyl-4-oxo-4,5-dihydro-isothiazolo[5,4-d]pyrimidin-6-yl)-propyl]-N-(3-dimethylamino-propyl)-4-methyl-benzamide;
N-[1-(5-Benzyl-3-methyl-4-oxo-4,5-dihydro-isothiazolo[5,4-d]pyrimidin-6-yl)propyl]-N-[3-(isopropylamino)propyl]-4-methylbenzamide;
N-[1-(5-benzyl-3-methyl-4-oxo-4,5-dihydro-isothiazolo[5,4-d]pyrimidin-6-yl)propyl]-N-[3-(cyclopropylamino)propyl]-4-methylbenzamide;
N-(3-azetidin-1-ylpropyl)-N-[1-(5-benzyl-3-methyl-4-oxo-4,5-dihydro-isothiazolo[5,4-d]pyrimidin-6-yl)propyl]-4-methylbenzamide;
N-[1-(5-benzyl-3-methyl-4-oxo-4,5-dihydro-isothiazolo[5,4-d]pyrimidin-6-yl)propyl]-4-methyl-N-[3-(3-pyrrolidin-1-ylpropyl) benzamide;
N-[1-(5-benzyl-3-methyl-4-oxo-4,5-dihydro-isothiazolo[5,4-d]pyrimidin-6-yl)propyl]-4-methyl-N-[3-(methylamino) propyl] benzamide;
N-[1-(5-Benzyl-3-methyl-4-oxo-4,5-dihydro-isothiazolo[5,4-d]pyrimidin-6-yl)-propyl]-N-(3-hydroxy-propyl)-4-methyl-benzamide;
N-(3-Amino-propyl)-N-[1-(5-benzyl-3-methyl-4-oxo-4,5-dihydro-isothiazolo[5,4-d]pyrimidin-6-yl)-propyl]-4-methyl-benzamide;
N-(3-Amino-propyl)-N-[1-(5-benzyl-3-methyl-4-oxo-4,5-dihydro-isothiazolo[5,4-d]pyrimidin-6-yl)-2-methyl-propyl]-4-methyl-benzamide;
N-(3-Amino-propyl)-N-{1-[5-(4-fluoro-benzyl)-3-methyl-4-oxo-4,5-dihydro-isothiazolo[5,4-d]pyrimidin-6-yl]-2-methyl-propyl}-4-methyl-benzamide;
5-Benzyl-6-{1-[(3-hydroxy-propyl)-(4-methyl-benzyl)-amino]-propyl}-3-methyl-5H-isothiazolo[5,4-d]pyrimidin-4-one;
N-(3-Amino-propyl)-N-{1-[5-(4-fluoro-benzyl)-3-methyl-4-oxo-4,5-dihydro-isoxazolo[5,4-d]pyrimidin-6-yl]-2-methyl-propyl}-4-methyl-benzamide;
N-(3-Amino-propyl)-3-fluoro-N-{1-[5-(4-fluoro-benzyl)-3-methyl-4-oxo-4,5-dihydro-isoxazolo[5,4-d]pyrimidin-6-yl]-2-methyl-propyl}-4-methyl-benzamide;
N-(3-Amino-propyl)-N-[1-(5-benzyl-3-methyl-4-oxo-4,5-dihydro-isoxazolo[5,4-d]pyrimidin-6-yl)-2-methyl-propyl]-3-fluoro-4-methyl-benzamide;
N-(3-Amino-propyl)-N-[1-(5-benzyl-3-methyl-4-oxo-4,5-dihydro-isoxazolo[5,4-d]pyrimidin-6-yl)-2-methyl-propyl]-4-methyl-benzamide;
N-(3-Amino-propyl)-N-[1-(5-benzyl-3-methyl-4-oxo-4,5-dihydro-isoxazolo[5,4-d]pyrimidin-6-yl)-propyl]-4-methyl-benzamide;
N-(3-Amino-propyl)-N-[1-(5-benzyl-3-methyl-4-oxo-4,5-dihydro-isoxazolo[5,4-d]pyrimidin-6-yl)-propyl]-4-fluoro-benzamide;
N-(3-Amino-propyl)-N-[1-(5-benzyl-3-methyl-4-oxo-4,5-dihydro-isoxazolo[5,4-d]pyrimidin-6-yl)-propyl]-2,3-dichloro-benzamide;
N-(3-Amino-propyl)-N-[1-(5-benzyl-3-methyl-4-oxo-4,5-dihydro-isoxazolo[5,4-d]pyrimidin-6-yl)-propyl]-3-fluoro-4-methyl-benzamide;
N-(3-Amino-propyl)-N-[1-(5-benzyl-3-methyl-4-oxo-4,5-dihydro-isoxazolo[5,4-d]pyrimidin-6-yl)-propyl]-4-methoxy-benzamide.

In a particular embodiment of section 1 of the present invention provides a compound according to any one of claims 1 to 27, for use as a medicament.

In a particular embodiment of section 1 of the present invention provides the use of a compound as defined in any one of claims 1 to 27, in the manufacture of a medicament for the treatment or prophylaxis of disorders associated with cancer.

In a particular embodiment of section 1 of the present invention provides a method for the treatment of cancer associated with comprising administering to a host in need of such treatment a therapeutically effective amount of a compound as defined in any one of claims 1 to 27.

In a particular embodiment of section 1 of the present invention provides a method for the prophylaxis treatment of cancers associated with comprising administering to a host in need of such treatment a therapeutically effective amount of a compound as defined in any one of claims 1 to 27.

In a particular embodiment of section 1 of the present invention provides a method for the treatment or prophylaxis of cancer comprising administering a therapeutically effective amount of a compound as defined in any one of claims 1 to 27 or a pharmaceutically acceptable salt as claimed in any one of claims 1 to 27.

In a particular embodiment of section 1 of the present invention provides a method of producing a cell cycle inhibitory (anti-cell-proliferation) effect in a warm-blooded animal, such as man, in need of such treatment with comprises administering to said animal an effective amount of a compound as claimed in any of claims 1 to 27.

In a particular embodiment of section 1 of the present invention provides a pharmaceutical composition comprising a compound as defined in any one of claims 1 to 27, or a pharmaceutically acceptable salt or in vivo hydrolysable ester thereof, together with at least one pharmaceutically acceptable carrier, diluent or excipient.

In a particular embodiment of section 1 of the present invention provides a process for preparing a compound of structural formula (I) as claimed in claim 1 or a pharmaceutically acceptable salt or an in vivo hydrolysable ester thereof which process comprises: 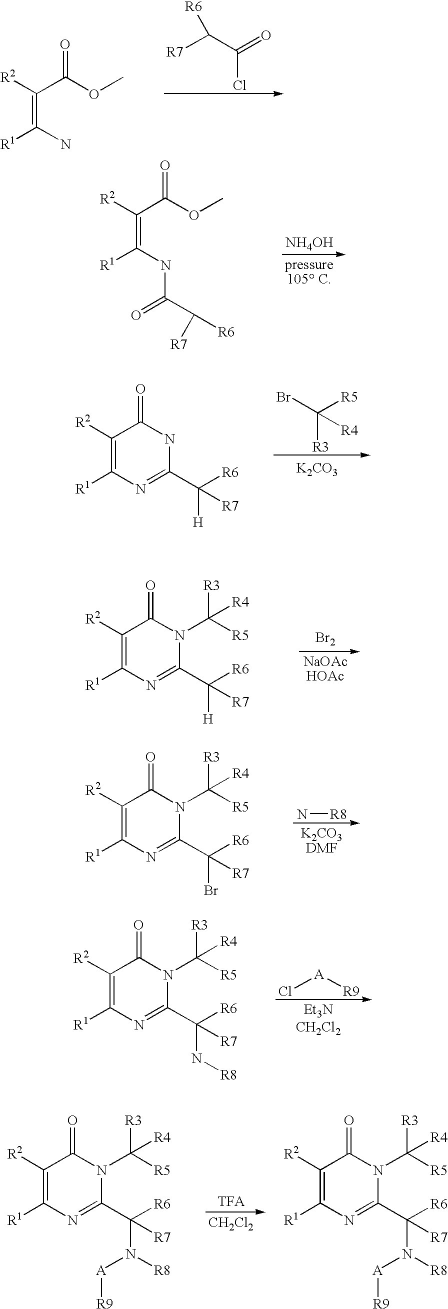
Section 1: Definitions

The definitions set forth in this section of section 1, are intended to clarify terms used throughout this application. In section 1 the term “herein” means within section 1.

Unless specified otherwise within section 1, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

In section 1, the term “Cₘ₋ₙ” or “Cₘ₋ₙ group” used alone or as a prefix, refers to any group having m to n carbon atoms. For example C₁₋₆ means 1, 2, 3, 4, 5, or 6 carbon atoms.

In section 1, the term “hydrocarbon” used alone or as a suffix or prefix, refers to any structure comprising only carbon and hydrogen atoms up to 14 carbon atoms.

In section 1, the term “hydrocarbon radical” or “hydrocarbyl” used alone or as a suffix or prefix, refers to any structure as a result of removing one or more hydrogens from a hydrocarbon.

In section 1, the term “alkyl” used alone or as a suffix or prefix, refers to monovalent straight or branched chain hydrocarbon radicals comprising 1 to about 12 carbon atoms. Unless otherwise specified, “alkyl” general includes both saturated alkyl and unsaturated alkyl.

In section 1, the term “alkylene” used alone or as suffix or prefix, refers to divalent straight or branched chain hydrocarbon radicals comprising 1 to about 12 carbon atoms, which serves to links two structures together.

In section 1, the term “alkenyl” used alone or as suffix or prefix, refers to a monovalent straight or branched chain hydrocarbon radical having at least one carbon-carbon double bond and comprising at least 2 up to about 12 carbon atoms.

In section 1, the term “alkynyl” used alone or as suffix or prefix, refers to a monovalent straight or branched chain hydrocarbon radical having at least one carbon-carbon triple bond and comprising at least 2 up to about 12 carbon atoms.

In section 1, the term “cycloalkyl,” used alone or as suffix or prefix, refers to a monovalent ring-containing hydrocarbon radical comprising at least 3 up to about 12 carbon atoms.

In section 1, the term “cycloalkenyl” used alone or as suffix or prefix, refers to a monovalent ring-containing hydrocarbon radical having at least one carbon-carbon double bond and comprising at least 3 up to about 12 carbon atoms.

In section 1, the term “cycloalkynyl” used alone or as suffix or prefix, refers to a monovalent ring-containing hydrocarbon radical having at least one carbon-carbon triple bond and comprising about 7 up to about 12 carbon atoms.

In section 1, the term “aryl” used alone or as suffix or prefix, refers to a hydrocarbon radical having one or more polyunsaturated carbon rings having aromatic character, (e.g., 4n+2 delocalized electrons) and comprising 5 up to about 14 carbon atoms, wherein the radical is located on a carbon of the aromatic ring.

In section 1, the term “non-aromatic group” or “non-aromatic” used alone, as suffix or as prefix, refers to a chemical group or radical that does not contain a ring having aromatic character (e.g., 4n+2 delocalized electrons).

In section 1, the term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to link two structures together.

In section 1, the term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). In section 1, heterocycle may be saturated or unsaturated, containing one or more double bonds, and heterocycle may contain more than one ring. When a heterocycle contains more than one ring, in section 1, the rings may be fused or unfused. Fused rings in section 1 generally refer to at least two rings share two atoms there between. Heterocycle in section 1 may have aromatic character or may not have aromatic character.

In section 1, the term “heteroalkyl” used alone or as a suffix or prefix, refers to a radical formed as a result of replacing one or more carbon atom of an alkyl with one or more heteroatoms selected from N, O, P and S.

In section 1, the term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

In section 1, the term “heterocyclic group,” “heterocyclic moiety,” “heterocyclic,” or “heterocyclo” used alone or as a suffix or prefix, refers to a radical derived from a heterocycle by removing one or more hydrogens therefrom.

In section 1, the term “heterocycle” used alone or as a suffix or prefix, refers a radical derived from a heterocycle by removing one hydrogen from a carbon of a ring of the heterocycle.

In section 1, the term “heterocycleene” used alone or as a suffix or prefix, refers to a divalent radical derived from a heterocycle by removing two hydrogens therefrom, which serves to links two structures together.

In section 1, the term “heteroaryl” used alone or as a suffix or prefix, refers to a heterocycle having aromatic character, wherein the radical of the heterocycle is located on a carbon of an aromatic ring of the heterocycle.

In section 1, the term “heterocylcoalkyl” used alone or as a suffix or prefix, refers to a heterocycle that does not have aromatic character.

In section 1, the term “heteroarylene” used alone or as a suffix or prefix, refers to a heterocyclylene having aromatic character.

In section 1, the term “heterocycloalkylene” used alone or as a suffix or prefix, refers to a heterocyclylene that does not have aromatic character.

In section 1, the term “six-membered” used as prefix refers to a group having a ring that contains six ring atoms.

In section 1, the term “five-membered” used as prefix refers to a group having a ring that contains five ring atoms.

A five-membered ring heteroaryl in section 1 is a heteroaryl with a ring having five ring atoms wherein 1, 2 or 3 ring atoms are independently selected from N, O and S.

Exemplary five-membered ring heteroaryls of section 1 are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

In section 1, a six-membered ring heteroaryl is a heteroaryl with a ring having six ring atoms wherein 1, 2 or 3 ring atoms are independently selected from N, O and S.

Exemplary six-membered ring heteroaryls of section 1 are pyridyl, pyrazinyl, pyrimidinyl, triazinyl and pyridazinyl.

As used in section 1, the term “optionally substituted,” as used herein, means that substitution is optional and therefore it is possible for the designated atom or molecule to be unsubstituted. In the event a substitution is desired in section 1 then such substitution means that any number of hydrogens on the designated atom is replaced with a selection from the indicated group, provided that the normal valency of the designated atom is not exceeded, and that the substitution results in a stable compound. For example in section 1 when a substituent is keto (i.e., ═O), then 2 hydrogens on the atom are replaced. If no selection is provided in section 1 then the substituent shall be selected from: —OC₁₋₆alkyl, —C₁₋₆alkyl, F, Cl, Br, I, N, O, S, P—NH₂, —OH, —NCH₃, —N(CH₃)₂, —N-cyclopropane, —N cyclobutane, azetidine, pyrrolidine, piperidine. Exemplary chemical groups containing one or more heteroatoms in section 1 include heterocycle, —NO₂, —OR, —CF₃, —C(═O)R, —C(═O)OH, —SH, —NHR, —NR₂, —SR, —SO₃H, —SO₂R, —S(═O)R, —CN, —C(═O)OR, —C(═O)NR₂, —NRC(═O)R, oxo (═O), imino (═NR), thio (═S), and oximino (═N—OR), wherein each “R” is a C₁₋₁₂hydrocarbyl. For example in section 1, substituted phenyl may refer to nitrophenyl, pyridylphenyl, methoxyphenyl, chlorophenyl, aminophenyl, etc., wherein the nitro, pyridyl, methoxy, chloro, and amino groups may replace any suitable hydrogen on the phenyl ring.

In section 1, the term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example in section 1, a “phenyl substituted by nitro” refers to nitrophenyl.

In section 1, heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition in section 1, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle in section 1 encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above in section 1, heterocycle in section 1 includes polycyclic heterocycles wherein the ring fusion between two or more rings includes more than one bond common to both rings and more than two atoms common to both rings. Examples of such bridged heterocycles of section 1 include quinuclidine, diazabicyclo[2.2.1]heptane and 7-oxabicyclo[2.2.1]heptane.

Heterocycle in section 1 includes, for example, monocyclic heterocycles, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocycle in section 1 includes aromatic heterocycles or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocycle in section 1 encompasses polycyclic heterocycles (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocycles described above in section 1, heterocycle in section 1 includes polycyclic heterocycles wherein the ring fusion between two or more rings includes more than one bond common to both rings and more than two atoms common to both rings. Examples of such bridged heterocycles in section 1 include quinuclidinyl, diazabicyclo[2.2.1]heptyl; and 7-oxabicyclo[2.2.1]heptyl.

In section 1, the term “alkoxy” used alone or as a suffix or prefix, refers to radicals of the general formula —O—R, wherein —R is selected from a hydrocarbon radical. Exemplary alkoxy in section 1 includes methoxy, ethoxy, propoxy, isopropoxy, butoxy, t-butoxy, isobutoxy, cyclopropylmethoxy, allyloxy, and propargyloxy.

In section 1, the term “aryloxy” used alone or as suffix or prefix, refers to radicals of the general formula —O—Ar, wherein —Ar is an aryl.

In section 1, the term “heteroaryloxy” used alone or as suffix or prefix, refers to radicals of the general formula —O—Ar′, wherein —Ar′ is a heteroaryl.

In section 1, the term “amine” or “amino” used alone or as a suffix or prefix, refers to radicals of the general formula —NRR′, wherein R and R′ are independently selected from hydrogen or a hydrocarbon radical.

In section 1, “acyl” used alone, as a prefix or suffix, means —C(═O)—R, wherein —R is an optionally substituted hydrocarbyl, hydrogen, amino or alkoxy. Acyl groups in section 1 include, for example, acetyl, propionyl, benzoyl, phenyl acetyl, carboethoxy, and dimethylcarbamoyl.

In section 1, halogen includes fluorine, chlorine, bromine and iodine.

In section 1, “halogenated,” used as a prefix of a group, means one or more hydrogens on the group is replaced with one or more halogens.

In section 1, “RT” or “rt” means room temperature.

A first ring group being “fused” with a second ring group in section 1 means the first ring and the second ring share at least two atoms therebetween.

“Link,” “linked,” or “linking,” unless otherwise specified, in section 1 means covalently linked or bonded.

In section 1, when a first group, structure, or atom is “directly connected” to a second group, structure or atom, at least one atom of the first group, structure or atom forms a chemical bond with at least one atom of the second group, structure or atom.

In section 1, “saturated carbon” means a carbon atom in a structure, molecule or group wherein all the bonds connected to this carbon atom are single bond. In other words, in section 1, there is no double or triple bonds connected to this carbon atom and this carbon atom generally adopts an sp³ atomic orbital hybridization.

In section 1, “unsaturated carbon” means a carbon atom in a structure, molecule or group wherein at least one bond connected to this carbon atom is not a single bond. In other words in section 1, there is at least one double or triple bond connected to this carbon atom and this carbon atom generally adopts a sp or sp² atomic orbital hybridization.

In section 1, when any variable (e.g., R¹, R⁴, R^{a}, R^{e} etc.) occurs more than one time in any constituent or formula for a compound, its definition at each occurrence is independent of its definition at every other occurrence. Thus, for example, in section 1 if a group is shown to be substituted with 0-3 R¹, then said group may optionally be substituted with 0,1, 2 or 3 R¹ groups and R^{e} at each occurrence is selected independently from the definition of R^{e}. Also in section 1, combinations of substituents and/or variables are permissible only if such combinations result in stable compounds.

In section 1, a variety of compounds in the present invention may exist in particular geometric or stereoisomeric forms. The present invention described in section 1 takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms in section 1 may be present in a substituent such as an alkyl group. All such isomers in section 1, as well as mixtures thereof, are intended to be included in this invention. The compounds described in section 1 may have asymmetric centres. Compounds of the present invention as described in section 1, containing an asymmetrically substituted atom may be isolated in optically active or racemic forms. It is well known in the art how to prepare optically active forms, such as by resolution of racemic forms or by synthesis from optically active starting materials. When required in section 1, separation of the racemic material can be achieved by methods known in the art. Many geometric isomers of olefins, C═N double bonds, and the like can also be present in the compounds described in section 1, and all such stable isomers are contemplated in the present invention. Cis and trans geometric isomers of the compounds of the present invention are described in section 1 and may be isolated as a mixture of isomers or as separated isomeric forms. All chiral, diastereomeric, racemic forms and all geometric isomeric forms of a structure in section 1 are intended, unless the specific stereochemistry or isomeric form is specifically indicated.

In section 1, when a bond to a substituent is shown to cross a bond connecting two atoms in a ring, then such substituent may be bonded to any atom on the ring. In section 1, when a substituent is listed without indicating the atom via which such substituent is bonded to the rest of the compound of a given formula, then such substituent may be bonded via any atom in such substituent. In section 1, combinations of substituents and/or variables are permissible only if such combinations result in stable compounds.

In section 1, as used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In section 1, as used herein, “pharmaceutically acceptable salts” refer to derivatives of the disclosed compounds wherein the parent compound is modified by making acid or base salts thereof. Examples of pharmaceutically acceptable salts in section 1 include, but are not limited to, mineral or organic acid salts of basic residues such as amines; alkali or organic salts of acidic residues such as carboxylic acids; and the like. The pharmaceutically acceptable salts in section 1 include the conventional non-toxic salts or the quaternary ammonium salts of the parent compound formed, for example, from non-toxic inorganic or organic acids. For example in section 1, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention as described in section 1 can be synthesized from the parent compound that contains a basic or acidic moiety by conventional chemical methods. Generally, in section 1 such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. In section 1, lists of suitable salts are found in Remington's Pharmaceutical Sciences, 17th ed., Mack Publishing Company, Easton, Pa., 1985, p. 1418, the disclosure of which is hereby incorporated by reference.

In section 1, “prodrugs” are intended to include any covalently bonded carriers that release the active parent drug according to formula (I) in vivo when such prodrug is administered to a mammalian subject. In section 1, prodrugs of a compound of formula (I) are prepared by modifying functional groups present in the compound in such a way that the modifications are cleaved, either in routine manipulation or in vivo, to the parent compound. In section 1, prodrugs include compounds of formula (I) wherein a hydroxy, amino, or sulfhydryl group is bonded to any group that, when the prodrug or compound of formula (I) is administered to a mammalian subject, cleaves to form a free hydroxyl, free amino, or free sulfhydryl group, respectively. In section 1, examples of prodrugs include, but are not limited to, acetate, formate and benzoate derivatives of alcohol and amine functional groups in the compounds of formula (I), and the like.

Section 1: Formulations

In section 1, compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

In section 1, the dosage will depend on the route of administration, the severity of the disease, age and weight of the patient and other factors normally considered by the attending physician, when determining the individual regimen and dosage level as the most appropriate for a particular patient.

In section 1, an effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

In section 1, for preparing pharmaceutical compositions from the compounds of this invention, inert, pharmaceutically acceptable carriers can be either solid or liquid. In section 1, solid form preparations include powders, tablets, dispersible granules, capsules, cachets, and suppositories.

In section 1, a solid carrier can be one or more substances, which may also act as diluents, flavoring agents, solubilizers, lubricants, suspending agents, binders, or tablet disintegrating agents; it can also be an encapsulating material.

In powders of section 1, the carrier is a finely divided solid, which is in a mixture with the finely divided active component. In tablets of section 1, the active component is mixed with the carrier having the necessary binding properties in suitable proportions and compacted in the shape and size desired.

In section 1, for preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

In section 1, suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention as described in section 1 are capable of forming salts with various inorganic and organic acids and bases and such salts are also within the scope of this invention. Examples of such acid addition salts of section 1 include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts of section 1 include ammonium salts, alkali metal salts such as sodium, lithium and potassium salts, alkaline earth metal salts such as aluminium, calcium and magnesium salts, salts with organic bases such as dicyclohexylamine salts, N-methyl-D-glucamine, and salts with amino acids such as arginine, lysine, ornithine, and so forth. In section 1, also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. In section 1, non-toxic physiologically-acceptable salts are preferred, although other salts are also useful, such as in isolating or purifying the product.

In section 1, the salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In section 1, in order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In section 1, in addition to the compounds of the present invention, the pharmaceutical composition of this invention may also contain, or be co-administered (simultaneously or sequentially) with, one or more pharmacological agents of value in treating one or more disease conditions referred to herein.

In section 1, the term composition is intended to include the formulation of the active component or a pharmaceutically acceptable salt with a pharmaceutically acceptable carrier. For example in section 1, this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

In section 1, liquid form compositions include solutions, suspensions, and emulsions. Sterile water or water-propylene glycol solutions of the active compounds in section 1 may be mentioned as an example of liquid preparations suitable for parenteral administration. In section 1, liquid compositions can also be formulated in solution in aqueous polyethylene glycol solution. In section 1, aqueous solutions for oral administration can be prepared by dissolving the active component in water and adding suitable colorants, flavoring agents, stabilizers, and thickening agents as desired. In section 1, aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

In section 1, the pharmaceutical compositions can be in unit dosage form. In such form, the composition is divided into unit doses containing appropriate quantities of the active component. In section 1, the unit dosage form can be a packaged preparation, the package containing discrete quantities of the preparations, for example, packeted tablets, capsules, and powders in vials or ampoules. In section 1, the unit dosage form can also be a capsule, cachet, or tablet itself, or it can be the appropriate number of any of these packaged forms.

Section 1: Combinations

The anti-cancer treatment defined in section 1 may be applied as a sole therapy or may involve, in addition to the compound of the invention, conventional surgery or radiotherapy or chemotherapy. Such chemotherapy in section 1 may include one or more of the following categories of anti-tumour agents:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In section 1, such conjoint treatment may be achieved by way of the simultaneous, sequential or separate dosing of the individual components of the treatment. In section 1, such combination products employ the compounds of this invention within the dosage range described hereinbefore and the other pharmaceutically-active agent within its approved dosage range.

Section 1: Synthesis

The compounds of the present invention as described in section 1 can be prepared in a number of ways well known to one skilled in the art of organic synthesis. The compounds of the present invention of section 1 can be synthesized using the methods described below, together with synthetic methods known in the art of synthetic organic chemistry, or variations thereon as appreciated by those skilled in the art. Such methods include in section 1, but are not limited to, those described below. All references cited in section 1 are hereby incorporated in their entirety by reference.

In section 1, the novel compounds of this invention may be prepared using the reactions and techniques described herein. In section 1, the reactions are performed in solvents appropriate to the reagents and materials employed and are suitable for the transformations being effected. Also, in the description of the synthetic methods described below in section 1, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule in section 1 must be compatible with the reagents and reactions proposed. Such restrictions to the substituents in section 1, which are compatible with the reaction conditions, will be readily apparent to one skilled in the art and alternate methods must then be used.

The starting materials for the Examples contained in section 1 are either commercially available or are readily prepared by standard methods from known materials. For example the following reactions are illustrations but not limitations of the preparation of some of the starting materials and examples used herein.

Chemical abbreviations used in the Examples of section 1 are defined as follows: “BOC” denotes N-tert-butoxycarbonyl, “CBZ” denotes carbobenzyloxy; “DIEA” denotes N,N-diisopropylethylamine, “DMF” denotes N,N-dimethylformamide; “THF” denotes tetrahydrofuran, “ether” denotes ethyl ether, “min.” denotes minutes; “h” or hr denotes hours; “RT” or “r.t”. denotes room temperature; “SM” denotes starting material, “MS” denotes mass spectrometry, “RM” denotes reaction mixture, “NMR” denotes nuclear magnetic resonance, “TLC” denotes thin layer chromatography, “LC” denotes liquid chromatography, “HPLC” denotes high pressure liquid chromatography, “TFA” denotes trifluoroacetic acid, “DMSO” denotes dimethyl sulfoxide, “EtOAc” denotes ethyl acetate. In section 1, unless otherwise noted, organic solutions were “dried” over anhydrous sodium sulfate.

Examples of such processes of section 1 are illustrated below: 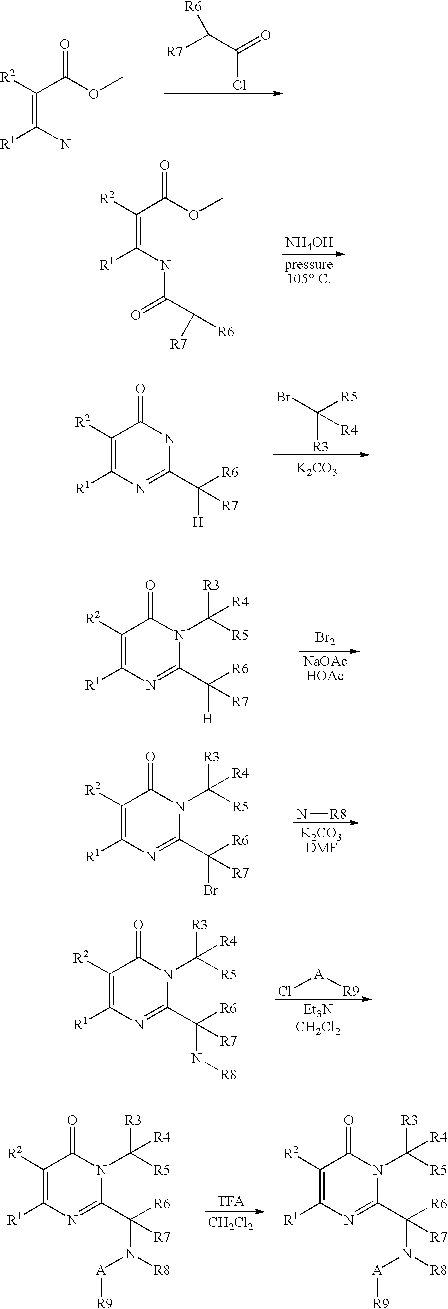
Section 1: Method 1

### 2-(1-Ethoxy-ethylidene)-malononitrile

Triethyl orthoacetate (97 g, 0.6 mol), malononitrile (33 g, 0.5 mol) and glacial acetic acid (1.5 g) were placed in a 1 L flask equipped with a stirrer, thermometer and a Vigreux column (20×1 in.) on top of which a distillation condenser was placed. The reaction mixture was heated and ethyl alcohol began to distill when the temperature of the reaction mixture was about 85-90° C. After about 40 min., the temperature of the reaction mixture reached 140° C. Then the reaction was concentrated in a rotary evaporator to remove the low-boiling materials and the residue was crystallized from absolute alcohol to yield the pure product (62.2 g, 91%) as a light yellow solid [mp 91.6° C. (lit. 90-92° C., MCCall. M. A. J. Org. Chem. 1962, 27, 2433-2439.)].

Section 1: Method 2

### (E)-2-Cyano-3-ethoxy-but-2-enethioic acid amide

2-(1-Ethoxy-ethylidene)-malononitrile (Section 1: Method 1) (62 g, 0.45 mol) was dissolved in anhydrous benzene (800 mL) and 1 mL of triethylamine was added as catalyst. The mixture was stirred and hydrogen sulfide was bubbled into this solution for 40 min and a solid formed. The precipitated solid was filtered off and dried. The solid was recrystallized from absolute alcohol (100 mL) filtered and dried to isolate the pure (E)-2-cyano-3-ethoxy-but-2-enethioic acid amide (19.3 g, 25%) as a light brown crystals.

Section 1: Method 3

### (E)-3-Amino-2-cyano-but-2-enethioic acid amide

(E)-2-Cyano-3-ethoxy-but-2-enethioic acid amide (Section 1: Method 2) (19.2 g, 0.136 mol) was dissolved in a saturated solution of ammonia in methanol (500 mL) and stirred at r.t. overnight. The reaction mixture was concentrated and the residue was dissolved in hot water (600 mL) and the undissoved solid was filtered and dried to recover 6 g of the starting thiocrotonamide. The aqueous solution on standing overnight provided the pure (E)-3-amino-2-cyano-but-2-enethioic acid amide (6.85 g, 63%) as off-white crystals. ¹H NMR (300 MHz, DMSO-d₆) δ 2.22 (s, 3H), 7.73 (bs, 1H), 8.53 (bs, 1H), 9.01 (bs, 1H), 11.60 (bs, 1H).

Section 1: Method 4

### 5-Amino-3-methylisothiazole-4-carbonitrile

To a stirred solution of (E)-3-amino-2-cyano-but-2-enethioic acid amide (Section 1: Method 3) (6.83 g, 48.4 mmol) in methanol (300 mL) was added dropwise 13.6 mL (124 mmol.) of 30% hydrogen peroxide. The mixture was stirred at 60° C. for 4 h and evaporated to 60 mL in a rotary evaporator and cooled in an ice-bath. The crystallized product was filtered off and recrystallized from ethyl acetate to provide the pure product 5-amino-3-methylisothiazole-4-carbonitrile (5.41 g, 80%) as a white crystalline solid. ¹H NMR (300 MHz, DMSO-d₆) δ 2.24 (s, 3H), 8.00 (bs, 2H).

Section 1: Method 5

### N-(4-Cyano-3-methyl-isothiazol-5-yl)-butyramide

To a solution of the amine (Section 1: Method 4) (5.31 g, 38.2 mmol) in CH₂Cl₂ (200 mL) at 0° C., NEt₃ (5 g, 50 mmol) was added followed by the dropwise addition of a solution of the butyryl chloride (4.88 g, 45.8 mmol) in CH₂Cl₂ (50 mL). After the completion of the addition the reaction mixture was allowed to warm to r.t. and stirred overnight. The reaction mixture was washed with water (100 mL), 1N HCl (100 mL), brine (200 mL) and dried over Na₂SO₄. Concentration of the CH₂Cl₂ layer provided the product which was triturated from CH₂Cl₂/hexanes (1/10) and filtered off to isolate the pure N-(4-cyano-3-methyl-isothiazol-5-yl)-butyramide (7.57 g, 95%) as an orange solid.

Section 1: Method 6

### 5-Butyrylamino-3-methyl-isothiazole-4-carboxylic acid amide

To a solution of N-(4-cyano-3-methyl-isothiazol-5-yl)-butyramide (Section 1: Method 5) (4.18 g, 20 mmol) in 30% aqueous NH₄OH (250 mL), was added dropwise 100 mL of hydrogen peroxide at r.t. After the completion of the addition the reaction mixture was stirred at 60° C. overnight after which the TLC showed the complete disappearance of SM. The reaction mixture was cooled and extracted with chloroform (3×100 mL). The organic layer was dried (Na₂SO₄) and concentrated to get the pure 5-butyrylamino-3-methyl-isothiazole-4-carboxylic acid amide (2.9 g, 72%) as a white solid. ¹H NMR (300 MHz, CDCl₃) δ 1.03 (t, 3H), 1.79 (m, 2H), 2.54 (t, 3H), 2.69 (s, 3H), 5.97 (bs, 2H), 11.78 (bs, 1H).

Section 1: Method 7

### 3-Methyl-6-propyl-SH-isothiazolo[5,4-d]pyrimidin-4-one

5-Butyrylamino-3-methyl-isothiazole-4-carboxylic acid amide (Section 1: Method 6) (1.9 g, 8.3 mmol) was suspended in 75 mL of 30% NH₃ and then was heated to 140° C. for 4 h in a pressure reactor. The mixture was cooled and neutralized to pH 8. The precipitated 3-methyl-6-propyl-5H-isothiazolo[5,4-d]pyrimidin-4-one was filtered off, washed with water (100 mL) and dried in vacuum oven at 40° C. overnight to get 800 mg (34%) of pure product. ¹H NMR (300 MHz, CDCl₃) δ 1.03 (t, 3H), 1.74 (m, 2H), 2.67 (t, 3H), 2.78 (s, 3H).

Section 1: Method 8

### 5-Benzyl-3-methyl-6-propyl-5H-isothiazolo[5,4-d]pyrimidin-4-one

To a solution of 3-methyl-6-propyl-5H-isothiazolo[5,4-d]pyrimidin-4-one (Section 1: Method 7) (800 mg, 3.8 mmol) in 20 mL of anhydrous DMF was added 1.38 g (10 mmol) of anhydrous K₂CO₃ followed by benzyl bromide (655 mg, 3.8 mmol) and the mixture was stirred at room temperature overnight. The TLC of the reaction mixture showed the complete disappearance of the SM. The reaction mixture was poured into ice cold water and extracted with EtOAc (3×100 mL). The combined extracts were washed with water (100 mL), brine (100 mL), dried (Na₂SO₄) and concentrated. The TLC and the 1H NMR showed the presence of two products N alkylated as well as O-alkylated products in a ratio of 1:1. The products were separated by column (silica gel, 116 g) chromatography using 10-20% EtOAc in hexanes. The desired N-alkylated product 5-benzyl-3-methyl-6-propyl-5H-isothiazolo[5,4-d]pyrimidin-4-one was isolated as white crystalline solid (369 mg, 32%). ¹H NMR (300 MHz, CDCl₃) δ 0.96 (t, 3H), 1.71-1.84 (m, 2H), 2.73 (t, 3H), 2.81 (s, 3H), 5.38 (s, 2H), 7.14-7.38 (m, 5H):

The following compounds were synthesized according to Section 1: Method 8:

| Section 1: | | | |
|---|---|---|---|
| Method # | Compound Name | m/z | Alkylating agent |
| 8a | 5-(4-Fluoro-benzyl)-3-methyl-6- | 318 | 4-fluorobenzyl |
| | propyl-isothiazolo[5,4- | (MH⁺) | bromide |
| | d]pyrimidin-4-one | | |

Section 1: Method 9

### 5-Benzyl-6-(1-bromo-propyl)-3-methyl-5-H-isothiazolo[5,4-d]pyrimidin-4-one

To a solution of 5-benzyl-3-methyl-6-propyl-5H-isothiazolo[5,4-d]pyrimidin-4-one (Section 1: Method 8) (369 mg, 1.23 mmol) and sodium acetate (1 g) in acetic acid (5 mL) at 100° C., a solution of the bromine (318 mg, 2 mmol) in acetic acid (10 mL) was added dropwise [The next drop of bromine was added only after the previous drop had reacted completely by monitoring the decolorization] over a period of 20 minutes. The reaction mixture was cooled after the addition and the TLC (eluent 10% EtOAc in hexanes) and MS showed the complete disappearance of the SM and only the product. The reaction mixture was poured into ice water and extracted with EtOAc (3×60 mL) and the organic layers were combined and washed with 2% sodium thiosulfate solution (60 mL), water (100 mL), brine (100 mL) and dried over Na₂SO₄. Concentration of the organic layer provided the pure 5-benzyl-6-(1-bromo-propyl)-3-methyl-5-H-isothiazolo[5,4-d]pyrimidin-4-one, (460 mg, 100%) as white crystalline solid. ¹H NMR (300 MHz, CDCl₃) δ 0.76 (t, 3H), 2.1-2.47 (m, 2H), 2.84 (s, 3H), 4.62 (t, 1H), 4.88 (d, 1H), 6.20 (d, 1H), 7.10-7.40 (m, 5H).

The following compounds were synthesized according to Section 1: Method 9:

| Section 1: | | | |
|---|---|---|---|
| Method # | Compound Name | m/z | SM |
| 9a | 6-(1-bromopropyl)-5-[(4- | 397 | Section 1: |
| | fluorophenyl)methyl)]-3-methyl- | (MH⁺) | Method 8a |
| | isothiazolo[5,4-d]pyrimidin-4(5H)-one | | |

Section 1: Method 10

### {3-[1-(5-Benzyl-3-methyl-4-oxo-4,5-dihydro-isothiazolo[5,4-d]pyrimidin-6-yl)-propylamino]-propyl}-carbamic Acid tert-Butyl Ester

To a solution of the bromide (Section 1: Method 9) (0.46 g, 1.22 mmol) in anhydrous ethanol (20 mL), was added tert-butyl 3-aminopropyl-carbamate (0.211 g, 1.22 mmol) followed by the addition of anhydrous diisopropylethylamine (0.258 g, 2 mmol) and the mixture was stirred at reflux for 16 hours. The TLC of the RM showed the complete disappearance of the starting bromide. The reaction mixture was poured into ice water (200 mL) and extracted with EtOAc (3×100 mL). The organic layer was washed with water (100 mL), brine (100 mL) and dried (Na₂SO₄). Concentration of the organic layer provided the product which was purified by column (silica gel) chromatography using 30-50% EtOAc in hexanes to isolate the pure amine {3-[1-(5-Benzyl-3-methyl-4-oxo-4,5-dihydro-isothiazolo[5,4-d]pyrimidin-6-yl)-propylamino]-propyl}-carbamic Acid tert-Butyl Ester (0.1 g, 17%) as a white foam. ¹H NMR (300 MHz, CDCl₃) δ 0.95 (t, 3H), 1.33 (t, 2H), 1.42 (s, 9H), 1.49-1.51 (m, 2H), 1.87-1.99 (m, 1H), 2.35-2.45 (m, 1H), 2.83 (s, 3H), 2.92-3.20 (m, 2H), 3.64-3.70 (m, 1H), 4.98 (d, 114), 5.17 (bs, 1H), 5.85 (d, 1H), 7.10-7.40 (m, 5H).

The following compounds were synthesized according to Section 1: Method 10:

| Section | | | | |
|---|---|---|---|---|
| 1: | | | | |
| Method | | | | |
| # | Compound Name | m/z | SM | Amine |
| 10a | [3-({1-[5-(4-fluorobenzyl)- | 490 | Section | tert-butyl |
| | 3-methyl-4-oxo-4,5- | (MH⁺) | 1: | 3- |
| | dihydro-isothiazolo[5,4- | | Method | aminopropyl- |
| | d]pyrimidin-6-yl]- | | 9a | carbamate |
| | propyl}amino)-propyl}- | | | |
| | carbamic Acid tert-Butyl | | | |
| | Ester | | | |

Section 1: Method 11

### {3-[1-(5-Benzyl-3-methyl-4-oxo-4,5-dihydro-isothiazolo[5,4-d]pyrimidin-6-yl)-propylamino]-propyl}-carbamic acid tert-butyl ester

To a solution of the bromide (Section 1: Method 9) (0.1 g, 0.26 mmol) in anhydrous dichloromethane (5 mL), was added anhydrous diisopropylethylamine (100 μl, 0.52 mmol) followed by tert-butyl 3-aminopropyl-carbamate (0.10 g, 0.52 mmol). The reaction mixture was microwaved at 120° C. for 2 h. The LC/MS of the RM showed the complete disappearance of the starting bromide. The reaction mixture was evaporated to dryness the product was purified by column (silica gel) chromatography using 40-60% EtOAc in hexanes to isolate the pure amine {3-[1-(5-Benzyl-3-methyl-4-oxo-4,5-dihydro-isothiazolo[5,4-d]pyrimidin-6-yl)-propylamino]-propyl}-carbamic Acid tert-Butyl Ester (0.085 g, 64%). m/z 472 (MH⁺).

The following compounds were synthesized according to Section 1: Method 11:

| Section 1: | | | | |
|---|---|---|---|---|
| Method # | Compound Name | m/z | SM | Amine |
| 11a | {2-[1-(5-Benzyl-3-methyl-4-oxo-4,5-dihydro- | 458 | Section | (2-Amino- |
| | isothiazolo[5,4-d]pyrimidin-6-yl)- | (MH⁺) | 1: | ethyl)- |
| | propylamino]-ethyl}-carbamic acid tert-butyl | | Method 9 | carbamic |
| | ester | | | acid tert- |
| | | | | butyl ester |
| 11b | 3-{[1-(5-Benzyl-3-methyl-4-oxo-4,5-dihydro- | 484 | Section | 3- |
| | isothiazolo[5,4-d]pyrimidin-6-yl)- | (MH⁺) | 1: | aminomethyl- |
| | propylamino]-methyl}-azetidine-1-carboxylic | | Method 9 | azetidine- |
| | acid tert-butyl ester | | | 1- |
| | | | | carboxylic |
| | | | | acid tert- |
| | | | | butyl ester |
| 11c | 3-{[1-(5-Benzyl-3-methyl-4-oxo-4,5-dihydro- | 512 | Section | 3- |
| | isothiazolo[5,4-d]pyrimidin-6-yl)- | (MH⁺) | 1: | aminomethyl- |
| | propylamino]-methyl}-piperidine-1-carboxylic | | Method 9 | piperidine- |
| | acid tert-butyl ester | | | 1- |
| | | | | carboxylic |
| | | | | acid tert- |
| | | | | butyl ester |
| 11d | 5-Benzyl-6-[1-(2-dimethylamino-ethylamino)- | 386 | Section | Dimethylethane- |
| | propyl]-3-methyl-5H-isothiazolo[5,4- | (MH⁺) | 1: | 1,2- |
| | d]pyrimidin-4-one | | Method 9 | diamine |
| 11e | 5-Benzyl-6-[1-(3-dimethylamino- | 400 | Section | Dimethylpropane- |
| | propylamino)-propyl]-3-methyl-5H- | (MH⁺) | 1: | 1,3- |
| | isothiazolo[5,4-d]pyrimidin-4-one | | Method 9 | diamine |
| 11f | 5-Benzyl-6-[1-(3-hydroxy-propylamino)- | 373 | Section | 3-Amino- |
| | propyl]-3-methyl-5H-isothiazolo[5,4- | (MH⁺) | 1: | propan-1- |
| | d]pyrimidin-4-one | | Method 9 | ol |

Section 1: Method 12

### N-(3-Amino-propyl)-N-[1-(5-benzyl-3-methyl-4-oxo-4,5-dihydro-isothiazolo-[5,4-d]pyrimidin-6-yl)-propyl]-4-methyl-benzamide

To a solution of the amine 13 (Section 1: Method 10) (0.1 g, 0.21 mmol) and triethylamine (0.303 g, 3 mmol) in dichloromethane (20 mL) at r.t. was added dropwise a solution of p-toluoyl chloride (0.1 g, 0.6 mmol) in dichloromethane (10 mL). The resulting solution was stirred at r.t. for 30 min. after which the TLC showed the disappearance of the SM. The reaction mixture was diluted with CH₂Cl₂ (60 mL) washed with satd. NaHCO₃ (100 mL), water (100 mL), brine (100 mL) and dried (Na₂SO₄). Concentration of the organic layer provided the product which was purified by column (silica gel) chromatography using 20-30% EtOAc in hexanes as eluent. Yield=0.117 g (94%). The acylated product was dissolved in 2M HCl in ether and the mixture was stirred at r.t. for 20 h. The precipitated product was filtered off and washed with ether and dried in vacuo to yield the pure N-(3-amino-propyl)-N-[1-(5-benzyl-3-methyl-4-oxo-4,5-dihydro-isothiazolo-[5,4-d]pyrimidin-6-yl)-propyl]-4-methyl-benzamide chloride salt (91 mg, 87%). White powder, mp. 127.8-129.2° C. m/z 490 (MH⁺), ¹H NMR (DMSO-d6 300 MHz, 96° C.) δ: 7.79 (bs, 3H), 7.37-6.95 (m, 9H), 5.77 (d, 1H), 5.50 (bs, 1H), 4.83 (d, 1H), 3.36 (t, 2H), 2.72 (s, 3H), 2.46 (t, 2H), 2.39 (s, 3H), 2.20-2.05 (m, 1H), 1.96-1.75 (m, 1H), 1.74-1.40 (m, 2H), 0.63 (t, 3H).

The following compounds were synthesized according to Section 1: Method 12:

| Section 1: | | | | Acylating |
|---|---|---|---|---|
| Method # | Compound Name | m/z | SM | agent |
| 12a | N-(3-Amino-propyl)-N-[1-(5- | 507 | Section | 4-methyl- |
| | {4-fluorobenzyl}-3-methyl- | (MH⁺) | 1: | benzoyl |
| | 4-oxo-4,5-dihydro- | | Method | chloride |
| | isothiazolo[5,4- | | 10a | |
| | d]pyrimidin-6-yl)-propyl]- | | | |
| | 4-methyl-benzamide | | | |

Section 1: Method 13

### N-(3-Amino-propyl)-N-[1-(5-benzyl-3-methyl-4-oxo-4,5-dihydro-isothiazolo[5,4-d]pyrimidin-6-yl)-propyl]-4-bromo-benzamide

To a solution of {3-[1-(5-Benzyl-3-methyl-4-oxo-4,5-dihydro-isothiazolo[5,4-d]pyrimidin-6-yl)-propylamino]-propyl}-carbamic acid tert-butyl ester (Section 1: Method 11) (0.085 g, 0.167 mmol) in dichloromethane (8 mL) at r.t. was added a saturated solution of potassium carbonate (8 ml) followed by the dropwise addition of p-bromo benzoyl chloride (0.044 g, 0.2 mmol). The resulting solution was stirred at r.t. for 16 h after which the LC/MS showed the disappearance of the SM. The reaction mixture was evaporated to dryness and resuspended in 3 ml MeOH and purified by Gilson HPLC using a 20-99% H₂O/CH₃CN (0.05% TFA) gradient. Concentration of the desired fractions gave {3-[1-(5-Benzyl-3-methyl-4-oxo-4,5-dihydro-isothiazolo[5,4-d]pyrimidin-6-yl)-propyl] (4-bromobenzoyl)amino]propyl}-carbamic acid tert-butyl ester.

The product was dissolved in 2M HCl in 1,4 dioxane and the mixture was stirred at r.t. for 1 h. The reaction mixture was evaporated to dryness, washed with ether and dried in vacuo to yield the pure N-(3-Amino-propyl)-N-[1-(5-benzyl-3-methyl-4-oxo-4,5-dihydro-isothiazolo[5,4-d]pyrimidin-6-yl)-propyl]-4-bromo-benzamide hydrogen chloride salt (33 mg, 34%). m/z 556 (MH⁺), ¹H NMR (DMSO-d6 500 MHz, 96° C.) δ: 7.80 (br, 3H), 7.64 (d, 2H), 7.36-7.28 (m, 5H), 7.13 (m, 2H), 5.80 (d, 1H), 5.57 (bs, 1H), 4.95 (d, 1H), 3.38 (t, 2H), 2.77 (s, 3H), 2.47 (t, 2H), 2.17-2.13 (m, 1H), 1.96-1.91 (m, 1H), 1.72-1.50 (m, 2H), 0.68 (t, 3H).

The following compounds were synthesized according to Section 1: Method 13:

| Section 1: | | | | Acylating |
|---|---|---|---|---|
| Method # | Compound Name | m/z | SM | agent |
| 13a | N-(3-Amino-propyl)-N-[1-(5-benzyl-3- | 510 | Section 1: | 4-chloro- |
| | methyl-4-oxo-4,5-dihydro-isothiazolo[5,4- | (MH⁺) | Method | benzoyl |
| | d]pyrimidin-6-yl)-propyl]-4-chloro- | | 11 | chloride |
| | benzamide hydrogen chloride | | | |
| 13b | N-(3-Amino-propyl)-N-[1-(5-benzyl-3- | 508 | Section 1: | 3-fluoro- |
| | methyl-4-oxo-4,5-dihydro-isothiazolo[5,4- | (MH⁺) | Method | 4-methyl- |
| | d]pyrimidin-6-yl)-propyl]-3-fluoro-4- | | 11 | benzoyl |
| | methyl-benzamide hydrogen chloride | | | chloride |
| 13c | N-(3-Amino-propyl)-N-[1-(5-benzyl-3- | 545 | Section 1: | 2,3- |
| | methyl-4-oxo-4,5-dihydro-isothiazolo[5,4- | (MH⁺) | Method | dichloro- |
| | d]pyrimidin-6-yl)-propyl]-2,3-dichloro- | | 11 | benzoyl |
| | benzamide hydrogen chloride | | | chloride |
| 13d | Naphthalene-2-carboxylic acid (3-amino- | 526 | Section 1: | 2- |
| | propyl)-[1-(5-benzyl-3-methyl-4-oxo-4,5- | (MH⁺) | Method | napthoyl- |
| | dihydro-isothiazolo[5,4-d]pyrimidin-6-yl)- | | 11 | chloride |
| | propyl]-amide hydrogen chloride | | | |
| 13e | Benzo[b]thiophene-2-carboxylic acid (3- | 532 | Section 1: | 1- |
| | amino-propyl)-[1-(5-benzyl-3-methyl-4- | (MH⁺) | Method | benzothio |
| | oxo-4,5-dihydro-isothiazolo[5,4- | | 11 | phene-2- |
| | d]pyrimidin-6-yl)-propyl]-amide hydrogen | | | carbonyl |
| | chloride | | | chloride |
| 13f | N-Azetidin-3-ylmethyl-N-[1-(5-benzyl-3- | 502 | Section 1: | 4-methyl- |
| | methyl-4-oxo-4,5-dihydro-isothiazolo[5,4- | (MH⁺) | Method | benzoyl |
| | d]pyrimidin-6-yl)-propyl]-4-methyl- | | 11 | chloride |
| | benzamide hydrogen chloride | | | |
| 13g | N-[1-(5-Benzyl-3-methyl-4-oxo-4,5- | 530 | Section 1: | 4-methyl- |
| | dihydro-isothiazolo[5,4-d]pyrimidin-6-yl)- | (MH⁺) | Method | benzoyl |
| | propyl]-4-methyl-N-piperidin-3-ylmethyl- | | 11 | chloride |
| | benzamide hydrogen chloride | | | |
| 13h | N-(2-Amino-ethyl)-N-[1-(5-benzyl-3- | 476 | Section 1: | 4-methyl- |
| | methyl-4-oxo-4,5-dihydro-isothiazolo[5,4- | (MH⁺) | Method | benzoyl |
| | d]pyrimidin-6-yl)-propyl]-4-methyl- | | 11 | chloride |
| | benzamide hydrogen chloride | | | |

Section 1: Method 14

### N-[1-(5-Benzyl-3-methyl-4-oxo-4,5-dihydro-isothiazolo[5,4-d]pyrimidin-6-yl)-propyl]-N-(3-dimethylamino-propyl)-4-methyl-benzamide

To a solution of 5-Benzyl-6-[1-(3-dimethylamino-propylamino)-propyl]-3-methyl-5H-isothiazolo[5,4-d]pyrimidin-4-one (Section 1: Method 11e) (0.104 g, 0.26 mmol) in dichloromethane (10 mL) at r.t. was added a saturated solution of potassium carbonate (10 ml) followed by the dropwise addition of p-toluoyl chloride (34 μL, 0.26 mmol). The resulting solution was stirred at r.t. for 16 h after which the LC/MS showed the disappearance of the SM. The reaction mixture was evaporated to dryness and resuspended in 3 ml MeOH and purified by Gilson HPLC using a 20-99% H₂O/CH₃CN (0.05% TFA) gradient. Concentration of the desired fractions gave N-[1-(5-Benzyl-3-methyl-4-oxo-4,5-dihydro-isothiazolo[5,4-d]pyrimidin-6-yl)-propyl]-N-(3-dimethylamino-propyl)-4-methyl-benzamide (65 mg, 48%). m/z 518 (MH⁺), ¹H NMR (DMSO-d6 300 MHz, 96° C.) δ: 7.44-7.00 (m, 9H), 5.82 (d, 1H), 5.51 (bs, 1H), 4.86 (d, 1H), 3.41 (t, 2H), 2.75 (s, 3H), 2.50 (s, 6H), 2.39 (bm, 2H), 2.12-2.05 (m, 1H), 1.93-1.90 (m, 1H), 1.75 (m, 1H), 1.50 (m, 1H), 0.66 (t, 3H).

The following compounds were synthesized according to Section 1: Method 14:

| Section 1: | | | | Acylating |
|---|---|---|---|---|
| Method # | Compound Name | m/z | SM | agent |
| 14a | N-[1-(5-Benzyl-3-methyl-4-oxo-4,5- | 504 | Section 1: | 4-methyl- |
| | dihydro-isothiazolo[5,4-d]pyrimidin-6-yl)- | (MH⁺) | Method | benzoyl |
| | propyl]-N-(2-dimethylamino-ethyl)-4- | | 11d | chloride |
| | methyl-benzamide | | | |
| 14b | N-[1-(5-Benzyl-3-methyl-4-oxo-4,5- | 491 | Section 1: | 4-methyl- |
| | dihydro-isothiazolo[5,4-d]pyrimidin-6-yl)- | (MH⁺) | Method | benzoyl |
| | propyl]-N-(3-hydroxy-propyl)-4-methyl- | | 11f | chloride |
| | benzamide | | | |

Section 1: Method 15

### Methanesulfonic acid 3-[[1-(5-benzyl-3-methyl-4-oxo-4,5-dihydro-isothiazolo [5,4-d]pyrimidin-6-yl)-propyl]-(4-methyl-benzoyl)-amino]-propyl ester

To a solution of N-[1-(5-Benzyl-3-methyl-4-oxo-4,5-dihydro-isothiazolo[5,4-d]pyrimidin-6-yl)-propyl]-N-(3-hydroxy-propyl)-4-methyl-benzamide (Section 1: Method 14b) (0.42 g, 0.85 mmol) in anhydrous dichloromethane (57 mL), was added anhydrous diisopropylethylamine (295 μl, 1.70 mmol) followed by dropwise addition of methanesulphonyl chloride (71 μl, 0.935 mmol). The reaction mixture was stirred at r.t. for 2 h. The LC/MS of the RM showed the complete disappearance of the starting material and complete conversion to the methanesulfonic acid 3-[[1-(5-benzyl-3-methyl-4-oxo-4,5-dihydro-isothiazolo [5,4-d]_pyrimidin-6-yl)-propyl]-(4-methyl-benzoyl)-amino]-propyl ester. The reaction mixture was evaporated to dryness and used further crude.

Section 1: Method 16

### N-(3-Azetidin-1-yl-propyl)-N-[1-(5-benzyl-3-methyl-4-oxo-4,5-dihydro-isothiazolo[5,4-d]pyrimidin-6-yl)-propyl]-4-methyl-benzamide

To a solution of methanesulfonic acid 3-[[1-(5-benzyl-3-methyl-4-oxo-4,5-dihydro-isothiazolo [5,4-d]pyrimidin-6-yl)-propyl]-(4-methyl-benzoyl)-amino]-propyl ester (Section 1: Method 15) ((assumed from previous reaction) 0.080 g, 0.14 mmol) in DMF (25 mL) at r.t. was added an excess of potassium carbonate (0.097 g, 0.70 mmol) followed by the dropwise addition of azetidine (19 μl, 0.28 mmol). The reaction mixture was stirred at 38° C. for 16 h after which the LC/MS showed the disappearance of the SM. The reaction mixture was evaporated to dryness on a GeneVac HT12 and resuspended in 3 ml MeOH and purified by Gilson HPLC using a 20-99% H₂O/CH₃CN (0.05% HCl) gradient. Concentration of the desired fractions gave N-(3-Azetidin-1-yl-propyl)-N-[1-(5-benzyl-3-methyl-4-oxo-4,5-dihydro-isothiazolo[5,4-d]pyrimidin-6-yl)-propyl]-4-methyl-benzamide (51 mg, 69%). m/z 530 (MH⁺), ¹H NMR (DMSO-d6 400 MHz, 96° C.) δ: 7.40-7.00 (m, 9H), 5.85 (d, 1H), 5.55 (bs, 1H), 4.85 (d, 1H), 3.40 (b, 2H), 2.90 (b, 2H), 2.78 (s, 3H), 2.50 (b, 2H), 2.40 (s, 3H), 2.35 (bm, 2H), 2.20-2.00 (m, 1H), 1.96-1.80 (m, 1H), 1.65-1.50 (m, 1H), 1.40-1.30 (m, 3H), 0.65 (t, 3H)

The following compounds were synthesized according to Section 1: Method 16:

| Section 1: | | | | |
|---|---|---|---|---|
| Method # | Compound Name | m/z | SM | Amine |
| 16a | N-[1-(5-benzyl-3-methyl-4-oxo-4,5- | 532 | Section | Isopropyl- |
| | dihydroisothiazolo-[5,4-d]pyrimidin-6- | (MH⁺) | 1: | amine |
| | yl)propyl]-N-[3-(isopropylamino)propyl]- | | Method | |
| | 4-methylbenzamide | | 15 | |
| 16b | N-[1-(5-benzyl-3-methyl-4-oxo-4,5- | 530 | Section | Cyclopropyl- |
| | dihydroisothiazolo-[5,4-d]pyrimidin-6- | (MH⁺) | 1: | amine |
| | yl)propyl]-N-[3- | | Method | |
| | (cyclopropylamino)propyl]-4- | | 15 | |
| | methylbenzamide | | | |
| 16c | N-[1-(5-benzyl-3-methyl-4-oxo-4,5- | 504 | Section | Methyl- |
| | dihydroisothiazolo-[5,4-d]pyrimidin-6- | (MH⁺) | 1: | amine |
| | yl)propyl]-4-methyl-N-[3-(methylamino) | | Method | |
| | propyl] benzamide | | 15 | |
| 16d | N-[1-(5-benzyl-3-methyl-4-oxo-4,5- | 544 | Section | Pyrrolidine |
| | dihydroisothiazolo-[5,4-d]pyrimidin-6- | (MH⁺) | 1: | |
| | yl)propyl]-4-methyl-N-[3-(3-pyrrolidin- | | Method | |
| | 1-ylpropyl) benzamide | | 15 | |

Section 1: Method 17

### 5-Benzyl-6-{1-[(3-hydroxy-propyl)-(4-methyl-benzyl)-amino]-propyl}-3-methyl-5H-isothiazolo[5,4-d]pyrimidin-4-one

To a solution of 5-Benzyl-6-[1-(3-hydroxy-propylamino)-propyl]-3-methyl-5H-isothiazolo[5,4-d]pyrimidin-4-one (Section 1: Method 11f) (0.098 g, 0.26 mmol) in anhydrous DMF (3 mL) was added potassium carbonate (0.108 g, 0.78 mmol) followed by the dropwise addition of 4-methyl benzyl bromide (0.048 g, 0.26 mmol). The resulting solution was shaken at 40° C. for 4 h after which the LC/MS showed the disappearance of the SM. The reaction mixture was evaporated to dryness and resuspended in 3 ml MeOH and purified by Gilson HPLC using a 20-99% H₂O/CH₃CN (0.05% TFA) gradient. Concentration of the desired fractions gave 5-Benzyl-6-{1-[(3-hydroxy-propyl)-(4-methyl-benzyl)-amino]-propyl}-3-methyl-5H-isothiazolo[5,4-d]pyrimidin-4-one (48 mg, 39%). m/z 477 (MH⁺), ¹H NMR (DMSO-d6 500 MHz, 96° C.) δ: 8.20 (s, 1H), 7.40-6.85 (m, 9H), 5.80 (d, 1H), 5.20 (d, 1H), 3.80 (d, 1H), 3.70 (m, 1H), 3.62 (d, 1H), 3.50-3.30 (m, 2H), 2.90 (m, 1H), 2.75 (s, 3H), 2.33 (m, 2H), 2.25 (s, 3H), 2.20-2.16 (m, 1H), 1.90-1.80 (m, 1H), 1.50 (m, 2H), 0.65 (t, 3H).

Section 1: Method 18

### 5-Butyrylamino-3-methyl-isoxazole-4-carboxylic acid amide

A mixture of 5-amino-3-methyl-isoxazole-4-carboxylic acid amide (2 g, 14.18 mmol) in 10 ml of butyric anhydride was stirred at 150° C. for 0.5˜1 h. The brown solution was diluted with hexane (100 ml) and cooled to room temperature. The solid crushed out from the mixture was filtered and washed with hexane, dried in vacuo. The title amide (2.6 g) was obtained as white solid.

Section 1: Method 19

### 3-Methyl-6-propyl-5H-isoxazolo[5,4-d]pyrimidin-4-one

A suspension of 5-Butyrylamino-3-methyl-isoxazole-4-carboxylic acid amide (Section 1: Method 18) (2.6 g, split into 20 vials) in 3.5 ml of 2N NaOH aq was subjected to microwave irradiation under the temperature of 140° C. for 20 min. The resulting solution was cooled with an ice bath, and the pH was adjusted to 1˜3 with concentrated HCl. The crushed out solid was filtered, washed with water, dried over vacuum at 40° C. overnight. The title pyrimidinone (1.749 g) was obtained as white solid. ¹H NMR (400 MHz, DMSO-d₆): 0.91 (t, 3H), 1.71 (m, 2H), 2.44 (s, 3H), 2.64 (t, 2H), 12.78 (s, 1H).

Section 1: Method 20

### 5-Benzyl-3-methyl-6-propyl-5H-isoxazolo[5,4-d]pyrimidin-4-one

A suspension of 3-methyl-6-propyl-5H-isoxazolo[5,4-d]pyrimidin-4-one (Section 1: Method 19) (1.698 g, 8.8 mmol), benzylbromide (1.5 g, 8.8 mmol), potassium carbonate (2.43 g, 17.6 mmol) in 10 ml DMF was stirred at room temperature overnight. The mixture was diluted with water, extracted with ethyl acetate (50 ml×3), the combined organic phases were dried over anhydrous sodium sulfate, concentrated, purified by flash column chromatography (elute:hexane-ethyl acetate=5:1). 1.69 g (68%) of the title compound was obtained as white solid. ¹H NMR (400 MHz, DMSO-d₆): 0.80 (t, 3H), 1.61 (m, 2H), 2.43 (s, 3H), 2.73 (t, 2H), 5.35 (s, 2H), 7.12-7.35 (m, 5H).

Section 1: Method 21

### 5-Benzyl-6-(1-bromo-propyl)-3-methyl-5H-isoxazolo[5,4-d]pyrimidin-4-one

A solution of 5-benzyl-3-methyl-6-propyl-5H-isoxazolo[5,4-d]pyrimidin-4-one (Section 1: Method 20) (3.167 g, 11.2 mmol) and sodium acetate (4.59 g, 56 mmol, 5 eq) in glacial acetic acid (26 ml) was treated with a preformed bromine solution (0.7 ml bromine in 10 ml of glacial acetic acid) (8.64 ml, 22.4 mmol, 2 eq). The mixture was stirred at 100° C. for 24 hrs. Excess bromine (8.64 ml, 22.4 mmol, 2 eq) was added to the mixture. The mixture was then stirred at 100° C. for another 24 hrs. Water was added to the reaction mixture, followed by aq. potassium carbonate. The mixture was extracted with methylene chloride (50 ml×3), the combined organic phases were washed with water and dried over anhydrous sodium sulfate, then concentrated to give the product which was purified by flash chromatography (elute: hexane-ethyl acetate). 2.5 g product was furnished as a white solid. ¹H NMR (400 MHz, DMSO-d6): 0.79 (t, 3H), 2.18 (m, 1H), 2.35 (m, 1H), 2.58 (s, 3H), 5.12 (t, 1H), 5.25 (d, 1H), 5.80 (d, 1H), 7.27-7.42 (m, 5H).

Section 1: Method 22

### 5-Benzyl-6-(1-butylamino-propyl)-3-methyl-H-isoxazolo[5,4-d]pyrimidin-4-one

To a suspension of 5-benzyl-6-(1-bromo-propyl)-3-methyl-5H-isoxazolo[5,4-d]pyrimidin-4-one (Section 1: Method 21) (2.8 g, 7.73 mmol) and potassium carbonate (2.67 g, 19.38 mmol) in acetonitrile (100 ml) was added tert-butyl-N-(3-aminopropyl)-carbamate (1.345 g, 7.73 mmol). The mixture was stirred at 100° C. overnight. Water (30 ml) was added to the mixture, which was extracted with ethyl acetate (3×50 ml). The combined organic phases were washed with brine (10 ml), dried over sodium sulfate, concentrated to obtain the title amine which was purified by flash chromatography column (elute: ethyl acetate-hexane=1-4˜1-1) to give 2.6 g (74%) of product as white solid. ¹H NMR (400 MHz, DMSO-d₆): 0.85 (t, 3H), 1.32 (m, 2H), 1.41 (s, 9H), 1.58 (m, 1H), 1.65 (m, 1H), 2.09 (m, 1H), 2.40 (m, 1H), 2.60 (s, 3H), 2.81 (m, 2H), 3.29 (m, 1H), 3.75 (m, 1H), 5.42 (d, 1H), 5.63 (d, 1H), 6.72 (br, 1H), 7.25-7.45 (m, 5H).

Section 1: Method 23

### N-[[1-(5-Benzyl-3-methyl-4-oxo-4,5-dihydro-isoxazolo[5,4-d]pyrimidin-6-yl)-propyl]-(4-methyl-benzoyl)-amino]-propyl)-carbamic acid tert-butyl ester

A solution of 5-benzyl-6-(1-butylamino-propyl)-3-methyl-5H-isoxazolo[5,4-d]pyrimidin-4-one (Section 1: Method 22) (135 mg, 0.297 mmol) in dichloromethane (4 ml) was added to p-toluoyl chloride (46 mg, 0.297 mmol) followed by triethylamine (60 mg, 0.594 mmol). The mixture was stirred at room temperature for 1 hr. Then diluted with dichloromethane, washed with saturated aq. sodium bicarbonate. The organic phase was dried over sodium sulfate, filtered, and concentrated. The crude oil was purified by flash column chromatography (solvent: ethyl acetate-hexane) to furnish N-[[1-(5-benzyl-3-methyl-4-oxo-4,5-dihydro-isoxazolo[5,4-d]pyrimidin-6-yl)-propyl]-(4-methyl-benzoyl)-amino]-propyl)-carbamic acid tert-butyl ester (130 mg) as a white solid.

¹H NMR (500 MHz, 100° C., DMSO-d₆): 0.71 (t, 3H), 1.12 (m, 1H), 1.35 (s, 9H), 1.47 (m, 1H), 1.92 (m, 1H), 2.14 (m, 1H), 2.37 (s, 3H), 2.56 (s, 3H), 2.57 (m, 2H), 3.29 (m, 2H), 5.01 (d, 1H), 5.68 (m, br, 1H), 5.79 (d, 1H), 6.06 (br, 1H), 7.14-7.36 (m, 9H).

Section 1: Method 24

### N-(3-Amino-propyl)-N-[1-(5-benzyl-3-methyl-4-oxo-4,5-dihydro-isoxazolo[5,4-d]pyrimidin-6-yl)-propyl]-4-methyl-benzamide

A solution of N-[[1-(5-benzyl-3-methyl-4-oxo-4,5-dihydro-isoxazolo[5,4-d]pyrimidin-6-yl)-propyl]-(4-methyl-benzoyl)-amino]-propyl)-carbamic acid tert-butyl ester (Section 1: Method 23) (0.223 mmol) in 3 ml of 4 M HCl in dioxane was stirred at room temperature for 2 hr. The solvent was distilled off by vacuo, the residue was dried at 40˜50° C. for overnight under vacuum. The corresponding amine chloride salt was obtained. m/z 474 (MH⁺) ¹H NMR (500 MHz, 100° C., DMSO-d₆): 0.68 (t, 3H), 1.52 (m, 1H), 1.72 (m, 1H), 1.92 (m, 1H), 2.10 (m, 1H), 2.39 (s, 3H), 2.51 (m, 2H), 2.57 (s, 3H), 3.41 (m, 2H), 4.85 (br, 1H), 5.50 (br, 1H), 5.77 (d, 1H), 7.07 (br, 2H), 7.24-7.35 (m, 7H), 7.73 (br, 3H).

The following compounds were synthesized according to Section 1: Method 24:

| Section 1: | | | | Acylating |
|---|---|---|---|---|
| Method # | Compound Name | m/z | SM | agent |
| 24a | N-(3-Amino-propyl)-N-[1-(5-benzyl-3- | 478 | Section 1: | 4-fluoro- |
| | methyl-4-oxo-4,5-dihydro-isoxazolo[5,4- | (MH⁺) | Method 23 | benzoyl |
| | d]pyrimidin-6-yl)-propyl]-4-fluoro- | | | chloride |
| | benzamide hydrogen chloride | | | |
| 24b | N-(3-Amino-propyl)-N-[1-(5-benzyl-3- | 529 | Section 1: | 2,3- |
| | methyl-4-oxo-4,5-dihydro-isoxazolo[5,4- | (MH⁺) | Method 23 | dichloro- |
| | d]pyrimidin-6-yl)-propyl]-2,3-dichloro- | | | benzoyl |
| | benzamide hydrogen chloride | | | chloride |
| 24c | N-(3-Amino-propyl)-N-[1-(5-benzyl-3- | 492 | Section 1: | 3-fluoro- |
| | methyl-4-oxo-4,5-dihydro-isoxazolo[5,4- | (MH⁺) | Method 23 | 4-methyl- |
| | d]pyrimidin-6-yl)-propyl]-3-fluoro-4-methyl- | | | benzoyl |
| | benzamide hydrogen chloride | | | chloride |
| 24d | N-(3-Amino-propyl)-N-[1-(5-benzyl-3- | 490 | Section 1: | 4- |
| | methyl-4-oxo-4,5-dihydro-isoxazolo[5,4- | (MH⁺) | Method 23 | methoxy- |
| | d]pyrimidin-6-yl)-propyl]-4-methoxy- | | | benzoyl |
| | benzamide hydrogen chloride | | | chloride |

Section 1: Method 25

### N-(4-Cyano-3-methyl-isothiazol-5-yl)-3-methyl-butyramide

To a solution of 5-amino-3-methyl-isothiazole-4-carbonitrile (Section 1: Method 4) (6.38 g, 45.9 mmol) in pyridine (20 mL) at 0° C., isovaleryl chloride (6.65 g, 55 mmol) was added dropwise. After the completion of the addition the reaction mixture was allowed to warm to r.t. and stirred overnight. The TLC and the MS showed the complete disappearance of the starting material and the reaction mixture was diluted with CHCl₃ (200 mL), washed with water (200 mL), 2N HCl (225 mL), satd. NaHCO₃ (200 mL), brine (200 mL) and dried over Na₂SO₄. Concentration of the CHCl₃ layer provided the product which was triturated from CH₂Cl₂/hexanes (1/10) and filtered off to isolate N-(4-cyano-3-methyl-isothiazol-5-yl)-3-methyl-butyramide (8.1 g, 79%) as an off-white crystalline solid. ¹H NMR (300 MHz, CDCl₃) δ 1.04 (d, 6H), 2.18-2.32 (m, 1H), 2.46 (d, 2H), 2.53 (s, 3H), 9.87 (bs, 1H).

Section 1: Method 26

### 3-Methyl-5-(3-methyl-butyrylamino)-isothiazole-4-carboxylic acid amide

To a solution of N-(4-cyano-3-methyl-isothiazol-5-yl)-3-methyl-butyramide (Section 1: Method 25) (8 g, 35.8 mmol) in 30% aqueous NH₄OH (200 mL), was added dropwise 100 mL of hydrogen peroxide at r.t. After the completion of the addition the reaction mixture was stirred at 60° C. overnight after which the TLC showed the complete disappearance of SM. The reaction mixture was concentrated to 40 mL and extracted with chloroform (3×100 mL). The organic layer was dried (Na₂SO₄) and concentrated to obtain 3-methyl-5-(3-methyl-butyrylamino)-isothiazole-4-carboxylic acid amide (6.1 g, 71%) as a light yellow solid. ¹H NMR (300 MHz, CDCl₃) δ 1.03 (d, 6H), 2.24 (m, 1H), 2.43 (d, 2H), 2.69 (s, 3H), 5.98 (bs, 2H), 11.77 (bs, 1H).

Section 1: Method 27

### 6-Isobutyl-3-methyl-5H-isothiazolo[5,4-d]pyrimidin-4-one

3-Methyl-5-(3-methyl-butyrylamino)-isothiazole-4-carboxylic acid amide (Section 1: Method 26) (6 g, 25 mmol) was suspended in 150 mL of 30% NH₃ and then was heated to 140° C. for 5 h in a pressure reactor. The mixture was cooled and neutralized to pH 7. The reaction mixture was extracted with EtOAc (3×100 mL) and the combined organic layers were washed with water (100 mL), brine (100 mL) and concentrated to get the product which was further purified by column (silica gel) chromatography using 30% EtOAc in hexanes as eluent. Concentration of the pure product fractions provided 6-isobutyl-3-methyl-5H-isothiazolo[5,4-d]pyrimidin-4-one (2.2 g, 38%) as an off-white powder. ¹H NMR (300 MHz, CDCl₃) δ 1.05 (d, 6H), 2.32 (m, 1H), 2.69 (d, 2H), 2.82 (s, 3H).

Section 1: Method 28

### 5-Benzyl-6-isobutyl-3-methyl-5H-isothiazolo[5,4-d]pyrimidin-4-one

To a solution of 6-isobutyl-3-methyl-5H-isothiazolo[5,4-d]pyrimidin-4-one (Section 1: Method 27) (1.31 g, 5.8 mmol) in 20 mL of anhydrous DMF was added 1.38 g (10 mmol) of anhydrous K₂CO₃ followed by benzyl bromide (1.18 g, 6.9 mmol) and the mixture was stirred at room temperature overnight. The TLC of the reaction mixture showed the complete disappearance of the SM. The reaction mixture was poured into ice-cold water and extracted with EtOAc (3×100 mL). The combined extracts were washed with water (100 mL), brine (100 mL), dried (Na₂SO₄) and concentrated. The TLC and the ¹H NMR showed the presence of two products N alkylated as well as O-alkylated products in a ratio of 7:3. The products were separated by column (silica gel, 116 g) chromatography using 10% EtOAc in hexanes. 5-Benzyl-6-isobutyl-3-methyl-5H-isothiazolo[5,4-d]pyrimidin-4-one was isolated as white crystalline solid (1.3 g, 70%). m/z 314 (MH⁺), ¹H NMR (300 MHz, CDCl₃) δ 0.94 (d, 6H), 2.23-2.37 (m, 1H), 2.64 (d, 2H), 2.82 (s, 3H), 5.38 (s, 2H), 7.10-7.38 (m, 5H).

The following compounds were synthesized according to Section 1: Method 28:

| Section 1: | | | |
|---|---|---|---|
| Method # | Compound Name | m/z | Alkylating agent |
| 28a | 5-(4-Fluoro-benzyl)-6-isobutyl- | 332 | 4-fluorobenzyl |
| | 3-methyl-5H-isothiazolo[5,4- | (MH⁺) | bromide |
| | d]pyrimidin-4-one | | |

Section 1: Method 29

### 5-Benzyl-6-(1-bromo-2-methyl-propyl)-3-methyl-5H-isothiazolo[5,4-d]pyrimidin-4-one

To a solution of 5-benzyl-6-isobutyl-3-methyl-5H-isothiazolo[5,4-d]pyrimidin-4-one (Section 1: Method 28) (1.3 g, 4.2 mmol) and sodium acetate (2 g) in acetic acid (10 mL) at 100° C., a solution of the bromine (1.32 g, 8.4 mmol) in acetic acid (10 mL) was added dropwise over a period of 20 minutes. The reaction mixture was stirred at that temperature for 30 min and cooled and the TLC (eluent 10% EtOAc in hexanes) and MS showed the complete disappearance of the SM and only the product. The reaction mixture was poured into ice water and extracted with EtOAc (3×60 mL) and the organic layers were combined and washed with 2% sodium thiosulfate solution (60 mL), water (100 mL), brine (100 mL) and dried over Na₂SO₄. Concentration of the organic layer provided 5-benzyl-6-(1-bromo-2-methyl-propyl)-3-methyl-5H-isothiazolo[5,4-d]pyrimidin-4-one (1.61 g, 99%) as white crystalline solid. m/z 394 (MH⁺), ¹H NMR (300 MHz, CDCl₃) δ 0.54 (d, 3H), 1.11 (d, 3H), 2.62-2.76 (m, 1H), 2.83 (s, 3H), 4.42 (d, 1H), 4.80 (d, 1H), 6.22 (d, 1H), 7.12-7.42 (m, 5H).

The following compounds were synthesized according to Section 1: Method 29:

| Section 1: | | |
|---|---|---|
| Method # | Compound Name | m/z |
| 29a | 6-(1-Bromo-2-methyl-propyl)-5-(4-fluoro- | 412 |
| | benzyl)-3-methyl-5H-isothiazolo[5,4- | (MH⁺) |
| | d]pyrimidin-4-one | |

Section 1: Method 30

### 6-(1-Azido-2-methyl-propyl)-5-benzyl-3-methyl-5H-isothiazolo[5,4-d]pyrimidin-4-one

To a solution of 5-benzyl-6-(1-bromo-2-methyl-propyl)-3-methyl-5H-isothiazolo[5,4-d]pyrimidin-4-one (Section 1: Method 29) (0.6 g, 1.52 mmol) in anhydrous DMF (20 mL), sodium azide (0.65 g, 10 mmol) was added and the mixture was stirred at room temperature for 1 hour. The TLC of the RM showed the complete disappearance of the starting bromide. The reaction mixture was poured into ice water (300 mL) and extracted with EtOAc (3×100 mL). The organic layer was washed with water (100 mL), brine (100 mL) and dried (Na₂SO₄). Concentration of the organic layer provided the product which was purified by column (silica gel) chromatography using 30% EtOAc in hexanes as eluent to isolate 6-(1-azido-2-methyl-propyl)-5-benzyl-3-methyl-5H-isothiazolo[5,4-d]pyrimidin-4-one (0.506 g, 94%) as a low melting solid. m/z 355 (MH⁺), 1H NMR (300 MHz, CDCl₃) δ 0.57 (d, 3H), 1.07 (d, 3H), 2.50-2.74 (m, 1H), 2.98 (s, 3H), 3.71 (d, 1H), 5.05 (d, 1H), 5.78 (d, 1H), 7.12-7.40 (m, 5H).

The following compounds were synthesized according to Section 1: Method 30:

| Section 1: | | |
|---|---|---|
| Method # | Compound Name | m/z |
| 30a | 6-(1-Azido-2-methyl-propyl)-5-(4-fluoro-benzyl)-3- | 373 |
| | methyl-5H-isothiazolo[5,4-d]pyrimidin-4-one | (MH⁺) |

Section 1: Method 31

### 6-(1-Amino-2-methyl-propyl)-5-benzyl-3-methyl-5H-isothiazolo[5,4-d]pyrimidin-4-one

To a solution of 6-(1-azido-2-methyl-propyl)-5-benzyl-3-methyl-5H-isothiazolo[5,4-d]pyrimidin-4-one (Section 1: Method 30) (0.5 g, 1.41 mmol) in methanol (20 mL) was added 5% Pd/C (20% by wt.) and the resulting mixture was stirred at r.t. in an atmosphere of H₂ and the progress of the reaction was monitored by MS. After the disappearance of the starting material the reaction mixture was filtered through celite and washed with EtOAc. Concentration of the filtrate provided 6-(1-amino-2-methyl-propyl)-5-benzyl-3-methyl-5H-isothiazolo[5,4-d]pyrimidin-4-one as a thick oil. The product was used as such in the next reaction with out further purification. m/z 349 (MH⁺)

The following compounds were synthesized according to Section 1: Method 31:

| Section 1: | | |
|---|---|---|
| Method # | Compound Name | m/z |
| 31a | 6-(1-Amino-2-methyl-propyl)-5-(4-fluoro-benzyl)-3- | 367 |
| | methyl-5H-isothiazolo[5,4-d]pyrimidin-4-one | (MH⁺) |

Section 1: Method 32

### {3-[1-(5-Benzyl-3-methyl-4-oxo-4,5-dihydro-isothiazolo [5,4-d]pyrimidin-6-yl)-2-methyl-propylamino]-propyl}-carbamic acid tert-butyl ester

To a solution of 6-(1-amino-2-methyl-propyl)-5-benzyl-3-methyl-5H-isothiazolo[5,4-d]pyrimidin-4-one (Section 1: Method 31) in dichloromethane (30 mL), 4 Å molecular sieves (5 g) was added followed by (3-oxo-propyl)-carbamic acid tert-butyl ester (1.2 eq) and the reaction mixture was stirred at r.t. for 3 h and the progress of the reaction was monitored by MS. After the complete disappearance of the starting amine, a catalytic amount of acetic acid was added to the reaction followed by sodium triacetoxyborohydride (1.2 eq) and the reaction mixture was stirred at r.t. overnight. After the completion of the reaction (MS), the reaction mixture was filtered and the residue was washed with dichloromethane and the filtrate was washed with water (100 mL), brine (100 mL) and concentrated to get the product which was used as such for the next reaction. m/z 486 (MH⁺)

The following compounds were synthesized according to Section 1: Method 32:

| Section 1: | | |
|---|---|---|
| Method # | Compound Name | m/z |
| 32a | (3-{1-[5-(4-Fluoro-benzyl)-3-methyl-4-oxo- | 504 |
| | 4,5-dihydro-isothiazolo[5,4-d]pyrimidin-6-yl]- | (MH⁺) |
| | 2-methyl-propylamino}-propyl)-carbamic | |
| | acid tert-butyl ester | |

Section 1: Method 33

### N-(3-Amino-propyl)-N-[1-(5-benzyl-3-methyl-4-oxo-4,5-dihydro-isothiazolo[5,4-d]pyrimidin-6-yl)-2-methyl-propyl]-4-methyl-benzamide

To a solution of the {3-[1-(5-benzyl-3-methyl-4-oxo-4,5-dihydro-isothiazolo[5,4-d]pyrimidin-6-yl)-2-methyl-propylamino]-propyl}-carbamic acid tert-butyl ester (Section 1: Method 31) in pyridine (10 mL) at r.t., a solution of the p-toluoyl chloride (0.616 g, 4 mmol) in dichloromethane (10 mL) was added dropwise and the resulting solution was stirred at r.t. for 2 days after which the TLC showed the disappearance of most of the SM. The reaction mixture was diluted with CH₂Cl₂ (100 mL) washed with water (2×100 mL), brine (100 mL) and dried (Na₂SO₄). Concentration of the organic layer provided the product which was purified by column (silica gel) chromatography using 20-30% EtOAC in hexanes as eluent. Yield=0.276 g of amide. The acylated product was dissolved in 4M HCl in 1,4-dioxane and the mixture was stirred at r.t. for 20 min and the TLC showed the complete disappearance of the starting material. The reaction mixture was concentrated in a rotory evaporator and the residue was triturated with ether. The precipitated product was filtered off and washed with ether and dried under vacuo to yield N-(3-amino-propyl)-N-[1-(5-benzyl-3-methyl-4-oxo-4,5-dihydro-isothiazolo[5,4-d]pyrimidin-6-yl)-2-methyl-propyl]-4-methyl-benzamide as the hydrochloride salt (196 mg, 99%). White powder, mp. 139-140° C. m/z 504 (MH⁺), ¹H NMR (DMSO-d6 300 MHz, 96° C.) δ: 0.45 (d, 3H), 0.90 (d, 3H), 1.12-1.30 (m, 1H), 1.46-1.63 (m, 1H), 2.25 (t, 2H), 2.36 (s, 3H), 2.64-2.7 (m, 1H), 2.68 (s, 3H), 3.34 (t, 2H), 5.06 (d, 1H), 5.59 (d, 1H), 5.90 (d, 1H), 7.20-7.40 (m, 9H), 7.71 (bs, 3H).

The following compounds were synthesized according to Section 1: Method 33:

| Section 1: | | |
|---|---|---|
| Method # | Compound Name | m/z |
| 33a | N-(3-Amino-propyl)-N-{1-[5-(4-fluoro-benzyl)-3- | 522 |
| | methyl-4-oxo-4,5-dihydro-isothiazolo[5,4- | (MH⁺) |
| | d]pyrimidin-6-yl]-2-methyl-propyl}- | |
| | 4-methyl-benzamide | |

Section 1: Method 34

### 3-Methyl-5-(3-methyl-butyryl)-isoxazol-4-carboxylic acid amide

A mixture of 5-amino-3-methyl-isoxazole-4-carboxylic acid amide (10 g, 70 mmol) in 25 ml of isovaleric anhydride was stirred at 110-145° C. for 1 h. The brown solution was diluted with hexane (500 ml) and cooled down. The precipitated gum was separated from the mixture and washed with hexane, dried in vacuo. 3-Methyl-5-(3-methyl-butyryl)-isoxazole-4-carboxylic acid amide was obtained as a yellow gum. Further used without purification in Section 1: Method 35.

Section 1: Method 35

### 6-Isobutyl-3-methyl-5H-isoxazolo[5,4-d]pyrimidin-4-one

A suspension of 3-methyl-5-(3-methyl-butyryl)-isoxazole-4-carboxylic acid amide (Section 1: Method 34) (split into 40 vials) in 3.5 ml of 2N NaOH aq was subjected to microwave irradiation at 140° C. for 20 min. The resulting solution was cooled with an ice bath, and the pH was adjusted to 1˜3 with concentrated HCl. The solid was filtered, washed with water, dried over vacuum at 40° C. overnight. 6-Isobutyl-3-methyl-5H-isoxazolo[5,4-d]pyrimidin-4-one (8 g) was obtained as a white solid. 55% yield for two steps. m/z: 208 (MH⁺), ¹H NMR (400 MHz, DMSO-d6): 0.76 (d, 6H), 1.95 (m, 1H), 2.25 (s, 3H), 2.32 (d, 2H), 12.55 (s, 1H).

Section 1: Method 36

### 5-Benzyl-6-isobutyl-3-methyl-5H-isoxazolo[5,4-d]pyrimidin-4-one

A suspension of 6-isobutyl-3-methyl-5H-isoxazolo[5,4-d]pyrimidin-4-one (Section 1: Method 35) (5 g, 24.4 mmol), benzylbromide (4.17 g, 24.4 mmol), potassium carbonate (6.7 g, 48.8 mmol) in 20 ml DMF was stirred at room temperature for 2 days. The mixture was diluted with water, extracted with ethyl acetate (100 ml×3), the combined organic phases were dried over anhydrous sodium sulfate, concentrated, purified by flash column chromatography (elute: hexane-ethyl acetate=7:1). 5-benzyl-6-isobutyl-3-methyl-5H-isoxazolo[5,4-d]pyrimidin-4-one was obtained as white solid (3 g, 10.1 mmol) (41%).

m/z: 298 (MH⁺), ¹H NMR (400 MHz, DMSO-d6): 0.90 (d, 6H), 2.30 (m, 1H), 2.55 (s, 3H), 2.75 (d, 2H), 5.42 (s, 2H), 7.22-7.43 (m, 5H).

The following compounds were synthesized according to Section 1: Method 36:

| Section 1: | | |
|---|---|---|
| Method # | Compound Name | m/z |
| 36a | 5-(4-Fluoro-benzyl)-6-isobutyl-3-methyl-5H- | 316 |
| | isoxazolo[5,4-d]pyrimidin-4-one | (MH⁺) |

Section 1: Method 37

### 5-Benzyl-6-(1-bromo-2-methyl-propyl)-3-methyl-5H-isoxazolo[5,4-d pyrimidin-4-one

A solution of 5-benzyl-6-isobutyl-3-methyl-5H-isoxazolo[5,4-d]pyrimidin-4-one (Section 1: Method 36) (130 mg, 0.44 mmol) and sodium acetate (90 mg, 1.09 mmol, 2.5 eq) in glacial acetic acid (2 ml) was treated with a preformed bromine solution (0.7 ml bromine in 10 ml of glacial acetic acid) (1.54 ml, 2 mmol). The mixture was stirred at 110-120° C. for 1 day. Excess bromine (1.54 ml, 2 mmol) was added to the mixture every 4 hours for two times at 110-120° C. Water was added to the mixture to which was subsequently added potassium carbonate and extracted with methylene chloride (20 ml×3), the combined organic phases were washed with water and dried over anhydrous sodium sulfate, then concentrated to give the product which was purified by ISCO (elute: hexane-ethyl acetate). 100 mg (60%) of 5-benzyl-6-(1-bromo-2-methyl-propyl)-3-methyl-5H-isoxazolo[5,4-d]pyrimidin-4-one was obtained as a yellow gum. m/z: 377 (MH⁺), ¹HNMR (400 MHz, DMSO-d6): 0.55 (d, 3H), 1.02 (d, 3H), 2.48 (m, 4H), 4.75 (d, 1H), 5.60 (d, 1H), 5.70 (d, 1H), 7.16-7.30 (m, 5H).

The following compounds were synthesized according to Section 1: Method 37:

| Section 1: | | |
|---|---|---|
| Method # | Compound Name | m/z |
| 37a | 6-(1-Bromo-2-methyl-propyl)-5-(4-fluoro-benzyl)-3- | 396 |
| | methyl-5H-isoxazolo[5,4-d]pyrimidin-4-one | (MH⁺) |

Section 1: Method 38

### 6-(1-Azido-2-methyl-propyl)-5-benzyl-3-methyl-5H-isoxazolo[5,4-d]pyrimidin-4-one

A suspension of 5-benzyl-6-(1-bromo-2-methyl-propyl)-3-methyl-5H-isoxazolo[5,4-d]pyrimidin-4-one (Section 1: Method 37) (100 mg, 0.266 mmol) and sodium azide (34.5 mg, 0.53 mmol) in DMF (2 ml) was stirred at 60° C. for 1 h. Water (5 ml) was added to the mixture and then extracted with ethyl acetate (3×20 ml). The combined organic phases were washed with brine (10 ml), dried over sodium sulfate, concentrated to obtain 6-(1-azido-2-methyl-propyl)-5-benzyl-3-methyl-5H-isoxazolo[5,4-d]pyrimidin-4-one which was purified by ISCO (Hexane-Ethyl acetate). 50 mg (56%) of a colorless oil was obtained. m/z: 339 (MH⁺), ¹H NMR (400 MHz, DMSO-d6): 0.60 (d, 3H), 0.95 (d, 3H), 2.25 (m, 1H), 2.45 (s, 3H), 4.19 (d, 1H), 5.30 (d, 1H), 5.42 (d, 1H), 7.12-7.30 (m, 5H).

The following compounds were synthesized according to Section 1: Method 38:

| Section 1: | | |
|---|---|---|
| Method # | Compound Name | m/z |
| 38a | 6-(1-Azido-2-methyl-propyl)-5-(4-fluoro-benzyl)-3- | 357 |
| | methyl-5H-isoxazolo[5,4-d]pyrimidin-4-one | (MH⁺) |

Section 1: Method 39

### 6-(1-Amino-2-methyl-propyl)-5-benzyl-3-methyl-5H-isoxazolo[5,4-d]pyrimidin-4-one

A mixture of 6-(1-azido-2-methyl-propyl)-5-benzyl-3-methyl-5H-isoxazolo[5,4-d]pyrimidin-4-one (Section 1: Method 38) (40 mg, 1.118 mmol), triphenylphosphine (62 mg, 0.237 mmol) and water (4 μl) in THF was stirred at 60° C. for 5 hours. Excess amount of water (30 μl) was added to the mixture and stirred at 60° C. for another 10 hours. The volatile solvent was distilled out, the product was purified by ISCO (Ethyl acetate:hexane=60%. 25 mg (68%) of 6-(1-amino-2-methyl-propyl)-5-benzyl-3-methyl-5H-isoxazolo[5,4-d]pyrimidin-4-one was obtained as colorless oil. m/z: 313 (MH⁺), ¹H NMR (400 MHz, DMSO-d6): 0.55 (d, 3H), 0.95 (d, 3H), 2.02 (m, 1H), 2.15 (br, 2H), 2.55 (s, 3H), 3.59 (d, 1H), 5.38 (d, 1H), 5.65 (d, 1H), 7.25-7.42 (m, 5H).

The following compounds were synthesized according to Section 1: Method 39:

| Section 1: | | |
|---|---|---|
| Method # | Compound Name | m/z |
| 39a | 6-(1-Amino-2-methyl-propyl)-5-(4-fluoro-benzyl)-3- | 331 |
| | methyl-5H-isoxazolo[5,4-d]pyrimidin-4-one | (MH⁺) |

Section 1: Method 40

### {3-[1-(5-Benzyl-3-methyl-4-oxo-4,5-dihydro-isoxazolo[5,4-d]pyrimidin-6-yl)-2-methyl-propylamino]-propyl}-carbamic acid tert-butyl ester

A mixture of 6-(1-amino-2-methyl-propyl)-5-benzyl-3-methyl-5H-isoxazolo [5,4-d]pyrimidin-4-one (Section 1: Method 39) (20 mg, 0.064 mmol) and (3-oxo-propyl)-carbamic acid tert-butyl ester (11 mg, 0.064 mmol) in methylene chloride (5 ml) with dried 4 ÅMS was stirred for 1 h at room temperature. Then sodium triacetoxyborohydride (2 eq) and 1 drop of acetic acid were added to the mixture. The mixture was stirred at room temperature for 1 day. The mixture was filtered through a 2μ cartridge, the filtrate was concentrated, the mixture was purified by ISCO (elute: ethyl acetate-hexane=30%˜60%) to give 18 mg (60%) of {3-[1-(5-benzyl-3-methyl-4-oxo-4,5-dihydro-isoxazolo[5,4-d]pyrimidin-6-yl)-2-methyl-propylamino]-propyl}-carbamic acid tert-butyl ester as a white solid. m/z: 470 (MH⁺), ¹H NMR (400 MHz, DMSO-d6): 0.65 (d, 3H), 0.80 (d, 3H), 1.10 (m, 2H), 1.25 (s, 9H), 1.32 (d, 1H), 1.70-1.90 (m, 2H), 2.18 (m, 1H), 2.49 (s, 3H), 2.70 (m, 2H), 3.48 (d, 1H), 5.15 (d, 1H), 5.51 (d, 1H), 6.55 (br, 1H), 7.12-7.32 (m, 5H).

The following compounds were synthesized according to Section 1: Method 40:

| Section 1: | | |
|---|---|---|
| Method # | Compound Name | m/z |
| 40a | (3-{1-[5-(4-Fluoro-benzyl)-3-methyl-4-oxo-4,5- | 488 |
| | dihydro-isoxazolo[5,4-d]pyrimidin-6-yl]-2-methyl- | (MH⁺) |
| | propylamino}-propyl)-carbamic acid tert-butyl ester | |

Section 1: Method 41

### {3-[[1-(5-Benzyl-3-methyl-4-oxo-4,5-dihydro-isoxazolo[5,4-d]pyrimidin-6-yl)-2-methyl-propyl]-(4-methyl-benzoyl)-amino]-propyl}-carbamic acid tert-butyl ester

A solution of {3-[1-(5-benzyl-3-methyl-4-oxo-4,5-dihydro-isoxazolo[5,4-d]pyrimidin-6-yl)-2-methyl-propylamino]-propyl}-carbamic acid tert-butyl ester (Section 1: Method 40) (100 mg, 0.213 mmol) in dichloromethane (4 ml) was added toluoyl chloride (66 mg, 0.426 mmol) followed by triethylamine (65 mg, 0.639 mmol). The mixture was stirred at 30-40° C. for 2 days. The mixture was then diluted with dichloromethane, washed with saturated sodium bicarbonate aq. The organic phase was dried over sodium sulfate, filtered, and concentrated. The crude oil was purified by ISCO (solvent: ethyl acetate-hexane) to give {3-[[1-(5-benzyl-3-methyl-4-oxo-4,5-dihydro-isoxazolo[5,4-d]pyrimidin-6-yl)-2-methyl-propyl]-(4-methyl-benzoyl)-amino]-propyl}-carbamic acid tert-butyl ester as white solid (115 mg, 0.196 mmol). m/z: 588 (MH⁺)

The following compounds were synthesized according to Section 1: Method 41:

| Section 1: | | | | |
|---|---|---|---|---|
| Method # | Compound Name | m/z | SM | Acylating agent |
| 41a | {3-[{1-[5-(4-Fluoro-benzyl)-3-methyl-4- | 606 | Section | 4-methyl-benzoyl |
| | oxo-4,5-dihydro-isoxazolo[5,4- | (MH⁺) | 1: | chloride |
| | d]pyrimidin-6-yl]-2-methyl-propyl}-(4- | | Method | |
| | methyl-benzoyl)-amino]-propyl}- | | 40a | |
| | carbamic acid tert-butyl ester | | | |
| 41b | {3-[[1-(5-Benzyl-3-methyl-4-oxo-4,5- | 606 | Section | 3-Fluoro-4- |
| | dihydro-isoxazolo[5,4-d]pyrimidin-6-yl)- | (MH⁺) | 1: | methyl-benzoyl |
| | 2-methyl-propyl]-(3-fluoro-4-methyl- | | Method | chloride |
| | benzoyl)-amino]-propyl}-carbamic acid | | 40 | |
| | tert-butyl ester | | | |
| 41c | {3-[{1-[5-(4-Fluoro-benzyl)-3-methyl-4- | 624 | Section | 3-Fluoro-4- |
| | oxo-4,5-dihydro-isoxazolo[5,4- | (MH⁺) | 1: | methyl-benzoyl |
| | d]pyrimidin-6-yl]-2-methyl-propyl}-(3- | | Method | chloride |
| | fluoro-4-methyl-benzoyl)-amino]- | | 40a | |
| | propyl}-carbamic acid tert-butyl ester | | | |

Section 1: Method 42

### N-(3-Amino-propyl)-N-[1-(5-benzyl-3-methyl-4-oxo-4,5-dihydro-isoxazolo[5,4-d]pyrimidin-6-yl)-2-methyl-propyl]-4-methyl-benzamide

A solution of {3-[[1-(5-benzyl-3-methyl-4-oxo-4,5-dihydro-isoxazolo[5,4-d]pyrimidin-6-yl)-2-methyl-propyl]-(4-methyl-benzoyl)-amino]-propyl}-carbamic acid tert-butyl ester (Section 1: Method 41) (0.2 mmol) in 3 ml of 4 M HCl in dioxane was stirred at room temperature for 2 hr. The solvent was distilled off by vacuo, the residue was dried at 40˜50° C. for overnight under vacuum. N-(3-Amino-propyl)-N-[1-(5-benzyl-3-methyl-4-oxo-4,5-dihydro-isoxazolo[5,4-d]pyrimidin-6-yl)-2-methyl-propyl]-4-methyl-benzamide was obtained as the HCl salt. m/z 488 (MH⁺), ¹HNMR (500 MHz, 100° C., DMSO-d₆): 0.48 (d, 3H), 0.94 (d, 3H), 1.30 (m, 1H), 1.60 (m, 1H), 2.35 (m, 2H), 2.38 (s, 3H), 2.58 (s, 3H), 2.70 (m, 1H), 3.37 (m, 2H), 5.11 (d, 1H), 5.64 (d, 1H), 5.90 (d, 1H), 7.23-7.39 (m, 9H), 7.63 (br, 3H).

The following compounds were synthesized according to Section 1: Method 42:

| Section 1: | | |
|---|---|---|
| Method # | Compound Name | m/z |
| 42a | N-(3-Amino-propyl)-N-{1-[5-(4-fluoro-benzyl)-3- | 506 |
| | methyl-4-oxo-4,5-dihydro-isoxazolo[5,4- | (MH⁺) |
| | d]pyrimidin-6-yl]-2-methyl-propyl}- | |
| | 4-methyl-benzamide | |
| 42b | N-(3-Amino-propyl)-N-[1-(5-benzyl-3-methyl- | 506 |
| | 4-oxo-4,5-dihydro-isoxazolo[5,4-d]pyrimidin-6-yl)- | (MH⁺) |
| | 2-methyl-propyl]-3-fluoro-4-methyl-benzamide | |
| 42c | N-(3-Amino-propyl)-3-fluoro-N-{1-[5-(4- | 524 |
| | fluoro-benzyl)-3-methyl-4-oxo-4,5-dihydro- | (MH⁺) |
| | isoxazolo[5,4-d]pyrimidin-6-yl]-2-methyl- | |
| | propyl}-4-methyl-benzamide | |

### EXAMPLE A-1

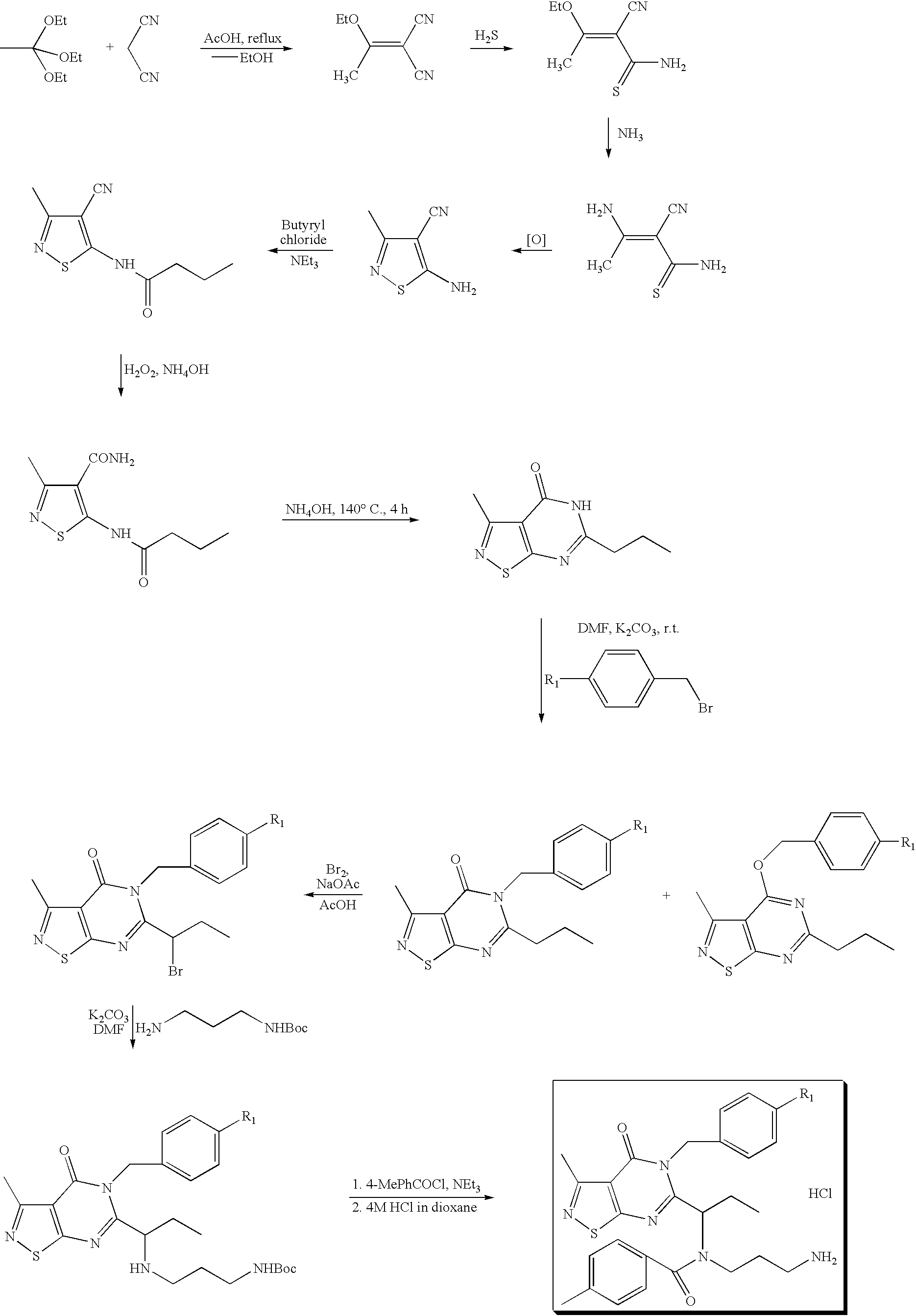

### SECTION 1: EXAMPLES A1

The following compounds were synthesized according to synthetic scheme A-1 above:

| Ex. | Compound | ¹H NMR | m/z | SM |
|---|---|---|---|---|
| A1-1 | N-(3-Amino-propyl)-N-[1-(5- | ¹H NMR(DMSO-d6 300MHz, | m/z | Section |
| | benzyl-3-methyl-4-oxo-4,5- | 96° C.) δ: 7.79(bs, 3H), 7.37-6.95(m, | 490 | 1: |
| | dihydro-isothiazolo-[5,4- | 9H), 5.77(d, 1H), | (MH⁺) | Method |
| | d]pyrimidin-6-yl)-propyl]-4- | 5.50(bs, 1H), 4.83(d, 1H), 3.36(t, | | 11 |
| | methyl-benzamide hydrogen | 2H), 2.72(s, 3H), 2.46(t, 2H), | | |
| | chloride | 2.39(s, 3H), 2.20-2.05(m, 1H), | | |
| | | 1.96-1.75(m, 1H), 1.74-1.40(m, | | |
| | | 2H), 0.63(t, 3H) | | |
| A1-2 | N-(3-Amino-propyl)-N-{1-[5- | ¹H NMR(DMSO-d6 300MHz, | m/z | Section |
| | (4-fluoro-benzyl)-3-methyl-4- | 96° C.) δ: 7.76(bs, 3H), 7.27-7.05(m, | 507 | 1: |
| | oxo-4,5-dihydro- | 8H), 5.70(d, 1H), 5.49(bs, | (MH⁺) | Method |
| | isothiazolo[5,4-d]pyrimidin-6- | 1H), 4.85(d, 1H), 3.36(t, | | 11 |
| | yl]-propyl}-4-methyl- | 2H), 2.99(s, 1H), 2.72(s, 3H), | | |
| | benzamide hydrogen chloride | 2.42(t, 2H), 2.34(s, 3H), 2.20-2.05(m, | | |
| | | 1H), 1.98-1.82(m, | | |
| | | 1H), 1.74-1.38(m, 2H), 0.66(t, 3H) | | |

### EXAMPLE A-2

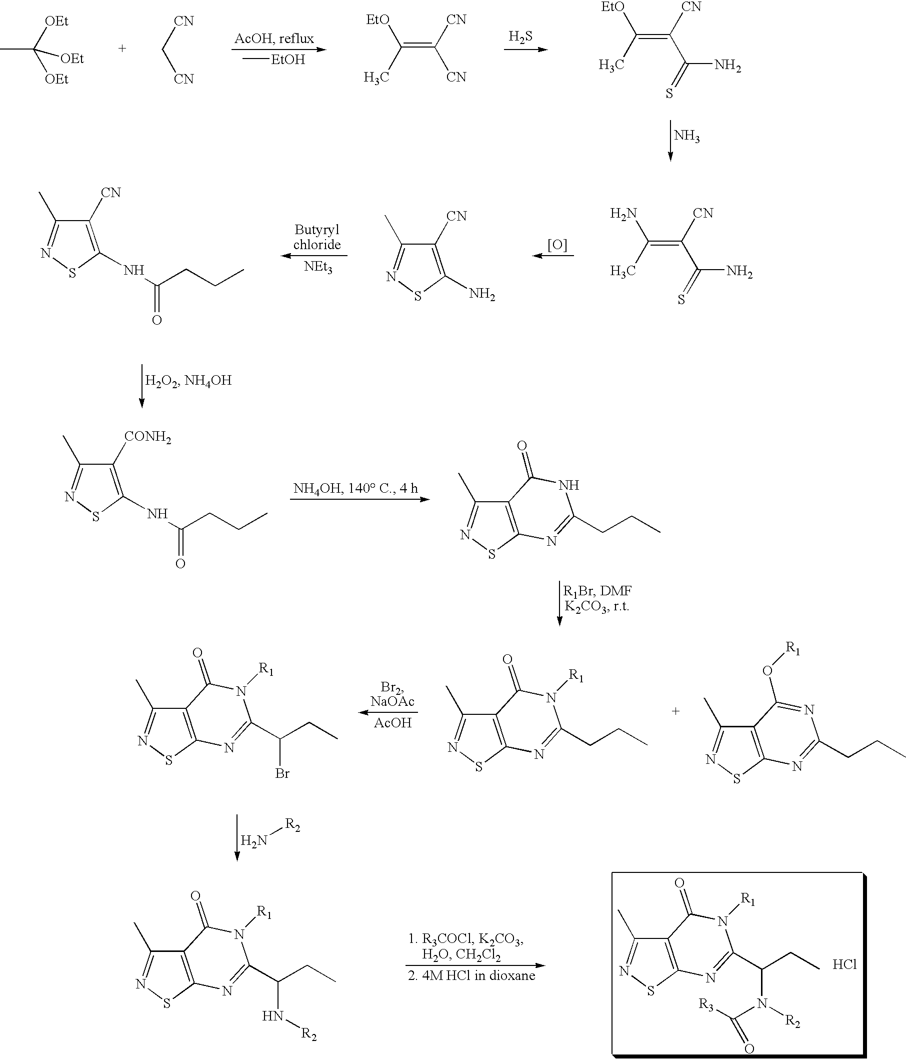

### SECTION 1: EXAMPLES A2

The following compounds were synthesized according to synthetic scheme A-2 above:

| Ex. | Compound | ¹H NMR | m/z | SM |
|---|---|---|---|---|
| A2-1 | N-(3-Amino-propyl)-N-[1-(5- | ¹H NMR(DMSO-d6 500MHz, | m/z | Section |
| | benzyl-3-methyl-4-oxo-4,5- | 96° C.) δ: 7.80(br, 3H), 7.64(d, | 556 | 1: |
| | dihydro-isothiazolo[5,4- | 2H), 7.36-7.28(m, 5H), 7.13(m, | (MH⁺) | Method |
| | d]pyrimidin-6-yl)-propyl]-4- | 2H), 5.80(d, 1H), 5.57(bs, | | 13 |
| | bromo-benzamide hydrogen | 1H), 4.95(d, 1H), 3.38(t, 2H), | | |
| | chloride | 2.77(s, 3H), 2.47(t, 2H), 2.17-2.13(m, | | |
| | | 1H), 1.96-1.91(m, | | |
| | | 1H), 1.72-1.50(m, 2H), 0.68(t, 3H) | | |
| A2-2 | N-(3-Amino-propyl)-N-[1-(5- | ¹H NMR(DMSO-d6 300MHz, | m/z | Section |
| | benzyl-3-methyl-4-oxo-4,5- | 96° C.) δ: 7.56-7.12(m, 9H), | 510 | 1: |
| | dihydro-isothiazolo[5,4- | 5.81(d, 1H), 5.58(bs, 1H), 4.94(d, | (MH⁺) | Method |
| | d]pyrimidin-6-yl)-propyl]-4- | 1H), 3.39(bm, 2H), 2.77(s, | | 13 |
| | chloro-benzamide hydrogen | 3H), 2.46(hidden by DMSO), | | |
| | chloride | 2.15(m, 1H), 1.93(m, 1H), | | |
| | | 1.83-1.420(m, 2H), 0.69(t, 3H) | | |
| A2-3 | N-(3-Amino-propyl)-N-[1-(5- | ¹H NMR(DMSO-d6 300MHz, | m/z | Section |
| | benzyl-3-methyl-4-oxo-4,5- | 96° C.) δ: 7.52-7.09(m, 9H), | 508 | 1: |
| | dihydro-isothiazolo[5,4- | 5.81(d, 1H), 5.54(bs, 1H), 4.93(d, | (MH⁺) | Method |
| | d]pyrimidin-6-yl)-propyl]-3- | 1H), 3.39(t, 2H), 2.76(s, | | 13 |
| | fluoro-4-methyl-benzamide | 3H), 2.46(2H, hidden by | | |
| | hydrogen chloride | DMSO), 2.31(s, 3H), 2.16(m, | | |
| | | 1H), 1.92(m, 1H), 1.70(m, | | |
| | | 1H), 1.45(m, 1H), 0.67(t, 3H) | | |
| A2-4 | N-(3-Amino-propyl)-N-[1-(5- | ¹H NMR(DMSO-d6 300MHz, | m/z | Section |
| | benzyl-3-methyl-4-oxo-4,5- | 96° C.) δ: 7.72-7.29(m, 9H), | 545 | 1: |
| | dihydro-isothiazolo[5,4- | 5.93(d, 1H), 5.82(bs, 1H), 5.10(d, | (MH⁺) | Method |
| | d]pyrimidin-6-yl)-propyl]-2,3- | 1H), 3.30(m, 2H), 2.78(s, | | 13 |
| | dichloro-benzamide hydrogen | 3H), 2.46(m, 2H), 2.20(m, | | |
| | chloride | 1H), 1.94(m, 1H), 1.70-1.50(m, | | |
| | | 2H), 0.70(t, 3H) | | |
| A2-5 | Naphthalene-2-carboxylic acid | ¹H NMR(DMSO-d6 300MHz, | m/z | Section |
| | (3-amino-propyl)-[1-(5-benzyl- | 96° C.) δ: 8.00-7.98(m, 3H), | 526 | 1: |
| | 3-methyl-4-oxo-4,5-dihydro- | 7.87(s, 1H), 7.63-7.61(m, 2H), | (MH⁺) | Method |
| | isothiazolo[5,4-d]pyrimidin-6- | 7.45(d, 1H), 7.27(m, 3H), 7.10(m, | | 13 |
| | yl)-propyl]-amide hydrogen | 2H), 5.83(d, 1H), 5.67(bs, | | |
| | chloride | 1H), 4.98(d, 1H), 3.46(m, 2H), | | |
| | | 2.77(s, 3H), 2.40(m, 2H), 2.24(m, | | |
| | | 1H), 2.04(m, 1H), 1.70(m, | | |
| | | 1H), 1.45(m, 1H), 0.74(t, 3H) | | |
| A2-6 | Benzo[b]thiophene-2-carboxylic | ¹H NMR(DMSO-d6 300MHz, | m/z | Section |
| | acid (3-amino-propyl)-[1-(5- | 96° C.) δ: 7.99-7.85(m, 3H), | 532 | 1: |
| | benzyl-3-methyl-4-oxo-4,5- | 7.60(s, 1H), 7.50-7.30(m, 3H), | (MH⁺) | Method |
| | dihydro-isothiazolo[5,4- | 7.25(bm, 2H), 7.05(bm, 1H), | | 13 |
| | d]pyrimidin-6-yl)-propyl]-amide | 5.78(d, 1H), 5.66(bs, 1H), 4.96(d, | | |
| | hydrogen chloride | 1H), 3.65(t, 2H), 2.76(s, | | |
| | | 3H), 2.62(t, 2H), 2.23-2.21(m, | | |
| | | 1H), 1.97-1.93(m, 1H), 1.92-1.88(m, | | |
| | | 1H), 1.61-1.46(m, | | |
| | | 1H), 0.713(t, 3H) | | |
| A2-7 | N-Azetidin-3-ylmethyl-N-[1-(5- | ¹H NMR(DMSO-d6 500MHz, | m/z | Section |
| | benzyl-3-methyl-4-oxo-4,5- | 96° C.) δ: 7.75-7.20(m, 9H), | 502 | 1: |
| | dihydro- | 5.50(m, 1H), 5.38(m, 1H), | (MH⁺) | Method |
| | isothiazolo[5,4d]pyrimidin-6- | 4.25(m, 2H), 4.00(m, 1H), | | 13 |
| | yl)-propyl]-4-methyl-benzamide | 3.10(t, 2H), 2.75(s, 3H), | | |
| | | 2.73(m, 1H), 2.39(s, 3H), 2.32(m, | | |
| | | 1H), 1.85-1.75(m, 1H), 1.60-1.50(m, | | |
| | | 1H), 0.82(t, 3H) | | |
| A2-8 | N-[1-(5-Benzyl-3-methyl-4-oxo- | ¹H NMR(DMSO-d6 300MHz, | m/z | Section |
| | 4,5-dihydro-isothiazolo[5,4- | 96° C.) δ: 7.31-6.94(m, 9H), | 530 | 1: |
| | d]pyrimidin-6-yl)-propyl]-4- | 5.81(m, 1H), 5.32(bs, 1H), | (MH⁺) | Method |
| | methyl-N-piperidin-3-ylmethyl- | 4.72(bm, 1H), 3.05(bm, 2H), | | 13 |
| | benzamide | 2.76(s, 3H), 2.65(bm, 2H), | | |
| | | 2.37(s, 3H), 2.25(m, 2H), | | |
| | | 1.90(m, 32H), 1.78(m, 3H), 1.55(m, | | |
| | | 1H), 0.65(t, 3H) | | |
| A2-9 | N-(2-Amino-ethyl)-N-[1-(5- | ¹H NMR(DMSO-d6 500MHz, | m/z | Section |
| | benzyl-3-methyl-4-oxo-4,5- | 96° C.) δ: 7.35-6.88(m, 9H), | 476 | 1: |
| | dihydro-isothiazolo[5,4- | 5.80(d, 1H), 5.35(bs, 1H), 4.70(d, | (MH⁺) | Method |
| | d]pyrimidin-6-yl)-propyl]-4- | 1H), 3.80(m, 2H), 2.90(m, | | 13 |
| | methyl-benzamide | 2H), 2.76(s, 3H), 2.45(s, 3H), | | |
| | | 2.05-1.85(m, 2H), 0.60(t, 3H) | | |
| A2- | N-[1-(5-Benzyl-3-methyl-4-oxo- | ¹H NMR(DMSO-d6 500MHz, | m/z | Section |
| 10 | 4,5-dihydro-isothiazolo[5,4- | 96° C.) δ: 7.35-6.88(m, 9H), | 504 | 1: |
| | d]pyrimidin-6-yl)-propyl]-N-(2- | 5.80(d, 1H), 5.40(bs, 1H), 4.80(d, | (MH⁺) | Method |
| | dimethylamino-ethyl)-4-methyl- | 1H), 3.75(m, 2H), 3.00(m, | | 14 |
| | benzamide | 1H), 2.80(s, 3H), 2.70(m, 1H), | | |
| | | 2.52-2.50(2s, 6H), 2.35(s, 3H), | | |
| | | 2.15-1.98(m, 2H), 0.70(t, 3H) | | |
| A2- | N-[1-(5-Benzyl-3-methyl-4-oxo- | ¹H NMR(DMSO-d6 300MHz, | m/z | Section |
| 11 | 4,5-dihydro-isothiazolo[5,4- | 96° C.) δ: 7.44-7.00(m, 9H), | 518 | 1: |
| | d]pyrimidin-6-yl)-propyl]-N-(3- | 5.82(d, 1H), 5.51(bs, 1H), 4.86(d, | (MH⁺) | Method |
| | dimethylamino-propyl)-4- | 1H), 3.41(t, 2H), 2.75(s, | | 14 |
| | methyl-benzamide | 3H), 2.50(s, 6H), 2.39(bm, | | |
| | | 2H), 2.12-2.05(m, 1H), 1.93-1.90(m, | | |
| | | 1H), 1.75(m, 1H), | | |
| | | 1.50(m, 1H), 0.66(t, 3H) | | |

### EXAMPLE B

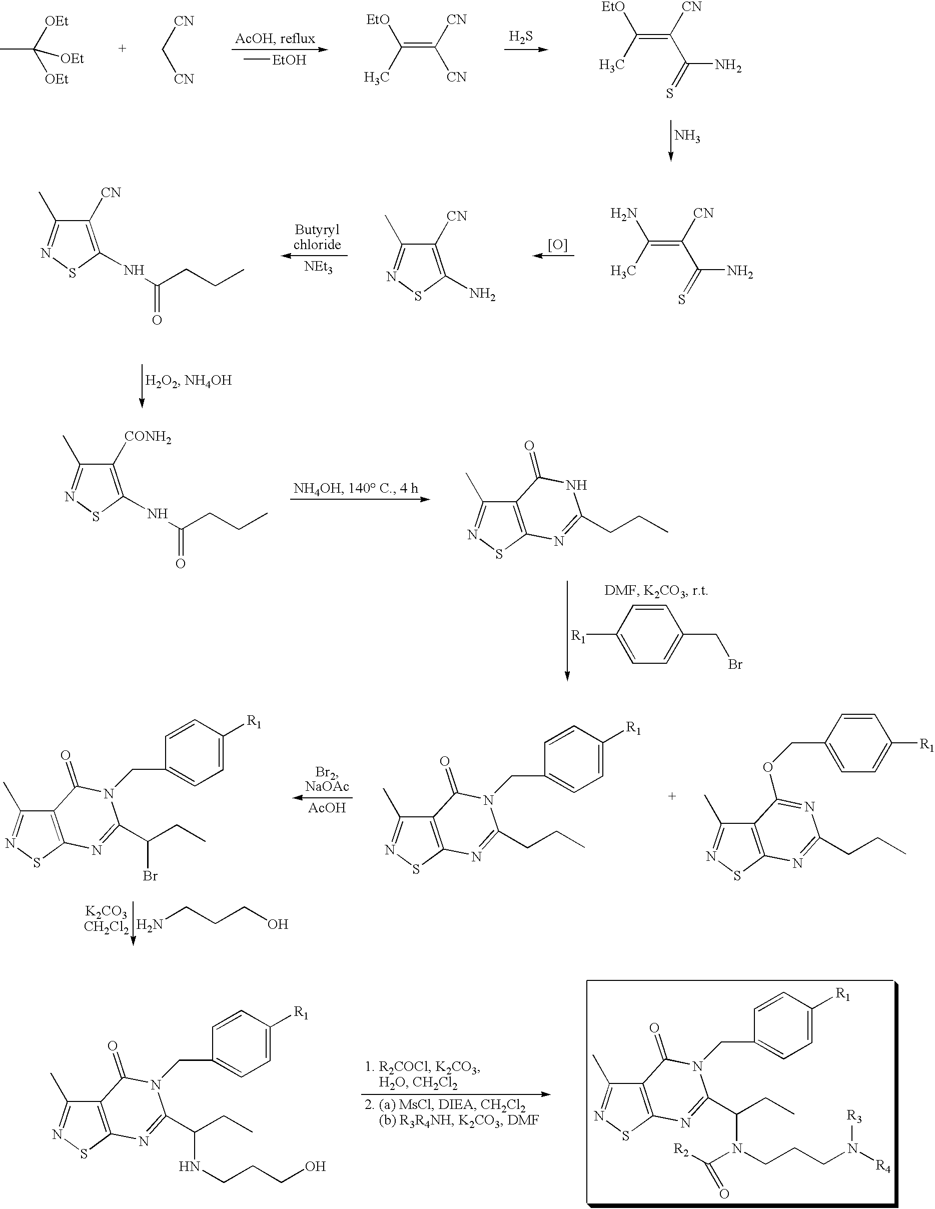

### SECTION 1: EXAMPLES B

The following compounds were synthesized according to Section 1: Example B above:

| Ex. | Compound | ¹H NMR | m/z | SM |
|---|---|---|---|---|
| B-1 | N-[1-(5-benzyl-3-methyl-4-oxo- | ¹H NMR(DMSO-d6 400MHz, | m/z | Section |
| | 4,5-dihydroisothiazolo-[5,4- | 96° C.) δ: 7.35-7.00(m, 9H), | 532 | 1: |
| | d]pyrimidin-6-yl)propyl]-N-[3- | 5.80(d, 1H), 5.50(bs, 1H), 4.90(d, | (MH⁺) | Method |
| | (isopropylamino)propyl]-4- | 1H), 3.40(t, 2H), 3.05(b, | | 16 |
| | methylbenzamide | 1H), 2.80(s, 3H), 2.63(b, 2H), | | |
| | | 2.35(s, 3H), 2.20-2.05(m, 1H), | | |
| | | 2.00-1.85(m, 1H), 1.74-1.70(m, | | |
| | | 1H), 1.60-1.40(m, 1H), | | |
| | | 1.10(d, 6H), 0.65(t, 3H) | | |
| B-2 | N-[1-(5-benzyl-3-methyl-4-oxo- | ¹H NMR(DMSO-d6 400MHz, | m/z | Section |
| | 4,5-dihydroisothiazolo-[5,4- | 96° C.) δ: 7.4-7.00(m, 9H), | 530 | 1: |
| | d]pyrimidin-6-yl)propyl]-N-[3- | 5.80(d, 1H), 5.52(bs, 1H), 4.85(d, | (MH⁺) | Method |
| | (cyclopropylamino)propyl]-4- | 1H), 3.43(b, 2H), 2.78(s, | | 16 |
| | methylbenzamide | 3H), 2.65(b, 2H), 2.35(s, 3H), | | |
| | | 2.20-1.15(various m, 9H), 0.65(t, 3H) | | |
| B-3 | N-(3-azetidin-1-ylpropyl)-N-[1- | ¹H NMR(DMSO-d6 400MHz, | m/z | Section |
| | (5-benzyl-3-methyl-4-oxo-4,5- | 96° C.) δ: 7.40-7.00(m, 9H), | 530 | 1: |
| | dihydroisothiazolo-[5,4- | 5.85(d, 1H), 5.55(bs, 1H), 4.85(d, | (MH⁺) | Method |
| | d]pyrimidin-6-yl)propyl]-4- | 1H), 3.40(b, 2H), 2.90(b, | | 16 |
| | methylbenzamide | 2H), 2.78(s, 3H), 2.50(b, 2H), | | |
| | | 2.40(s, 3H), 2.35(bm, 2H), | | |
| | | 2.20-2.00(m, 1H), 1.96-1.80(m, | | |
| | | 1H), 1.65-1.50(m, 1H), | | |
| | | 1.40-1.30(m, 3H), 0.65(t, 3H) | | |
| B-4 | N-[1-(5-benzyl-3-methyl-4-oxo- | ¹H NMR(DMSO-d6 500MHz, | m/z | Section |
| | 4,5-dihydroisothiazolo-[5,4- | 96° C.) δ: 7.35-7.07(m, 9H), | 544 | 1: |
| | d]pyrimidin-6-yl)propyl]-4- | 5.82(d, 1H), 5.57(bs, 1H), | (MH⁺) | Method |
| | methyl-N-[3-(3-pyrrolidin-1- | 4.91(d, 1H), 3.60(b, 2H), 2.76(s, | | 16 |
| | ylpropyl) benzamide | 3H), 2.50(6H hidden by | | |
| | | DMSO), 2.38(s, 3H), 2.20-2.10(m, | | |
| | | 1H), 2.00-1.65(various m, | | |
| | | 6H), 1.55-1.40(m, 1H), 0.66(t, 3H) | | |
| B-5 | N-[1-(5-benzyl-3-methyl-4-oxo- | ¹H NMR(DMSO-d6 400MHz, | m/z | Section |
| | 4,5-dihydroisothiazolo-[5,4- | 96° C.) δ: 7.50-6.85(m, 9H), | 504 | 1: |
| | d]pyrimidin-6-yl)propyl]-4- | 5.74(d, 1H), 5.30(bs, 1H), 4.60(d, | (MH⁺) | Method |
| | methyl-N-[3-(methylamino) | 1H), 3.80(t, 2H), 2.82(s, | | 16 |
| | propyl] benzamide | 3H), 2.60(s, 3H), 2.41(s, 3H), | | |
| | | 2.39-2.00(m, 2H), 1.99-1.80(m, | | |
| | | 2H), 0.62(t, 3H) | | |

### EXAMPLE C

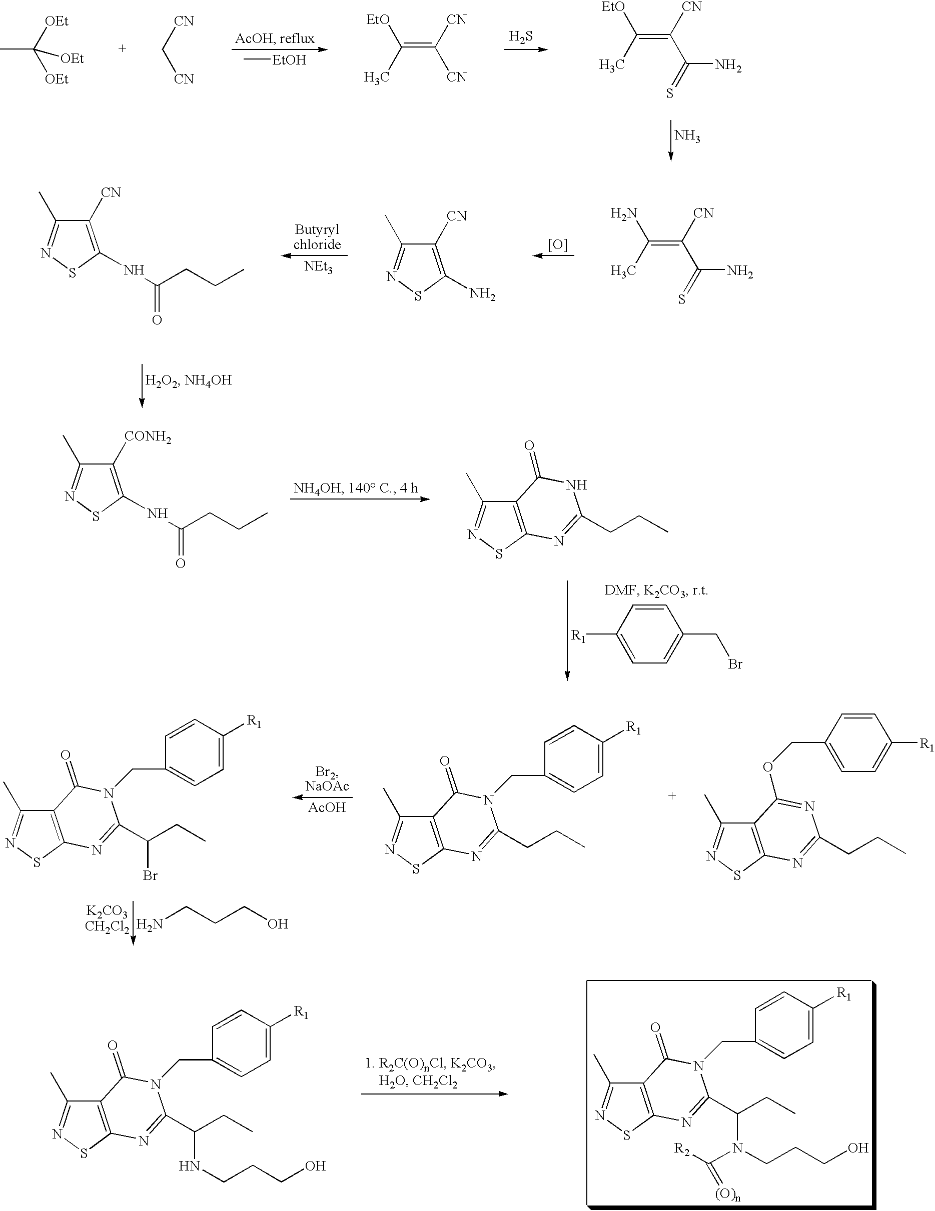

### SECTION 1: EXAMPLES C

The following compounds were synthesized according to synthetic scheme C above:

| Ex. | Compound | ¹H NMR | m/z | SM |
|---|---|---|---|---|
| C-1 | N-[1-(5-Benzyl-3-methyl-4-oxo- | ¹H NMR(DMSO-d6 500MHz, | m/z | Section |
| | 4,5-dihydro-isothiazolo[5,4- | 96° C.) δ: 7.40-7.10(m, 9H), | 491 | 1: |
| | d]pyrimidin-6-yl)-propyl]-N-(3- | 5.85(d, 1H), 5.69(bs, 1H), | (MH⁺) | Method |
| | hydroxy-propyl)-4-methyl- | 5.00(d, 1H), 3.37(t, 2H), 3.05(bm, | | 14 |
| | benzamide | 2H), 2.77(s, 3H), 2.52(s, 1H), | | |
| | | 2.40(s, 3H), 2.16(m, 1H), 1.94(m, | | |
| | | 1H), 1.50-1.40(m, 1H), | | |
| | | 1.20-1.10(m, 1H), 0.71(t, 3H) | | |
| C-2 | 5-Benzyl-6-{1-[(3-hydroxy- | ¹H NMR(DMSO-d6 500MHz, | m/z | Section |
| | propyl)-(4-methyl-benzyl)- | 96° C.) δ: 7.40-6.85(m, 9H), | 477 | 1: |
| | amino]-propyl}-3-methyl-5H- | 5.80(d, 1H), 5.20(d, 1H), | (MH⁺) | Method |
| | isothiazolo[5,4-d]pyrimidin-4- | 3.80(d, 1H), 3.70(m, 1H), 3.62(d, | | 17 |
| | one | 1H), 3.50-3.30(m, 2H), 2.90(m, | | |
| | | 1H), 2.75(s, 3H), 2.33(m, | | |
| | | 2H), 2.25(s, 3H), 2.20-2.16(m, | | |
| | | 1H), 1.90-1.80(m, 1H), | | |
| | | 1.50(m, 2H), 0.65(t, 3H) | | |

### EXAMPLE D

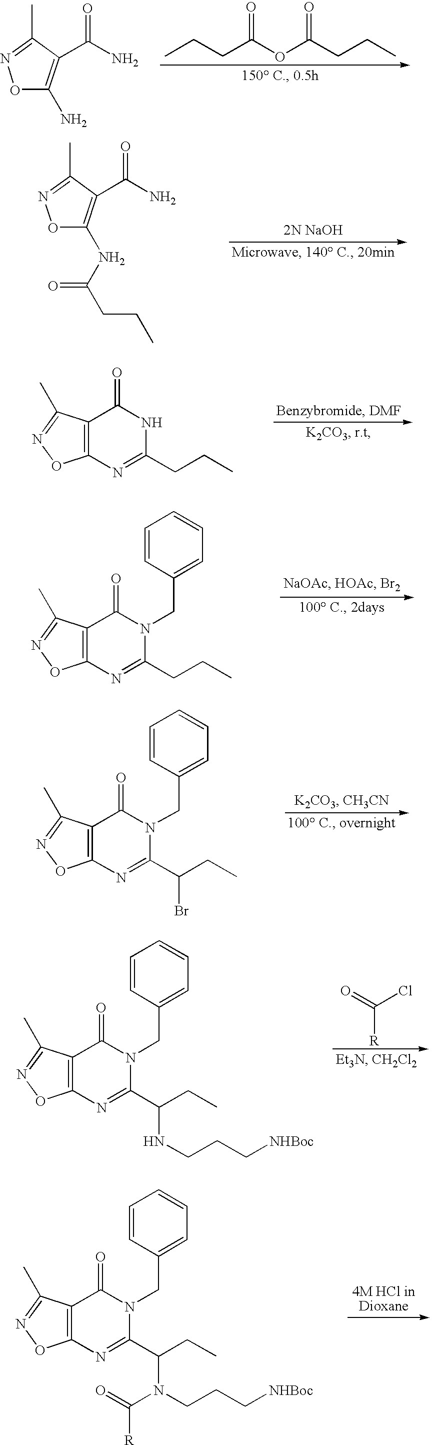

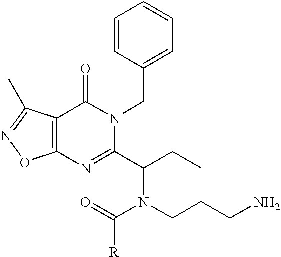

### SECTION 1: EXAMPLES D

The following compounds were synthesized according to synthetic scheme D above:

| Ex. | Compound | ¹H NMR | m/z | SM |
|---|---|---|---|---|
| D-1 | N-(3-Amino-propyl)-N-[1-(5- | ¹H NMR(500MHz, | m/z | Section |
| | benzyl-3-methyl-4-oxo-4,5- | 100° C., DMSO-d₆) δ: 7.73(br, | 474 | 1: |
| | dihydro-isoxazolo[5,4- | 3H), 7.24-7.35(m, 7H), 7.07(br, | (MH⁺) | Method |
| | d]pyrimidin-6-yl)-propyl]-4- | 2H), 5.77(d, J=16.4Hz, | | 24 |
| | methyl-benzamide hydrogen | 1H), 5.50(br, 1H), 4.85(br, | | |
| | chloride | 1H), 3.41(m, 2H), 2.57(s, 3H), | | |
| | | 2.51(m, 2H), 2.39(s, 3H), 2.10(m, | | |
| | | 1H), 1.92(m, 1H), 1.72(m, | | |
| | | 1H), 1.52(m, 1H), 0.68(t, 3H) | | |
| D-2 | N-(3-Amino-propyl)-N-[1-(5- | ¹H NMR(500MHz, 100° C., | m/z | Section |
| | benzyl-3-methyl-4-oxo-4,5- | DMSO-d₆) δ: 7.85(br, 3H), | 478 | 1: |
| | dihydro-isoxazolo[5,4- | 7.13-7.42(m, 9H), 5.78(d, | (MH⁺) | Method |
| | d]pyrimidin-6-yl)-propyl]-4- | J=16.2Hz, 1H), 5.55(br, 1H), | | 24 |
| | fluoro-benzamide hydrogen | 4.98(d, J=16.2Hz, 1H), 3.39(m, | | |
| | chloride | 2H), 2.57(s, 3H), 2.48(m, | | |
| | | 2H), 2.11(m, 1H), 1.94(m, | | |
| | | 1H), 1.72(m, 1H), 1.51(m, | | |
| | | 1H), 0.70(t, 3H) | | |
| D-3 | N-(3-Amino-propyl)-N-[1-(5- | ¹H NMR(500MHz, 100° C., | m/z | Section |
| | benzyl-3-methyl-4-oxo-4,5- | DMSO-d₆): δ: 7.67-7.80(m, | 529 | 1: |
| | dihydro-isoxazolo[5,4- | 4H), 7.29-7.47(m, 7H), 5.92(d, | (MH⁺) | Method |
| | d]pyrimidin-6-yl)-propyl]-2,3- | 1H), 5.83(br, 1H), 5.14(d, 1H), | | 24 |
| | dichloro-benzamide hydrogen | 3.20(m, 2H), 2.58(s, 3H), 2.39(m, | | |
| | chloride | 2H), 2.16(m, 1H), 1.95(m, | | |
| | | 1H), 1.68(m, 1H), 1.41(m, | | |
| | | 1H), 0.70(br, 3H) | | |
| D-4 | N-(3-Amino-propyl)-N-[1-(5- | ¹H NMR(500MHz, 100° C., | m/z | Section |
| | benzyl-3-methyl-4-oxo-4,5- | DMSO-d₆): δ: 7.77(m, 3H), | 492 | 1: |
| | dihydro-isoxazolo[5,4- | 7.31-7.37(m, 4H), 7.06-7.12(m, | (MH⁺) | Method |
| | d]pyrimidin-6-yl)-propyl]-3- | 4H), 5.77(d, 1H), 5.50(br, | | 24 |
| | fluoro-4-methyl-benzamide | 1H), 4.90(br, 1H), 3.40(m, | | |
| | hydrogen chloride | 2H), 2.57(s, 3H), 2.49(m, 2H), | | |
| | | 2.31(d, 3H), 2.13(m, 1H), 1.94(m, | | |
| | | 1H), 1.73(m, 1H), 1.51(m, | | |
| | | 1H), 0.69(t, 3H) | | |
| D-5 | N-(3-Amino-propyl)-N-[1-(5- | ¹H NMR(500MHz, 100° C., | m/z | Section |
| | benzyl-3-methyl-4-oxo-4,5- | DMSO-d₆): δ: 7.71(br, 1H), | 490 | 1: |
| | dihydro-isoxazolo[5,4- | 6.99-7.35(m, 9H), 5.77(d, | (MH⁺) | Method |
| | d]pyrimidin-6-yl)-propyl]-4- | J=16.4Hz, 1H), 5.55(br, 1H), | | 24 |
| | methoxy-benzamide hydrogen | 4.90(d, J=16.4Hz, 1H), 3.85(s, | | |
| | chloride | 3H), 3.43(m, 2H), 2.57(s, 3H), | | |
| | | 2.51(m, 2H), 2.11(m, 1H), | | |
| | | 1.92(m, 1H), 1.72(m, 1H), | | |
| | | 1.52(m, 1H), 0.68(t, 3H) | | |

### EXAMPLE E

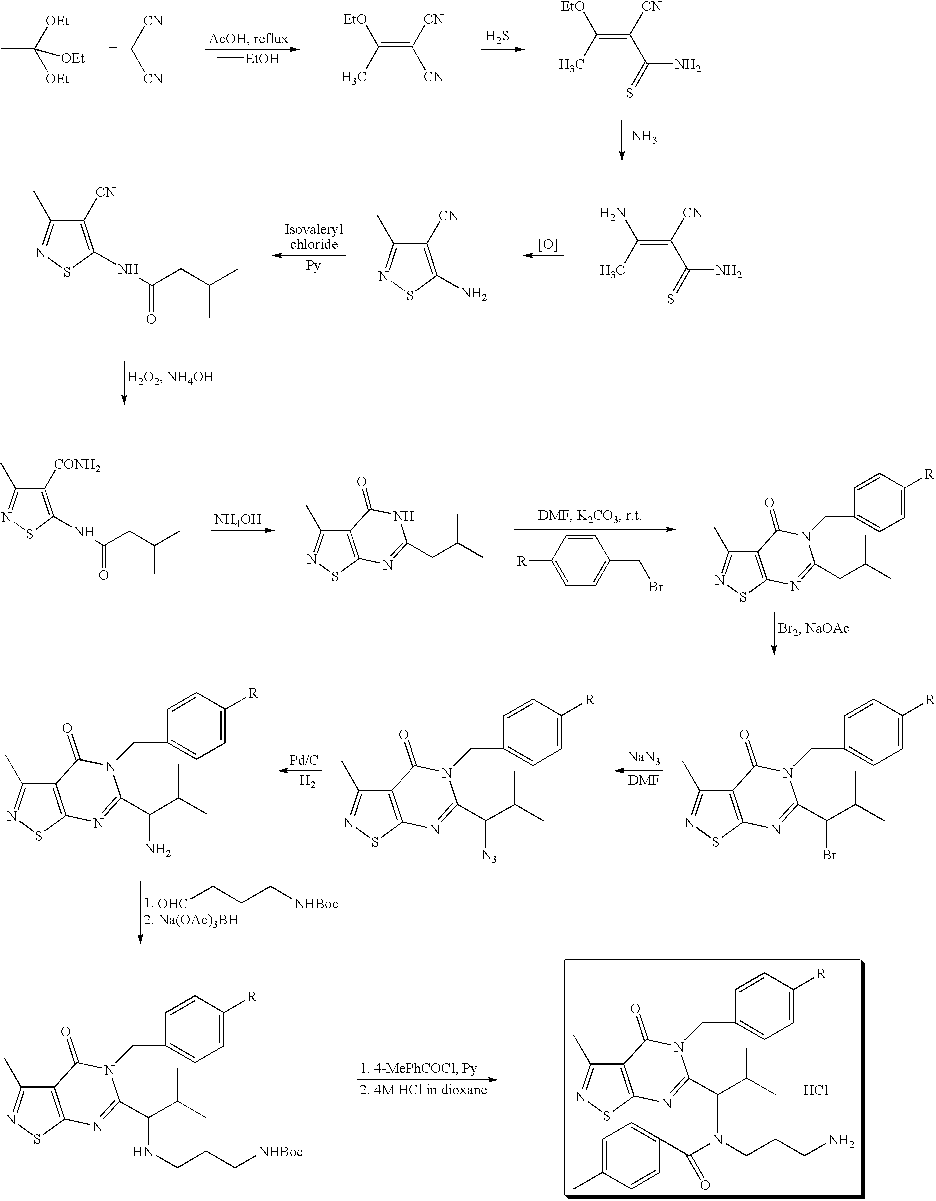

### SECTION 1: EXAMPLES E

The following compounds were synthesized according to synthetic scheme E above:

| Ex. | Compound | ¹H NMR | m/z | SM |
|---|---|---|---|---|
| E-1 | N-(3-Amino-propyl)-N-[1-(5- | ¹H NMR(300MHz, 96° C., | m/z | Section |
| | benzyl-3-methyl-4-oxo-4,5- | DMSO-d₆) δ: 7.71(bs, 3H), | 504 | 1: |
| | dihydro-isothiazolo[5,4- | 7.20-7.40(m, 9H), 5.90(d, 1H), | (MH⁺) | Method |
| | d]pyrimidin-6-yl)-2-methyl- | 5.59(d, 1H), 5.06(d, 1H), 3.34(t, | | 33 |
| | propyl]-4-methyl-benzamide | 2H), 2.68(s, 3H), 2.64-2.7(m, | | |
| | hydrogen chloride | 1H), 2.36(s, 3H), 2.25(t, | | |
| | | 2H), 1.46-1.63(m, 1H), 1.12-1.30(m, | | |
| | | 1H), 0.90(d, 3H), 0.45(d, 3H) | | |
| E-2 | N-(3-Amino-propyl)-N-{1-[5- | ¹H NMR(300MHz, 96° C., | m/z | Section |
| | (4-fluoro-benzyl)-3-methyl-4- | DMSO-d₆) δ: 7.12-7.67(m, | 522 | 1: |
| | oxo-4,5-dihydro-isothiazolo[5,4- | 11H), 5.86(d, 1H), 5.57(d, | (MH⁺) | Method |
| | d]pyrimidin-6-yl]-2-methyl- | 1H), 5.04(d, 1H), 3.35(t, 2H), | | 33 |
| | propyl}-4-methyl-benzamide | 2.75(s, 3H), 2.66-2.72(m, 1H), | | |
| | hydrogen chloride | 2.36(s, 3H), 2.27(t, 2H), 1.44-1.56(m, | | |
| | | 1H), 1.10-1.28(m, | | |
| | | 1H), 0.92(d, 3H), 0.47(d, 3H) | | |

### SECTION 1: EXAMPLE F

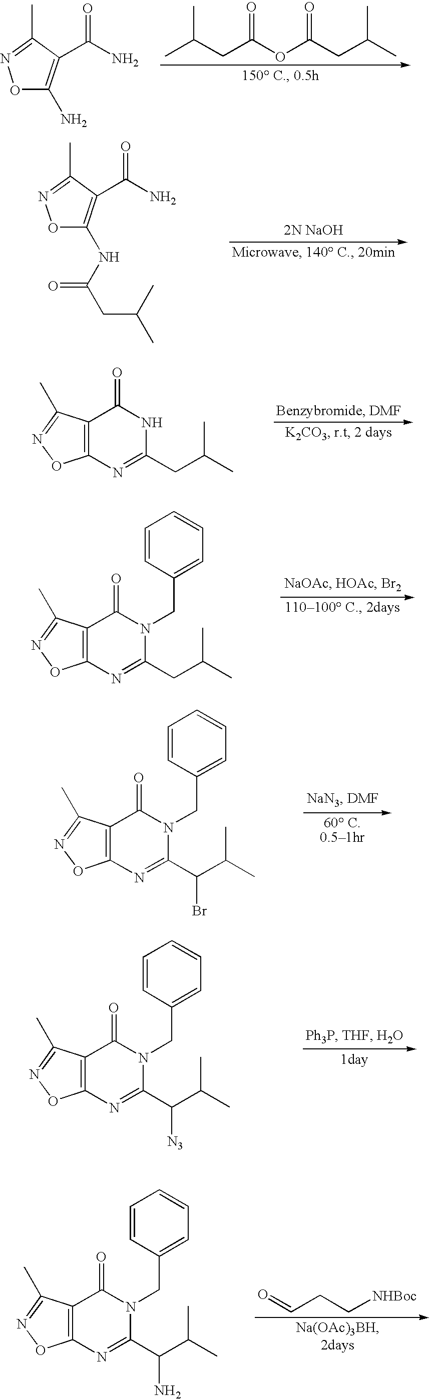

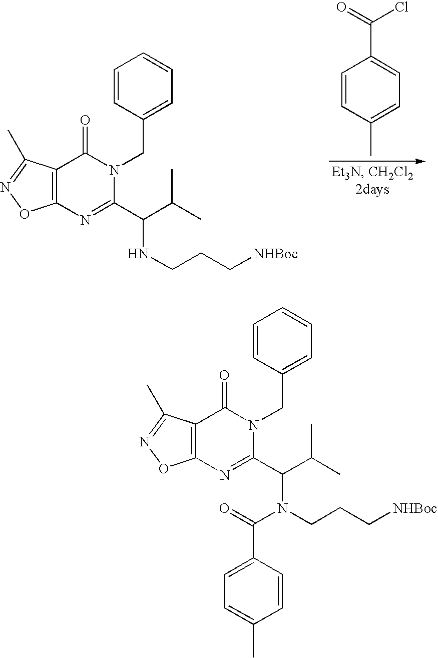

### SECTION 1: EXAMPLES F

The following compounds were synthesized according to synthetic scheme F above:

| Ex. | Compound | ¹H NMR | m/z | SM |
|---|---|---|---|---|
| F-1 | N-(3-Amino-propyl)-N-[1-(5- | ¹H NMR(500MHz, 100° C., | m/z | Section |
| | benzyl-3-methyl-4-oxo-4,5- | DMSO-d₆): δ: 7.63(br, 3H), | 488 | 1: |
| | dihydro-isoxazolo[5,4- | 7.23-7.39(m, 9H), 5.90(d, 1H), | (MH⁺) | Method |
| | d]pyrimidin-6-yl)-2-methyl- | 5.64(d, 1H), 5.11(d, 1H), 3.37(m, | | 42 |
| | propyl]-4-methyl-benzamide | 2H), 2.70(m, 1H), 2.58(s, | | |
| | hydrogen chloride | 3H), 2.38(s, 3H), 2.35(m, 2H), | | |
| | | 1.60(m, 1H), 1.30(m, 1H), | | |
| | | 0.94(d, 3H), 0.48(d, 3H) | | |
| F-2 | N-(3-Amino-propyl)-N-[1-(5- | ¹H NMR(500MHz, 100° C., | m/z | Section |
| | benzyl-3-methyl-4-oxo-4,5- | DMSO-d₆): δ: 7.63(br, 3H), | 506 | 1: |
| | dihydro-isoxazolo[5,4- | 7.10-7.40(m, 8H), 5.90(d, 1H), | (MH⁺) | Method |
| | d]pyrimidin-6-yl)-2-methyl- | 5.64(d, J=10Hz, 1H), 5.10(d, | | 42 |
| | propyl]-3-fluoro-4-methyl- | J=16Hz, 1H), 3.38(m, 2H), | | |
| | benzamide hydrogen chloride | 2.70(m, 1H), 2.58(s, 3H), 2.36(m, | | |
| | | 2H), 2.31(s, 3H), 1.60(m, | | |
| | | 1H), 1.25(m, 1H), 0.93(d, 3H), | | |
| | | 0.48(d, 3H) | | |
| F-3 | N-(3-Amino-propyl)-N-{1-[5-(4- | ¹H NMR(500MHz, 100° C., | m/z | Section |
| | fluoro-benzyl)-3-methyl-4-oxo- | DMSO-d₆) δ: 7.60(br, 3H) | 506 | 1: |
| | 4,5-dihydro-isoxazolo [5,4- | 7.17-7.34(m, 8H), 5.88(d, | (MH⁺) | Method |
| | d]pyrimidin-6-yl]-2-methyl- | J=16Hz, 1H), 5.60(d, J=10Hz, | | 42 |
| | propyl}-4-methyl-benzamide | 1H), 5.10(d, J=16Hz, 1H), | | |
| | hydrogen chloride | 3.40(m, 2H), 2.75(m, 1H), 2.60(s, | | |
| | | 3H), 2.40(s, 3H), 2.32(m, 2H), | | |
| | | 1.55(m, 1H), 1.25(m, 1H), | | |
| | | 0.95(d, 3H), 0.50(d, 3H) | | |
| F-4 | N-(3-Amino-propyl)-3-fluoro-N- | ¹H NMR(500MHz, 100° C., | m/z | Section |
| | {1-[5-(4-fluoro-benzyl)-3- | DMSO-d₆): δ: 7.57(br, 3H) | 524 | 1: |
| | methyl-4-oxo-4,5-dihydro- | 7.11-7.40(m, 7H), 5.86(d, | (MH⁺) | Method |
| | isoxazolo[5,4-d]pyrimidin-6-yl]- | J=16Hz, 1H), 5.61(d, J=10Hz, | | 42 |
| | 2-methyl-propyl}-4-methyl- | 1H), 5.08(d, J=16Hz, 1H), 3.39(m, | | |
| | benzamide hydrogen chloride | 2H), 2.71(m, 1H), 2.58(s, | | |
| | | 3H), 2.36(m, 2H), 2.31(s, 3H), | | |
| | | 1.58(m, 1H), 1.25(m, 1H), | | |
| | | 0.95(d, 3H), 0.50(d, 3H) | | |

Section 1: Utility

The compounds of the invention described in section 1 have utility for the treatment of neoplastic disease by inhibiting the microtubule motor protein HsEg5. In section 1, methods of treatment target Eg5 activity, which is required for the formation of a mitotic spindle and therefore for cell division. Thus, inhibitors of Eg5 have been shown to block cells in the metaphase of mitosis leading to apoptosis of effected cells, and to therefore have anti-proliferative effects. Thus Eg5 inhibitors act as modulators of cell division and are expected to be active against neoplastic disease such as carcinoma of the breast, ovary, lung, colon, prostate or other tissues, as well as leukemias and lymphomas, tumours of the central and peripheral nervous system, and other tumour types such as melanoma, fibrosarcoma and osteosarcoma. Eg5 inhibitors are also expected to be useful for the treatment other proliferative diseases including but not limited to autoimmune, inflammatory, neurological, and cardiovascular diseases.

Compounds of the present invention as described in section 1, have been shown to inhibit Eg5, as determined by Malachite Green Assay described herein.

Compounds provided by this invention in section 1 should also be useful as standards and reagents in determining the ability of a potential pharmaceutical to inhibit Eg5. These would be provided in commercial kits comprising a compound of this invention

Section 1: Assays

Malachite Green Assay

Enzymatic activity of the Eg5 motor and effects of inhibitors was measured using a malchite green assay, which measures phosphate liberated from ATP, and has been used previously to measure the activity of kinesin motors (Hackney and Jiang, 2001). Enzyme was recombinant HsEg5 motor domain (amino acids 1-369-8His) and was added at a final concentration of 6 nM to 100 μl reactions. Buffer consisted of 25 mM PIPES/KOH, pH 6.8, 2 mM MgCl₂, 1 mM EGTA, 1 mM dtt, 0.01% Triton X-100 and 5 μM paclitaxel. Malachite green/ammonium molybdate reagent was prepared as follows: for 800 ml final volume, 0.27 g of Malachite Green (J. T. Baker) was dissolved in 600 ml of H₂O in a polypropylene bottle. 8.4 g ammonium molybdate (Sigma) was dissolved in 200 ml 4N HCl. The solutions were mixed for 20 min and filtered through 0.02 μm filter directly into a polypropylene container.

5 μl of compound diluted in 12% DMSO was added to the wells of 96 well plates. 80 μl of enzyme diluted in buffer solution above was added per well and incubated with compound for 20 min. After this pre-incubation, substrate solution containing 2 mM ATP (final concentration: 300 μM) and 6.053 μM polymerized tubulin (final concentration: 908 nM) in 15 μl of buffer were then added to each well to start reaction. Reaction was mixed and incubated for a particular 20 min at room temperature. The reactions were then quenched by the addition of 150 μl malachite green/ammonium molybdate reagent, and absorbance read at 650 nanometers exactly 5 min after quench using a Spectramax Plus plate reader (Molecular Devices). Data was graphed and IC₅₀s calculated using ExCel Fit (Microsoft).

### Section 2

Section 2: Field of the Invention

The invention as described in section 2 relates to novel fused heterocycles, their pharmaceutical compositions and methods of use. In addition, the invention as described in section 2 relates to therapeutic methods for the treatment and prevention of cancers and to the use of these chemical compounds in the manufacture of a medicament for use in the treatment and prevention of cancers. Section 2: Background of the Invention

One sub-class of anti-cancer drugs (taxanes, vinca-alkaloids) now used extensively in the clinic is directed at microtubules and blocks the cell division cycle by interfering with normal assembly or disassembly of the mitotic spindle (see Chabner, B. A., Ryan, D. P., Paz-Ares, l., Garcia-Carbonero, R., and Calabresi, P: Antineoplastic agents. In Hardman, J. G., Limbird, L. E., and Gilman, A. G., eds. Goodman and Gilman's The Pharmacological Basis of Therapeutics, 10^{th} edition, 2001, The MacGraw-Hill Companies, Inc). Taxol® (paclitaxel), one of the most effective drugs of this class, is a microtubule stabilizer. It interferes with the normal growth and shrinkage of microtubules thus blocking cells in the metaphase of mitosis. Mitotic block is often followed by slippage into the next cell cycle without having properly divided, and eventually by apoptosis of these abnormal cells (Blagosklonny, M. V. and Fojo, T.: Molecular effects of paclitaxel: myths and reality (a critical review). Int J Cancer 1999, 83:151-156.).

Some of the side effects of treatment with paclitaxel are neutropenia and peripheral neuropathy. Paclitaxel is known to cause abnormal bundling of microtubules in interphase cells. In addition, some tumor types are refractory to treatment with paclitaxel, and other tumors become insensitive during treatment. Paclitaxel is also a substrate for the multi-drug resistance pump, P-glycoprotein ((see Chabner et al., 2001).

Thus, there is a need for effective anti-mitotic agents that have fewer side effects than anti-microtubule drugs, and also for agents that are effective against taxane-resistant tumors.

Kinesins are a large family of molecular motor proteins, which use the energy of adenosine 5′-triphosphate (ATP) hydrolysis to move in a stepwise manner along microtubules. For a review, see Sablin, E. P.: Kinesins and microtubules: their structures and motor mechanisms. Curr Opin Cell Biol 2000, 12:35-41 and Schief, W. R. and Howard, J.: Conformational changes during kinesin motility. Curr Opin Cell Biol 2001, 13:19-28.

Some members of this family transport molecular cargo along microtubules to the sites in the cell where they are needed. For example, some kinesins bind to vesicles and transport them along microtubules in axons. Several family members are mitotic kinesins, as they play roles in the reorganization of microtubules that establishes a bipolar mitotic spindle. The minus ends of the microtubules originate at the centrosomes, or spindle poles, whilst the plus ends bind to the kinetochore at the centromeric region of each chromosome. The mitotic spindle lines up the chromosomes at metaphase of mitosis and coordinates their movement apart and into individual daughter cells at anaphase and telophase (cytokinesis). See Alberts, B., Bray, D., Lewis, J., Raff, M., Roberts, K., and Watson, J. D., Molecular Biology of the Cell, 3^{rd} edition, Chapter 18, The Mechanics of Cell Division, 1994, Garland Publishing, Inc. New York.

HsEg5 (homo sapiens Eg5) (Accession X85137; see Blangy, A., Lane H. A., d'Heron, P., Harper, M., Kress, M. and Nigg, E. A.: Phosphorylation by p34cdc2 regulates spindle association of human Eg5, a kinesin-related motor essential for bipolar spindle formation in vivo. Cell 1995, 83(7): 1159-1169) or, KSP (kinesin spindle protein), is a mitotic kinesin whose homologs in many organisms have been shown to be required for centrosome separation in the prophase of mitosis, and for the assembly of a bipolar mitotic spindle. For a review see Kashina, A. S., Rogers, G. C., and Scholey, J. M.: The bimC family of kinesins: essential bipolar mitotic motors driving centrosome separation. Biochem Biophys Acta 1997, 1357: 257-271. Eg5 forms a tetrameric motor, and it is thought to cross-link microtubules and participate in their bundling (Walczak, C. E., Vernos, I., Mitchison, T. J., Karsenti, E., and Heald, R.: A model for the proposed roles of different microtubule-based motor proteins in establishing spindle bipolarity. Curr Biol 1998, 8:903-913). Several reports have indicated that inhibition of Eg5 function leads to metaphase block in which cells display monastral spindles. Recently an Eg5 inhibitor called monastrol was isolated in a cell-based screen for mitotic blockers (Mayer, T. U., Kapoor, T. M., Haggarty, S. J., King, R. W., Schreiber, S. L., and Mitchison, T. J.: Small molecule inhibitor of mitotic spindle bipolarity identified in a phenotype-based screen. Science 1999, 286: 971-974).

Monastrol treatment was shown to be specific for Eg5 over kinesin heavy chain, another closely related motor with different functions (Mayer et al., 1999). Monastrol blocks the release of ADP (adenosine 5′-diphosphate) from the Eg5 motor (Maliga, Z., Kapoor, T. M., and Mitchison, T. J.: Evidence that monastrol is an allosteric inhibitor of the mitotic kinesin Eg5. Chem & Biol 2002, 9: 989-996 and DeBonis, S., Simorre, J.-P., Crevel, I., Lebeau, L, Skoufias, D. A., Blangy, A., Ebel, C., Gans, P., Cross, R., Hackney, D. D., Wade, R. H., and Kozielski, F.: Interaction of the mitotic inhibitor monastrol with human kinesin Eg5. Biochemistry 2003, 42: 338-349) an important step in the catalytic cycle of kinesin motor proteins (for review, see Sablin, 2000; Schief and Howard, 2001). Treatment with monastrol was shown to be reversible and to activate the mitotic spindle checkpoint which stops the progress of the cell division cycle until all the DNA is in place for appropriate division to occur (Kapoor, T. M., Mayer, T. U., Coughlin, M. L., and Mitchison, T. J.: Probing spindle assembly mechanisms with monastrol, a small molecule inhibitor of the mitotic kinesin, Eg54. J Cell Biol 2000, 150(5): 975-988). Recent reports also indicate that inhibitors of Eg5 lead to apoptosis of treated cells and are effective against several tumor cell lines and tumor models (Mayer et al., 1999).

Although Eg5 is thought to be necessary for mitosis in all cells, one report indicates that it is over-expressed in tumor cells (International Patent Application WO 01/31335), suggesting that they may be particularly sensitive to its inhibition. Eg5 is not present on the microtubules of interphase cells, and is targeted to microtubules by phosphorylation at an early point in mitosis (Blangy et al., 1995). See also; Sawin, K. E. and Mitchison, T. J.: Mutations in the kinesin-like protein Eg5 disrupting localization to the mitotic spindle. Proc Natl Acad Sci USA 1995, 92(10): 4289-4293, thus monastrol has no detectable effect on microtubule arrays in interphase cells (Mayer et al., 1999). Another report suggests that Eg5 is involved in neuronal development in the mouse, but it disappears from neurons soon after birth, and thus Eg5 inhibition may not produce the peripheral neuropathy associated with treatment with paclitaxel and other anti-microtubule drugs (Ferhat, L., Expression of the mitotic motor protein Eg5 in postmitotic neurons: implications for neuronal development. J Neurosci 1998, 18(19): 7822-7835). Herein we describe the isolation of a class of specific and potent inhibitors of Eg5, expected to be useful in the treatment of neoplastic disease.

Certain pyrimidones have recently been described as being inhibitors of KSP (WO 03/094839, WO 03/099211, WO 03/050122, WO 03/050064, WO 03/049679, WO 03/049527, WO 04/078758, WO 04/106492 and WO 04/111058).

In section 2, in accordance with the present invention, the present inventors have discovered novel chemical compounds which possess Eg5 inhibitory activity and are accordingly useful for their anti-cell-proliferation (such as anti-cancer) activity and are therefore useful in methods of treatment of the human or animal body.

Section 2: Summary of the Invention

A compound of formula (I): 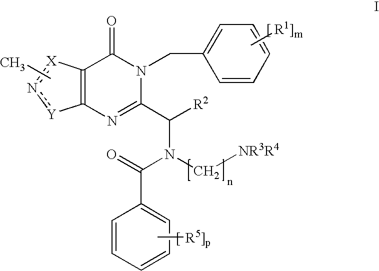
including a pharmaceutically acceptable salt or in vivo hydrolysable ester thereof, wherein:
X is selected from C or S provided that when X is S then Y is C;
Y is selected from C or O or S provided that when Y is C then X is not C;
m is 0 or 1;
R¹ is F when m is 1;
R² is selected from C₁₋₃alkyl;
n is 2 or 3;
R³ and R⁴ are independently selected from H or C₁₋₂alkyl;
R⁵ is selected from F, Cl, Br or C₁₋₂alkyl;
p is 1 or 2;
   selected from:
N-(3-amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide;
N-(3-amino-propyl)-N-{1-[5-(3-fluoro-benzyl)-3-methyl-4-oxo-4,5-dihydro-isothiazolo[5,4-d]pyrimidin-6-yl]-2-methyl-propyl}-4-methyl-benzamide;
N-(2-amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide;
N-(2-amino-ethyl)-N-[1-(5-benzyl-3-methyl-4-oxo-4,5-dihydro-isothiazolo[5,4-d]pyrimidin-6-yl)-2-methyl-propyl]-4-methyl-benzamide;
N-(2-amino-ethyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
N-(3-amino-propyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
N-(3-amino-propyl)-N-[1-(5-benzyl-3-methyl-4-oxo-4,5-dihydro-isothiazolo[5,4-d]pyrimidin-6-yl)-2-methyl-propyl]-4-bromo-benzamide;
N-[1-(5-benzyl-3-methyl-4-oxo-4,5-dihydro-isothiazolo[5,4-d]pyrimidin-6-yl)-2-methyl-propyl]-N-(3-dimethylamino-propyl)-4-methyl-benzamide;
N-[1-(5-benzyl-3-methyl-4-oxo-4,5-dihydro-isothiazolo[5,4-d]pyrimidin-6-yl)-2-methyl-propyl]-N-(3-dimethylamino-propyl)-4-bromo-benzamide;
N-[1-(5-benzyl-3-methyl-4-oxo-4,5-dihydro-isothiazolo[5,4-d]pyrimidin-6-yl)-2-methyl-propyl]-N-(3-dimethylamino-propyl)-3-fluoro-4-methyl-benzamide;
N-(3-amino-propyl)-N-{1-[5-(3-fluoro-benzyl)-3-methyl-4-oxo-4,5-dihydro-isoxazolo[5,4-d]pyrimidin-6-yl]-2-methyl-propyl}-4-methyl-benzamide;
N-(3-amino-propyl)-N-[1-(6-benzyl-3-methyl-7-oxo-6,7-dihydro-isothiazolo[4,5-d]pyrimidin-5-yl)-propyl]-4-methyl-benzamide.

In section 2, the invention also encompasses stereoisomers, enantiomers, in vivo-hydrolysable precursors and pharmaceutically-acceptable salts of compounds of formula (I), pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

Section 2: Detailed Description of the Invention

In section 2, in a first embodiment, the present invention provides a novel compound having structural formula (I): 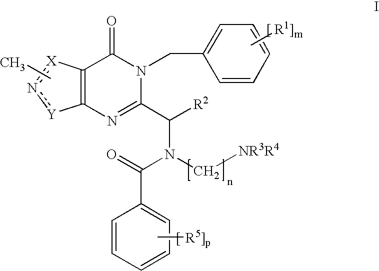
including a pharmaceutically acceptable salt or in vivo hydrolysable ester thereof, wherein:
X is selected from C or S provided that when X is S then Y is C;
Y is selected from C or O or S provided that when Y is C then X is not C;
m is 0, or 1;
R¹ is F, when m is 1;
R² is selected from C₁₋₃alkyl;
n is 2 or 3;
R³ and R⁴ are independently selected from H or C₁₋₂alkyl;
R⁵ is selected from F, Cl, Br, or C₁₋₂alkyl;
p is 1 or 2;
   selected from:
N-(3-amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide;
N-(3-amino-propyl)-N-{1-[5-(3-fluoro-benzyl)-3-methyl-4-oxo-4,5-dihydro-isothiazolo[5,4-d]pyrimidin-6-yl]-2-methyl-propyl}-4-methyl-benzamide;
N-(2-amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide;
N-(2-amino-ethyl)-N-[1-(5-benzyl-3-methyl-4-oxo-4,5-dihydro-isothiazolo[5,4-d]pyrimidin-6-yl)-2-methyl-propyl]-4-methyl-benzamide;
N-(2-amino-ethyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
N-(3-amino-propyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
N-(3-amino-propyl)-N-[1-(5-benzyl-3-methyl-4-oxo-4,5-dihydro-isothiazolo[5,4-d]pyrimidin-6-yl)-2-methyl-propyl]-4-bromo-benzamide;
N-[1-(5-benzyl-3-methyl-4-oxo-4,5-dihydro-isothiazolo[5,4-d]pyrimidin-6-yl)-2-methyl-propyl]-N-(3-dimethylamino-propyl)-4-methyl-benzamide;
N-[1-(5-benzyl-3-methyl-4-oxo-4,5-dihydro-isothiazolo[5,4-d]pyrimidin-6-yl)-2-methyl-propyl]-N-(3-dimethylamino-propyl)-4-bromo-benzamide;
N-[1-(5-benzyl-3-methyl-4-oxo-4,5-dihydro-isothiazolo[5,4-d]pyrimidin-6-yl)-2-methyl-propyl]-N-(3-dimethylamino-propyl)-3-fluoro-4-methyl-benzamide;
N-(3-amino-propyl)-N-{1-[5-(3-fluoro-benzyl)-3-methyl-4-oxo-4,5-dihydro-isoxazolo[5,4-d]pyrimidin-6-yl]-2-methyl-propyl}-4-methyl-benzamide;
N-(3-amino-propyl)-N-[1-(6-benzyl-3-methyl-7-oxo-6,7-dihydro-isothiazolo[4,5-d]pyrimidin-5-yl)-propyl]-4-methyl-benzamide.

In another embodiment of section 2, the present invention provides a novel compound having structural formula (I): 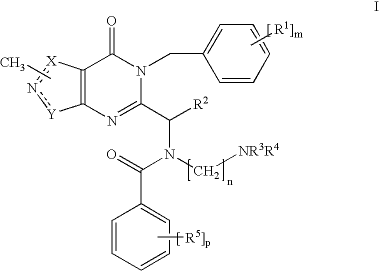
including a pharmaceutically acceptable salt or in vivo hydrolysable ester thereof, wherein:
X is selected from C or S provided that when X is S then Y is C;
Y is selected from C or O or S provided that when Y is C then X is not C;
m is 0, or 1;
R¹ is F, when m is 1;
R² is selected from C₁₋₃alkyl;
n is 2 or 3;
R³ and R⁴ are independently selected from H or C₁₋₂alkyl;
R⁵ is selected from F, Cl, Br, or C₁₋₂alkyl;
p is 1 or 2.

In section 2, in formula (I) the dotted line represents a single or a double bond—the bond between the nitrogen and whichever of X and Y is C is double, the other bond is a single bond.

In section 2, in an additional embodiment the present invention provides a compound of formula (I) wherein X is C or a pharmaceutically acceptable salt or in vivo hydrolysable ester thereof.

In section 2, in an additional embodiment the present invention provides a compound of formula (I) wherein X is S or a pharmaceutically acceptable salt or in vivo hydrolysable ester thereof.

In section 2, in an additional embodiment the present invention provides a compound of formula (I) wherein Y is C or a pharmaceutically acceptable salt or in vivo hydrolysable ester thereof.

In section 2, in an additional embodiment the present invention provides a compound of formula (I) wherein Y is S or a pharmaceutically acceptable salt or in vivo hydrolysable ester thereof.

In section 2, in an additional embodiment the present invention provides a compound of formula (I) wherein Y is 0 or a pharmaceutically acceptable salt or in vivo hydrolysable ester thereof.

In section 2, in an additional embodiment the present invention provides a compound of formula (I) wherein m is 0 or a pharmaceutically acceptable salt or in vivo hydrolysable ester thereof.

In section 2, in an additional embodiment the present invention provides a compound of formula (I) wherein m is 1 or a pharmaceutically acceptable salt or in vivo hydrolysable ester thereof.

In section 2, in an additional embodiment the present invention provides a compound of formula (I) wherein R¹ is F or a pharmaceutically acceptable salt or in vivo hydrolysable ester thereof.

In section 2, in an additional embodiment the present invention provides a compound of formula (I) wherein R² is methyl or a pharmaceutically acceptable salt or in vivo hydrolysable ester thereof.

In section 2, in an additional embodiment the present invention provides a compound of formula (I) wherein R² is ethyl or a pharmaceutically acceptable salt or in vivo hydrolysable ester thereof.

In section 2, in an additional embodiment the present invention provides a compound of formula (I) wherein R² is propyl or a pharmaceutically acceptable salt or in vivo hydrolysable ester thereof.

In section 2, in an additional embodiment the present invention provides a compound of formula (I) wherein R² is isopropyl or a pharmaceutically acceptable salt or in vivo hydrolysable ester thereof.

In section 2, in an additional embodiment the present invention provides a compound of formula (I) wherein n is 2 or a pharmaceutically acceptable salt or in vivo hydrolysable ester thereof.

In section 2, in an additional embodiment the present invention provides a compound of formula (I) wherein n is 3 or a pharmaceutically acceptable salt or in vivo hydrolysable ester thereof.

In section 2, in an additional embodiment the present invention provides a compound of formula (I) wherein R³ and R⁴ are independently H or a pharmaceutically acceptable salt or in vivo hydrolysable ester thereof.

In section 2, in an additional embodiment the present invention provides a compound of formula (I) wherein R³ and R⁴ are independently methyl or a pharmaceutically acceptable salt or in vivo hydrolysable ester thereof.

In section 2, in an additional embodiment the present invention provides a compound of formula (I) wherein R³ and R⁴ are independently ethyl or a pharmaceutically acceptable salt or in vivo hydrolysable ester thereof.

In section 2, in an additional embodiment the present invention provides a compound of formula (I) wherein R⁵ is F or a pharmaceutically acceptable salt or in vivo hydrolysable ester thereof.

In section 2, in an additional embodiment the present invention provides a compound of formula (I) wherein R⁵ is Cl or a pharmaceutically acceptable salt or in vivo hydrolysable ester thereof.

In section 2, in an additional embodiment the present invention provides a compound of formula (I) wherein R⁵ is Br or a pharmaceutically acceptable salt or in vivo hydrolysable ester thereof.

In section 2, in an additional embodiment the present invention provides a compound of formula (I) wherein R⁵ is methyl or a pharmaceutically acceptable salt or in vivo hydrolysable ester thereof.

In section 2, in an additional embodiment the present invention provides a compound of formula (I) wherein R⁵ is ethyl or a pharmaceutically acceptable salt or in vivo hydrolysable ester thereof.

In section 2, in an additional embodiment the present invention provides a compound of formula (I) wherein p is 1 or a pharmaceutically acceptable salt or in vivo hydrolysable ester thereof.

In section 2, in an additional embodiment the present invention provides a compound of formula (I) wherein p is 2 or a pharmaceutically acceptable salt or in vivo hydrolysable ester thereof.

In section 2, in an additional embodiment the present invention provides a compound of formula (I) or a pharmaceutically acceptable salt or in vivo hydrolysable ester thereof as recited above wherein:
X is C;
Y is selected from or O or S;
m is 0, or 1;
R¹ is F, when m is 1;
R² is selected from C₁₋₃alkyl;
n is 2 or 3;
R³ and R⁴ are independently selected from H or C₁₋₂alkyl;
R⁵ is selected from F, Cl, Br, or C₁₋₂alkyl;
p is 1 or 2.

In section 2, in an additional embodiment the present invention provides a compound of formula (I) or a pharmaceutically acceptable salt or in vivo hydrolysable ester thereof as recited above wherein:
X is C;
Y is selected from O or S;
m is 0;
R² is selected from C₂₋₃alkyl;
n is 2 or 3;
R³ and R⁴ are independently selected from H or C₁₋₂alkyl;
R⁵ is selected from F, Cl, Br, or C₁₋₂alkyl;
p is 1 or 2.

In section 2, in an additional embodiment the present invention provides a compound of formula (I) or a pharmaceutically acceptable salt or in vivo hydrolysable ester thereof as recited above wherein:
X is C;
Y is S;
m is 0, or 1;
R¹ is F when m is 1;
R² is selected from C₁₋₃alkyl;
n is 2 or 3;
R³ and R⁴ are independently selected from H or C₁₋₂alkyl;
R⁵ is selected from F, Cl, Br, or C₁₋₂alkyl;
p is 1 or 2.

In section 2, in an additional embodiment the present invention provides a compound of formula (I) or a pharmaceutically acceptable salt or in vivo hydrolysable ester thereof as recited above wherein:
X is C;
Y is O;
m is 0, or 1;
R¹ is F when m is 1;
R² is selected from C₁₋₃alkyl;
n is 2 or 3;
R³ and R⁴ are independently selected from H or C₁₋₂alkyl;
R⁵ is selected from F, Cl, Br, or C₁₋₂alkyl;
p is 1 or 2.

In section 2, in an additional embodiment the present invention provides a compound of formula (I) or a pharmaceutically acceptable salt or in vivo hydrolysable ester thereof as recited above wherein:
X is C;
Y is S;
m is 0;
R² is selected from C₁₋₃alkyl;
n is 2 or 3;
R³ and R⁴ are independently selected from H or C₁₋₂alkyl;
R⁵ is selected from F, Cl, Br, or C₁₋₂alkyl;
p is 1 or 2.

In section 2, in an additional embodiment the present invention provides a compound of formula (I) or a pharmaceutically acceptable salt or in vivo hydrolysable ester thereof as recited above wherein:
X is C;
Y is S;
m is 1;
R¹ is F;
R² is selected from C₁₋₃alkyl;
n is 2 or 3;
R³ and R⁴ are independently selected from H or C₁₋₂alkyl;
R⁵ is selected from F, Cl, Br, or C₁₋₂alkyl;
p is 1 or 2.

In section 2, in an additional embodiment the present invention provides a compound of formula (I) or a pharmaceutically acceptable salt or in vivo hydrolysable ester thereof as recited above wherein:
X is C;
Y is S;
m is 0;
R² is selected from ethyl or isopropyl;
n is 2 or 3;
R³ and R⁴ are independently selected from H or methyl;
R⁵ is selected from F, Cl, Br, or C₁₋₂alkyl;
p is 1 or 2.

In section 2, in a further aspect of the invention there is provided a compound of formula (I) or a pharmaceutically acceptable salt thereof.

In section 2, in an additional embodiment the present invention provides a compound of formula (I) as recited above selected from the following:
N-(3-Amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide hydrogen chloride;
N-(3-Amino-propyl)-N-{1-[5-(3-fluoro-benzyl)-3-methyl-4-oxo-4,5-dihydro-isothiazolo[5,4-d]pyrimidin-6-yl]-2-methyl-propyl}-4-methyl-benzamide hydrogen chloride;
N-(2-Amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide hydrogen chloride;
N-(2-Amino-ethyl)-N-[1-(5-benzyl-3-methyl-4-oxo-4,5-dihydro-isothiazolo[5,4-d]pyrimidin-6-yl)-2-methyl-propyl]-4-methyl-benzamide hydrogen chloride;
N-(2-Amino-ethyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide hydrogen chloride;
N-(3-Amino-propyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide hydrogen chloride;
N-(3-Amino-propyl)-N-[1-(5-benzyl-3-methyl-4-oxo-4,5-dihydro-isothiazolo[5,4-d]pyrimidin-6-yl)-2-methyl-propyl]-4-bromo-benzamide hydrogen chloride;
N-[1-(5-Benzyl-3-methyl-4-oxo-4,5-dihydro-isothiazolo[5,4-d]pyrimidin-6-yl)-2-methyl-propyl]-N-(3-dimethylamino-propyl)₄-methyl-benzamide hydrogen chloride;
N-[1-(5-Benzyl-3-methyl-4-oxo-4,5-dihydro-isothiazolo[5,4-d]pyrimidin-6-yl)-2-methyl-propyl]-N-(3-dimethylamino-propyl)-4-bromo-benzamide hydrogen chloride;
N-[1-(5-Benzyl-3-methyl-4-oxo-4,5-dihydro-isothiazolo[5,4-d]pyrimidin-6-yl)-2-methyl-propyl]-N-(3-dimethylamino-propyl)-3-fluoro-4-methyl-benzamide hydrogen chloride;
N-(3-Amino-propyl)-N-{1-[5-(3-fluoro-benzyl)-3-methyl-4-oxo-4,5-dihydro-isoxazolo[5,4-d]pyrimidin-6-yl]-2-methyl-propyl}-4-methyl-benzamide hydrogen chloride;
N-(3-Amino-propyl)-N-[1-(6-benzyl-3-methyl-7-oxo-6,7-dihydro-isothiazolo[4,5-d]pyrimidin-5-yl)-propyl]-4-methyl-benzamide hydrogen chloride.

In section 2, in a further embodiment the present invention provides a compound of formula (I) or a pharmaceutically acceptable salt or an in vivo hydrolysable ester thereof for use as a medicament.

In section 2, in a further embodiment the present invention provides a compound of formula (I) or a pharmaceutically acceptable salt thereof for use as a medicament.

In section 2, according to a further aspect of the invention there is provided the use of a compound of the formula (I), or a pharmaceutically acceptable salt thereof, as defined hereinbefore in the manufacture of a medicament for use in the production of an Eg5 inhibitory effect in a warm-blooded animal such as man.

In section 2, according to a further aspect of the invention there is provided the use of a compound of the formula (I), or a pharmaceutically acceptable salt thereof, as defined hereinbefore in the manufacture of a medicament for use in the production of an anti-proliferative effect in a warm-blooded animal such as man.

In section 2, according to this aspect of the invention there is provided the use of a compound of the formula (I), or a pharmaceutically acceptable salt thereof, as defined hereinbefore in the manufacture of a medicament for use in the production of an anti-cancer effect in a warm-blooded animal such as man.

In section 2, according to a further feature of the invention, there is provided the use of a compound of the formula (I), or a pharmaceutically acceptable salt thereof, as defined herein before in the manufacture of a medicament for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma.

In section 2, in a further embodiment the present invention provides a compound of formula (I) or a pharmaceutically acceptable salt or an in vivo hydrolysable ester thereof, in the manufacture of a medicament for the treatment or prophylaxis of disorders associated with cancer.

In section 2, in a further embodiment the present invention provides a compound of formula (I) or a pharmaceutically acceptable salt thereof, in the manufacture of a medicament for the treatment or prophylaxis of disorders associated with cancer.

In section 2, according to a further feature of this aspect of the invention there is provided a method for producing an Eg5 inhibitory effect in a warm-blooded animal, such as man, in need of such treatment which comprises administering to said animal an effective amount of a compound of formula (I), or a pharmaceutically acceptable salt thereof, as defined above.

In section 2, according to a further feature of this aspect of the invention there is provided a method of producing an anti-proliferative effect in a warm-blooded animal, such as man, in need of such treatment which comprises administering to said animal an effective amount of a compound of formula (I), or a pharmaceutically acceptable salt thereof, as defined above.

In section 2, according to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

In section 2, in a further embodiment the present invention provides a method for the prophylaxis treatment of cancer comprising administering to a human in need of such treatment a therapeutically effective amount of a compound of formula (I) or a pharmaceutically acceptable salt or an in vivo hydrolysable ester thereof.

In section 2, in a further embodiment the present invention provides a method for the prophylaxis treatment of cancer comprising administering to a human in need of such treatment a therapeutically effective amount of a compound of formula (I) or a pharmaceutically acceptable salt thereof.

In section 2, in a further embodiment the present invention provides a method of producing a cell cycle inhibitory (anti-cell-proliferation) effect in a warm-blooded animal, such as man, in need of such treatment with comprises administering to said animal an effective amount of a compound of formula (I) or a pharmaceutically acceptable salt or an in vivo hydrolysable ester thereof.

In section 2, in a further embodiment the present invention provides a method of producing a cell cycle inhibitory (anti-cell-proliferation) effect in a warm-blooded animal, such as man, in need of such treatment with comprises administering to said animal an effective amount of a compound of formula (I) or a pharmaceutically acceptable salt thereof.

In section 2, in a further embodiment the present invention provides a method for the treatment of cancer comprising administering to a human a therapeutically effective amount of a compound of formula (I) or a pharmaceutically acceptable salt or an in vivo hydrolysable ester thereof.

In section 2, in a further embodiment the present invention provides a method for the treatment of cancer comprising administering to a human a therapeutically effective amount of a compound of formula (I) or a pharmaceutically acceptable salt thereof.

In section 2, in a further embodiment the present invention provides a method for the treatment of breast cancer, colorectal cancer, ovarian cancer, lung (non small cell) cancer, malignant brain tumors, sarcomas, melanoma and lymphoma by administering a compound of formula (I) or a pharmaceutically acceptable salt or an in vivo hydrolysable ester thereof.

In section 2, in a further embodiment the present invention provides a method for the treatment of breast cancer, colorectal cancer, ovarian cancer, lung (non small cell) cancer, malignant brain tumors, sarcomas, melanoma and lymphoma by administering a compound of formula (I) or a pharmaceutically acceptable salt thereof.

In section 2, according to an additional feature of this aspect of the invention there is provided a method of treating carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma, in a warm-blooded animal, such as man, in need of such treatment which comprises administering to said animal an effective amount of a compound of formula (I) or a pharmaceutically acceptable salt thereof as defined herein before.

In section 2, in a further embodiment the present invention provides a method for the treatment of cancer by administering to a human a compound of formula (I) or a pharmaceutically acceptable salt or an in vivo hydrolysable ester thereof and an anti-tumor agent.

In section 2, in a further embodiment the present invention provides a method for the treatment of cancer by administering to a human a compound of formula (I) or a pharmaceutically acceptable salt thereof and an anti-tumor agent.

In section 2, in a further embodiment the present invention provides a pharmaceutical composition comprising a compound of formula (I) or a pharmaceutically acceptable salt or an in vivo hydrolysable ester thereof together with at least one pharmaceutically acceptable carrier, diluent or excipient.

In section 2, in a further embodiment the present invention provides a pharmaceutical composition comprising a compound of formula (I) or a pharmaceutically acceptable salt thereof together with at least one pharmaceutically acceptable carrier, diluent or excipient.

In section 2, in a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an Eg5 inhibitory effect in a warm-blooded animal such as man.

In section 2, in a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-proliferative effect in a warm-blooded animal such as man.

In section 2, in a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In section 2, in a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma in a warm-blooded animal such as man.

In section 2, according to a further aspect of the invention there is provided the use of a compound of the formula (I), or a pharmaceutically acceptable salt thereof, as defined hereinbefore in the production of an Eg5 inhibitory effect in a warm-blooded animal such as man.

In section 2, according to a further aspect of the invention there is provided the use of a compound of the formula (I), or a pharmaceutically acceptable salt thereof, as defined hereinbefore for use in the production of an anti-proliferative effect in a warm-blooded animal such as man.

In section 2, according to this aspect of the invention there is provided the use of a compound of the formula (I), or a pharmaceutically acceptable salt thereof, as defined hereinbefore for use in the production of an anti-cancer effect in a warm-blooded animal such as man.

In section 2, according to a further feature of the invention, there is provided the use of a compound of the formula (I), or a pharmaceutically acceptable salt thereof, as defined herein before for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma.

In section 2, according to a further aspect of the invention there is provided the use of a compound of the formula (I), or a pharmaceutically acceptable salt thereof, as defined hereinbefore in the production of an Eg5 inhibitory effect in a warm-blooded animal such as man.

In section 2, according to a further aspect of the invention there is provided the use of a compound of the formula (I), or a pharmaceutically acceptable salt thereof, as defined hereinbefore for use in the production of an anti-proliferative effect in a warm-blooded animal such as man.

In section 2, according to this aspect of the invention there is provided the use of a compound of the formula (I), or a pharmaceutically acceptable salt thereof, as defined hereinbefore for use in the production of an anti-cancer effect in a warm-blooded animal such as man.

In section 2, according to a further feature of the invention, there is provided the use of a compound of the formula (I), or a pharmaceutically acceptable salt thereof, as defined herein before for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma.

In section 2, in a further embodiment the present invention provides the use of a compound of formula (I) or a pharmaceutically acceptable salt thereof, for the treatment or prophylaxis of disorders associated with cancer.

In section 2, in a further embodiment the present invention provides the use of a compound of formula (I) or a pharmaceutically acceptable salt thereof, for the treatment or prophylaxis of disorders associated with cancer.

The definitions set forth in this section of section 2 are intended to clarify terms used throughout section 2. The term “herein” means the entire section 2.

In section 2, the term “Cₘ₋ₙ” or “Cₘ₋ₙ group” used alone or as a prefix, refers to any group having m to n carbon atoms.

In section 2, the term “hydrocarbon” used alone or as a suffix or prefix, refers to any structure comprising only carbon and hydrogen atoms up to 14 carbon atoms.

In section 2, the term “hydrocarbon radical” or “hydrocarbyl” used alone or as a suffix or prefix, refers to any structure as a result of removing one or more hydrogens from a hydrocarbon.

In section 2, the term “alkyl” used alone or as a suffix or prefix, refers to monovalent straight or branched chain hydrocarbon radicals comprising, unless otherwise indicated, 1 to about 12 carbon atoms. In section 2, unless otherwise specified, “alkyl” includes both saturated alkyl and unsaturated alkyl. Particularly “alkyl” in section 2 refers to saturated alkyl.

In section 2, the term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, in section 2, a “phenyl substituted by nitro” refers to nitrophenyl.

In section 2, “RT” or “rt” means room temperature.

In section 2, when any variable (e.g., R¹, R⁴ etc.) occurs more than one time in any constituent or formula for a compound, its definition at each occurrence is independent of its definition at every other occurrence. Thus in section 2, for example, if a group is shown to be substituted with 0-3 R¹, then said group may optionally be substituted with 0, 1, 2 or 3 R¹ groups and R¹ at each occurrence is selected independently from the definition of R¹. Also in section 2, combinations of substituents and/or variables are permissible only if such combinations result in stable compounds.

A variety of compounds in the present invention of section 2 may exist in particular geometric or stereoisomeric forms. The present invention of section 2 takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms in section 2 may be present in a substituent such as an alkyl group. All such isomers in section 2, as well as mixtures thereof, are intended to be included in this invention. The compounds described in section 2 may have asymmetric centers. Compounds of the present invention in section 2 containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described in section 2, and all such stable isomers are contemplated in the present invention. Cis and trans geometric isomers of the compounds of the present invention are described in section 2 and may be isolated as a mixture of isomers or as separated isomeric forms. All chiral, diastereomeric, racemic forms and all geometric isomeric forms of a structure in section 2 are intended, unless the specific stereochemistry or isomeric form is specifically indicated.

In section 2, when a bond to a substituent is shown to cross a bond connecting two atoms in a ring, then such substituent may be bonded to any atom on the ring. In section 2, when a substituent is listed without indicating the atom via which such substituent is bonded to the rest of the compound of a given formula, then such substituent may be bonded via any atom in such substituent. In section 2, combinations of substituents and/or variables are permissible only if such combinations result in stable compounds.

In section 2, as used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In section 2, as used herein, “pharmaceutically acceptable salts” refer to derivatives of the disclosed compounds wherein the parent compound is modified by making acid or base salts thereof. Examples of pharmaceutically acceptable salts of section 2 include, but are not limited to, mineral or organic acid salts of basic residues such as amines; alkali or organic salts of acidic residues such as carboxylic acids; and the like. The pharmaceutically acceptable salts of section 2 include the conventional non-toxic salts or the quaternary ammonium salts of the parent compound formed, for example, from non-toxic inorganic or organic acids. For example, in section 2 such conventional non-toxic salts include those derived from inorganic acids such as hydrochloric, phosphoric, and the like; and the salts prepared from organic acids such as lactic, maleic, citric, benzoic, methanesulfonic, and the like. In section 2, the pharmaceutically acceptable salts of the invention also include salts prepared with one of the following acids benzene sulfonic acid, fumaric acid, methanesulfonic acid, naphthalene-1,5-disulfonic acid, naphthalene-2-sulfonic acid or L-tartaric acid.

In section 2, in one aspect of the invention there is provided a compound of the invention, particularly one of the Examples described herein, as a pharmaceutically acceptable salt, particularly a benzene sulfonic acid, fumaric acid, methanesulfonic acid, naphthalene-1,5-disulfonic acid, naphthalene-2-sulfonic acid or L-tartaric acid salt.

In section 2, the pharmaceutically acceptable salts of the present invention can be synthesized from the parent compound that contains a basic or acidic moiety by conventional chemical methods. Generally in section 2, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

In section 2, as used herein, “in vivo hydrolysable ester” means an in vivo hydrolysable (or cleavable) ester of a compound of the formula (I) that contains a carboxy or a hydroxy group. For example amino acid esters, C₁₋₆alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

In section 2, all chemical names were generated using a software system known as AutoNom Name accessed through ISIS draw.

Section 2: Combinations

The anti-cancer treatment defined in section 2 may be applied as a sole therapy or may involve, in addition to the compound of the invention, conventional surgery or radiotherapy or chemotherapy. Such chemotherapy in section 2 may include one or more of the following categories of anti-tumour agents:
(i) antiproliferative/antineoplastic drugs and combinations thereof, as used in medical oncology, such as alkylating agents (for example cis-platin, carboplatin, oxaliplatin, cyclophosphamide, nitrogen mustard, melphalan, chlorambucil, busulphan, temozolomide and nitrosoureas); antimetabolites (for example gemcitabine and antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere) polokinase inhibitors; and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function or inhibitors of SRC kinase (like 4-(6-chloro-2,3-methylenedioxyanilino)-7-[2-(4-methylpiperazin-1-yl)ethoxy]-5-tetrahydropyran-4-yloxyqyuinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661)) or antibodies to Heparanase);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [Erbitux, C225]), Ras/Raf signalling inhibitors such as farnesyl transferase inhibitors (for example sorafenib (BAY 43-9006) and tipifamib), tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), for example inhibitors of the platelet-derived growth factor family such as imatinib, and for example inhibitors of the hepatocyte growth factor family, c-kit inhibitors, abl kinase inhibitors, IGF receptor (insulin-like growth factor) kinase inhibitors and inhibitors of cell signalling through MEK, AKT and/or P13K kinases;
(v) antiangiogenic agents such as those which inhibit the effects of vascular endothelial growth factor, (for example the anti-vascular endothelial cell growth factor antibody bevacizumab [Avastin™], and VEGF receptor tyrosine kinase inhibitors such as those disclosed in International Patent Applications WO 97/22596, WO 97/30035, WO 97/32856, WO 98/13354, 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SUI 1248 (sunitinib; WO 01/60814)) and compounds that work by other mechanisms (for example linomide, inhibitors of integrin αvβ3 function and angiostatin), ang1 and 2 inhibitors;
(vi) vascular damaging agents such as Combretastatin A4 and compounds disclosed in International Patent Applications WO 99/02166, WO 00/40529, WO 00/41669, WO 01/92224, WO 02/04434 and WO 02/08213, anti bcl2;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;
x) cell cycle agents such as aurora kinase inhibitors (for example PH739358, VX-680, MLN8054, R763, MP235, MP529, VX-528, AX39459 and the specific examples mentioned in WO02/00649, WO03/055491, WO2004/058752, WO2004/058781, WO2004/058782, WO2004/094410, WO2004/105764, WO2004/113324 which are incorporated herein by reference), and cyclin dependent kinase inhibitors such as CDK2 and/or CDK4 inhibitors (for example the specific examples of WO01/14375, WO01/72717, WO02/04429, WO02/20512, WO02/66481, WO02/096887, WO03/076435, WO03/076436, WO03/076434, WO03/076433, WO04/101549 and WO04/101564 which are incorporated herein by reference); and
xi) cytotoxic agents such as gemcitibine, topoisomerase 1 inhibitors (adriamycin, etoposide) and topoisomerase II inhibitors.

In section 2, such conjoint treatment may be achieved by way of the simultaneous, sequential or separate dosing of the individual components of the treatment. Such combination products of section 2 employ the compounds of this invention within the dosage range described hereinbefore and the other pharmaceutically-active agent within its approved dosage range.

In a further aspect of section 2 there is provided a compound of formula (I) or a pharmaceutically acceptable salt or an in vivo hydrolysable ester thereof in combination with simultaneous, sequential or separate dosing of an anti-tumor agent or class selected from the list herein above.

Therefore in a further embodiment of section 2 the present invention provides a method for the treatment of cancer by administering to a human a compound of formula (I) or a pharmaceutically acceptable salt or an in vivo hydrolysable ester thereof in combination with simultaneous, sequential or separate dosing of an anti-tumor agent or class selected from the list herein above.

In a further aspect of section 2 of the present invention there is provided the use of a compound of formula (I) or a pharmaceutically acceptable salt or an in vivo hydrolysable ester thereof in combination with simultaneous, sequential or separate dosing of an anti-tumor agent or class selected from the list herein above for use in the manufacture of a medicament for use in the treatment of cancer.

In a further aspect of section 2 of the present invention there is provided the use of a compound of formula (I) or a pharmaceutically acceptable salt or an in vivo hydrolysable ester thereof in combination with simultaneous, sequential or separate dosing of an anti-tumor agent or class selected from the list herein above for use in the treatment of cancer.

The anti-cancer treatment defined in section 2 may also include one or more of the following categories of pharmaceutical agents:
i) an agent useful in the treatment of anemia, for example, a continuous eythropoiesis receptor activator (such as epoetin alfa);
ii) an agent useful in the treatment of neutropenia, for example, a hematopoietic growth factor which regulates the production and function of neutrophils such as a human granulocyte colony stimulating factor, (G-CSF), for example filgrastim; and
iii) an anti-emetic agent to treat nausea or emesis, including acute, delayed, late-phase, and anticipatory emesis, which may result from the use of a compound of the present invention, alone or with radiation therapy, suitable examples of such anti emetic agents include neurokinin-1 receptor antagonists, 5H13 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid or Benecorten, an antidopaminergic, such as the phenothiazines (for example prochlorperazine, fluphenazine, thioridazine and mesoridazine), metoclopramide or dronabinol.

In section 2, such conjoint treatment may be achieved by way of the simultaneous, sequential or separate dosing of the individual components of the treatment. Such conjoint treatment employs the compounds of this invention within the dosage range described hereinbefore and the other pharmaceutically-active agent within its approved dosage range.

In a further aspect of section 2 of the present invention there is provided a compound of formula (I) or a pharmaceutically acceptable salt or an in vivo hydrolysable ester thereof in combination with simultaneous, sequential or separate dosing of another pharmaceutical agent or class selected from the list herein above.

Therefore in a further embodiment of section 2 of the present invention provides a method for the treatment of cancer by administering to a human a compound of formula (I) or a pharmaceutically acceptable salt or an in vivo hydrolysable ester thereof in combination with simultaneous, sequential or separate dosing of another pharmaceutical agent or class selected from the list herein above.

In a further aspect of section 2 of the present invention there is provided the use of a compound of formula (I) or a pharmaceutically acceptable salt or an in vivo hydrolysable ester thereof in combination with simultaneous, sequential or separate dosing of another pharmaceutical agent or class selected from the list herein above for use in the manufacture of a medicament for use in the treatment of cancer.

In a further aspect of section 2 of the present invention there is provided the use of a compound of formula (I) or a pharmaceutically acceptable salt or an in vivo hydrolysable ester thereof in combination with simultaneous, sequential or separate dosing of another pharmaceutical agent or class selected from the list herein above for use in the treatment of cancer.

In section 2, in addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of Eg5 in laboratory animals such as cats, dogs, rabbits, monkeys, rats and mice, as part of the search for new therapeutic agents.

In the above section 2, other pharmaceutical composition, process, method, use and medicament manufacture features, the alternative and preferred embodiments of the compounds of the invention described herein also apply.

Section 2: Formulations

Compounds of the present invention of section 2, may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

In section 2, the dosage will depend on the route of administration, the severity of the disease, age and weight of the patient and other factors normally considered by the attending physician, when determining the individual regimen and dosage level as the most appropriate for a particular patient.

In section 2, an effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

In section 2, for preparing pharmaceutical compositions from the compounds of this invention, inert, pharmaceutically acceptable carriers can be either solid or liquid. Solid form preparations include powders, tablets, dispersible granules, capsules, cachets, and suppositories.

In section 2, a solid carrier can be one or more substances, which may also act as diluents, flavoring agents, solubilizers, lubricants, suspending agents, binders, or tablet disintegrating agents; it can also be an encapsulating material.

In section 2, in powders, the carrier is a finely divided solid, which is in a mixture with the finely divided active component. In tablets, the active component is mixed with the carrier having the necessary binding properties in suitable proportions and compacted in the shape and size desired.

In section 2, for preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

In section 2, suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In section 2, some of the compounds of the present invention are capable of forming salts with various inorganic and organic acids and bases and such salts are also within the scope of this invention. Examples of such acid addition salts in section 2 include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. In section 2, base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also in section 2, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred in section 2, although other salts are also useful, such as in isolating or purifying the product.

In section 2, the salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In section 2, in order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In section 2, in addition to the compounds of the present invention, the pharmaceutical composition of this invention may also contain, or be co-administered (simultaneously or sequentially) with, one or more pharmacological agents of value in treating one or more disease conditions referred to herein.

In section 2, the term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions of section 2 include solutions, suspensions, and emulsions. Sterile water or water-propylene glycol solutions of the active compounds may be mentioned as an example of liquid preparations suitable for parenteral administration. Liquid compositions of section 2 can also be formulated in solution in aqueous polyethylene glycol solution. In section 2, aqueous solutions for oral administration can be prepared by dissolving the active component in water and adding suitable colorants, flavoring agents, stabilizers, and thickening agents as desired. In section 2, aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

In section 2, the pharmaceutical compositions can be in unit dosage form. In such form in section 2, the composition is divided into unit doses containing appropriate quantities of the active component. In section 2, the unit dosage form can be a packaged preparation, the package containing discrete quantities of the preparations, for example, packeted tablets, capsules, and powders in vials or ampoules. In section 2, the unit dosage form can also be a capsule, cachet, or tablet itself, or it can be the appropriate number of any of these packaged forms.

Section 2: Synthesis

In section 2, the compounds of the present invention can be prepared in a number of ways well known to one skilled in the art of organic synthesis. In section 2, the compounds of the present invention can be synthesized using the methods described herein, together with synthetic methods known in the art of synthetic organic chemistry, or variations thereon as appreciated by those skilled in the art. Such methods in section 2 include, but are not limited to, those described herein. All references cited herein are hereby incorporated in their entirety by reference.

In section 2, the novel compounds of this invention may be prepared using the reactions and techniques described herein. In section 2, the reactions are performed in solvents appropriate to the reagents and materials employed and are suitable for the transformations being effected. Also, in the description of the synthetic methods described in section 2, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule in section 2 must be compatible with the reagents and reactions proposed. Such restrictions to the substituents in section 2, which are compatible with the reaction conditions, will be readily apparent to one skilled in the art and alternate methods must then be used.

In section 2, the starting materials for the examples contained herein are either commercially available or are readily prepared by standard methods from known materials. For example the following reactions are illustrations but not limitations of the preparation of some of the starting materials and examples used in section 2.

### SECTION 2: EXAMPLES

Section 2 of the invention will now be illustrated by the following non limiting examples in which, unless stated otherwise:
(i) temperatures are given in degrees Celsius (° C.); operations were carried out at room or ambient temperature, that is, at a temperature in the range of 18-30° C.;
(ii) organic solutions were dried over anhydrous sodium sulphate; evaporation of solvent was carried out using a rotary evaporator under reduced pressure (600-4000 Pascals; 4.5-30 mmHg) with a bath temperature of up to 60° C.;
(iii) in general, the course of reactions was followed by TLC or MS and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz using deuterated chloroform (CDCl₃) as solvent unless otherwise indicated;
(vii) chemical symbols have their usual meanings; SI units and symbols are used;
   (viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB); electrospray (ESP); or atmospheric pressure chemical ionisation (APCI); values for m/z are given; generally, only ions which indicate the parent mass are reported;
(x) where a synthesis is described as being analogous to that described in a previous example the amounts used are the millimolar ratio equivalents to those used in the previous example;
(xi) the following abbreviations have been used:
   THF tetrahydrofuran;
   DMF N,N-dimethylformamide;
   EtOAc ethyl acetate;
   AcOH acetic acid;
   DCM dichloromethane; and
   DMSO dimethylsulphoxide; and
(xii) a Vigreux column is a glass tube with a series of indentations such that alternate sets of indentations point downward at an angle of 45 degree in order to promote the redistribution of liquid from the walls to the center of the column; The Vigreux column used herein is 150 mm long (between indents) with a 20 mm diameter and it was manufactured by Lab Glass.
   Section 2: Method 1

### 2-(1-Ethoxy-ethylidene)-malononitrile

Triethyl orthoacetate (97 g, 0.6 mol), malononitrile (33 g, 0.5 mol) and glacial acetic acid (1.5 g) were placed in a 1 L flask equipped with a stirrer, thermometer and a Vigreux column (20×1 in.) on top of which a distillation condenser was placed. The reaction mixture was heated and ethyl alcohol began to distill when the temperature of the reaction mixture was about 85-90° C. After about 40 min., the temperature of the reaction mixture reached 140° C. Then the reaction was concentrated in a rotary evaporator to remove the low-boiling materials and the residue was crystallized from absolute alcohol to yield the pure product (62.2 g, 91%) as a light yellow solid mp 91.6° C.

Section 2: Method 2

### (2E)-2-Cyano-3-ethoxybut-2-enethioamide

2-(1-Ethoxy-ethylidene)-malononitrile (Section 2: Method 1) (62 g, 0.45 mol) was dissolved in anhydrous benzene (800 mL) and 1 mL of triethylamine was added as catalyst. The mixture was stirred and hydrogen sulfide was bubbled into this solution for 40 min and a solid formed. The precipitated solid was filtered off and dried. The solid was recrystallized from absolute alcohol (100 mL) filtered and dried to isolate the pure (2E)-2-cyano-3-ethoxybut-2-enethioamide (19.3 g, 25%) as light brown crystals.

Section 2: Method 3

### (2E)-3-Amino-2-cyanobut-2-enethioamide

(2E)-2-cyano-3-ethoxybut-2-enethioamide (Section 2: Method 2) (19.2 g, 0.136 mol) was dissolved in a saturated solution of ammonia in methanol (500 mL) and stirred at r.t. overnight. The reaction mixture was concentrated and the residue was dissolved in hot water (600 mL) and the undissoved solid was filtered and dried to recover 6 g of the starting thiocrotonamide. The aqueous solution on standing overnight provided the pure (2E)-3-amino-2-cyanobut-2-enethioamide (6.85 g, 63%) as off-white crystals. Having the following properties ¹H NMR (300 MHz, DMSO-d₆) δ 2.22 (s, 3H), 7.73 (bs, 1H), 8.53 (bs, 1H), 9.01 (bs, 1H), 11.60 (bs, 1H).

Section 2: Method 4

### 5-Amino-3-methylisothiazole-4-carbonitrile

To a stirred solution of (2E)-3-amino-2-cyanobut-2-enethioamide (Section 2: Method 3) (6.83 g, 48.4 mmol) in methanol (300 mL) was added dropwise 13.6 mL (124 mmol.) of 30% hydrogen peroxide. The mixture was stirred at 60° C. for 4 h and evaporated to 60 mL in a rotary evaporator and cooled in an ice-bath. The crystallized product was filtered off and recrystallized from EtOAc to provide the pure product 5-amino-3-methylisothiazole-4-carbonitrile (5.41 g, 80%) as a white crystalline solid. Having the following properties ¹H NMR (300 MHz, DMSO-d₆) δ 2.24 (s, 3H), 8.00 (bs, 2H).

Section 2: Method 5

### N-(4-Cyano-3-methyl-isothiazol-5-yl)-butyramide

To a solution of 5-amino-3-methylisothiazole-4-carbonitrile (Section 2: Method 4) (5.31 g, 38.2 mmol) in DCM (200 mL) at 0° C., NEt₃ (5 g, 50 mmol) was added followed by the dropwise addition of a solution of the butyryl chloride (4.88 g, 45.8 mmol) in DCM (50 mL). After the completion of the addition the reaction mixture was allowed to warm to r.t. and stirred overnight. The reaction mixture was washed with water (100 mL), 1N HCl (100 mL), brine (200 mL) and dried over Na₂SO₄. Concentration of the DCM layer provided the crude product which was triturated from DCM/hexanes (1/10) and filtered off to isolate the pure N-(4-cyano-3-methyl-isothiazol-5-yl)-butyramide (7.57 g, 95%) as an orange solid.

Section 2: Method 6

### 5-Butyrylamino-3-methyl-isothiazole-4-carboxylic acid amide

To a solution of N-(4-cyano-3-methyl-isothiazol-5-yl)-butyramide (Section 2: Method 5) (4.18 g, 20 mmol) in 30% aqueous NH₄OH (250 mL), was added dropwise 100 mL of hydrogen peroxide at r.t. After the completion of the addition the reaction mixture was stirred at 60° C. overnight after which the TLC showed the complete disappearance of SM. The reaction mixture was cooled and extracted with chloroform (3×100 mL). The organic layer was dried (Na₂SO₄) and concentrated to get the pure 5-butyrylamino-3-methyl-isothiazole-4-carboxylic acid amide (2.9 g, 72%) as a white solid. Having the following properties ¹H NMR (300 MHz) δ 1.03 (t, 3H), 1.79 (m, 2H), 2.54 (t, 3H), 2.69 (s, 3H), 5.97 (bs, 2H), 11.78 (bs, 1H).

Section 2: Method 7

### 3-Methyl-6-propyl-5H-isothiazolo[5,4-d]pyrimidin-4-one

5-Butyrylamino-3-methyl-isothiazole-4-carboxylic acid amide (Section 2: Method 6) (1.9 g, 8.3 mmol) was suspended in 75 mL of 30% NH₃ and then was heated to 140° C. for 4 h in a pressure reactor. The mixture was cooled and neutralized to pH 8. The precipitated 3-methyl-6-propyl-5H-isothiazolo[5,4-d]pyrimidin-4-one was filtered off, washed with water (100 mL) and dried in vacuum oven at 40° C. overnight to get 800 mg (34%) of pure product. Having the following properties ¹H NMR (300 MHz) δ 1.03 (t, 3H), 1.74 (m, 2H), 2.67 (t, 3H), 2.78 (s, 3H).

Section 2: Method 8

### 5-(3-Fluoro-benzyl)-3-methyl-6-propyl-5H-isothiazolo[5,4-d]pyrimidin-4-one

To a solution of 3-methyl-6-propyl-5H-isothiazolo[5,4-d]pyrimidin-4-one (Section 2: Method 7) (2.09 g, 10 mmol) in 20 mL of anhydrous DMF was added anhydrous K₂CO₃ (2.76 g, 20 mmol) followed by 3-fluorobenzyl bromide (2.79 g, 15 mmol) and the mixture was stirred at room temperature overnight. Solvents were removed by evaporation. The residue obtained was triturated with water (60 mL) and stirred for 30 minutes. The solid separated was collected by filtration and subsequently purified by crystallization from a mixture of EtOAc and hexanes (1:5) and dried. Yield 1.78 g (56%). Having the following properties ¹H NMR (300 MHz, DMSO-d₆) δ:0.87 (t, 3H), 1.65-1.67 (m, 2H), 2.73 (s, 3H), 2.75 (t, 2H), 5.39 (s, 2H), 7.04 (d, 1H), 7.05-7.09 (m, 2H), 7.13-7.38 (m, 1H).

Section 2: Method 9

### 6-(1-Bromo-propyl)-5-(3-fluoro-benzyl)-3-methyl-5H-isothiazolo[5,4-d]pyrimidin-4-one

To a solution of 5-(3-fluoro-benzyl)-3-methyl-6-propyl-5H-isothiazolo[5,4-d]pyrimidin-4-one (Section 2: Method 8) (1.78 g, 5.62 mmol) and sodium acetate (4.6 g, 56.2 mmol) in acetic acid (40 mL) at 100° C., a solution of the bromine (1.8 g, 11.24 mmol) in acetic acid (10 mL) was added dropwise over a period of 30 minutes. The mixture was stirred for additional 15 minutes and cooled to 25° C. The solvents were removed by evaporation and the residue was dissolved in EtOAc (100 mL) and washed with 100 mL each of water, 10% sodium thiosulfate solution and brine. Solvents were removed by evaporation and the residue was purified by column chromatography on silica, eluting with 10-15% of EtOAc in hexanes. Yield 890 mg (41%). Having the following properties ¹H NMR (300 MHz, DMSO-d₆) δ: 0.87 (t, 3H), 2.05-2.20 (m, 1H), 2.30-2.40 (m, 1H), 2.70 (s, 3H), 5.07 (t, 1H), 5.27 (d, 1H), 5.66 (d, 1H), 7.05-7.25 (m, 3H), 7.38-7.40 (m, 1H).

Section 2: Method 10

### (3-{1-[5-(3-Fluoro-benzyl)-3-methyl-4-oxo-4,5-dihydro-isothiazolo[5,4-d]pyrimidin-6-yl]-propylamino}-propyl)-carbamic acid tert-butyl ester

To a suspension of 6-(1-bromo-propyl)-5-(3-fluoro-benzyl)-3-methyl-5H-isothiazolo[5,4-d]pyrimidin-4-one (Section 2: Method 9) (90 mg, 2.25 mmol) in DMF (10 mL) was added (3-amino-propyl)-carbamic acid tert-butyl ester (700 mg, 4.02 mmol) and diisopropyl ethyl amine (740 mg, 5.74 mmol, 1 mL). The mixture was then stirred for 30 minutes. It was diluted with EtOAc (100 mL) and washed with water (2×100 mL). The EtOAc layer was then dried over MgSO₄ and evaporated to dryness. The product was used in the next step without purification. Having the following properties m/z 490 (MH⁺).

Section 2: Method 11

### {3-[{1-[5-(3-Fluoro-benzyl)-3-methyl-4-oxo-4,5-dihydro-isothiazolo[5,4-d]pyrimidin-6-yl]-propyl}-(4-methyl-benzoyl)-amino]-propyl}-carbamic acid tert-butyl ester

To a solution of (3-{1-[5-(3-fluoro-benzyl)-3-methyl-4-oxo-4,5-dihydro-isothiazolo[5,4-d]pyrimidin-6-yl]-propylamino}-propyl)-carbamic acid tert-butyl ester (Section 2: Method 10) (amount isolated from Section 2: Method 10 used) in chloroform (20 mL) was added diisopropyl ethyl amine (740 mg, 5.74 mmol, 1 mL). The reaction mixture was brought to 50° C. and a solution of p-toluoyl chloride (521 mg, 3.38 mmol) in chloroform (5 mL) was added dropwise. The mixture was maintained at the same temperature for 2 h and then at 25° C. for 18 h and subsequently diluted with chloroform and washed with water (2×25 mL). The organic layer was evaporated to dryness and the residue was purified by column chromatography on silica, eluting with 15% EtOAc in hexane gave the product. Yield 590 mg (43%). Having the following properties m/z 608 (MH⁺); ¹H NMR (300 MHz, DMSO-d₆, 95° C.) δ 0.70 (t, 3H), 1.10-1.15 (m, 1H), 1.26 (s, 9H), 1.29-1.37 (m, 1H), 1.89-1.90 (m, 1H), 2.32-2.47 (m, 1H), 2.46 (s, 3H), 2.47-2.49 (m, 2H), 2.71 (s, 3H), 3.22-3.25 (m, 2H), 4.99 (d, 1H), 5.59(bs, 1H), 5.73 (d, 1H), 6.03 (t, 1H), 6.96-7.18 (m, 3H), 7.18-7.20 (m, 4H), 7.20-7.22 (m, 1H).

Section 2: Method 12

### N-(4-Cyano-3-methyl-isothiazol-5-yl)-3-methyl-butyramide

To a solution of 5-amino-3-methyl-isothiazole-4-carbonitrile (Section 2: Method 4) (6.38 g, 45.9 mmol) in pyridine (20 mL) at 0° C., isovaleryl chloride (6.65 g, 55 mmol) was added dropwise. After the completion of the addition the reaction mixture was allowed to warm to r.t. and stirred overnight. The TLC and the MS showed the complete disappearance of the starting material and the reaction mixture was diluted with CHCl₃ (200 mL), washed with water (200 mL), 2N HCl (225 mL), satd. NaHCO₃ (200 mL), brine (200 mL) and dried over Na₂SO₄. Concentration of the CHCl₃ layer provided the crude product which was triturated from DCM/hexanes (1/10) and filtered off to isolate N-(4-cyano-3-methyl-isothiazol-5-yl)-3-methyl-butyramide (8.1 g, 79%) as an off-white crystalline solid. Having the following properties ¹H NMR (300 MHz) δ 1.04 (d, 6H), 2.18-2.32 (m, 1H), 2.46 (d, 2H), 2.53 (s, 3H), 9.87 (bs, 1H).

Section 2: Method 13

### 3-Methyl-5-(3-methyl-butylamino)-isothiazole-4-carboxylic acid amide

To a solution of N-(4-cyano-3-methyl-isothiazol-5-yl)-3-methyl-butyramide (Section 2: Method 12) (8 g, 35.8 mmol) in 30% aqueous NH₄OH (200 mL), was added dropwise 100 mL of hydrogen peroxide at r.t. After the completion of the addition the reaction mixture was stirred at 60° C. overnight after which the TLC showed the complete disappearance of SM. The reaction mixture was concentrated to 40 mL and extracted with chloroform (3×100 mL). The organic layer was dried (Na₂SO₄) and concentrated to obtain 3-methyl-5-(3-methyl-butyrylamino)-isothiazole-4-carboxylic acid amide (6.1 g, 71%) as a light yellow solid. Having the following properties ¹H NMR (300 MHz) δ 1.03 (d, 6H), 2.24 (m, 1H), 2.43 (d, 2H), 2.69 (s, 3H), 5.98 (bs, 2H), 11.77 (bs, 1H).

Section 2: Method 14

### 6-Isobutyl-3-methyl-5H-isothiazolo[5,4-d]pyrimidin-4-one

3-Methyl-5-(3-methyl-butyrylamino)-isothiazole-4-carboxylic acid amide (Section 2: Method 13) (6 g, 25 mmol) was suspended in 150 mL of 30% NH₃ and then was heated to 140° C. for 5 h in a pressure reactor. The mixture was cooled and neutralized to pH 7. The reaction mixture was extracted with EtOAc (3×100 mL) and the combined organic layers were washed with water (100 mL), brine (100 mL) and concentrated to get the crude product which was further purified by column (silica gel) chromatography using 30% EtOAc in hexanes as eluent. Concentration of the pure product fractions provided 6-isobutyl-3-methyl-5H-isothiazolo[5,4-d]pyrimidin-4-one (2.2 g, 38%) as an off-white powder. Having the following properties ¹H NMR (300 MHz) δ 1.05 (d, 6H), 2.32 (m, 1H), 2.69 (d, 2H), 2.82 (s, 3H).

Section 2: Method 15

### 5-Benzyl-6-isobutyl-3-methyl-5H-isothiazolo[4-d]pyrimidin-4-one

To a solution of 6-isobutyl-3-methyl-5H-isothiazolo[5,4-d]pyrimidin-4-one (Section 2: Method 14) (1.31 g, 5.8 mmol) in 20 mL of anhydrous DMF was added 1.38 g (10 mmol) of anhydrous K₂CO₃ followed by benzyl bromide (1.18 g, 6.9 mmol) and the mixture was stirred at room temperature overnight. The TLC of the reaction mixture showed the complete disappearance of the SM. The reaction mixture was poured into ice-cold water and extracted with EtOAc (3×100 mL). The combined extracts were washed with water (100 mL), brine (100 mL), dried (Na₂SO₄) and concentrated. The TLC and the ¹H NMR showed the presence of two products (N alkylated as well as O-alkylated products) in a ratio of 7:3. The products were separated by column (silica gel, 116 g) chromatography using 10% EtOAc in hexanes. 5-Benzyl-6-isobutyl-3-methyl-5H-isothiazolo[5,4-d]pyrimidin-4-one was isolated as white crystalline solid (1.3 g, 70%). Having the following properties m/z 314 (MH⁺), ¹H NMR (300 MHz) δ 0.94 (d, 6H), 2.23-2.37 (m, 1H), 2.64 (d, 2H), 2.82 (s, 3H), 5.38 (s, 2H), 7.10-7.38 (m, 5H).

Section 2: Method 15a

The following compounds were synthesized according to Section 2: Method 15:

| Section 2: | | | Alkylating |
|---|---|---|---|
| Method # | Compound Name | m/z | agent |
| 15a | 5-(3-Fluoro-benzyl)-6-isobutyl-3- | 332 | 3-fluorobenzyl |
| | methyl-5H-isothiazolo[5,4- | (MH⁺) | bromide |
| | d]pyrimidin-4-one | | |

Section 2: Method 16

### 5-Benzyl-6-(1-bromo-2-methyl-propyl)-3-methyl-5H-isothiazolo[5,4-d]pyrimidin-4-one

To a solution of 5-benzyl-6-isobutyl-3-methyl-5H-isothiazolo[5,4-d]pyrimidin-4-one (Section 2: Method 15) (1.3 g, 4.2 mmol) and sodium acetate (2 g) in acetic acid (10 mL) at 100° C., a solution of the bromine (1.32 g, 8.4 mmol) in acetic acid (10 mL) was added dropwise over a period of 20 minutes. The reaction mixture was stirred at that temperature for 30 min and cooled and the TLC (eluent 10% EtOAc in hexanes) and MS showed the complete disappearance of the SM and only the product. The reaction mixture was poured into ice water and extracted with EtOAc (3×60 mL) and the organic layers were combined and washed with 2% sodium thiosulfate solution (60 mL), water (100 mL), brine (100 mL) and dried over Na₂SO₄. Concentration of the organic layer provided 5-benzyl-6-(1-bromo-2-methyl-propyl)-3-methyl-5H-isothiazolo[5,4-d]pyrimidin-4-one (1.61 g, 99%) as white crystalline solid. Having the following properties m/z 392, 394 (MH⁺), ¹H NMR (300 MHz) δ 0.54 (d, 3H), 1.11 (d, 3H), 2.62-2.76 (m, 1H), 2.83 (s, 3H), 4.42 (d, 1H), 4.80 (d, 1H), 6.22 (d, 1H), 7.12-7.42 (m, 5H).

Section 2: Method 16a

The following compounds were synthesized according to Section 2: Method 16 starting from 5-(3-fluoro-benzyl)-6-isobutyl-3-methyl-5H-isothiazolo[5,4-d]pyrimidin-4-one (Section 2: Method 15a):

| Section 2: | | |
|---|---|---|
| Method # | Compound Name | m/z |
| 16a | 6-(1-Bromo-2-methyl-propyl)-5-(3-fluoro- | 410, 412(MH⁺) |
| | benzyl)-3-methyl-5H-isothiazolo[5,4-d] | |
| | pyrimidin-4-one | |

Section 2: Method 17

### 6-(1-Azido-2-methyl-propyl)-5-benzyl-3-methyl-5H-isothiazolo[5,4-d]pyrimidin-4-one

To a solution of 5-benzyl-6-(1-bromo-2-methyl-propyl)-3-methyl-5H-isothiazolo[5,4-d]pyrimidin-4-one (Section 2: Method 16) (0.6 g, 1.52 mmol) in anhydrous DMF (20 mL), sodium azide (0.65 g, 10 mmol) was added and the mixture was stirred at room temperature for 1 hour. The TLC of the RM showed the complete disappearance of the starting bromide. The reaction mixture was poured into ice water (300 mL) and extracted with EtOAc (3×100 mL). The organic layer was washed with water (100 mL), brine (100 mL) and dried (Na₂SO₄). Concentration of the organic layer provided the crude product which was purified by column (silica gel) chromatography using 30% EtOAc in hexanes as eluent to isolate 6-(1-azido-2-methyl-propyl)-5-benzyl-3-methyl-5H-isothiazolo[5,4-d]pyrimidin-4-one (0.506 g, 94%) as a low melting solid. Having the following properties m/z 355 (MH⁺), ¹H NMR (300 MHz) δ 0.57 (d, 3H), 1.07 (d, 3H), 2.50-2.74 (m, 1H), 2.98 (s, 3H), 3.71 (d, 1H), 5.05 (d, 1H), 5.78 (d, 1H), 7.12-7.40 (m, 5H).

Section 2: Method 17a

The following compounds were synthesized according to Section 2: Method 17 starting from 6-(1-bromo-2-methyl-propyl)-5-(3-fluoro-benzyl)-3-methyl-5H-isothiazolo[5,4-d]pyrimidin-4-one (Section 2: Method 16a):

| Section 2: | | |
|---|---|---|
| Method # | Compound Name | m/z |
| 17a | 6-(1-Azido-2-methyl-propyl)-5-(3-fluoro-benzyl)- | 373(MH⁺) |
| | 3-methyl-5H-isothiazolo[5,4-d]pyrimidin-4-one | |

Section 2: Method 18

### 6-(1-Amino-2-methyl-propyl)-5-benzyl-3-methyl-5H-isothiazolo[5,4-d]pyrimidin-4-one

To a solution of 6-(1-azido-2-methyl-propyl)-5-benzyl-3-methyl-5H-isothiazolo[5,4-d]pyrimidin-4-one (Section 2: Method 17) (0.5 g, 1.41 mmol) in methanol (20 mL) was added 5% Pd/C (20% by wt.) and the resulting mixture was stirred at r.t. in an atmosphere of H₂ and the progress of the reaction was monitored by MS. After the disappearance of the starting material the reaction mixture was filtered through celite and washed with EtOAc. Concentration of the filtrate provided 6-(1-amino-2-methyl-propyl)-5-benzyl-3-methyl-5H-isothiazolo[5,4-d]pyrimidin-4-one as a thick oil. The product was used as such in the next reaction with out further purification. Having the following properties m/z 349 (MH).

Section 2: Method 18a

The following compounds were synthesized according to Section 2: Method 18 starting from 6-(1-azido-2-methyl-propyl)-5-(3-fluoro-benzyl)-3-methyl-5H-isothiazolo[5,4-d]pyrimidin-4-one (Section 2: Method 17a):

| Section 2: | | |
|---|---|---|
| Method # | Compound Name | m/z |
| 18a | 6-(1-Amino-2-methyl-propyl)-5-(3-fluoro- | 367(MH⁺) |
| | benzyl)-3-methyl-5H-isothiazolo[5,4-d] | |
| | pyrimidin-4-one | |

Section 2: Method 19

### {2-[1-(5-Benzyl-3-methyl-4-oxo-4,5-dihydro-isothiazolo[5,4-d]pyrimidin-6-yl)-2-methyl-propylamino]-ethyl}-carbamic acid tert-butyl ester

To a mixture of 6-(1-amino-2-methyl-propyl)-5-benzyl-3-methyl-5H-isothiazolo[5,4-d]pyrimidin-4-one (Section 2: Method 18) (1.1 g, 3.35 mmol) and molecular sieves (4 A, 20 g) in DCM was added the solution of (2-oxo-ethyl)-carbamic acid tert-butyl ester (0.53 g, 3.35 mmol). The resulting reaction mixture was stirred at rt for 7 h. After addition of AcOH (2 drops), sodium triacetoxy borohydride (0.71 g, 3.35 mmol) was added. The reaction mixture was stirred overnight at rt. It was filtered through a pad of celite and celite cake was washed with DCM. The filtrate was washed with sat. NaHCO₃ (15 ml) and org. layer was separated. Aq. layer was re-extracted with DCM (100 mL). The combined org. layers were dried over MgSO4, filtered and concentrated in vacuo to yield {2-[1-(5-Benzyl-3-methyl-4-oxo-4,5-dihydro-isothiazolo[5,4-d]pyrimidin-6-yl)-2-methyl-propylamino]-ethyl}-carbamic acid tert-butyl ester (1.50 g, white foam). The crude product was used in next step. Having the following properties m/z 472 (MH⁺).

Section 2: Methods 19a-19b

The following compounds were synthesized according to Section 2: Method 19:

| Section 2: | | | |
|---|---|---|---|
| Method # | Compound Name | m/z | SM |
| 19a | (3-{1-[5-(3-Fluoro-benzyl)-3- | 504(MH⁺) | (3-oxo-propyl)- |
| | methyl-4-oxo-4,5-dihydro- | | carbamic acid |
| | isothiazolo[5,4-d]pyrimidin-6- | | tert-butyl ester |
| | yl]-2-methyl-propylamino}- | | and Section 2: |
| | propyl)-carbamic acid tert-butyl | | Method 18a |
| | ester | | |
| 19b | {3-[1-(5-Benzyl-3-methyl-4- | 486(MH⁺) | (3-oxo-propyl)- |
| | oxo-4,5-dihydro-isothiazolo[5,4- | | carbamic acid |
| | d]pyrimidin-6-yl)-2-methyl- | | tert-butyl ester |
| | propylamino]-propyl}-carbamic | | and Section 2: |
| | acid tert-butyl ester | | Method 18 |

Section 2: Method 20

### 5-Benzyl-6-[1-(2-[1,3]dioxolan-2-yl-ethylamino)-2-methyl-propyl]-3-methyl-5H-isothiazolo[4 d]pyrimidin-4-one

To a solution of 6-(1-amino-2-methyl-propyl)-5-benzyl-3-methyl-5H-isothiazolo[5,4-d]pyrimidin-4-one (Section 2: Method 18) (1.6 g, 4.88 mmol) in anhydrous DMF (20 mL), 2-(2-bromo-ethyl)-[1,3]dioxolane (0.88 g, 4.88 mmol) was added and the resulting solution was heated at 70° C. for 2 h. The reaction mixture was cooled, diluted with water and extracted with EtOAc (3×60 mL). The combined organic extracts were dried (Na₂SO₄) and concentrated to provide the crude product (2 g), which was used as such in the next reaction. Having the following properties m/z 429 (MH⁺); ¹H-NMR (300 MHz) δ 0.88 (d, 3H), 0.96 (d, 3H), 1.54-1.62 (m, 2H), 1.86-2.05 (m, 2H), 2.18 (bs, 1H), 2.38-2.46 (m, 1H), 2.84 (s, 3H), 3.57 (d, 1H), 3.74-3.94 (m, 4H), 4.78 (t, 1H), 4.99 (d, 1H), 5.85 (d, 1H), 7.15-7.38 (m, 5H).

Section 2: Method 21

### {2-[[1-(5-Benzyl-3-methyl-4-oxo-4,5-dihydro-isothiazolo[5,4-d]pyrimidin-6-yl)-2-methyl-propyl]-(4-methyl-benzoyl)-amino]-ethyl}-carbamic acid tert-butyl ester

To a solution of {2-[1-(5-benzyl-3-methyl-4-oxo-4,5-dihydro-isothiazolo[5,4-d]pyrimidin-6-yl)-2-methyl-propylamino]-ethyl}-carbamic acid tert-butyl ester (Section 2: Method 19) (1.50 g, 3.20 mmol), DIEA (0.75 g, 5.8 mmol) in CHCl₃ (30 mL) at 60° C. under nitrogen atmosphere was added a solution of p-toluoyl chloride (0.74 g, 4.8 mmol) in CHCl₃ (60 mL). The reaction mixture was refluxed for 27 h and then cooled to rt. The reaction mixture was treated with sat. NaHCO₃ (50 ml). The organic layer was separated and the aqueous layer was re-extracted with CHCl₃ (150 mL). The combined org. layers were dried over MgSO4, filtered and concentrated in vacuo to yield {2-[[1-(5-Benzyl-3-methyl-4-oxo-4,5-dihydro-isothiazolo[5,4-d]pyrimidin-6-yl)-2-methyl-propyl]-(4-methyl-benzoyl)-amino]-ethyl}-carbamic acid tert-butyl ester (1.10 g, 69% overall yield). m/z 590 (MH⁺).

Section 2: Methods 21a-21 h

The following compounds were synthesized according to Section 2: Method 21:

| Section 2: | | | | Acylating |
|---|---|---|---|---|
| Method # | Compound Name | m/z | SM | agent |
| 21a | {3-[{1-[5-(3-Fluoro-benzyl)-3-methyl-4- | 622(MH⁺) | Section 2: | 4-methyl- |
| | oxo-4,5-dihydro-isothiazolo[5,4- | | Method | benzoyl |
| | d]pyrimidin-6-yl]-2-methyl-propyl}-(4- | | 19a | chloride |
| | methyl-benzoyl)-amino]-propyl}-carbamic | | | |
| | acid tert-butyl ester | | | |
| 21b | {2-[[1-(5-Benzyl-3-methyl-4-oxo-4,5- | 654, | Section 2: | 4-bromo- |
| | dihydro-isothiazolo[5,4-d]pyrimidin-6-yl)- | 656(MH⁺) | Method | benzoyl |
| | 2-methyl-propyl]-(4-bromo-benzoyl)- | | 19 | chloride |
| | amino]-ethyl}-carbamic acid tert-butyl | | | |
| | ester | | | |
| 21c | {2-[[1-(5-Benzyl-3-methyl-4-oxo-4,5- | 608(MH⁺) | Section 2: | 3-fluoro-4- |
| | dihydro-isothiazolo[5,4-d]pyrimidin-6-yl)- | | Method | methyl- |
| | 2-methyl-propyl]-(3-fluoro-4-methyl- | | 19 | benzoyl |
| | benzoyl)-amino]-ethyl}-carbamic acid tert- | | | chloride |
| | butyl ester | | | |
| 21d | {3-[[1-(5-Benzyl-3-methyl-4-oxo-4,5- | 622(MH⁺) | Section 2: | 3-fluoro-4- |
| | dihydro-isothiazolo[5,4-d]pyrimidin-6-yl)- | | Method | methyl- |
| | 2-methyl-propyl]-(3-fluoro-4-methyl- | | 19b | benzoyl |
| | benzoyl)-amino]-propyl}-carbamic acid | | | chloride |
| | tert-butyl ester | | | |
| 21e | {3-[[1-(5-Benzyl-3-methyl-4-oxo-4,5- | 668, | Section 2: | 4-bromo- |
| | dihydro-isothiazolo[5,4-d]pyrimidin-6-yl)- | 670(MH⁺) | Method | benzoyl |
| | 2-methyl-propyl]-(4-bromo-benzoyl)- | | 19b | chloride |
| | amino]-propyl}-carbamic acid tert-butyl | | | |
| | ester | | | |
| 21f | N-[1-(5-Benzyl-3-methyl-4-oxo-4,5- | 547(MH⁺) | Section 2: | 4-methyl- |
| | dihydro-isothiazolo[5,4-d]pyrimidin-6-yl)- | | Method | benzoyl |
| | 2-methyl-propyl]-N-(2-[1,3]dioxolan-2-yl- | | 20 | chloride |
| | ethyl)-4-methyl-benzamide | | | |
| 21g | N-[1-(5-Benzyl-3-methyl-4-oxo-4,5- | 611, | Section 2: | 4-bromo- |
| | dihydro-isothiazolo[5,4-d]pyrimidin-6-yl)- | 613(MH⁺) | Method | benzoyl |
| | 2-methyl-propyl]-4-bromo-N-(2- | | 20 | chloride |
| | [1,3]dioxolan-2-yl-ethyl)-benzamide | | | |
| 21h | N-[1-(5-Benzyl-3-methyl-4-oxo-4,5- | 565(MH⁺) | Section 2: | 3-fluoro-4- |
| | dihydro-isothiazolo[5,4-d]pyrimidin-6-yl)- | | Method | methyl- |
| | 2-methyl-propyl]-N-(2-[1,3]dioxolan-2-yl- | | 20 | benzoyl |
| | ethyl)-3-fluoro-4-methyl-benzamide | | | chloride |

Section 2: Method 22

### N-[1-(5-Benzyl-3-methyl-4-oxo-4,5-dihydro-isothiazolo[5,4-d]pyrimidin-6-yl)-2-methyl-propyl]-4-bromo-N-(3-oxo-propyl)-benzamide

N-[1-(5-Benzyl-3-methyl-4-oxo-4,5-dihydro-isothiazolo[5,4-d]pyrimidin-6-yl)-2-methyl-propyl]-4-bromo-N-(2-[1,3]dioxolan-2-yl-ethyl)-benzamide (Section 2: Method 21 g) (1.1 g, 1.8 mmol) was dissolved in 20 mL of 80% acetic acid and the solution was heated at 80° C. for 2 h. The reaction mixture was cooled in an ice bath and neutralized slowly by the addition of solid NaHCO₃ until pH 8. The thus obtained mixture was extracted with DCM (3×100 mL). The combined organic layers was washed with brine (100 mL) and dried (Na₂SO₄). Concentration of the DCM layer provided a yellow foam (1 g crude yield) and it was used as such in the next reaction. m/z 567, 569 (MH⁺).

Section 2: Methods 22a-22b

The following compounds were synthesized according to Section 2: Method 22:

| Section 2: | | | |
|---|---|---|---|
| Method # | Compound Name | m/z | SM |
| 22a | N-[1-(5-Benzyl-3-methyl-4-oxo-4,5- | 503(MH⁺) | Section 2: |
| | dihydroisothiazolo[5,4-d]pyrimidin-6-yl)-2-methyl- | | Method |
| | propyl]-4-methyl-N-(3-oxo-propyl)-benzamide | | 21f |
| 22b | N-[1-(5-Benzyl-3-methyl-4-oxo-4,5- | 521(MH⁺) | Section 2: |
| | dihydroisothiazolo[5,4-d]pyrimidin-6-yl)-2-methyl- | | Method |
| | propyl]-3-fluoro-4-methyl-N-(3-oxo-propyl)-benzamide | | 21h |

Section 2: Method 23

3-Methyl-5-(3-methyl-butyryl)-isoxazole-4-carboxylic acid amide

A mixture of 5-amino-3-methyl-isoxazole-4-carboxylic acid amide (10 g, 70 mmol) in 25 ml of isovaleric anhydride was stirred at 110-145° C. for 1 h. The brown solution was diluted with hexane (500 ml) and cooled down. The precipitated gum was separated from the mixture and washed with hexane, dried in vacuo. 3-Methyl-5-(3-methyl-butyryl)-isoxazole-4-carboxylic acid amide was obtained as a yellow gum. Further used without purification in Section 2: Method 24.

Section 2: Method 24

### 6-Isobutyl-3-methyl-5H-isoxazolo[5,4-d]pyrimidin-4-one

A suspension of 3-methyl-5-(3-methyl-butyryl)-isoxazole-4-carboxylic acid amide (Section 2: Method 23) (split into 40 vials) in 3.5 ml of 2N NaOH aq was subjected to microwave irradiation at 140° C. for 20 min. The resulting solution was cooled with an ice bath, and the pH was adjusted to 1˜3 with concentrated HCl. The solid was filtered, washed with water, dried over vacuum at 40° C. overnight. 6-Isobutyl-3-methyl-5H-isoxazolo[5,4-d]pyrimidin-4-one (8 g) was obtained as a white solid. 55% yield for two steps. Having the following properties m/z: 208 (MH⁺), ¹H NMR (DMSO-d₆): 0.76 (d, 6H), 1.95 (m, 1H), 2.25 (s, 3H), 2.32 (d, 2H), 12.55 (s, 1H).

Section 2: Method 25

### 5-(3-Fluoro-benzyl)-6-isobutyl-3-methyl-5H-isoxazolo[5,4-d]pyrimidin-4-one

A suspension of 6-isobutyl-3-methyl-5H-isoxazolo[5,4-d]pyrimidin-4-one (Section 2: Method 24) (1.24 g, 6.0 mmol), 3-fluorobenzylbromide (1.13 g, 6.0 mmol), potassium carbonate (1.38 g, 10.0 mmol) in 20 ml DMF was stirred at room temperature for 2 days. The mixture was diluted with water, extracted with EtOAc (100 ml×3), the combined organic phases were dried, concentrated, purified by flash column chromatography (elute: hexane-EtOAc=10:3). 5-(3-Fluoro-benzyl)-6-isobutyl-3-methyl-5H-isoxazolo[5,4-d]pyrimidin-4-one was obtained as white solid (1.0 g, 3.17 mmol) (53%). Having the following properties m/z: 316 (MH⁺), 1H-NMR (300 MHz) δ: 0.96 (d, 6H), 2.27-2.41 (heptet, 1H), 2.59 (s, 3H), 2.65 (d, 2H), 5.37 (s, 2H), 6.80-7.05 (m, 3H), 7.30-7.40 (m, 1H).

Section 2: Method 26

### 6-(1-Bromo-2-methyl-propyl)-5-(3-fluoro-benzyl)-3-methyl-5H-isoxazolo[5,4-d]pyrimidin-4-one

A solution of 5-(3-fluoro-benzyl)-6-isobutyl-3-methyl-5H-isoxazolo[5,4-d]pyrimidin-4-one (Section 2: Method 25) (1.0 g, 3.17 mmol) and sodium acetate (1.0 g, 12.1 mmol) in glacial acetic acid (20 ml) was treated with a preformed bromine solution (1.0 g bromine in 20 ml of glacial acetic acid) (0.32 ml, 6.29 mmol). The mixture was stirred at 110-120° C. for 1 day. Water was added to the mixture to which was subsequently added potassium carbonate and extracted with DCM (20 ml×3), the combined organic phases were washed with water and dried, then concentrated to give the crude product which was purified by ISCO (elute: hexane-EtOAc). 6-(1-Bromo-2-methyl-propyl)-5-(3-fluoro-benzyl)-3-methyl-5H-isoxazolo[5,4-d]pyrimidin-4-one was obtained as a yellow gum (1.1 g, 2.79 mmol) (88%). Having the following properties m/z: 394, 396 (MH⁺), ¹H-NMR (300 MHz) δ: 0.61 (d, 3H), 1.14 (d, 3H), 2.64 (s, 3H), 2.71-2.80 (m, 1H), 4.35 (d, 2H), 4.82 (d, 1H), 6.13 (d, 1H), 6.82-7.03 (m, 3H), 7.32-7.39 (m, 1H).

Section 2: Method 27

### 6-(1-Azido-2-methyl-propl)-5-(3-fluoro-benzyl)-3-methyl-5H-isoxazolo[5,4-d]pyrimidin-4-one

A suspension of 6-(1-bromo-2-methyl-propyl)-5-(3-fluoro-benzyl)-3-methyl-5H-isoxazolo[5,4-d]pyrimidin-4-one (Section 2: Method 26) (10.10 g, 2.79 mmol) and sodium azide (0.88 g, 13.9 mmol, 5 eq.) in DMF (10 ml) was stirred at 60° C. for 1 h. Water (10 ml) was added to the mixture and then extracted with EtOAc (3×20 ml). The combined organic phases were washed with brine (20 ml), dried, concentrated and purified by ISCO (Hexane-EtOAc). 6-(1-Azido-2-methyl-propyl)-5-(3-fluoro-benzyl)-3-methyl-5H-isoxazolo[5,4-d]pyrimidin-4-one was obtained (0.98 g, 2.72 mmol (97%) as a colourless oil. Having the following properties m/z: 357 (MH⁺), 1H-NMR (300 MHz) δ: 0.59 (d, 3H), 1.10 (d, 3H), 2.62 (s, 3H), 2.58-2.70 (m, 1H), 3.65 (d, 2H), 5.05 (d, 1H), 5.75 (d, 1H), 6.82-7.03 (m, 3H), 7.31-7.39 (m, 1H).

Section 2: Method 28

### 6-(1-Amino-2-methyl-propyl)-5-(3-fluoro-benzyl)-3-methyl-5H-isoxazolo[5,4-d]pyrimidin-4-one

A mixture of 6-(1-azido-2-methyl-propyl)-5-(3-fluoro-benzyl)-3-methyl-5H-isoxazolo[5,4-d]pyrimidin-4-one (Section 2: Method 27) (0.9 g, 2.72 mmol), triphenylphosphine (0.78 g, 3.0 mmol) in anhydrous toluene (20 ml) was stirred at 110° C. for 3 hours. Excess amount of water (50 μl) was added to the mixture and stirred at 60° C. for 16 hours. The volatile solvent was distilled off and the crude product was used in the next step without purification. Having the following properties m/z: 331 (MH⁺).

Section 2: Method 29

### (3-{1-[5-(3-Fluoro-benzyl)-3-methyl-4-oxo-4,5-dihydro-isoxazolo[5,4-d]pyrimidin-6-yl]-2-methyl-propylamino}-propyl)-carbamic acid tert-butyl ester

A mixture of 6-(1-amino-2-methyl-propyl)-5-(3-fluoro-benzyl)-3-methyl-5H-isoxazolo[5,4-d]pyrimidin-4-one (Section 2: Method 28) (0.89 g, 2.72 mmol) and (3-oxo-propyl)-carbamic acid tert-butyl ester (1.0 g, 6.0 mmol) in DCM (20 ml) with dried 4 ÅMS was stirred for 1 h at room temperature. Then sodium triacetoxyborohydride (0.63 g, 3 mmol, 1.2 eq) and 1 drop of acetic acid were added to the mixture. The mixture was stirred at room temperature for 1 day. The mixture was filtered through a 2μ cartridge, the filtrate was concentrated, the crude mixture was purified by ISCO (elute: EtOAc-hexane=30%-70%) to give 300 mg, 0.61 mmol (22% yield for 2 steps) of (3-{1-[5-(3-Fluoro-benzyl)-3-methyl-4-oxo-4,5-dihydro-isoxazolo[5,4-d]pyrimidin-6-yl]-2-methyl-propylamino}-propyl)-carbamic acid tert-butyl ester as a white solid. Having the following properties m/z: 488 (MH⁺), 1H-NMR (300 MHz) δ: 0.92 (d, 3H), 0.97 (d, 3H), 1.42 (s, 9H), 1.32-1.48 (m, 1H), 1.77-2.01 (m, 3H), 2.36-2.43 (m, 1H), 2.62 (s, 3H), 2.96-3.12 (m, 2H), 3.54 (d, 1H), 2.62 (s, 3H), 2.58-2.70 (m, 1H), 3.65 (d, 2H), 4.89 (d, 1H), 5.22 (d, 1H), 5.88 (d, 1H), 6.82-7.03 (m, 3H), 7.31-7.39 (m, 1H).

Section 2: Method 30

### {3-[{1-[5-(3-Fluoro-benzyl)-3-methyl-4-oxo-4,5-dihydro-isoxazolo[5,4-d]pyrimidin-6-yl]-2-methyl-propyl}-(4-methyl-benzoyl)-amino]-propyl}-carbamic acid tert-butyl ester

A solution of (3-{1-[5-(3-fluoro-benzyl)-3-methyl-4-oxo-4,5-dihydro-isoxazolo[5,4-d]pyrimidin-6-yl]-2-methyl-propylamino}-propyl)-carbamic acid tert-butyl ester (Section 2: Method 29) (300 mg, 0.61 mmol) in DCM (10 ml) was added p-toluoyl chloride (1.54 g, 1.0 mmol, 1.6 eq) followed by diisopropylethylamine (0.26 g, 2.0 mmol). The mixture was stirred at 30-40° C. for 1 day. The mixture was then diluted with DCM, washed with saturated sodium bicarbonate aq. The organic phase was dried, filtered, and concentrated. The crude oil was purified by ISCO (solvent: EtOAc-hexane) to give {3-[{1-[5-(3-Fluoro-benzyl)-3-methyl-4-oxo-4,5-dihydro-isoxazolo[5,4-d]pyrimidin-6-yl]-2-methyl-propyl}-(4-methyl-benzoyl)-amino]-propyl}-carbamic acid tert-butyl ester as white solid (204 mg, 0.34 mmol) (54% yield). Having the following properties: m/z: 606 (MH⁺) and 1H-NMR (300 MHz) δ: 0.36 (d, 3H), 0.96 (d, 3H), 1.43 (s, 9H), 1.39-1.48 (m, 1H), 2.39 (s, 3H), 2.66 (s, 3H), 2.56-2.76 (m, 4H), 3.43 (t, 2H), 4.01 (m, 1H), 5.30 (d, 1H), 5.72 (d, 1H), 6.05 (d, 1H), 6.92-7.31 (m, 9H).

Section 2: Method 31

### 3-Amino-2-thioformyl-but-2-enoic acid ethyl ester

To an ice cold solution of phosphoryl chloride (20 mL, 220 mmol), anhydrous DMF (60 mL) was added dropwise and the resulting solution was added dropwise during 30 min to a stirred solution of the ethyl crotonate (25.83 g, 200 mmol) in anhydrous THF (400 mL) with the temperature maintained at 0° C. The resulting mixture was allowed to warm to room temperature and stirred overnight and then for 4 h at 30° C.; it was then allowed to stand overnight in a refrigerator. Addition of ether (200 mL) resulted in a yellow oil from which the ether layer was decanted. The resulting oil was washed several times with ether until the ether layer became clear. The oily product was dissolved in DCM (800 mL) and was vigorously shaken with aqueous sodium hydrogen sulfide (2M; 500 mL). The organic layer was separated and the aqueous layer washed with DCM (100 mL). The combined organic layers were washed with water (600 mL), brine (400 mL), dried (Na₂SO₄) and concentrated to get orange crystals. The obtained product was triturated with DCM/hexanes to get pure product as orange crystals (25.6 g, 74%). Having the following properties ¹H NMR (300 MHz) δ: 1.33 (t, 3H), 2.57 (s, 3H), 4.23 (q, 2H), 6.83 (bs, 1H), 10.97 (s, 1H), 13.93 (s, 1H).

Section 2: Method 32

### 3-Methyl-isothiazole-4-carboxylic acid ethyl ester

To a solution of 3-amino-2-thioformyl-but-2-enoic acid ethyl ester (Section 2: Method 31) (25.6 g, 147 mmol) in ethanol (300 mL), was added m-chloroperbenzoic acid (33.3 g, 77%, 149 mmol) in ethanol (200 mL) dropwise with stirring at room temperature. After the completion of the addition the reaction mixture was heated at 75° C. for 2 h after which the MS showed the complete disappearance of the starting material. The reaction mixture was diluted with ether (500 mL) and the ethereal solution was washed with 0.1 M NaOH solution (3×500 mL) and once with water (400 mL) dried (Na₂SO₄) and concentrated to get the pure product as light brown oil. Yield 23.5 g (93%). Having the following properties: ¹H NMR (300 MHz) δ: 1.40 (t, 3H), 2.73 (s, 3H), 5.07 (t, 1H), 4.36 (q, 2H), 9.24 (s, 1H).

Section 2: Method 33

### 3-Methyl-isothiazole-4-carboxylic acid

To a solution of 3-methyl-isothiazole-4-carboxylic acid ethyl ester (Section 2: Method 32) (23.3 g, 136 mmol) in THF (200 mL) aqueous NaOH (6.5 g, 162 mmol, in 100 ml of water) was added and the mixture was stirred at room temperature for 16 h. The TLC of the reaction mixture showed the complete disappearance of the starting material. The reaction mixture was cooled in an ice bath and acidified to pH 5 using 6M HCl and the resultant mixture was extracted with ether (3×100 mL). The ether layers were combined, washed with water (100 mL), brine (100 mL), dried (Na₂SO₄) and concentrated to about 10 mL. Addition of hexanes to the above mixture resulted in the precipitation of the product, which was filtered off, washed with hexanes and dried to provide the pure product as a tan powder. Yield 15.3 g (79%). Having the following properties:

¹H NMR (300 MHz) δ 2.39 (s, 3H), 8.98 (s, 1H).

Section 2: Method 34

### (3-Methyl-isothiazol-4-yl)-carbamic acid tert-butyl ester

To a solution of 3-methyl-isothiazole-4-carboxylic acid (Section 2: Method 33) (14.8 g, 103 mmol) in anhydrous t-BuOH (100 mL) triethyl amine (10.5 g, 104 mmol) was added followed by the dropwise addition of diphenylphosphoryl azide (28.6 g, 104 mmol) and the resulting mixture was heated at reflux overnight after which the TLC showed the complete disappearance of the starting material. The reaction mixture was cooled to room temperature and poured into ice cold water (500 mL). The aqueous layer was extracted with ether (3×100 mL) and the combined organic layers were washed with satd, NaHCO₃ (100 mL), brine (100 mL) and dried (Na₂SO₄). Concentration of the ether solution provided the crude product, which was purified by column chromatography to get the pure product as light brown crystals. Yield 21.4 g (97%). Having the following properties ¹H NMR (300 MHz) δ 1.53 (s, 9H), 2.40 (s, 3H), 6.50 (s, 1H), 8.66 (s, 1H).

Section 2: Method 35

### 4-tert-Butoxycarbonylamino-3-methyl-isothiazole-5-carboxylic acid

To a solution of (3-methyl-isothiazol-4-yl)-carbamic acid tert-butyl ester (Section 2: Method 34) (21.4 g, 100 mmol) in anhydrous THF (200 mL) at −78° C., LDA (139 mL, 1.8 M solution, 250 mmol) was added dropwise over a period of 1 h. The reaction mixture was stirred at −78° C. for a further 3 h after which powdered dry ice was added and the reaction slowly allowed to warm to room temperature overnight. The reaction mixture was quenched by adding saturated NH₄Cl solution and extracted with ether (3×100 mL) and the combined ether layers were back extracted with satd. NaHCO₃ (3×100 mL). The aqueous layers were combined and acidified to pH 5 using 6M HCl and extracted with ether (4×100 mL). The combined ether layers were dried (Na₂CO₃) and concentrated to give the pure acid as an off white powder. Yield 11 g (39%). Having the following properties: ¹H NMR (300 MHz) δ 1.47 (s, 9H), 2.44 (s, 3H), 8.53 (bs, 1H), 9.68 (bs, 1H).

Section 2: Method 36

### 4-Amino-3-methyl-isothiazole-5-carboxylic acid

4-tert-Butoxycarbonylamino-3-methyl-isothiazole-5-carboxylic acid (Section 2: Method 35) (11 g, 45 mmol) was dissolved in 50 mL of 4M solution of HCl in 1,4-dioxane (200 mmol) and the resulting solution was stirred at room temperature overnight. The TLC showed the complete disappearance of the starting acid. The reaction was concentrated and the residue was triturated with ether and the precipitated hydrochloride salt was filtered off and washed with ether and dried to provide the product as a light brown powder. Yield 8.2 g (100%). Having the following properties: ¹H NMR (300 MHz, DMSO-d6) δ 2.30 (s, 3H), 8.85 (bs, 3H).

Section 2: Method 37

### 3-Methyl-5-propyl-isothiazolo[4,5-d][1,3]oxazin-7-one

To a solution of 4-amino-3-methyl-isothiazole-5-carboxylic acid (Section 2: Method 36) (2.91 g, 15 mmol) in pyridine (20 mL) at 0° C., was added dropwise a solution of butyryl chloride (3.18 g, 30 mmol) in chloroform (30 mL). The reaction mixture was allowed to warm to room temperature and stirred overnight. Chloroform (200 mL) was added to the reaction mixture followed by 2M HCl (200 mL) and the mixture was stirred. The chloroform layer was further washed with 2M HCl (100 mL), water (100 mL), brine (100 mL) and concentrated. Column purification of the thus obtained crude product provided the pure product as light brown solid. Yield 2 g (64%). Having the following properties: ¹H NMR (300 MHz) δ 1.03 (t, 3H), 1.80-1.92 (m, 2H), 2.65 (s, 3H), 2.76 (t, 2H).

Section 2: Method 38

### 6-Benzyl-3-methyl-5-propyl-6H-isothiazolo[4,5-d]pyrimidin-7-one

3-Methyl-5-propyl-isothiazolo[4,5-d][1,3]oxazin-7-one (Section 2: Method 37) (200 mg, 1.02 mmol) was taken in a 10 mL microwavable pyrex tube and benzyl amine (1 g, 9.34 mmol) was added to it. The resulting mixture was heated in a microwave synthesizer (CEM's Discoverer) at 200° C. for 20 min. The MS of the reaction mixture showed the complete disappearance of the starting material and the presence of the product peak at 286 (MH⁺). The reaction mixture was diluted with 1N HCl (10 mL) and extracted with EtOAc (2×30 mL). The combined EtOAc layers were washed with water, brine, dried and concentrated. The thus obtained crude product was purified by column chromatography to isolate the pure product as a white solid. Yield 208 mg (71%). Having the following properties: ¹H NMR (300 MHz) δ 0.98 (t, 3H), 1.76-1.88 (m, 2H), 2.68 (s, 3H), 2.74 (t, 2H), 5.42 (s, 2H), 7.10-7.19 (m, 2H), 7.28-7.39 (m, 3H).

Section 2: Method 39

### 6-Benzyl-5-(1-bromo-propyl)-3-methyl-6H-isothiazolo[4,5-d]pyrimidin-7-one

To a solution of 6-benzyl-3-methyl-5-propyl-6H-isothiazolo[4,5-d]pyrimidin-7-one (Section 2: Method 38) (208 mg, 0.69 mmol) and sodium acetate (0.5 g, 5 mmol) in acetic acid (10 mL) at 100° C., a solution of the bromine (0.232 g, 1.46 mmol) in acetic acid (20 mL) was added dropwise over a period of 30 min. The reaction mixture was cooled after the addition and the TLC (eluent 10% EtOAc in hexanes) and MS showed the complete disappearance of the SM and only the product. The reaction mixture was poured into ice water and extracted with EtOAc (3×30 mL) and the organic layers were combined and washed with 2% sodium thiosulfate solution (30 mL), water (50 mL), brine (50 mL) and dried (Na₂SO₄). Concentration of the organic layer provided the product and it was pure enough to be used in the next step. Yield 260 mg (99%). Having the following properties: ¹H NMR (300 MHz) δ 0.77 (t, 3H), 2.20-2.54 (m, 2H), 2.70 (s, 3H), 4.67 (t, 1H), 4.95 (d, 1H), 6.25 (d, 1H) 7.10-7.19 (m, 2H), 7.30-7.39 (m, 3H).

Alternative Procedures to Prepare Certain Starting Materials

Section 2: Method 1

### 2-(1-Ethoxy-ethylidene)-malononitrile (alternative procedure)

Triethyl orthoacetate (1.6 L, 9 mol), malononitrile (500 g, 7.57 mol) and glacial acetic acid (25 ml) were placed in a 5 l RB flask equipped with a stirrer, thermometer and a Vigreux column (20×1 in.) on top of which a distillation condenser was placed. The reaction mixture was heated and ethyl alcohol began to distil when the temperature of the reaction mixture was about 85-90° C. After about 3 h., the temperature of the reaction mixture reached 140° C. Then the reaction was concentrated in a rotary evaporator to remove the low-boiling materials and the residue was stirred with isopropyl alcohol (1 l) and cooled in an ice bath. The crystallized product was filtered off washed with isopropyl alcohol (200 ml), hexanes (600 ml) and dried at 50° C. in a vacuum oven overnight to yield 2-(1-ethoxy-ethylidene)-malononitrile (974 g, 94%) as a golden yellow solid [mp 92. ° C. (lit. 90-92° C., MCCall. M. A. J. Org. Chem. 1962, 27, 2433-2439.)].

Section 2: Method 2

### (2E)-2-Cyano-3-ethoxybut-2-enethioamide (alternative procedure)

2-(1-Ethoxy-ethylidene)-malononitrile (Section 2: Method 1) (300 g, 2.2 mol) was dissolved in anhydrous benzene (3.1 l, slight warming required) and 20 ml of triethylamine was added. The mixture was mechanically stirred and hydrogen sulfide was bubbled into this solution for 2 h and a solid formed. Then N₂ was bubbled through the reaction mixture for 40 min. The precipitated solid was filtered off, washed with cold benzene (200 ml) and dried in a vacuum oven overnight to isolate (2E)-2-cyano-3-ethoxybut-2-enethioamide (332 g, 88%) as light brown crystals.

Section 2: Method 3

### (2E)-3-Amino-2-cyanobut-2-enethioamide (alternative procedure)

(2E)-2-Cyano-3-ethoxybut-2-enethioamide (Section 2: Method 2) (150 g, 0.88 mol) was dissolved in 7M solution of ammonia in methanol (2.9 L) and stirred at r.t. overnight. The reaction mixture was concentrated and the residue was crystallized from hot water (1. L) to provide (2E)-3-amino-2-cyanobut-2-enethioamide (111.6 g, 89%) as brown crystals. ¹H NMR (300 MHz, DMSO-d₆) δ 2.22 (s, 3H), 7.73 (bs, 1H), 8.53 (bs, 1H), 9.01 (bs, 1H), 11.60 (bs, 1H).

Section 2: Method 4

### 5-Amino-3-methylisothiazole-4-carbonitrile (alternative procedure)

To a stirred solution of (2E)-3-amino-2-cyanobut-2-enethioamide (Section 2: Method 3) (111 g, 0.78 mol) in methanol (2 L) was added dropwise 200 ml of 35% hydrogen peroxide over a period of 30 min. After the completion of the addition the mixture was stirred at 60° C. for 3 h after which the TLC showed the completion of the reaction. The reaction mixture was evaporated to 300 ml in a rotary evaporator and cooled in an ice-bath. The crystallized product was filtered off and washed with isopropyl alcohol (100 ml) and dried in vacuum at 50° C. overnight to provide 5-amino-3-methylisothiazole-4-carbonitrile (105.63 g, 96%) as a light yellow crystalline solid. ¹H NMR (300 MHz, DMSO-d₆) δ 2.24 (s, 3H), 8.00 (bs, 2H).

Section 2: Method 12

### N-(4-Cyano-3-methyl-isothiazol-5-yl)-3-methyl-butyramide (alternative procedure)

To a solution of 5-amino-3-methylisothiazole-4-carbonitrile (Section 2: Method 4) (105.6 g, 0.76 mol) in pyridine (250 ml) at 0° C., isovaleryl chloride (100 g, 0.83 mol) in chloroform (300 ml) was added dropwise. After the completion of the addition the reaction mixture was allowed to warm to r.t. and stirred overnight. The TLC and the MS showed the complete disappearance of the starting material and the reaction mixture was diluted with CHCl₃ (600 ml), washed with water (200 ml), 2N HCl (600 ml), satd. NaHCO₃ (200 ml), brine (200 ml) and dried over Na₂SO₄. Concentration of the CHCl₃ layer provided the crude product which was triturated from DCM/hexanes (1/10) and filtered off to isolate N-(4-cyano-3-methyl-isothiazol-5-yl)-3-methyl-butyramide (149.7 g, 88%) as an off-white crystalline solid. ¹H NMR (300 MHz) δ 1.04 (d, 6H), 2.18-2.32 (m, 1H), 2.46 (d, 2H), 2.53 (s, 3H), 9.87 (bs, 1H).

Section 2: Method 13

### 3-Methyl-5-(3-methyl-butyrylamino)-isothiazole-4-carboxylic acid amide (alternative procedure)

To a solution of N-(4-cyano-3-methyl-isothiazol-5-yl)-3-methyl-butyramide (Section 2: Method 12) (72 g, 322 mmol) in 30% aqueous NH₄OH (2.1 L), was added dropwise 1.3 L of hydrogen peroxide at 40° C. After 20 min the temperature of the reaction mixture rose to 60° C. The addition was completed in 1.5 h. After an additional 2 h the MS showed the completion of the reaction. The reaction mixture was cooled in ice and con HCl was slowly added with cooling till the pH of the reaction mixture turns 7.6. The precipitated product was filtered and dried in vacuum oven to get the pore amide (36 g, 46%). The filtrate was saturated with NaCl and extracted with super solvent (34:66, t-butanol: 1,2-dichloroethane) and the combined organic extracts were washed with water (500 ml), brine (600 ml) and dried (Na₂SO₄) and concentrated. The residue on trituration with EtOAc/hexanes (1/4) provided an additional 9.8 g of pure product. Total yield of 45.8 g (58%) 3-methyl-5-(3-methyl-butyrylamino)-isothiazole-4-carboxylic acid amide. ¹H NMR (300 MHz) δ 1.03 (d, 6H), 2.24 (m, 1H), 2.43 (d, 2H), 2.69 (s, 3H), 5.98 (bs, 2H), 11.77 (bs, 1H).

Section 2: Method 14

### 6-Isobutyl-3-methyl-5H-isothiazolo[5,4-d]pyrimidin-4-one (alternative procedure)

The 3-methyl-5-(3-methyl-butyrylamino)-isothiazole-4-carboxylic acid amide (Section 2: Method 13) (45.8 g, 190 mmol) was suspended in 700 ml of 30% NH₃ and then was heated to 140° C. for 5 h in a pressure reactor. The mixture was poured into a 4 L beaker and cooled in an ice bath. To the cold solution con HCl (560 ml) was added dropwise to pH 7.5 and a white precipitate was formed. The precipitated product was filtered off, washed with water (100 ml) and dried under vacuum overnight. 6-Isobutyl-3-methyl-5H-isothiazolo[5,4-d]pyrimidin-4-one (11 g, 26%) was isolated as an off-white powder. ¹H NMR (300 MHz) δ 1.05 (d, 6H), 2.32 (m, 1H), 2.69 (d, 2H), 2.82 (s, 3H).

Section 2: Method 15

### 5-Benzyl-6-isobutyl-3-methyl-5H-isothiazolo[5,4-d]pyrimidin-4-one (alternative procedure)

To a solution of 6-isobutyl-3-methyl-5H-isothiazolo[5,4-d]pyrimidin-4-one (Section 2: Method 14) (11 g, 49 mmol) in 60 ml of anhydrous DMF at 0° C., was added 13.8 g (100 mmol) of anhydrous K₂CO₃ followed by benzyl bromide (9.3 g, 54 mmol) and the mixture was stirred at 0-20° C. overnight. The TLC of the reaction mixture showed the complete disappearance of the SM. The reaction mixture was poured into ice-cold water and extracted with EtOAc (3×100 ml). The combined extracts were washed with water (100 ml), brine (100 ml), dried (Na₂SO₄) and concentrated. The TLC and the ¹H NMR showed the presence of two products N alkylated as well as O-alkylated products in a ratio of 75:25. The products were separated by column (silica gel) chromatography using 10% EtOAc in hexanes. The major N-alkylated product 5-benzyl-6-isobutyl-3-methyl-5H-isothiazolo[5,4-d]pyrimidin-4-one was isolated as white crystalline solid (10.8 g, 70%). ¹H NMR (300 MHz) δ 0.94 (d, 6H), 2.23-2.37 (m, 1H), 2.64 (d, 2H), 2.82 (s, 3H), 5.38 (s, 2H), 7.10-7.38 (m, 5H).

Section 2: Method 16

### 5-Benzyl-6-(1-bromo-2-methyl-propyl)-3-methyl-5H-isothiazolo[5,4-d]pyrimidin-4-one (alternative procedure)

To a solution of 5-benzyl-6-isobutyl-3-methyl-5H-isothiazolo[5,4-d]pyrimidin-4-one (Section 2: Method 15) (5.81 g, 18.5 mmol) and sodium acetate (10 g) in acetic acid (100 ml) at 100° C., a solution of the bromine (6 g, 38 mmol) in acetic acid (60 ml) was added dropwise over a period of 20 minutes. The reaction mixture was stirred at that temperature for 30 min and cooled and the TLC (eluent 10% EtOAc in hexanes) and MS showed the complete disappearance of the SM and only the product. The reaction mixture was poured into ice water and extracted with EtOAc (3×60 ml) and the organic layers were combined and washed with 2% sodium thiosulfate solution (60 ml), water (100 ml), brine (100 ml) and dried over Na₂SO₄. Concentration of the organic layer provided 5-benzyl-6-(1-bromo-2-methyl-propyl)-3-methyl-5H-isothiazolo[5,4-d]pyrimidin-4-one (7.27 g, 99%) as white crystalline solid. ¹H NMR (300 MHz) δ 0.54 (d, 3H), 1.11 (d, 3H), 2.62-2.76 (m, 1H), 2.83 (s, 3H), 4.42 (d, 1H), 4.80 (d, 1H), 6.22 (d, 1H), 7.12-7.42 (m, 5H).

Section 2: Method 17

### 6-(1-Azido-2-methyl-propyl)-5-benzyl-3-methyl-5H-isothiazolo[5,4-d]pyrimidin-4-one (alternative procedure)

To a solution of 5-benzyl-6-(1-bromo-2-methyl-propyl)-3-methyl-5H-isothiazolo[5,4-d]pyrimidin-4-one (Section 2: Method 16) (7.27 g, 18.5 mmol) in anhydrous DMF (60 ml), sodium azide (2.33 g, 37 mmol) was added and the mixture was stirred at room temperature for 2 hour. The TLC of the RM showed the complete disappearance of the starting bromide. The reaction mixture was poured into ice water (300 ml) and extracted with EtOAc (3×100 ml). The organic layer was washed with water (100 ml), brine (100 ml) and dried (Na₂SO₄). Concentration of the organic layer provided the crude product which was purified by column (silica gel) chromatography using 30% EtOAc in hexanes as eluent to isolate 6-(1-azido-2-methyl-propyl)-5-benzyl-3-methyl-5H-isothiazolo[5,4-d]pyrimidin-4-one (6.16 g, 94%) as a low melting solid. ¹H NMR (300 MHz) δ 0.57 (d, 3H), 1.07 (d, 3H), 2.50-2.74 (m, 1H), 2.98 (s, 3H), 3.71 (d, 1H), 5.05 (d, 1H), 5.78 (d, 1H), 7.12-7.40 (m, 5H).

Section 2: Method 18

### 6-(1-Amino-2-methyl-propyl)-5-benzyl-3-methyl-5H-isothiazolo[5,4-d]pyrimidin-4-one (alternative procedure)

To a solution of 6-(1-azido-2-methyl-propyl)-5-benzyl-3-methyl-5H-isothiazolo[5,4-d]pyrimidin-4-one (Section 2: Method 17) (6.8 g, 19.2 mmol) in methanol (400 ml) was added 5% Pd/C (1 g, 20% by wt.) and the resulting mixture was stirred at r.t. in an atmosphere of H₂ and the progress of the reaction was monitored by MS. After the disappearance of the starting material the reaction mixture was filtered through celite and washed with EtOAc. Concentration of the filtrate provided 6-(1-amino-2-methyl-propyl)-5-benzyl-3-methyl-5H-isothiazolo[5,4-d]pyrimidin-4-one (5.42 g, 86%).

Section 2: Method 40

### 5-Amino-3-methylisothiazole-4-carboxamide

To a chilled solution of sulfuric acid (7.2 volumes, 12.9 equivs) was charged 5-amino-3-methylisothiazole-4-carbonitrile (Section 2: Method 4) (1.0 equiv). The temperature was maintained below 55° C. The reaction mixture was heated to 70° C. and held for 1 hour until TLC showed disappearance of starting material. The mixture was cooled to 60-65° C. before the ammonia (21 volumes) was charged to pH 10. The mixture was cooled to 20° C., aged overnight and filtered. The resulting solid was washed with dilute ammonia (3.6 volumes) and dried at 40° C. to give a pale brown solid (typical yield 80%). ¹H NMR (300 MHz, DMSO-d₆) δ 2.46(s, 3H), 6.28 (s, 1H).

Section 2: Method 14

### 6-Isobutyl-3-methyl-5H-isothiazolo[5,4-d]pyrimidin-4-one (alternative procedure)

To a 2 L flask equipped with Dean Stark was charged 5-amino-3-methylisothiazole-4-carboxamide (Section 2: Method 41) (1 equiv), p-toluene sulphonic acid (0.049 equiv), DMF (9.75 volumes). The reaction was stirred until a solution was obtained and isovaleraldehyde (1.10 equiv) and toluene (4.9 volumes) were added. The resulting mixture was heated to 130° C. and held at reflux for 1 hour removing water via a Dean Stark apparatus. Once the reaction was complete toluene was removed under vacuum distillation. Sodium bisulfite (2.50 equiv) was charged and the mixture was held at 115° C. for 7 hours, then cooled to room temperature overnight. The solid was removed by filtration through harborlite and washed with DMF (1 volume). Analysis showed conversion to product and the reaction was heated to 50° C., water (15 volumes) was added and the resulting precipitate was cooled to room temperature and held for 1 h. The product was isolated by filtration and washed with water (2×0.5 volumes), dried to give a pale brown solid (typical yield 89%).

### EXAMPLE A

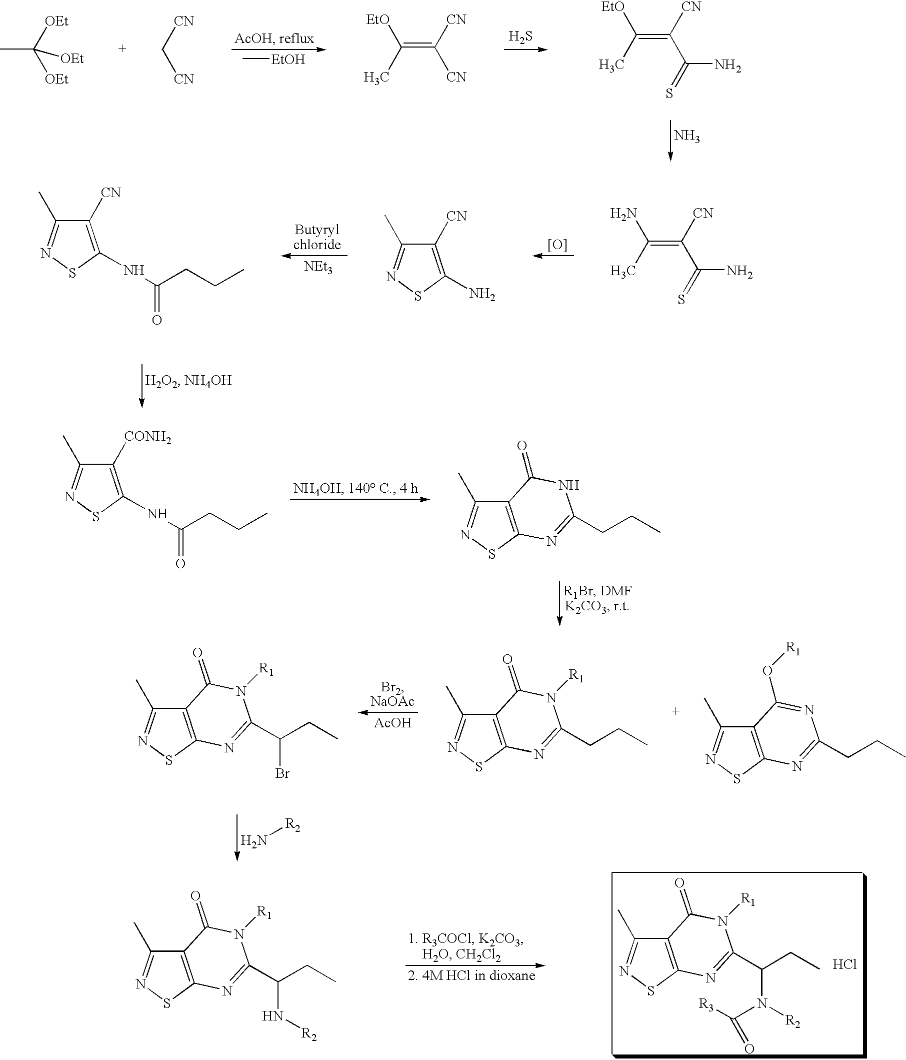

### SECTION 2: EXAMPLE A1

The following compound was synthesized according to synthetic scheme A above:

### SECTION 2: EXAMPLE A1

### N-(3-Amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide hydrogen chloride

{3-[{1-[5-(3-Fluoro-benzyl)-3-methyl-4-oxo-4,5-dihydro-isothiazolo[5,4-d]pyrimidin-6-yl]-propyl}-(4-methyl-benzoyl)-amino]-propyl}-carbamic acid tert-butyl ester (Section 2: Method 11) (0.030 g, 0.049 mmol) was dissolved in 4M HCl in 1,4-dioxane and the mixture was stirred at r.t. for 30 min and the LC/MS showed the complete disappearance of the starting material. The reaction mixture was concentrated in a rotary evaporator and the residue was triturated with ether. The precipitated product was filtered off and dried in vacuo to yield N-(3-amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide hydrogen chloride (0.0215 g, 80%). m/z 508 (MH⁺), ¹H NMR (DMSO-d₆, 90° C.) δ ppm 0.41-0.0.46 (t, 3H), 1.13-1.28 (m, 1H), 1.38-1.53 (m, 1H), 1.61-1.78 (m, 1H), 1.88-2.01 (m, 1H), 2.11 (s, 3H), 2.14-2.23 (m, 2H), 2.50 (s, 3H), 3.08-3.18 (m, 2H), 4.63 (br, 1H), 5.22 (br, 1H), 5.45-5.55 (d, 1H), 6.60-7.16 (m, 8H), 7.43-7.63 (br s, 3H).

| Ex. | Compound | ¹H NMR | m/z | SM |
|---|---|---|---|---|
| A1 | N-(3-Amino-propyl)-N- | (DMSO-d₆, 90° C.) δ ppm | m/z | Section |
| | {1-[5-(3-fluoro-benzyl)- | 0.41-0.46(t, 3H), 1.13-1.28(m, 1H), | 508(MH⁺) | 2: |
| | 3-methyl-4-oxo-4,5- | 1.38-1.53(m, 1H), 1.61-1.78(m, 1H), | | Method |
| | dihydro-isothiazolo[5,4- | 1.88-2.01(m, 1H), 2.11(s, 3H), | | 11 |
| | d]pyrimidin-6-yl]- | 2.14-2.23(m, 2H), ) 2.50(s, 3H), | | |
| | propyl}-4-methyl- | 3.08-3.18(m, 2H), 4.63(br, 1H), | | |
| | benzamide hydrogen | 5.22(br, 1H), 5.45-5.55(d, 1H), | | |
| | chloride | 6.60-7.16(m, 8H), 7.43-7.63(br s, 3H) | | |

### EXAMPLE B

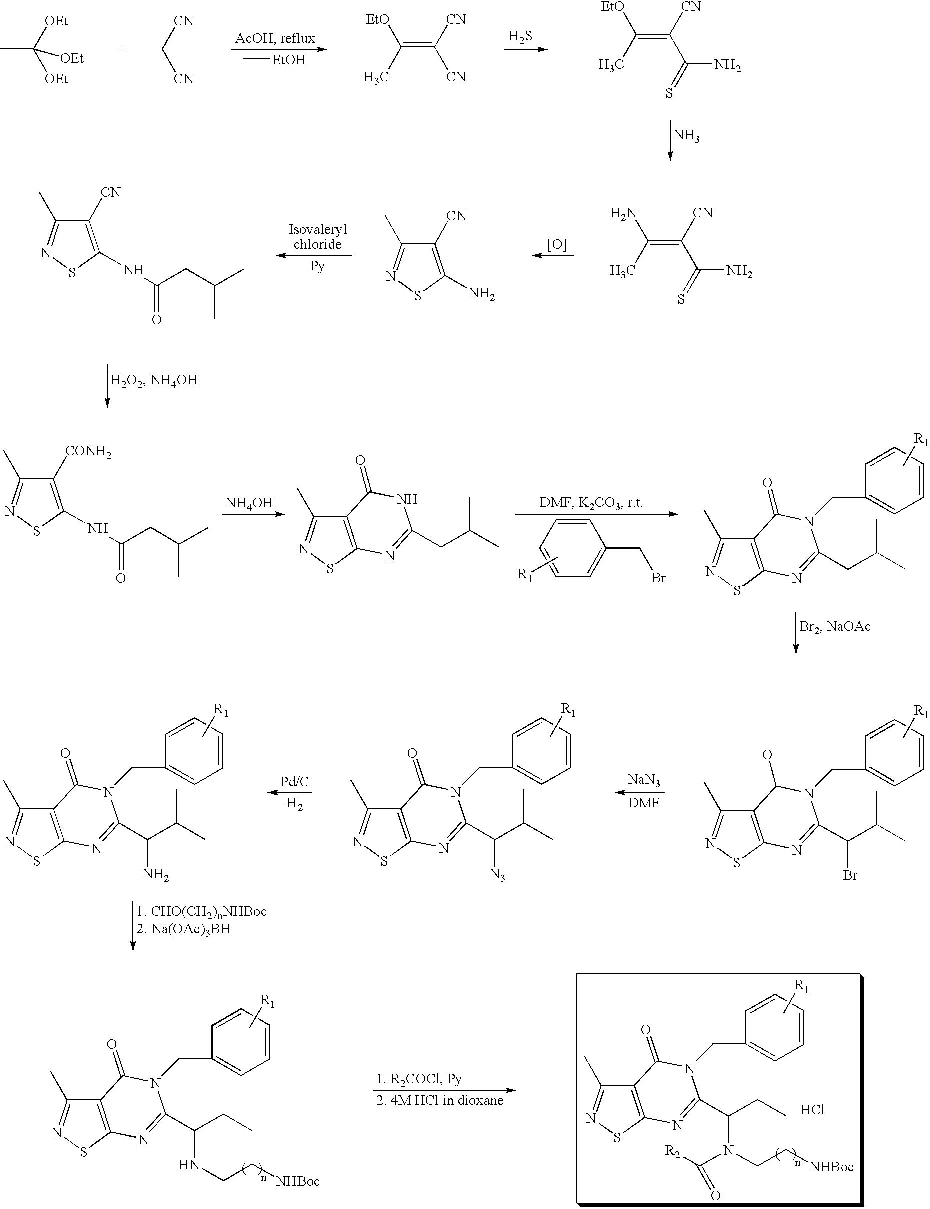

### SECTION 2: EXAMPLES B1-B6

The following compounds were synthesized according to synthetic scheme B above:

### SECTION 2: EXAMPLE B1

### N-(2-Amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide hydrogen chloride

{2-[[1-(5-Benzyl-3-methyl-4-oxo-4,5-dihydro-isothiazolo[5,4-d]pyrimidin-6-yl)-2-methyl-propyl]-(4-bromo-benzoyl)-amino]-ethyl}-carbamic acid tert-butyl ester (Section 2: Method 21b) (0.040 g, 0.061 mmol) was dissolved in 4M HCl in 1,4-dioxane and the mixture was stirred at r.t. for 30 min and the LC/MS showed the complete disappearance of the starting material. The reaction mixture was concentrated in a rotary evaporator and the residue was triturated with ether. The precipitated product was filtered off and dried in vacuo to yield N-(2-amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide hydrogen chloride (0.0345 g, 96%). m/z 554, 556 (MH⁺), ¹H NMR (DMSO-d₆, 90° C.) δ: 0.37-0.0.38 (d, 3H), 0.89-0.91 (d, 3H), 2.28-2.38 (m, 1H), 2.58-2.69 (m, 2H), 2.77 (s, 3H), 3.61-3.71 (m, 2H), 4.99 (br, 1H), 5.54 (br d, 1H), 5.89-5.93 (d, 1H), 7.20-7.70 (m, 9H), 7.84 (br, 3H).

The following compounds were prepared by the procedure of Section 2: Example B1.

| Ex. | Compound | ¹H NMR | m/z | SM |
|---|---|---|---|---|
| B1 | N-(2-Amino-ethyl)-N-[1- | (DMSO-d₆, 90° C.) δ: | m/z | Section |
| | (5-benzyl-3-methyl-4-oxo- | 0.37-0.0.38(d, 3H), 0.89-0.91(d, 3H), | 554, | 2: |
| | 4,5-dihydro-isothiazolo[5,4- | 2.28-2.38(m, 1H), 2.58-2.69(m, 2H), 2.77(s, 3H), | 556(MH⁺) | Method |
| | d]pyrimidin-6-yl)-2-methyl- | 3.61-3.71(m, 2H), 4.99(br, 1H), | | 21b |
| | propyl]-4-bromo- | 5.54(br d, 1H), 5.89-5.93(d, 1H), | | |
| | benzamide hydrogen | 7.20-7.70(m, 9H), 7.84(br, 3H) | | |
| | chloride | | | |
| B2 | N-(2-Amino-ethyl)-N-[1- | (DMSO-d₆, 90° C.) δ: | m/z | Section |
| | (5-benzyl-3-methyl-4-oxo- | 0.14-0.16(d, 3H), 0.66-0.68(d, 3H), | 508(MH⁺) | 2: |
| | 4,5-dihydro-isothiazolo[5,4- | 2.10-2.20(m and s, 4H), 2.40-2.50(m, 2H), | | Method |
| | d]pyrimidin-6-yl)-2-methyl- | 2.55(s, 3H), 3.40-3.50(m, 2H), | | 21c |
| | propyl]-3-fluoro-4-methyl- | 4.78(b, 1H), 5.30(b, 1H), 5.60-5.70(d, 1H), | | |
| | benzamide hydrogen | 6.90-7.23(m, 8H), 7.50-7.70(bs, 3H) | | |
| | chloride | | | |
| B3 | N-(3-Amino-propyl)-N-[1- | (DMSO-d₆, 90° C.) δ: | m/z | Section |
| | (5-benzyl-3-methyl-4-oxo- | 0.46-0.48(d, 3H), 0.90-0.92(d, 3H), | 522(MH⁺) | 2: |
| | 4,5-dihydro-isothiazolo[5,4- | 1.2-1.48(m, 1H), 1.51-1.72(m, 1H), | | Method |
| | d]pyrimidin-6-yl)-2-methyl- | 2.29-2.40(m, 5H), 2.70-2.77(m, s, 4H), | | 21d |
| | propyl]-3-fluoro-4-methyl- | 3.35-3.40(t, 2H), 5.00-5.10(d, 1H), | | |
| | benzamide hydrogen | 5.60-5.65(d, 1H), 5.90-5.94(d, 1H), | | |
| | chloride | 7.07-7.38(m, 8H), 7.71(b, 2H) | | |
| B4 | N-(3-Amino-propyl)-N-[1- | (DMSO-d₆, 90° C.) δ: | m/z | Section |
| | (5-benzyl-3-methyl-4-oxo- | 0.46-0.47(d, 3H), 0.90-0.92(m, 3H), | 568, | 2: |
| | 4,5-dihydro-isothiazolo[5,4- | 1.15-1.25(m, 1H), 1.45-1.60(m, 1H), | 570(MH⁺) | Method |
| | d]pyrimidin-6-yl)-2-methyl- | 2.29-2.33(t, 2H), 2.65-2.77(m, s, 4H), | | 21e |
| | propyl]-4-bromo- | 3.34-3.38(m, 2H), 5.05-5.10(d, 1H), | | |
| | benzamide hydrogen | 5.60-5.66(m, 1H), 5.90-5.94(d, 1H), | | |
| | chloride | 7.27-7.38(m, 7H), 7.55-7.66(br m, 4H) | | |

The following compounds may be prepared by the procedure of Section 2: Example B1.

| Ex. | Compound | SM |
|---|---|---|
| B5 | N-(3-Amino-propyl)-N-{1-[5-(3-fluoro-benzyl)-3-methyl-4- | Section |
| | oxo-4,5-dihydro-isothiazolo[5,4-d]pyrimidin-6-yl]-2- | 2: |
| | methyl-propyl}-4-methyl-benzamide hydrogen chloride | Method |
| | | 21a |
| B6 | N-(2-Amino-ethyl)-N-[1-(5-benzyl-3-methyl-4-oxo-4,5- | Section |
| | dihydro-isothiazolo[5,4-d]pyrimidin-6-yl)-2-methyl-propyl]- | 2: |
| | 4-methyl-benzamide hydrogen chloride | Method |
| | | 21 |

### EXAMPLE C

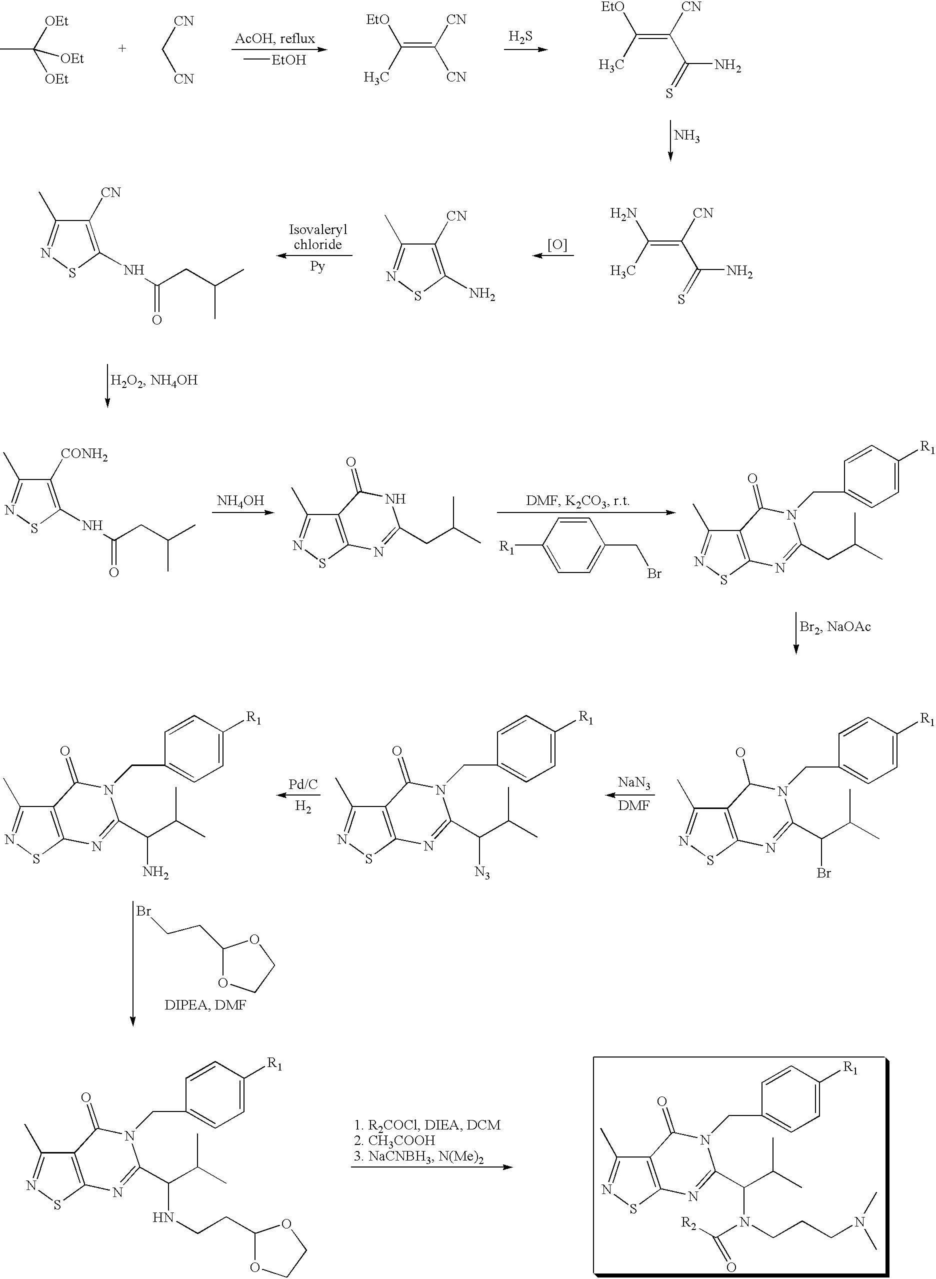

### SECTION 2: EXAMPLES C1-C3

The following compounds were synthesized according to synthetic scheme C above:

### SECTION 2: EXAMPLE C2

### N-[1-(5-Benzyl-3-methyl-4-oxo-4,5-dihydro-isothiazolo[5,4-d]pyrimidin-6-yl)-2-methyl-propyl]-4-bromo-N-(3-dimethylamino-propyl)-benzamide

To a solution of N-[1-(5-benzyl-3-methyl-4-oxo-4,5-dihydro-isothiazolo[5,4-d]pyrimidin-6-yl)-2-methyl-propyl]-4-bromo-N-(3-oxo-propyl)-benzamide (Section 2: Method 22) (1 g, 1.76 mmol) in methanol (20 mL) two drops of acetic acid were added followed by the addition of dimethylamine (1 mL, 2M solution in THF) and sodium cyanoborohydride (0.314 g, 5 mmol) and the mixture was stirred at room temperature for 3 h. The reaction mixture was concentrated and the residue was dissolved in DCM (100 mL) and the organic layer was washed with satd. NaHCO₃ (3×100 mL). The organic layer was concentrated and the crude product was purified by column chromatography using 0-10% MeOH in EtOAc. The pure product fractions were concentrated and the thus obtained foam was crystallized from ether/hexanes to get the product as white crystalline solid. Yield=0.366 g (35%). Having the following properties m/z 596, 598 (MH⁺); ¹H-NMR (300 MHz, 25° C.) δ 0.31-0.36 (d, 3H), 0.67-0.77 (m, 1H), 0.89-0.94 (d, 3H), 1.19-1.27 (m, 1H), 1.65-1.83 (m, s, 8H), 2.66-2.76 (m, 1H), 2.89 (s, 3H), 3.30-3.40 (m, 2H), 5.17-5.23 (d, 1H), 5.71-5.75 (d, 1H), 6.12-6.17 (d, 1H), 7.28-7.41 (d, m, 7H), 7.55-7.58 (d, 2H).

The following compounds were synthesised according to Section 2: Example C2 above.

| Ex. | Compound | ¹H NMR | m/z | SM |
|---|---|---|---|---|
| C1 | N-[1-(5-Benzyl-3-methyl-4- | (300 MHz) δ: 0.34-0.36(d, 3H), | m/z | Section |
| | oxo-4,5-dihydro- | 0.68-0.75(m, 1H), 0.93-0.96(d, 3H), | 532(MH⁺) | 2: |
| | isothiazolo[5,4- | 1.22-1.30(m, 1H), 1.65-1.87(br m, s, s, 8H), | | Method |
| | d]pyrimidin-6-yl)-2-methyl- | 2.37(s, 3H), 2.66-2.72(m, 1H), | | 22a |
| | propyl]-N-(3- | 2.87(s, 3H), 3.35-3.41(m, 2H), | | |
| | dimethylamino-propyl)-4- | 5.22-5.27(d, 1H), 5.73-5.76(d, 1H), | | |
| | methyl-benzamide | 6.12-6.17(d, 1H), 7.22-7.41(m, 9H) | | |
| C2 | N-[1-(5-Benzyl-3-methyl-4- | (300 MHz, 25° C.) δ 0.31-0.36(d, 3H), | m/z | Section |
| | oxo-4,5-dihydro- | 0.67-0.77(m, 1H), 0.89-0.94(d, 3H), | 597(MH⁺) | 2: |
| | isothiazolo[5,4- | 1.19-1.27(m, 1H), 1.65-1.83(m, s, 8H), | | Method |
| | d]pyrimidin-6-yl)-2-methyl- | 2.66-2.76(m, 1H), | | 22 |
| | propyl]-N-(3- | 2.89(s, 3H), 3.30-3.40(m, 2H), 5.17-5.23(d, 1H), | | |
| | dimethylamino-propyl)-4- | 5.71-5.75(d, 1H), 6.12-6.17(d, 1H), | | |
| | bromo-benzamide | 7.28-7.41(d, m, 7H), 7.55-7.58(d, 2H) | | |
| C3 | N-[1-(5-Benzyl-3-methyl-4- | (300 MHz, 25° C.) δ: 0.35-0.40(d, 3H), | m/z | Section |
| | oxo-4,5-dihydro- | 0.68-0.78(m, 1H), 0.92-0.94(d, 3H), | 540(MH⁺) | 2: |
| | isothiazolo[5,4- | 1.20-1.30(m, 1H), 1.65-1.83(br m, s, s, 8H), | | Method |
| | d]pyrimidin-6-yl)-2-methyl- | 2.30(s, 3H), 2.67-2.75(m, 1H), | | 22b |
| | propyl]-N-(3- | 2.87(s, 3H), 3.35-3.44(t, 2H), | | |
| | dimethylamino-propyl)-3- | 5.17-5.23(d, 1H), 5.71-5.74(d, 1H), | | |
| | fluoro-4-methyl-benzamide | 6.11-6.16(d, 1H), 6.99-7.39(m, 8H) | | |

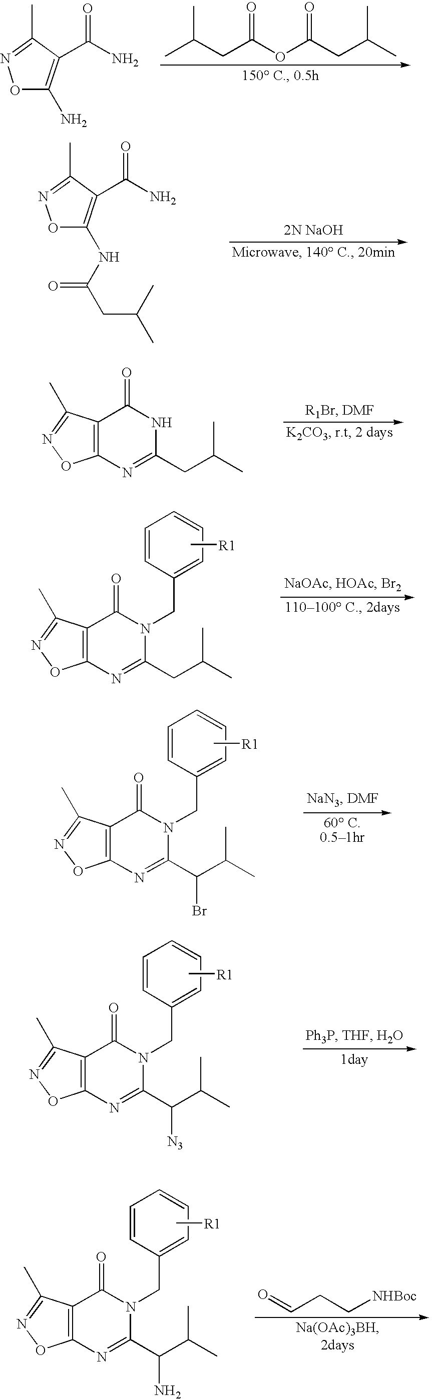

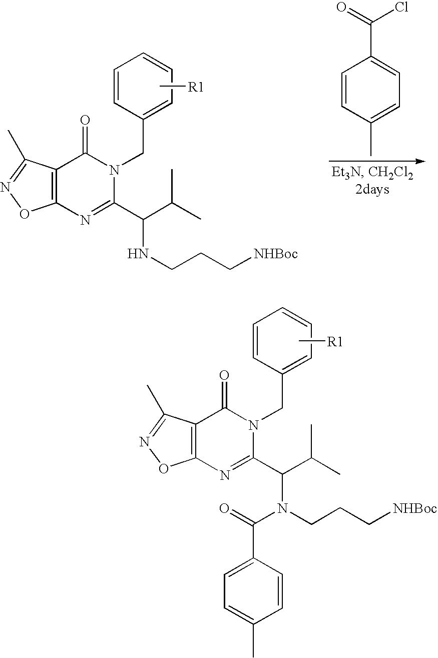

### SECTION 2: EXAMPLE D

The following compound may be synthesized according to synthetic scheme D above:

### SECTION 2: EXAMPLE D1

### N-(3-Amino-propyl)-N-{1-[5-(3-fluoro-benzyl)-3-methyl-4-oxo-4,5-dihydro-isoxazolo[5,4-d]pyrimidin-6-yl-2-methyl-propyl}-4-methyl-benzamide hydrogen chloride

A solution of {3-[{1-[5-(3-fluoro-benzyl)-3-methyl-4-oxo-4,5-dihydro-isoxazolo[5,4-d]pyrimidin-6-yl]-2-methyl-propyl}-(4-methyl-benzoyl)-amino]-propyl}-carbamic acid tert-butyl ester (Section 2: Method 30) (100 mg, 0.165 mmol) in 5 ml of 4 M HCl in dioxane could be stirred at room temperature for 2 hr. The solvent could be distilled off by vacuo and the residue dried at 40-50° C. overnight under vacuum to give N-(3-amino-propyl)-N-{1-[5-(3-fluoro-benzyl)-3-methyl-4-oxo-4,5-dihydro-isoxazolo[5,4-d]pyrimidin-6-yl]-2-methyl-propyl}-4-methyl-benzamide as the HCl salt.

| Ex. | Compound | SM |
|---|---|---|
| D1 | N-(3-Amino-propyl)-N-{1-[5-(3-fluoro-benzyl)-3-methyl-4- | Section |
| | oxo-4,5-dihydro-isoxazolo [5,4-d]pyrimidin-6-yl]- | 2: |
| | 2-methyl-propyl}-4-methyl-benzamide | Method |
| | hydrogen chloride | 30 |

### EXAMPLE E

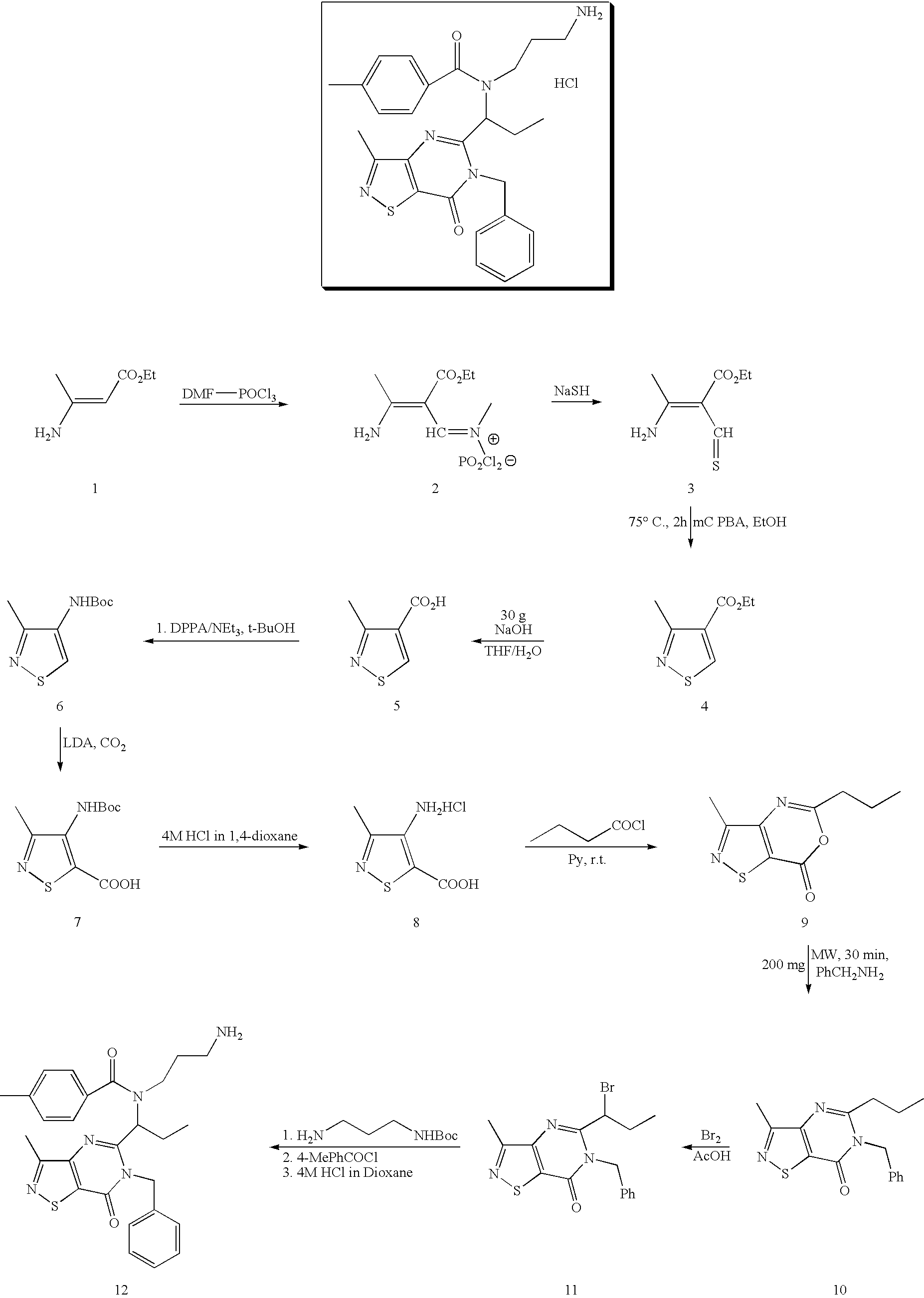

### SECTION 2: EXAMPLE E

The following compound was synthesized according to synthetic scheme E above:

### SECTION 2: EXAMPLE E1

### N-(3-Amino-propyl)-N-[1-(6-benzyl-3-methyl-7-oxo-6,7-dihydro-isothiazolo[4,5-d]pyrimidin-5-yl)-propyl]-4-methyl-benzamide hydrogen chloride

To a solution of 6-benzyl-5-(1-bromo-propyl)-3-methyl-6H-isothiazolo[4,5-d]pyrimidin-7-one (Section 2: Method 39) (260 mg, 0.70 mmol) in anhydrous DMF (10 mL), ethyl diisopropylamine (387 mg, 3 mmol) and N-(3-aminopropyl)carbamic acid tert-butyl ester (174 mg, 1 mmol) were added at room temperature and the mixture was stirred at room temperature for 1 h after which the MS analysis showed the complete disappearance of the starting bromide and only the product peak at 472 (MH⁺) was observed. The reaction mixture was diluted with water (100 mL) and extracted with EtOAc (3×60 mL). The combined organic extracts were dried and concentrated to get the crude amine, which was dissolved, in chloroform (40 mL) and diisopropylethylamine (387 mg, 3 mmol) was added and the mixture was heated to 60° C. To the stirred hot solution p-toluoyl chloride (154 mg, 1 mmol) in chloroform (20 mL) was added dropwise and the mixture was refluxed for 12 h after which the MS showed the complete disappearance of the amine and only the product peak at 590 (MH⁺). The reaction mixture was concentrated and the crude product was purified by column chromatography to isolate the pure acylated product (80 mg, 20% overall from bromide), which was treated with 4M HCl in 1,4-dioxane (10 mL) for 30 min. The dioxane was evaporated in a rotary evaporator and the residue was dissolved in water and freeze dried to get the pure product as a white fluffy solid. Yield 60 mg (16% overall from bromide). Having the following properties: m/z 490 (MH⁺); ¹H NMR (300 MHz, DMSO-d₆, 96° C.) δ 0.65 (t, 3H), 1.36-1.50 (m, 1H), 1.60-1.72 (m, 1H), 1.88-1.99 (m, 1H), 2.14-2.26 (m, 1H), 2.35 (s, 3H), 2.47 (t, 2H), 2.68 (s, 3H), 3.32-3.44 (m, 2H), 4.90 (d, 1H), 5.50 (bs, 1H), 5.76 (d, 1H), 6.96-7.34 (m, 9H), 7.68 (bs, 3H).

| Ex. | Compound | ¹H NMR | m/z | SM |
|---|---|---|---|---|
| E1 | N-(3-Amino-propyl)-N- | (300MHz, DMSO-d₆, 96° C.) δ 0.65(t, | m/z | Section |
| | [1-(6-benzyl-3-methyl- | 3H), 1.36-1.50(m, 1H), 1.60-1.72(m, | 490 | 2: |
| | 7-oxo-6,7-dihydro- | 1H), 1.88-1.99(m, 1H), 2.14-2.26(m, | (MH⁺) | Method |
| | isothiazolo[4,5- | 1H), 2.35(s, 3H), 2.47(t, 2H), 2.68(s, | | 39 |
| | d]pyrimidin-5-yl)- | 3H), 3.32-3.44(m, 2H), 4.90(d, 1H), | | |
| | propyl]-4-methyl- | 5.50(bs, 1H), 5.76(d, 1H), 6.96-7.34 (m, | | |
| | benzamide | 9H), 7.68(bs, 3H). | | |

In section 2, compounds of formula (I) have been shown to inhibit the microtubule motor protein HsEg5 in vitro. Inhibitors of Eg5 have been shown to inhibit the formation of a mitotic spindle and therefore for cell division. Inhibitors of Eg5 have been shown to block cells in the metaphase of mitosis leading to apoptosis of effected cells, and to therefore have anti-proliferative effects. It is believed that Eg5 inhibitors act as modulators of cell division and are expected to be active against neoplastic disease such as carcinomas of the brain, breast, ovary, lung, colon, prostate or other tissues, as well as multiple myeloma leukemias, for example myeloid leukemia, acute lymphoblastic leukemia, chronic myeloid leukemia, chronic lymphocytic leukemia, and lymphomas for example Hodgkins disease and non-Hodgkins lymphoma, tumors of the central and peripheral nervous system, and other tumor types such as melanoma, fibrosarcoma, Ewing's sarcoma and osteosarcoma. Therefore it is believed that the compounds of formula (I) in section 2 may be used for the treatment of neoplastic disease. Hence the compounds of formula (I) and their salts and their in vivo hydrolysable esters in section 2 are expected to be active against carcinomas of the brain, breast, ovary, lung, colon, prostate or other tissues, as well as leukemias and lymphomas, tumors of the central and peripheral nervous system, and other tumor types such as melanoma, fibrosarcoma and osteosarcoma. In section 2, the compounds of formula (I) and their salts and their in vivo hydrolysable esters are expected to be active against neoplastic disease such as carcinomas of the brain, breast, ovary, lung, colon, prostate or other tissues, as well as multiple myeloma leukemias, for example myeloid leukemia, acute lymphoblastic leukemia, chronic myeloid leukemia, chronic lymphocytic leukemia, and lymphomas for example Hodgkins disease and non-Hodgkins lymphoma, tumors of the central and peripheral nervous system, and other tumor types such as melanoma, fibrosarcoma, Ewing's sarcoma and osteosarcoma. It is expected that the compounds of formula (I) of sction 2 would most likely be used in combination with a broad range of agents but could also be used as a single agent.

In section 2, generally, the compounds of formula (I) have been identified in the Malachite Green Assay described herein as having an IC₅₀ value of 100 micromolar or less. For example compound of E1 has an IC₅₀ value of 90 nM.

In section 2, compounds provided by this invention should also be useful as standards and reagents in determining the ability of a potential pharmaceutical to inhibit Eg5. These would be provided in commercial kits comprising a compound of this invention.

Section 2: Malachite Green Assay

Enzymatic activity of the Eg5 motor and effects of inhibitors was measured using a malachite green assay, which measures phosphate liberated from ATP, and has been used previously to measure the activity of kinesin motors (Hackney and Jiang, 2001). Enzyme was recombinant HsEg5 motor domain (amino acids 1-369-8His) and was added at a final concentration of 6 nM to 100 μl reactions. Buffer consisted of 25 mM PIPES/KOH, pH 6.8, 2 mM MgCl₂, 1 mM EGTA, 1 mM dtt, 0.01% Triton X-100 and 5 μM paclitaxel. Malachite green/ammonium molybdate reagent was prepared as follows: for 800 ml final volume, 0.27 g of Malachite Green (J. T. Baker) was dissolved in 600 ml of H₂O in a polypropylene bottle. 8.4 g ammonium molybdate (Sigma) was dissolved in 200 ml 4N HCl. The solutions were mixed for 20 min and filtered through 0.02 μm filter directly into a polypropylene container. 5 μl of compound diluted in 12% DMSO was added to the wells of 96 well plates. 80 μl of enzyme diluted in buffer solution above was added per well and incubated with compound for 20 min. After this pre-incubation, substrate solution containing 2 mM ATP (final concentration: 300 μM) and 6.053 μM polymerized tubulin (final concentration: 908 nM) in 15 μl of buffer were then added to each well to start reaction. Reaction was mixed and incubated for an additional 20 min at room temperature. The reactions were then quenched by the addition of 150 μl malachite green/ammonium molybdate reagent, and absorbance read at 650 nanometers exactly 5 min after quench using a Spectramax Plus plate reader (Molecular Devices). Data was graphed and IC₅₀s calculated using ExCel Fit (Microsoft).

### Section 3

Section 3: Field of the Invention

In section 3, the invention relates to novel fused heterocycles, their pharmaceutical compositions and methods of use. In addition in section 3, the present invention relates to therapeutic methods for the treatment and prevention of cancers and to the use of these chemical compounds in the manufacture of a medicament for use in the treatment and prevention of cancers.

Section 3: Background of the Invention

One sub-class of anti-cancer drugs (taxanes, vinca-alkaloids) now used extensively in the clinic is directed at microtubules and block the cell division cycle by interfering with normal assembly or disassembly of the mitotic spindle (see Chabner, B. A., Ryan, D. P., Paz-Ares, I., Garcia-Carbonero; R., and Calabresi, P: Antineoplastic agents. In Hardman, J. G., Limbird, L. E., and Gilman, A. G., eds. Goodman and Gilman's The Pharmacological Basis of Therapeutics, 10^{th} edition, 2001, The MacGraw-Hill Companies, Inc). Taxol® (paclitaxel), one of the most effective drugs of this class, is a microtubule stabilizer. It interferes with the normal growth and shrinkage of microtubules thus blocking cells in the metaphase of mitosis. Mitotic block is often followed by slippage into the next cell cycle without having properly divided, and eventually by apoptosis of these abnormal cells (Blagosklonny, M. V. and Fojo, T.: Molecular effects of paclitaxel: myths and reality (a critical review). Int J Cancer 1999, 83:151-156.).

Some of the side effects of treatment with paclitaxel are neutropenia and peripheral neuropathy. Paclitaxel is known to cause abnormal bundling of microtubules in interphase cells. In addition, some tumor types are refractory to treatment with paclitaxel, and other tumors become insensitive during treatment. Paclitaxel is also a substrate for the multi-drug resistance pump, P-glycoprotein ((see Chabner et al., 2001).

Thus, there is a need for effective anti-mitotic agents that have fewer side effects than anti-microtubule drugs, and also for agents that are effective against taxane-resistant tumors.

Kinesins are a large family of molecular motor proteins, which use the energy of adenosine 5′-triphosphate (ATP) hydrolysis to move in a stepwise manner along microtubules. For a review, see Sablin, E. P.: Kinesins and microtubules: their structures and motor mechanisms. Curr Opin Cell Biol 2000, 12:35-41 and Schief, W. R. and Howard, J.: Conformational changes during kinesin motility. Curr Opin Cell Biol 2001, 13:19-28.

Some members of this family transport molecular cargo along microtubules to the sites in the cell where they are needed. For example, some kinesins bind to vesicles and transport them along microtubules in axons. Several family members are mitotic kinesins, as they play roles in the reorganization of microtubules that establishes a bipolar mitotic spindle. The minus ends of the microtubules originate at the centrosomes, or spindle poles, whilst the plus ends bind to the kinetochore at the centromeric region of each chromosome. The mitotic spindle lines up the chromosomes at metaphase of mitosis and coordinates their movement apart and into individual daughter cells at anaphase and telophase (cytokinesis). See Alberts, B., Bray, D., Lewis, J., Raff, M., Roberts, K., and Watson, J. D., Molecular Biology of the Cell, 3^{rd} edition, Chapter 18, The Mechanics of Cell Division, 1994, Garland Publishing, Inc. New York.

HsEg5 (homo sapiens Eg5) (Accession X85137; see Blangy, A., Lane H. A., d'Heron, P., Harper, M., Kress, M. and Nigg, E. A.: Phosphorylation by p34cdc2 regulates spindle association of human Eg5, a kinesin-related motor essential for bipolar spindle formation in vivo. Cell 1995, 83(7): 1159-1169) or, KSP (kinesin spindle protein), is a mitotic kinesin whose homologs in many organisms have been shown to be required for centrosome separation in the prophase of mitosis, and for the assembly of a bipolar mitotic spindle. For a review see Kashina, A. S., Rogers, G. C., and Scholey, J. M.: The bimC family of kinesins: essential bipolar mitotic motors driving centrosome separation. Biochem Biophys Acta 1997, 1357: 257-271. Eg5 forms a tetrameric motor, and it is thought to cross-link microtubules and participate in their bundling (Walczak, C. E., Vernos, I., Mitchison, T. J., Karsenti, E., and Heald, R.: A model for the proposed roles of different microtubule-based motor proteins in establishing spindle bipolarity. Curr Biol 1998, 8:903-913). Several reports have indicated that inhibition of Eg5 function leads to metaphase block in which cells display monastral spindles. Recently an Eg5 inhibitor called monastrol was isolated in a cell-based screen for mitotic blockers (Mayer, T. U., Kapoor, T. M., Haggarty, S. J., King, R. W., Schreiber, S. L., and Mitchison, T. J.: Small molecule inhibitor of mitotic spindle bipolarity identified in a phenotype-based screen. Science 1999, 286: 971-974).

Monastrol treatment was shown to be specific for Eg5 over kinesin heavy chain, another closely related motor with different functions (Mayer et al., 1999). Monastrol blocks the release of ADP (adenosine 5′-diphosphate) from the Eg5 motor (Maliga, Z., Kapoor, T. M., and Mitchison, T. J.: Evidence that monastrol is an allosteric inhibitor of the mitotic kinesin Eg5. Chem & Biol 2002, 9: 989-996 and DeBonis, S., Simorre, J.-P., Crevel, I., Lebeau, L, Skoufias, D. A., Blangy, A., Ebel, C., Gans, P., Cross, R., Hackney, D. D., Wade, R. H., and Kozielski, F.: Interaction of the mitotic inhibitor monastrol with human kinesin Eg5. Biochemistry 2003, 42: 338-349) an important step in the catalytic cycle of kinesin motor proteins (for review, see Sablin, 2000; Schief and Howard, 2001). Treatment with monastrol was shown to be reversible and to activate the mitotic spindle checkpoint which stops the progress of the cell division cycle until all the DNA is in place for appropriate division to occur (Kapoor, T. M., Mayer, T. U., Coughlin, M. L., and Mitchison, T. J.: Probing spindle assembly mechanisms with monastrol, a small molecule inhibitor of the mitotic kinesin, Eg5. J Cell Biol 2000, 150(5): 975-988). Recent reports also indicate that inhibitors of Eg5 lead to apoptosis of treated cells and are effective against several tumor cell lines and tumor models (Mayer et al., 1999).

Although Eg5 is thought to be necessary for mitosis in all cells, one report indicates that it is over-expressed in tumor cells (International Patent Application WO 01/31335), suggesting that they may be particularly sensitive to its inhibition. Eg5 is not present on the microtubules of interphase cells, and is targeted to microtubules by phosphorylation at an early point in mitosis (Blangy et al., 1995). See also; Sawin, K. E. and Mitchison, T. J.: Mutations in the kinesin-like protein Eg5 disrupting localization to the mitotic spindle. Proc Natl Acad Sci USA 1995, 92(10): 4289-4293, thus monastrol has no detectable effect on microtubule arrays in interphase cells (Mayer et al., 1999). Another report suggests that Eg5 is involved in neuronal development in the mouse, but it disappears from neurons soon after birth, and thus Eg5 inhibition may not produce the peripheral neuropathy associated with treatment with paclitaxel and other anti-microtubule drugs (Ferhat, L., Expression of the mitotic motor protein Eg5 in postmitotic neurons: implications for neuronal development. J Neurosci 1998, 18(19): 7822-7835). Herein we describe the isolation of a class of specific and potent inhibitors of Eg5, expected to be useful in the treatment of neoplastic disease.

Certain pyrimidones have recently been described as being inhibitors of KSP (WO 03/094839, WO 03/099211, WO 03/050122, WO 03/050064, WO 03/049679, WO 03/049527, WO 04/078758, WO 04/106492 and WO 04/111058).

In accordance with the present invention of section 3, the present inventors have discovered novel chemical compounds which possess Eg5 inhibitory activity and are accordingly useful for their anti-cell-proliferation (such as anti-cancer) activity and are therefore useful in methods of treatment of the human or animal body.

Section 3: Summary of the Invention

An enantiomer of a compound of formula (I): 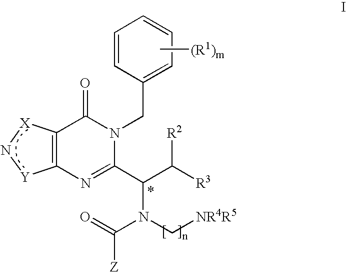
including a pharmaceutically acceptable salt or an in vivo hydrolysable ester thereof, wherein:
X is selected from —C(CH₃)— or —S— provided that when X is —S— then Y is —C(CH₃)—;
Y is selected from —C(CH₃)— or —O— or —S— provided that when Y is —C(CH₃)— then X is not —C(CH₃)—;
m is or 1;
R¹ is F when m is 1;
R² and R³ are independently selected from H or C₁₋₃alkyl; wherein if both R² and R³ are selected from C₁₋₃alkyl they are identical;
n is 2 or 3;
R⁴ and R⁵ are independently selected from H or C₁₋₃alkyl;
Z is optionally substituted phenyl, or optionally substituted benzothiophene wherein the number of optional substituents is 1 or 2 and each is independently selected from F, Cl, Br, CH₃ or CH₂CH₃; and

“*” represents a chiral center;

wherein said enantiomer is substantially free of the other enantiomer; and wherein the optical rotation of the enantiomer, when said enantiomer is dissolved at a concentration of 1 mg/ml in methanol, at 20.0° C. measured at 589 nM is (+).

In section 3, the invention encompasses stereoisomers, enantiomers, in vivo-hydrolysable precursors and pharmaceutically-acceptable salts of compounds of formula I, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

Section 3: Detailed Description of the Invention

In a first embodiment of section 3, the present invention provides an enantiomer of a novel compound having structural formula (I): 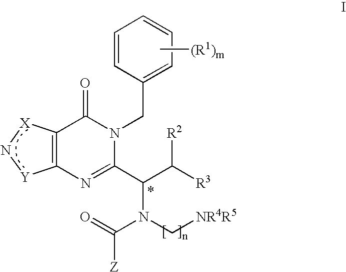
including a pharmaceutically acceptable salt or an in vivo hydrolysable ester thereof, wherein:
X is selected from —C(CH₃)— or —S— provided that when X is —S— then Y is —C(CH₃)—;
Y is selected from —C(CH₃)— or —O— or —S— provided that when Y is —C(CH₃)— then X is not —C(CH₃)—;
m is 0 or 1;
R¹ is F when m is 1;
R² and R³ are independently selected from H or C₁₋₃alkyl; wherein if both R² and R³ are selected from C₁₋₃alkyl they are identical;
n is 2 or 3;
R⁴ and R⁵ are independently selected from H or C₁₋₃alkyl;
Z is optionally substituted phenyl, or optionally substituted benzothiophene wherein the number of optional substituents is 1 or 2 and each is independently selected from F, Cl, Br, CH₃ or CH₂CH₃; and
“*” represents a chiral center;
   wherein said enantiomer is substantially free of the other enantiomer; and wherein the optical rotation of the enantiomer, when said enantiomer is dissolved at a concentration of 1 mg/ml in methanol, at 20.0° C. measured at 589 nM is (+).

In a further aspect of section 3 of the invention there is provided a compound of formula (I) having an optical rotation of (+) 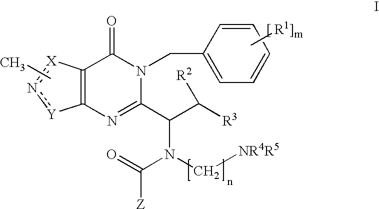
including a pharmaceutically acceptable salt or an in vivo hydrolysable ester thereof, wherein:
X is selected from C or S provided that when X is S then Y is C;
Y is selected from C or O or S provided that when Y is C then X is not C;
m is 0 or 1;
R¹ is F when m is 1;
R² and R³ are independently selected from H or C₁₋₃alkyl;
n is 2 or 3;
R⁴ and R⁵ are independently selected from H or C₁₋₃alkyl;
Z is optionally substituted phenyl, or optionally substituted benzothiophene wherein the number of substituents is 1 or 2 and each is independently selected from F, Cl, Br, CH₃ or CH₂CH₃.

In another embodiment of section 3, the present invention provides an (R) enantiomer of formula (Ia): 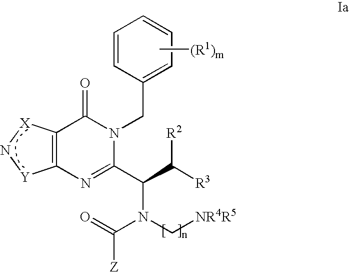
including a pharmaceutically acceptable salt or an in vivo hydrolysable ester thereof, wherein:
X is selected from —C(CH₃)— or —S— provided that when X is —S— then Y is —C(CH₃)—;
Y is selected from —C(CH₃)— or —O— or —S— provided that when Y is —C(CH₃)— then X is not —C(CH₃)—;
m is or 1;
R¹ is F when m is 1;
R² and R³ are independently selected from H or C₁₋₃alkyl; wherein if both R² and R³ are selected from C₁₋₃alkyl they are identical;
n is 2 or 3;
R⁴ and R⁵ are independently selected from H or C₁₋₃alkyl;
Z is optionally substituted phenyl, or optionally substituted benzothiophene wherein the number of optional substituents is 1 or 2 and each is independently selected from F, Cl, Br, CH₃ or CH₂CH₃;
   wherein said enantiomer is substantially free of the (S) enantiomer.

In another embodiment of section 3, the present invention provides an (S) enantiomer of formula (Ib): 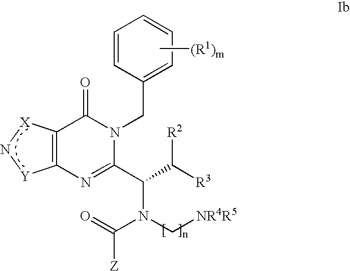
including a pharmaceutically acceptable salt or an in vivo hydrolysable ester thereof, wherein:
X is selected from —C(CH₃)— or —S— provided that when X is —S— then Y is —C(CH₃)—;
Y is selected from —C(CH₃)— or —O— or —S— provided that when Y is —C(CH₃)— then X is not —C(CH₃)—;
m is 0 or 1;
R¹ is F when m is 1;
R² and R³ are independently selected from H or C₁₋₃alkyl; wherein if both R¹ and R³ are selected from C₁₋₃alkyl they are identical;
n is 2 or 3;
R⁴ and R⁵ are independently selected from H or C₁₋₃alkyl;
Z is optionally substituted phenyl, or optionally substituted benzothiophene wherein the number of optional substituents is 1 or 2 and each is independently selected from F, Cl, Br, CH₃ or CH₂CH₃.
   wherein said enantiomer is substantially free of the (R) enantiomer.

In section 3, in formula (I) the dotted line represents a single or a double bond—the bond between the nitrogen and whichever of X and Y is C is double, the other bond is a single bond.

In an additional embodiment of section 3, the present invention provides an enantiomer of a compound of formula (I) wherein X is —C(CH₃)— or a pharmaceutically acceptable salt or an in vivo hydrolysable ester thereof.

In an additional embodiment of section 3, the present invention provides an enantiomer of a compound of formula (I) wherein X is —S— or a pharmaceutically acceptable salt or an in vivo hydrolysable ester thereof.

In an additional embodiment of section 3, the present invention provides an enantiomer of a compound of formula (I) wherein Y is —C(CH₃)— or a pharmaceutically acceptable salt or an in vivo hydrolysable ester thereof.

In an additional embodiment of section 3, the present invention provides an enantiomer of a compound of formula (I) wherein Y is —S— or a pharmaceutically acceptable salt or an in vivo hydrolysable ester thereof.

In an additional embodiment of section 3, the present invention provides an enantiomer of a compound of formula (I) wherein Y is —O— or a pharmaceutically acceptable salt or an in vivo hydrolysable ester thereof.

In an additional embodiment of section 3, the present invention provides an enantiomer of a compound of formula (I) wherein Y is —S— and X is —C(CH₃)— or a pharmaceutically acceptable salt or an in vivo hydrolysable ester thereof.

In an additional embodiment of section 3, the present invention provides an enantiomer of a compound of formula (I) wherein Y is —O— and X is —C(CH₃)— or a pharmaceutically acceptable salt or an in vivo hydrolysable ester thereof.

In an additional embodiment of section 3, the present invention provides an enantiomer of a compound of formula (I) wherein Y is —C(CH₃)— and X is —S— or a pharmaceutically acceptable salt or an in vivo hydrolysable ester thereof.

In an additional embodiment of section 3, the present invention provides an enantiomer of a compound of formula (I) wherein m is 0 or a pharmaceutically acceptable salt or an in vivo hydrolysable ester thereof.

In an additional embodiment of section 3, the present invention provides an enantiomer of a compound of formula (I) wherein m is 1 or a pharmaceutically acceptable salt or an in vivo hydrolysable ester thereof.

In an additional embodiment of section 3, the present invention provides an enantiomer of a compound of formula (I) wherein R² is H or a pharmaceutically acceptable salt or an in vivo hydrolysable ester thereof.

In an additional embodiment of section 3, the present invention provides an enantiomer of a compound of formula (I) wherein R² is methyl or a pharmaceutically acceptable salt or an in vivo hydrolysable ester thereof.

In an additional embodiment of section 3, the present invention provides an enantiomer of a compound of formula (I) wherein R² is ethyl or a pharmaceutically acceptable salt or an in vivo hydrolysable ester thereof.

In an additional embodiment of section 3, the present invention provides an enantiomer of a compound of formula (I) wherein R² is propyl or a pharmaceutically acceptable salt or an in vivo hydrolysable ester thereof.

In an additional embodiment of section 3, the present invention provides an enantiomer of a compound of formula (I) wherein R² is isopropyl or a pharmaceutically acceptable salt or an in vivo hydrolysable ester thereof.

In an additional embodiment of section 3, the present invention provides an enantiomer of a compound of formula (I) wherein R³ is methyl or a pharmaceutically acceptable salt or an in vivo hydrolysable ester thereof.

In an additional embodiment of section 3, the present invention provides an enantiomer of a compound of formula (I) wherein R³ is ethyl or a pharmaceutically acceptable salt or an in vivo hydrolysable ester thereof.

In an additional embodiment of section 3, the present invention provides an enantiomer of a compound of formula (I) wherein R³ is propyl or a pharmaceutically acceptable salt or an in vivo hydrolysable ester thereof.

In an additional embodiment of section 3, the present invention provides an enantiomer of a compound of formula (I) wherein R³ is isopropyl or a pharmaceutically acceptable salt or an in vivo hydrolysable ester thereof.

In an additional embodiment of section 3, the present invention provides an enantiomer of a compound of formula (I) wherein R² is H and R³ is methyl or a pharmaceutically acceptable salt or an in vivo hydrolysable ester thereof.

In an additional embodiment of section 3, the present invention provides an enantiomer of a compound of formula (I) wherein R² and R³ are methyl or a pharmaceutically acceptable salt or an in vivo hydrolysable ester thereof.

In an additional embodiment of section 3, the present invention provides an enantiomer of a compound of formula (I) wherein n is 2 or a pharmaceutically acceptable salt or an in vivo hydrolysable ester thereof.

In an additional embodiment of section 3, the present invention provides an enantiomer of a compound of formula (I) wherein n is 3 or a pharmaceutically acceptable salt or an in vivo hydrolysable ester thereof.

In an additional embodiment of section 3, the present invention provides an enantiomer of a compound of formula (I) wherein R³ is H or a pharmaceutically acceptable salt or an in vivo hydrolysable ester thereof.

In an additional embodiment of section 3, the present invention provides an enantiomer of a compound of formula (I) wherein R⁴ is H or a pharmaceutically acceptable salt or an in vivo hydrolysable ester thereof.

In an additional embodiment of section 3, the present invention provides an enantiomer of a compound of formula (I) wherein R⁴ is methyl or a pharmaceutically acceptable salt or an in vivo hydrolysable ester thereof.

In an additional embodiment of section 3, the present invention provides an enantiomer of a compound of formula (I) wherein R⁴ is ethyl or a pharmaceutically acceptable salt or an in vivo hydrolysable ester thereof.

In an additional embodiment of section 3, the present invention provides an enantiomer of a compound of formula (I) wherein R⁴ is propyl or a pharmaceutically acceptable salt or an in vivo hydrolysable ester thereof.

In an additional embodiment of section 3, the present invention provides an enantiomer of a compound of formula (I) wherein R⁴ is isopropyl or a pharmaceutically acceptable salt or an in vivo hydrolysable ester thereof.

In an additional embodiment of section 3, the present invention provides an enantiomer of a compound of formula (I) wherein R⁵ is H or a pharmaceutically acceptable salt or an in vivo hydrolysable ester thereof.

In an additional embodiment of section 3, the present invention provides an enantiomer of a compound of formula (I) wherein R⁵ is methyl or a pharmaceutically acceptable salt or an in vivo hydrolysable ester thereof.

In an additional embodiment of section 3, the present invention provides an enantiomer of a compound of formula (I) wherein R⁵ is ethyl or a pharmaceutically acceptable salt or an in vivo hydrolysable ester thereof.

In an additional embodiment of section 3, the present invention provides an enantiomer of a compound of formula (I) wherein R⁵ is propyl or a pharmaceutically acceptable salt or an in vivo hydrolysable ester thereof.

In an additional embodiment of section 3, the present invention provides an enantiomer of a compound of formula (I) wherein R⁵ is isopropyl or a pharmaceutically acceptable salt or an in vivo hydrolysable ester thereof.

In an additional embodiment of section 3, the present invention provides an enantiomer of a compound of formula (I) wherein R⁴ and R⁵ are both H or both methyl, or R⁴ is H and R⁵ is isopropyl or a pharmaceutically acceptable salt or an in vivo hydrolysable ester thereof.

In an additional embodiment of section 3, the present invention provides an enantiomer of a compound of formula (I) wherein Z is optionally substituted phenyl or a pharmaceutically acceptable salt or an in vivo hydrolysable ester thereof.

In an additional embodiment of section 3, the present invention provides an enantiomer of a compound of formula (I) wherein Z is optionally substituted benzothiophene or a pharmaceutically acceptable salt or an in vivo hydrolysable ester thereof.

In an additional embodiment of section 3, the present invention provides an enantiomer of a compound of formula (I) wherein Z is 4-methylphenyl or a pharmaceutically acceptable salt or an in vivo hydrolysable ester thereof.

In an additional embodiment of section 3, the present invention provides an enantiomer of a compound of formula (I) wherein Z is benzothiophen-2-yl or a pharmaceutically acceptable salt or an in vivo hydrolysable ester thereof.

In an additional embodiment of section 3, the present invention provides an enantiomer of a compound of formula (I) wherein Z is 4-chlorophenyl or a pharmaceutically acceptable salt or an in vivo hydrolysable ester thereof.

In an additional embodiment of section 3, the present invention provides an enantiomer of a compound of formula (I) wherein Z is 4-bromophenyl or a pharmaceutically acceptable salt or an in vivo hydrolysable ester thereof.

In an additional embodiment of section 3, the present invention provides an enantiomer of a compound of formula (I) wherein Z is 4-methyl-3-fluorophenyl or a pharmaceutically acceptable salt or an in vivo hydrolysable ester thereof.

In an additional embodiment of section 3, the present invention provides an enantiomer of a compound of formula (I) wherein Z is 2,3-dichlorophenyl or a pharmaceutically acceptable salt or an in vivo hydrolysable ester thereof.

In an additional embodiment of section 3, the present invention provides an enantiomer of a compound of formula (I) wherein Z is 4-methylphenyl, benzothiophen-2-yl, 4-chlorophenyl, 4-bromophenyl, 4-methyl-3-fluorophenyl or 2,3-dichlorophenyl or a pharmaceutically acceptable salt or an in vivo hydrolysable ester thereof.

Particular values of variable groups within section 3 are as follows. Such values may be used where appropriate with any of the definitions, claims or embodiments defined hereinbefore or hereinafter in section 3.

X is —C(CH₃)—.

X is S.

Y is C.

Y is S.

Y is O.

Y is —S— and X is —C(CH₃)—.

Y is —O— and X is —C(CH₃)—.

Y is —C(CH₃)— and X is —S—.

m is 0.

m is 1.

R² is H.

R² is methyl.

R² is ethyl.

R² is propyl.

R² is isopropyl.

R³ is methyl.

R³ is ethyl.

R³ is propyl.

R³ is isopropyl.

R² is H and R³ is methyl.

R² and R³ are methyl.

n is 2.

n is 3.

R³ is H.

R⁴ is H.

R⁴ methyl.

R⁴ is ethyl.

R⁴ is propyl.

R⁴ is isopropyl.

R⁵ is H.

R⁵ is methyl.

R⁵ is ethyl.

R⁵ is propyl.

R⁵ is isopropyl.

R⁴ and R⁵ are both H or both methyl, or R⁴ is H and R⁵ is isopropyl.

Z is optionally substituted phenyl.

Z is optionally substituted benzothiophene.

Z is 4-methylphenyl.

Z is benzothiophen-2-yl.

Z is 4-chlorophenyl.

Z is 4-bromophenyl.

Z is 4-methyl-3-fluorophenyl.

Z is 2,3-dichlorophenyl.

Z is 4-methylphenyl, benzothiophen-2-yl, 4-chlorophenyl, 4-bromophenyl, 4-methyl-3-fluorophenyl or 2,3-dichlorophenyl.

In a further aspect of section 3 of the invention there is provided an enantiomer of a compound of formula (I) (as depicted above) including a pharmaceutically acceptable salt or an in vivo hydrolysable ester thereof,

wherein:

X is selected from —C(CH₃)— or —S— provided that when X is —S— then Y is —C(CH₃)—;
Y is selected from —C(CH₃)— or —O— or —S— provided that when Y is —C(CH₃)— then X is not —C(CH₃)—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl;
Z is 4-methylphenyl, benzothiophen-2-yl, 4-chlorophenyl, 4-bromophenyl, 4-methyl-3-fluorophenyl or 2,3-dichlorophenyl; and
“*” represents a chiral center;
   wherein said enantiomer is substantially free of the other enantiomer; and wherein the optical rotation of the enantiomer, when said enantiomer is dissolved at a concentration of 1 mg/ml in methanol, at 20.0° C. measured at 589 nM is (+).

In a further aspect of section 3 of the invention there is provided an (R) enantiomer of a compound of formula (Ia) (as depicted above) including a pharmaceutically acceptable salt or an in vivo hydrolysable ester thereof,

wherein:

X is selected from —C(CH₃)— or —S— provided that when X is —S— then Y is —C(CH₃)—;
Y is selected from —C(CH₃)— or —O— or —S— provided that when Y is —C(CH₃)— then X is not —C(CH₃)—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl; and
Z is 4-methylphenyl, benzothiophen-2-yl, 4-chlorophenyl, 4-bromophenyl, 4-methyl-3-fluorophenyl or 2,3-dichlorophenyl;
   wherein said enantiomer is substantially free of the (S) enantiomer.

In a further aspect of the invention of section 3 there is provided an (S) enantiomer of a compound of formula (Ib) (as depicted above) including a pharmaceutically acceptable salt or an in vivo hydrolysable ester thereof,

wherein:

X is selected from —C(CH₃)— or —S— provided that when X is —S— then Y is —C(CH₃)—;
Y is selected from —C(CH₃)— or —O— or —S— provided that when Y is —C(CH₃)— then X is not —C(CH₃)—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl; and
Z is 4-methylphenyl, benzothiophen-2-yl, 4-chlorophenyl, 4-bromophenyl, 4-methyl-3-fluorophenyl or 2,3-dichlorophenyl;
   wherein said enantiomer is substantially free of the (R) enantiomer.

In a further aspect of section 3 of the invention there is provided an enantiomer of a compound of formula (I) (as depicted above) including a pharmaceutically acceptable salt or an in vivo hydrolysable ester thereof,

wherein:

Y is —S— and X is —C(CH₃)—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl;
Z is 4-methylphenyl, benzothiophen-2-yl, 4-chlorophenyl, 4-bromophenyl, 4-methyl-3-fluorophenyl or 2,3-dichlorophenyl; and
“*” represents a chiral center;
   wherein said enantiomer is substantially free of the other enantiomer; and wherein the optical rotation of the enantiomer, when said enantiomer is dissolved at a concentration of 1 mg/ml in methanol, at 20.0° C. measured at 589 nM is (+).

In a further aspect of section 3 of the invention there is provided an (R) enantiomer of a compound of formula (Ia) (as depicted above) including a pharmaceutically acceptable salt or an in vivo hydrolysable ester thereof,

wherein:

Y is —S— and X is —C(CH₃)—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl; and
Z is 4-methylphenyl, benzothiophen-2-yl, 4-chlorophenyl, 4-bromophenyl, 4-methyl-3-fluorophenyl or 2,3-dichlorophenyl;
   wherein said enantiomer is substantially free of the (S) enantiomer.

In a further aspect of section 3 of the invention there is provided an (S) enantiomer of a compound of formula (Ib) (as depicted above) including a pharmaceutically acceptable salt or an in vivo hydrolysable ester thereof,

wherein:

Y is —S— and X is —C(CH₃)—;
m is 0 or 1;
R¹ is F when m is 1;
one of R² and R³ is H and the other is methyl or both R¹ and R³ are methyl;
n is 2 or 3;
R⁴ and R⁵ are independently selected from H or C₁₋₃alkyl; and
Z is 4-methylphenyl, benzothiophen-2-yl, 4-chlorophenyl, 4-bromophenyl, 4-methyl-3-fluorophenyl or 2,3-dichlorophenyl;
   wherein said enantiomer is substantially free of the (R) enantiomer.

In a further aspect of section 3 of the invention there is provided an enantiomer of a compound of formula (I) (as depicted above) including a pharmaceutically acceptable salt or an in vivo hydrolysable ester thereof,

wherein:

Y is —O— and X is —C(CH₃)—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl;
Z is 4-methylphenyl, benzothiophen-2-yl, 4-chlorophenyl, 4-bromophenyl, 4-methyl-3-fluorophenyl or 2,3-dichlorophenyl; and
“*” represents a chiral center;
   wherein said enantiomer is substantially free of the other enantiomer; and wherein the optical rotation of the enantiomer, when said enantiomer is dissolved at a concentration of 1 mg/ml in methanol, at 20.0° C. measured at 589 nM is (+).

In a further aspect of section 3 of the invention there is provided an (R) enantiomer of a compound of formula (Ia) (as depicted above) including a pharmaceutically acceptable salt or an in vivo hydrolysable ester thereof,

wherein:

Y is —O— and X is —C(CH₃)—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl; and
Z is 4-methylphenyl, benzothiophen-2-yl, 4-chlorophenyl, 4-bromophenyl, 4-methyl-3-fluorophenyl or 2,3-dichlorophenyl;
   wherein said enantiomer is substantially free of the (S) enantiomer.

In a further aspect of section 3 of the invention there is provided an (S) enantiomer of a compound of formula (Ib) (as depicted above) including a pharmaceutically acceptable salt or an in vivo hydrolysable ester thereof,

wherein:

Y is —O— and X is —C(CH₃)—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl; and
Z is 4-methylphenyl, benzothiophen-2-yl, 4-chlorophenyl, 4-bromophenyl, 4-methyl-3-fluorophenyl or 2,3-dichlorophenyl;
   wherein said enantiomer is substantially free of the (R) enantiomer.

In a further aspect of section 3 of the invention there is provided an enantiomer of a compound of formula (I) (as depicted above) including a pharmaceutically acceptable salt or an in vivo hydrolysable ester thereof,

wherein:

Y is —C(CH₃)— and X is —S—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl;
Z is 4-methylphenyl, benzothiophen-2-yl, 4-chlorophenyl, 4-bromophenyl, 4-methyl-3-fluorophenyl or 2,3-dichlorophenyl; and
“*” represents a chiral center;
   wherein said enantiomer is substantially free of the other enantiomer; and wherein the optical rotation of the enantiomer, when said enantiomer is dissolved at a concentration of 1 mg/ml in methanol, at 20.0° C. measured at 589 nM is (+).

In a further aspect of section 3 of the invention there is provided an (R) enantiomer of a compound of formula (Ia) (as depicted above) including a pharmaceutically acceptable salt or an in vivo hydrolysable ester thereof,

wherein:

Y is —C(CH₃)— and X is —S—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl; and
Z is 4-methylphenyl, benzothiophen-2-yl, 4-chlorophenyl, 4-bromophenyl, 4-methyl-3-fluorophenyl or 2,3-dichlorophenyl;
   wherein said enantiomer is substantially free of the (S) enantiomer.

In a further aspect of section 3 of the invention there is provided an (S) enantiomer of a compound of formula (Ib) (as depicted above) including a pharmaceutically acceptable salt or an in vivo hydrolysable ester thereof, wherein:
Y is —C(CH₃)— and X is —S—;
m is 0 or 1;
R¹ is F when m is 1;
one of R² and R³ is H and the other is methyl or both R² and R³ are methyl;
n is 2 or 3;
R⁴ and R⁵ are independently selected from H or C₁₋₃alkyl; and
Z is 4-methylphenyl, benzothiophen-2-yl, 4-chlorophenyl, 4-bromophenyl, 4-methyl-3-fluorophenyl or 2,3-dichlorophenyl;
   wherein said enantiomer is substantially free of the (R) enantiomer.

In a further aspect of section 3 of the invention there is provided a compound of formula (I) or a pharmaceutically acceptable salt thereof.

In an additional embodiment of section 3, the present invention provides a compound of formula (I) or a pharmaceutically acceptable salt or an in vivo hydrolysable ester thereof selected from:
(+) N-(3-Amino-propyl)-N-[1-(5-benzyl-3-methyl-4-oxo-4,5-dihydro-isothiazolo[5,4-d]pyrimidin-6-yl)-propyl]-4-methyl-benzamide hydrogen chloride;
(+) N-(3-Amino-propyl)-N-{1-[5-(4-fluoro-benzyl)-3-methyl-4-oxo-4,5-dihydro-isothiazolo[5,4-d]pyrimidin-6-yl]-propyl}-4-methyl-benzamide hydrogen chloride;
(+) N-(3-Amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide hydrogen chloride;
(+) N-(3-Amino-propyl)-N-[1-(5-benzyl-3-methyl-4-oxo-4,5-dihydro-isothiazolo[5,4-d]pyrimidin-6-yl)-propyl]-4-bromo-benzamide hydrogen chloride;
(+) N-(3-Amino-propyl)-N-[1-(5-benzyl-3-methyl-4-oxo-4,5-dihydro-isothiazolo[5,4-d]pyrimidin-6-yl)-propyl]-4-chloro-benzamide hydrogen chloride;
(+) N-(3-Amino-propyl)-N-[1-(5-benzyl-3-methyl-4-oxo-4,5-dihydro-isothiazolo[5,4-d]pyrimidin-6-yl)-propyl]-3-fluoro-4-methyl-benzamide hydrogen chloride;
(+) N-(3-Amino-propyl)-N-[1-(5-benzyl-3-methyl-4-oxo-4,5-dihydro-isothiazolo[5,4-d]pyrimidin-6-yl)-propyl]-2,3-dichloro-benzamide hydrogen chloride;
(+) Benzo[b]thiophene-2-carboxylic acid (3-amino-propyl)-[1-(5-benzyl-3-methyl-4-oxo-4,5-dihydro-isothiazolo[5,4-d]pyrimidin-6-yl)-propyl]amide hydrogen chloride;
(+) N-(2-Amino-ethyl)-N-[1-(5-benzyl-3-methyl-4-oxo-4,5-dihydro-isothiazolo[5,4-d]pyrimidin-6-yl)-propyl]-4-methyl-benzamide;
(+) N-[1-(5-Benzyl-3-methyl-4-oxo-4,5-dihydro-isothiazolo[5,4-d]pyrimidin-6-yl)-propyl]-N-(3-dimethylamino-propyl)-4-methyl-benzamide;
(+) N-[1-(5-Benzyl-3-methyl-4-oxo-4,5-dihydro-isothiazolo[5,4-d]pyrimidin-6-yl)-propyl]-N-(3-isopropylamino-propyl)-4-methyl-benzamide;
(+) N-(3-Amino-propyl)-N-[1-(5-benzyl-3-methyl-4-oxo-4,5-dihydro-isoxazolo[5,4-d]pyrimidin-6-yl)-propyl]-4-methyl-benzamide hydrogen chloride;
(+) N-(3-Amino-propyl)-N-{1-[5-(4-fluoro-benzyl)-3-methyl-4-oxo-4,5-dihydro-isothiazolo[5,4-d]pyrimidin-6-yl]-2-methyl-propyl}-4-methyl-benzamide hydrogen chloride;
(+) N-(3-Amino-propyl)-N-[1-(5-benzyl-3-methyl-4-oxo-4,5-dihydro-isothiazolo[5,4-d]pyrimidin-6-yl)-2-methyl-propyl]-4-methyl-benzamide hydrogen chloride;
(+) N-(3-Amino-propyl)-N-{1-[5-(3-fluoro-benzyl)-3-methyl-4-oxo-4,5-dihydro-isothiazolo[5,4-d]pyrimidin-6-yl]-2-methyl-propyl}-4-methyl-benzamide hydrogen chloride;
(+) N-(2-Amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide hydrogen chloride;
(+) N-(2-Amino-ethyl)-N-[1-(5-benzyl-3-methyl-4-oxo-4,5-dihydro-isothiazolo[5,4-d]pyrimidin-6-yl)-2-methyl-propyl]-4-methyl-benzamide hydrogen chloride;
(+) N-(2-Amino-ethyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide hydrogen chloride;
(+) N-(3-Amino-propyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide hydrogen chloride;
(+) N-(3-Amino-propyl)-N-[1-(5-benzyl-3-methyl-4-oxo-4,5-dihydro-isothiazolo[5,4-d]pyrimidin-6-yl)-2-methyl-propyl]4-bromo-benzamide hydrogen chloride;
(+) N-[1-(5-Benzyl-3-methyl-4-oxo-4,5-dihydro-isothiazolo[5,4-d]pyrimidin-6-yl)-2-methyl-propyl]-N-(3-dimethylamino-propyl)-4-methyl-benzamide;
(+) N-[1-(5-Benzyl-3-methyl-4-oxo-4,5-dihydro-isothiazolo[5,4-d]pyrimidin-6-yl)-2-methyl-propyl]-N-(3-dimethylamino-propyl)-4-bromo-benzamide;
(+) N-[1-(5-Benzyl-3-methyl-4-oxo-4,5-dihydro-isothiazolo[5,4-d]pyrimidin-6-yl)-2-methyl-propyl]-N-(3-dimethylamino-propyl)-3-fluoro-4-methyl-benzamide;
(+) N-(3-Amino-propyl)-N-[1-(5-benzyl-3-methyl-4-oxo-4,5-dihydro-isoxazolo[5,4-d]pyrimidin-6-yl)-2-methyl-propyl]-4-methyl-benzamide hydrogen chloride;
(+) N-(3-Amino-propyl)-N-{1-[5-(4-fluoro-benzyl)-3-methyl-4-oxo-4,5-dihydro-isoxazolo[5,4-d]pyrimidin-6-yl]-2-methyl-propyl}-4-methyl-benzamide hydrogen chloride;
(+) N-(3-Amino-propyl)-N-{1-[5-(3-fluoro-benzyl)-3-methyl-4-oxo-4,5-dihydro-isoxazolo[5,4-d]pyrimidin-6-yl]-2-methyl-propyl}-4-methyl-benzamide hydrogen chloride;
(+) N-(3-Amino-propyl)-N-[1-(6-benzyl-3-methyl-7-oxo-6,7-dihydro-isothiazolo[4,5-d]pyrimidin-5-yl)-propyl]-4-methyl-benzamide.

In an additional embodiment of section 3, the present invention provides a compound of formula (I) or a pharmaceutically acceptable salt or an in vivo hydrolysable ester thereof selected from:
(+) N-(3-Amino-propyl)-N-[1-(5-benzyl-3-methyl-4-oxo-4,5-dihydro-isothiazolo[5,4-d]pyrimidin-6-yl)-propyl]-4-methyl-benzamide;
(+) N-(3-Amino-propyl)-N-{1-[5-(4-fluoro-benzyl)-3-methyl-4-oxo-4,5-dihydro-isothiazolo[5,4-d]pyrimidin-6-yl]-propyl}-4-methyl-benzamide;
(+) N-(3-Amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide;
(+) N-(3-Amino-propyl)-N-[1-(5-benzyl-3-methyl-4-oxo-4,5-dihydro-isothiazolo[5,4-d]pyrimidin-6-yl)-propyl]-4-bromo-benzamide;
(+) N-(3-Amino-propyl)-N-[1-(5-benzyl-3-methyl-4-oxo-4,5-dihydro-isothiazolo[5,4-d]pyrimidin-6-yl)-propyl]-4-chloro-benzamide;
(+) N-(3-Amino-propyl)-N-[1-(5-benzyl-3-methyl-4-oxo-4,5-dihydro-isothiazolo[5,4-d]pyrimidin-6-yl)-propyl]-3-fluoro-4-methyl-benzamide;
(+) N-(3-Amino-propyl)-N-[1-(5-benzyl-3-methyl-4-oxo-4,5-dihydro-isothiazolo[5,4-d]pyrimidin-6-yl)-propyl]-2,3-dichloro-benzamide;
(+) Benzo[b]thiophene-2-carboxylic acid (3-amino-propyl)-[1-(5-benzyl-3-methyl-4-oxo-4,5-dihydro-isothiazolo[5,4-d]pyrimidin-6-yl)-propyl]amide;
(+) N-(2-Amino-ethyl)-N-[1-(5-benzyl-3-methyl-4-oxo-4,5-dihydro-isothiazolo[5,4-d]pyrimidin-6-yl)-propyl]-4-methyl-benzamide;
(+) N-[1-(5-Benzyl-3-methyl-4-oxo-4,5-dihydro-isothiazolo[5,4-d]pyrimidin-6-yl)-propyl]-N-(3-dimethylamino-propyl)-4-methyl-benzamide;
(+) N-[1-(5-Benzyl-3-methyl-4-oxo-4,5-dihydro-isothiazolo[5,4-d]pyrimidin-6-yl)-propyl]-(3-isopropylamino-propyl)-4-methyl-benzamide;
(+) N-(3-Amino-propyl)-N-[1-(5-benzyl-3-methyl-4-oxo-4,5-dihydro-isoxazolo[5,4-d]pyrimidin-6-yl)-propyl]-4-methyl-benzamide;
(+) N-(3-Amino-propyl)-N-{1-[5-(4-fluoro-benzyl)-3-methyl-4-oxo-4,5-dihydro-isothiazolo[5,4-d]pyrimidin-6-yl]-2-methyl-propyl}-4-methyl-benzamide;
(+) N-(3-Amino-propyl)-N-[1-(5-benzyl-3-methyl-4-oxo-4,5-dihydro-isothiazolo[5,4-d]pyrimidin-6-yl)-2-methyl-propyl]-4-methyl-benzamide;
(+) N-(3-Amino-propyl)-N-{1-[5-(3-fluoro-benzyl)-3-methyl-4-oxo-4,5-dihydro-isothiazolo[5,4-d]pyrimidin-6-yl]-2-methyl-propyl}-4-methyl-benzamide;
(+) N-(2-Amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide;
(+) N-(2-Amino-ethyl)-N-[1-(5-benzyl-3-methyl-4-oxo-4,5-dihydro-isothiazolo[5,4-d]pyrimidin-6-yl)-2-methyl-propyl]-4-methyl-benzamide;
(+) N-(2-Amino-ethyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
(+) N-(3-Amino-propyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
(+) N-(3-Amino-propyl)-N-[1-(5-benzyl-3-methyl-4-oxo-4,5-dihydro-isothiazolo[5,4-d]pyrimidin-6-yl)-2-methyl-propyl]-4-bromo-benzamide;
(+) N-[1-(5-Benzyl-3-methyl-4-oxo-4,5-dihydro-isothiazolo[5,4-d]pyrimidin-6-yl)-2-methyl-propyl]-N-(3-dimethylamino-propyl)-4-methyl-benzamide;
(+) N-[1-(5-Benzyl-3-methyl-4-oxo-4,5-dihydro-isothiazolo[5,4-d]pyrimidin-6-yl)-2-methyl-propyl]-N-(3-dimethylamino-propyl)₄-bromo-benzamide;
(+) N-[1-(5-Benzyl-3-methyl-4-oxo-4,5-dihydro-isothiazolo[5,4-d]pyrimidin-6-yl)-2-methyl-propyl]-N-(3-dimethylamino-propyl)-3-fluoro-4-methyl-benzamide;
(+) N-(3-Amino-propyl)-N-[1-(5-benzyl-3-methyl-4-oxo-4,5-dihydro-isoxazolo[5,4-d]pyrimidin-6-yl)-2-methyl-propyl]-4-methyl-benzamide;
(+) N-(3-Amino-propyl)-N-{1-[5-(4-fluoro-benzyl)-3-methyl-4-oxo-4,5-dihydro-isoxazolo[5,4-d]pyrimidin-6-yl]-2-methyl-propyl}-4-methyl-benzamide;
(+) N-(3-Amino-propyl)-N-{1-[5-(3-fluoro-benzyl)-3-methyl-4-oxo-4,5-dihydro-isoxazolo[5,4-d]pyrimidin-6-yl]-2-methyl-propyl}-4-methyl-benzamide; or
(+) N-(3-Amino-propyl)-N-[1-(6-benzyl-3-methyl-7-oxo-6,7-dihydro-isothiazolo[4,5-d]pyrimidin-5-yl)-propyl]-4-methyl-benzamide.

In an additional embodiment of section 3, the present invention provides an enantiomer of formula (Ia) or a pharmaceutically acceptable salt or an in vivo hydrolysable ester thereof selected from:
(R) N-(3-Amino-propyl)-N-[1-(5-benzyl-3-methyl-4-oxo-4,5-dihydro-isothiazolo[5,4-d]pyrimidin-6-yl)-propyl]-4-methyl-benzamide;
(R) N-(3-Amino-propyl)-N-{1-[5-(4-fluoro-benzyl)-3-methyl-4-oxo-4,5-dihydro-isothiazolo[5,4-d]pyrimidin-6-yl]-propyl}-4-methyl-benzamide;
(R) N-(3-Amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide;
(R) N-(3-Amino-propyl)-N-[1-(5-benzyl-3-methyl-4-oxo-4,5-dihydro-isothiazolo[5,4-d]pyrimidin-6-yl)-propyl]-4-bromo-benzamide;
(R) N-(3-Amino-propyl)-N-[1-(5-benzyl-3-methyl-4-oxo-4,5-dihydro-isothiazolo[5,4-d]pyrimidin-6-yl)-propyl]-4-chloro-benzamide;
(R) N-(3-Amino-propyl)-N-[1-(5-benzyl-3-methyl-4-oxo-4,5-dihydro-isothiazolo[5,4-d]pyrimidin-6-yl)-propyl]-3-fluoro-4-methyl-benzamide;
(R) N-(3-Amino-propyl)-N-[1-(5-benzyl-3-methyl-4-oxo-4,5-dihydro-isothiazolo[5,4-d]pyrimidin-6-yl)-propyl]-2,3-dichloro-benzamide;
(R) Benzo[b]thiophene-2-carboxylic acid (3-amino-propyl)-[1-(5-benzyl-3-methyl-4-oxo-4,5-dihydro-isothiazolo[5,4-d]pyrimidin-6-yl)-propyl]amide;
(R) N-(2-Amino-ethyl)-N-[1-(5-benzyl-3-methyl-4-oxo-4,5-dihydro-isothiazolo[5,4-d]pyrimidin-6-yl)-propyl]-4-methyl-benzamide;
(R) N-[1-(5-Benzyl-3-methyl-4-oxo-4,5-dihydro-isothiazolo[5,4-d]pyrimidin-6-yl)-propyl]-N-(3-dimethylamino-propyl)-4-methyl-benzamide;
(R) N-[1-(5-Benzyl-3-methyl-4-oxo-4,5-dihydro-isothiazolo[5,4-d]pyrimidin-6-yl)-propyl]-N-(3-isopropylamino-propyl)-4-methyl-benzamide;
(R) N-(3-Amino-propyl)-N-[1-(5-benzyl-3-methyl-4-oxo-4,5-dihydro-isoxazolo[5,4-d]pyrimidin-6-yl)-propyl]-4-methyl-benzamide;
(R) N-(3-Amino-propyl)-N-{1-[5-(4-fluoro-benzyl)-3-methyl-4-oxo-4,5-dihydro-isothiazolo[5,4-d]pyrimidin-6-yl]-2-methyl-propyl}-4-methyl-benzamide;
(R) N-(3-Amino-propyl)-N-[1-(5-benzyl-3-methyl-4-oxo-4,5-dihydro-isothiazolo[5,4-d]pyrimidin-6-yl)-2-methyl-propyl]-4-methyl-benzamide;
(R) N-(3-Amino-propyl)-N-{1-[5-(3-fluoro-benzyl)-3-methyl-4-oxo-4,5-dihydro-isothiazolo[5,4-d]pyrimidin-6-yl]-2-methyl-propyl}-4-methyl-benzamide;
(R) N-(2-Amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide;
(R) N-(2-Amino-ethyl)-N-[1-(5-benzyl-3-methyl-4-oxo-4,5-dihydro-isothiazolo[5,4-d]pyrimidin-6-yl)-2-methyl-propyl]-4-methyl-benzamide;
(R) N-(2-Amino-ethyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
(R) N-(3-Amino-propyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
(R) N-(3-Amino-propyl)-N-[1-(5-benzyl-3-methyl-4-oxo-4,5-dihydro-isothiazolo[5,4-d]pyrimidin-6-yl)-2-methyl-propyl]-4-bromo-benzamide;
(R) N-[1-(5-Benzyl-3-methyl-4-oxo-4,5-dihydro-isothiazolo[5,4-d]pyrimidin-6-yl)-2-methyl-propyl]-N-(3-dimethylamino-propyl)-4-methyl-benzamide;
(R) N-[1-(5-Benzyl-3-methyl-4-oxo-4,5-dihydro-isothiazolo[5,4-d]pyrimidin-6-yl)-2-methyl-propyl]-N-(3-dimethylamino-propyl)-4-bromo-benzamide;
(R) N-[1-(5-Benzyl-3-methyl-4-oxo-4,5-dihydro-isothiazolo[5,4-d]pyrimidin-6-yl)-2-methyl-propyl]-N-(3-dimethylamino-propyl)-3-fluoro-4-methyl-benzamide;
(R) N-(3-Amino-propyl)-N-[1-(5-benzyl-3-methyl-4-oxo-4,5-dihydro-isoxazolo[5,4-d]pyrimidin-6-yl)-2-methyl-propyl]4-methyl-benzamide;
(R) N-(3-Amino-propyl)-N-{1-[5-(4-fluoro-benzyl)-3-methyl-4-oxo-4,5-dihydro-isoxazolo[5,4-d]pyrimidin-6-yl]-2-methyl-propyl}-4-methyl-benzamide;
(R) N-(3-Amino-propyl)-N-{1-[5-(3-fluoro-benzyl)-3-methyl-4-oxo-4,5-dihydro-isoxazolo[5,4-d]pyrimidin-6-yl]-2-methyl-propyl}-4-methyl-benzamide; or
(R) N-(3-Amino-propyl)-N-[1-(6-benzyl-3-methyl-7-oxo-6,7-dihydro-isothiazolo[4,5-d]pyrimidin-5-yl)-propyl]-4-methyl-benzamide.

In an additional embodiment of section 3, the present invention provides an enantiomer of formula (Ib) or a pharmaceutically acceptable salt or an in vivo hydrolysable ester thereof selected from:
(S) N-(3-Amino-propyl)-N-[1-(5-benzyl-3-methyl-4-oxo-4,5-dihydro-isothiazolo[5,4-d]pyrimidin-6-yl)-propyl]-4-methyl-benzamide;
(S) N-(3-Amino-propyl)-N-{1-[5-(4-fluoro-benzyl)-3-methyl-4-oxo-4,5-dihydro-isothiazolo[5,4-d]pyrimidin-6-yl]-propyl}-4-methyl-benzamide;
(S) N-(3-Amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide;
(S) N-(3-Amino-propyl)-N-[1-(5-benzyl-3-methyl-4-oxo-4,5-dihydro-isothiazolo[5,4-d]pyrimidin-6-yl)-propyl]-4-bromo-benzamide;
(S) N-(3-Amino-propyl)-N-[1-(5-benzyl-3-methyl-4-oxo-4,5-dihydro-isothiazolo[5,4-d]pyrimidin-6-yl)-propyl]-4-chloro-benzamide;
(S) N-(3-Amino-propyl)-N-[1-(5-benzyl-3-methyl-4-oxo-4,5-dihydro-isothiazolo[5,4-d]pyrimidin-6-yl)-propyl]-3-fluoro-4-methyl-benzamide;
(S) N-(3-Amino-propyl)-N-[1-(5-benzyl-3-methyl-4-oxo-4,5-dihydro-isothiazolo[5,4-d]pyrimidin-6-yl)-propyl]-2,3-dichloro-benzamide;
(S) Benzo[b]thiophene-2-carboxylic acid (3-amino-propyl)-[1-(5-benzyl-3-methyl-4-oxo-4,5-dihydro-isothiazolo[5,4-d]pyrimidin-6-yl)-propyl]amide;
(S) N-(2-Amino-ethyl)-N-[1-(5-benzyl-3-methyl-4-oxo-4,5-dihydro-isothiazolo[5,4-d]pyrimidin-6-yl)-propyl]-4-methyl-benzamide;
(S) N-[1-(5-Benzyl-3-methyl-4-oxo-4,5-dihydro-isothiazolo[5,4-d]pyrimidin-6-yl)-propyl]-N-(3-dimethylamino-propyl)-4-methyl-benzamide;
(S) N-[1-(5-Benzyl-3-methyl-4-oxo-4,5-dihydro-isothiazolo[5,4-d]pyrimidin-6-yl)-propyl]-N-(3-isopropylamino-propyl)₄-methyl-benzamide;
(S) N-(3-Amino-propyl)-N-[1-(5-benzyl-3-methyl-4-oxo-4,5-dihydro-isoxazolo[5,4-d]pyrimidin-6-yl)-propyl]4-methyl-benzamide;
(S) N-(3-Amino-propyl)-N-{1-[5-(4-fluoro-benzyl)-3-methyl-4-oxo-4,5-dihydro-isothiazolo[5,4-d]pyrimidin-6-yl]-2-methyl-propyl}-4-methyl-benzamide;
(S) N-(3-Amino-propyl)-N-[1-(5-benzyl-3-methyl-4-oxo-4,5-dihydro-isothiazolo[5,4-d]pyrimidin-6-yl)-2-methyl-propyl]-4-methyl-benzamide;
(S) N-(3-Amino-propyl)-N-{1-[5-(3-fluoro-benzyl)-3-methyl-4-oxo-4,5-dihydro-isothiazolo[5,4-d]pyrimidin-6-yl]-2-methyl-propyl}-4-methyl-benzamide;
(S) N-(2-Amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide;
(S) N-(2-Amino-ethyl)-N-[1-(5-benzyl-3-methyl-4-oxo-4,5-dihydro-isothiazolo[5,4-d]pyrimidin-6-yl)-2-methyl-propyl]-4-methyl-benzamide;
(S) N-(2-Amino-ethyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
(S) N-(3-Amino-propyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
(S) N-(3-Amino-propyl)-N-[1-(5-benzyl-3-methyl-4-oxo-4,5-dihydro-isothiazolo[5,4-d]pyrimidin-6-yl)-2-methyl-propyl]-4-bromo-benzamide;
(S) N-[1-(5-Benzyl-3-methyl-4-oxo-4,5-dihydro-isothiazolo[5,4-d]pyrimidin-6-yl)-2-methyl-propyl]-N-(3-dimethylamino-propyl)₄-methyl-benzamide;
(S) N-[1-(5-Benzyl-3-methyl-4-oxo-4,5-dihydro-isothiazolo[5,4-d]pyrimidin-6-yl)-2-methyl-propyl]-N-(3-dimethylamino-propyl)-4-bromo-benzamide;
(S) N-[1-(5-Benzyl-3-methyl-4-oxo-4,5-dihydro-isothiazolo [5,4-d]pyrimidin-6-yl)-2-methyl-propyl]-N-(3-dimethylamino-propyl)-3-fluoro-4-methyl-benzamide;
(S) N-(3-Amino-propyl)-N-[1-(5-benzyl-3-methyl-4-oxo-4,5-dihydro-isoxazolo[5,4-d]pyrimidin-6-yl)-2-methyl-propyl]-4-methyl-benzamide;
(S) N-(3-Amino-propyl)-N-{1-[5-(4-fluoro-benzyl)-3-methyl-4-oxo-4,5-dihydro-isoxazolo[5,4-d]pyrimidin-6-yl]-2-methyl-propyl}-4-methyl-benzamide;
(S) N-(3-Amino-propyl)-N-{1-[5-(3-fluoro-benzyl)-3-methyl-4-oxo-4,5-dihydro-isoxazolo[5,4-d]pyrimidin-6-yl]-2-methyl-propyl}-4-methyl-benzamide; or
(S) N-(3-Amino-propyl)-N-[1-(6-benzyl-3-methyl-7-oxo-6,7-dihydro-isothiazolo[4,5-d]pyrimidin-5-yl)-propyl]-4-methyl-benzamide.

A particular embodiment of section 3 of the invention refers to a compound of formula (I), (Ia) or (Ib) or a pharmaceutically acceptable salt thereof.

A compound of formula (I) of section 3 or a pharmaceutically acceptable salt or an in vivo hydrolysable ester thereof, which is substantially free of its corresponding (−) enantiomer.

In section 3, the term “substantially free” refers to less than 10% of the other isomer, more particularly less than 5%, in particular less than 2%, more particularly less than 1%, particularly less then 0.5%, in particular less than 0.2%.

A compound of formula (I) of section 3 or a pharmaceutically acceptable salt or an in vivo hydrolysable ester thereof having no more than about 1% w/w of the corresponding (−) enantiomer.

A compound of formula (I) of section 3 or a pharmaceutically acceptable salt or an in vivo hydrolysable ester thereof having no more than 1% w/w of the corresponding (−) enantiomer.

A compound of formula (I) of section 3 or a pharmaceutically acceptable salt or an in vivo hydrolysable ester thereof having no more than about 2% w/w of the corresponding (−) enantiomer.

A compound of formula (I) of section 3 or a pharmaceutically acceptable salt or an in vivo hydrolysable ester thereof having no more than 2% w/w of the corresponding (−) enantiomer.

In an additional embodiment of section 3, the present invention provides a compound of formula (I) or a pharmaceutically acceptable salt or an in vivo hydrolysable ester thereof for use as a medicament.

In section 3 where the use of a compound of formula (I), or a method of treatment comprising administering a compound of formula (I), or the use of a pharmaceutical composition comprising a compound of formula (I), is referred to it is to be understood that “a compound of formula (I)” refers to (i) an enantiomer of a compound of formula (I); or (ii) an (R) enantiomer of formula (Ia); or (iii) an (S) enantiomer of formula (Ib).

According to a further aspect of section 3 of the invention there is provided the use of a compound of the formula (I), or a pharmaceutically acceptable salt or an in vivo hydrolysable thereof, as defined hereinbefore in the manufacture of a medicament for use in the production of an Eg5 inhibitory effect in a warm-blooded animal such as man.

According to a further aspect of section 3 of the invention there is provided the use of a compound of the formula (I), or a pharmaceutically acceptable salt or an in vivo hydrolysable thereof, as defined hereinbefore in the manufacture of a medicament for use in the production of an anti-proliferative effect in a warm-blooded animal such as man.

According to this aspect of section 3 of the invention there is provided the use of a compound of the formula (I), or a pharmaceutically acceptable salt or an in vivo hydrolysable thereof, as defined hereinbefore in the manufacture of a medicament for use in the production of an anti-cancer effect in a warm-blooded animal such as man.

According to a further feature of section 3 of the invention, there is provided the use of a compound of the formula (I), or a pharmaceutically acceptable salt or an in vivo hydrolysable thereof, as defined herein before in the manufacture of a medicament for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma.

In an additional embodiment of section 3, the present invention provides the use of a compound of formula (I) or a pharmaceutically acceptable salt or an in vivo hydrolysable ester thereof, in the manufacture of a medicament for the treatment or prophylaxis of disorders associated with cancer.

According to a further feature of this aspect of section 3 of the invention there is provided a method for producing an Eg5 inhibitory effect in a warm-blooded animal, such as man, in need of such treatment which comprises administering to said animal an effective amount of a compound of formula (I), or a pharmaceutically acceptable salt or an in vivo hydrolysable thereof, as defined above.

According to a further feature of this aspect of section 3 of the invention there is provided a method of producing an anti-proliferative effect in a warm-blooded animal, such as man, in need of such treatment which comprises administering to said animal an effective amount of a compound of formula (I), or a pharmaceutically acceptable salt or an in vivo hydrolysable thereof, as defined above.

According to a further feature of this aspect of section 3 of the invention there is provided a method for producing an anti-cancer effect in a warm-blooded animal, such as man, in need of such treatment which comprises administering to said animal an effective amount of a compound of formula (I), or a pharmaceutically acceptable salt or an in vivo hydrolysable thereof, as defined above.

In an additional embodiment of section 3, the present invention provides a method for the prophylaxis treatment of cancers associated with comprising administering to a human in need of such treatment a therapeutically effective amount of a compound of formula (I).

In a further embodiment of section 3 the present invention provides a method for the prophylaxis treatment of cancers associated with comprising administering to a human in need of such treatment a therapeutically effective amount of a compound of formula (I) or a pharmaceutically acceptable salt or an in vivo hydrolysable thereof.

In an additional embodiment of section 3, the present invention provides a method of producing a cell cycle inhibitory (anti-cell-proliferation) effect in a warm-blooded animal, such as man, in need of such treatment with comprises administering to said animal an effective amount of a compound of formula (I).

In a further embodiment of section 3 the present invention provides a method of producing a cell cycle inhibitory (anti-cell-proliferation) effect in a warm-blooded animal, such as man, in need of such treatment with comprises administering to said animal an effective amount of a compound of formula (I) or a pharmaceutically acceptable salt or an in vivo hydrolysable thereof.

In an additional embodiment of section 3, the present invention provides a method for the treatment of cancer comprising administering to a human a therapeutically effective amount of a compound of formula (I) or a pharmaceutically acceptable salt or an in vivo hydrolysable ester thereof.

In a further embodiment of section 3 the present invention provides a method for the treatment of cancer comprising administering to a human a therapeutically effective amount of a compound of formula (I) or a pharmaceutically acceptable salt or an in vivo hydrolysable thereof.

In an additional embodiment of section 3, the present invention provides a method for the treatment of breast cancer, colorectal cancer, ovarian cancer, lung (non small cell) cancer, malignant brain tumors, sarcomas, melanoma and lymphoma by administring a compound of formula (I) or a pharmaceutically acceptable salt or an in vivo hydrolysable ester thereof.

In a further embodiment of section 3 the present invention provides a method for the treatment of breast cancer, colorectal cancer, ovarian cancer, lung (non small cell) cancer, malignant brain tumors, sarcomas, melanoma and lymphoma by administering a compound of formula (I) or a pharmaceutically acceptable salt or an in vivo hydrolysable thereof.

According to an additional feature of this aspect of section 3 of the invention there is provided a method of treating carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma, in a warm-blooded animal, such as man, in need of such treatment which comprises administering to said animal an effective amount of a compound of formula (I) or a pharmaceutically acceptable salt or an in vivo hydrolysable thereof as defined herein before.

In an additional embodiment of section 3, the present invention provides a method for the treatment of cancer by administering to a human a compound of formula (I) or a pharmaceutically acceptable salt or an in vivo hydrolysable ester thereof and an anti-tumor agent.

In an additional embodiment of section 3, the present invention provides a pharmaceutical composition comprising a compound of formula (I) or a pharmaceutically acceptable salt or an in vivo hydrolysable ester thereof together with at least one pharmaceutically acceptable carrier, diluent or excipient.

In a further aspect of section 3 of the invention there is provided a pharmaceutical composition which comprises a compound of the formula (I), or a pharmaceutically acceptable salt or an in vivo hydrolysable thereof, as defined herein before in association with a pharmaceutically-acceptable diluent or carrier for use in the production of an Eg5 inhibitory effect in a warm-blooded animal such as man.

In a further aspect of section 3 of the invention there is provided a pharmaceutical composition which comprises a compound of the formula (I), or a pharmaceutically acceptable salt or an in vivo hydrolysable thereof, as defined herein before in association with a pharmaceutically-acceptable diluent or carrier for use in the production of an anti-proliferative effect in a warm-blooded animal such as man.

In a further aspect of section 3 of the invention there is provided a pharmaceutical composition which comprises a compound of the formula (I), or a pharmaceutically acceptable salt or an in vivo hydrolysable thereof, as defined herein before in association with a pharmaceutically-acceptable diluent or carrier for use in the production of an anti-cancer effect in a warm-blooded animal such as man.

In a further aspect of section 3 of the invention there is provided a pharmaceutical composition which comprises a compound of the formula (I), or a pharmaceutically acceptable salt or an in vivo hydrolysable thereof, as defined herein before in association with a pharmaceutically-acceptable diluent or carrier for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma in a warm-blooded animal such as man.

According to a further aspect of section 3 of the invention there is provided the use of a compound of the formula (I), or a pharmaceutically acceptable salt or an in vivo hydrolysable thereof, as defined hereinbefore in the production of an Eg5 inhibitory effect in a warm-blooded animal such as man.

According to a further aspect of section 3 of the invention there is provided the use of a compound of the formula (I), or a pharmaceutically acceptable salt or an in vivo hydrolysable thereof, as defined hereinbefore for use in the production of an anti-proliferative effect in a warm-blooded animal such as man.

According to this aspect of section 3 of the invention there is provided the use of a compound of the formula (I), or a pharmaceutically acceptable salt or an in vivo hydrolysable thereof, as defined hereinbefore for use in the production of an anti-cancer effect in a warm-blooded animal such as man.

According to a further feature of section 3 of the invention, there is provided the use of a compound of the formula (I), or a pharmaceutically acceptable salt or an in vivo hydrolysable thereof, as defined herein before for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma.

In a further embodiment of section 3, the present invention provides the use of a compound of formula (I) or a pharmaceutically acceptable salt or an in vivo hydrolysable thereof, for the treatment or prophylaxis of disorders associated with cancer.

In section 3, the definitions set forth in this section are intended to clarify terms used throughout this application. The term “herein” means the entire application.

In section 3, the term “Cₘ₋ₙ” or “Cₘ₋ₙ group” used alone or as a prefix, refers to any group having m to n carbon atoms.

In section 3, the term “hydrocarbon” used alone or as a suffix or prefix, refers to any structure comprising only carbon and hydrogen atoms up to 14 carbon atoms.

In section 3, the term “hydrocarbon radical” used alone or as a suffix or prefix, refers to any structure as a result of removing one or more hydrogens from a hydrocarbon.

In section 3, the term “alkyl” used alone or as a suffix or prefix, refers to monovalent straight or branched chain hydrocarbon radicals comprising, unless otherwise indicated, 1 to about 12 carbon atoms. Unless otherwise specified in section 3, “alkyl” includes both saturated alkyl and unsaturated alkyl. Particularly “alkyl” in section 3 refers to saturated alkyl. Particularly “C₁₋₃alkyl” in section 3 refers to methyl, ethyl, propyl or isopropyl.

In section 3, the term “five-membered” used as prefix refers to a group having a ring that contains five ring atoms.

In section 3, the term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, in section 3 a “phenyl substituted by nitro” refers to nitrophenyl.

In section 3, “RT” or “rt” means room temperature.

In section 3, when any variable (e.g., R¹, R⁴ etc.) occurs more than one time in any constituent or formula for a compound, its definition at each occurrence is independent of its definition at every other occurrence. Thus, in section 3 for example, if a group is shown to be substituted with 0-3 R¹, then said group may optionally be substituted with 0, 1, 2 or 3 R¹ groups and R¹ at each occurrence is selected independently from the definition of R¹. Also in section 3, combinations of substituents and/or variables are permissible only if such combinations result in stable compounds.

In section 3, when a bond to a substituent is shown to cross a bond connecting two atoms in a ring, then such substituent may be bonded to any atom on the ring. In section 3, when a substituent is listed without indicating the atom via which such substituent is bonded to the rest of the compound of a given formula, then such substituent may be bonded via any atom in such substituent. Combinations of substituents and/or variables are permissible in section 3 only if such combinations result in stable compounds.

In section 3, as used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In section 3, as used herein, “pharmaceutically acceptable salts” refer to derivatives of the disclosed compounds wherein the parent compound is modified by making acid or base salts thereof. Examples of pharmaceutically acceptable salts in section 3 include, but are not limited to, mineral or organic acid salts of basic residues such as amines; alkali or organic salts of acidic residues such as carboxylic acids; and the like. The pharmaceutically acceptable salts in section 3 include the conventional non-toxic salts or the quaternary ammonium salts of the parent compound formed, for example, from non-toxic inorganic or organic acids. For example in section 3, such conventional non-toxic salts include those derived from inorganic acids such as hydrochloric, phosphoric, and the like; and the salts prepared from organic acids such as lactic, maleic, citric, benzoic, methanesulfonic, and the like. The pharmaceutically acceptable salts of section 3 of the invention also include salts prepared with one of the following acids benzene sulfonic acid, fumaric acid, methanesulfonic acid, naphthalene-1,5-disulfonic acid, naphthalene-2-sulfonic acid or L-tartaric acid.

Thus in one aspect of section 3 of the invention there is provided a compound of the invention, particularly one of the Examples described herein, as a pharmaceutically acceptable salt, particularly a benzene sulfonic acid, fumaric acid, methanesulfonic acid, naphthalene-1,5-disulfonic acid, naphthalene-2-sulfonic acid or L-tartaric acid salt.

In section 3, the pharmaceutically acceptable salts of the present invention can be synthesized from the parent compound that contains a basic or acidic moiety by conventional chemical methods. Generally, such salts of section 3 can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

In section 3, as used herein, “in vivo hydrolysable ester” means an in vivo hydrolysable (or cleavable) ester of a compound of the formula (I) that contains a carboxy or a hydroxy group. For example amino acid esters, C₁₋₆alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

In section 3, all chemical names were generated using a software system known as AutoNom Name accessed through ISIS draw.

Section 3: Combinations

The anti-cancer treatment defined in section 3 may be applied as a sole therapy or may involve, in addition to the compound of the invention, conventional surgery or radiotherapy or chemotherapy. In section 3, such chemotherapy may include one or more of the following categories of anti-tumour agents:
(i) antiproliferative/antineoplastic drugs and combinations thereof, as used in medical oncology, such as alkylating agents (for example cis-platin, carboplatin, oxaliplatin, cyclophosphamide, nitrogen mustard, melphalan, chlorambucil, busulphan, temozolomide and nitrosoureas); antimetabolites (for example gemcitabine and antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere) polokinase inhibitors; and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function or inhibitors of SRC kinase (like 4-(6-chloro-2,3-methylenedioxyanilino)-7-[2-(4-methylpiperazin-1-yl)ethoxy]-5-tetrahydropyran-4-yloxyqyuinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661)) or antibodies to Heparanase);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [Erbitux, C225]), Ras/Raf signalling inhibitors such as farnesyl transferase inhibitors (for example sorafenib (BAY 43-9006) and tipifarnib), tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), for example inhibitors of the platelet-derived growth factor family such as imatinib, and for example inhibitors of the hepatocyte growth factor family, c-kit inhibitors, abl kinase inhibitors, IGF receptor (insulin-like growth factor) kinase inhibitors and inhibitors of cell signalling through MEK, AKT and/or P13K kinases;
(v) antiangiogenic agents such as those which inhibit the effects of vascular endothelial growth factor, (for example the anti-vascular endothelial cell growth factor antibody bevacizumab [Avastin™], and VEGF receptor tyrosine kinase inhibitors such as those disclosed in International Patent Applications WO 97/22596, WO 97/30035, WO 97/32856, WO 98/13354, 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814)) and compounds that work by other mechanisms (for example linomide, inhibitors of integrin αvβ3 function and angiostatin), ang1 and 2 inhibitors;
(vi) vascular damaging agents such as Combretastatin A4 and compounds disclosed in International Patent Applications WO 99/02166, WO 00/40529, WO 00/41669, WO 01/92224, WO 02/04434 and WO 02/08213, anti bcl2;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;
x) cell cycle agents such as aurora kinase inhibitors (for example PH739358, VX-680, MLN8054, R763, MP235, MP529, VX-528, AX39459 and the specific examples mentioned in WO02/00649, WO03/055491, WO2004/058752, WO2004/058781, WO2004/058782, WO2004/094410, WO2004/105764, WO2004/113324 which are incorporated herein by reference), and cyclin dependent kinase inhibitors such as CDK2 and/or CDK4 inhibitors (for example the specific examples of WO01/14375, WO01/72717, WO02/04429, WO02/20512, WO02/66481, WO02/096887, WO03/076435, WO03/076436, WO03/076434, WO03/076433, WO04/101549 and WO04/101564 which are incorporated herein by reference); and
xi) cytotoxic agents such as gemcitibine, topoisomerase 1 inhibitors (adriamycin, etoposide) and topoisomerase II inhibitors.

In section 3, such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In a further aspect of section 3 of the present invention there is provided a compound of formula (I) or a pharmaceutically acceptable salt or an in vivo hydrolysable ester thereof in combination with simultaneous, sequential or separate dosing of an anti-tumor agent or class selected from the list herein above.

Therefore in a further embodiment of section 3 the present invention provides a method for the treatment of cancer by administering to a human a compound of formula (I) or a pharmaceutically acceptable salt or an in vivo hydrolysable ester thereof in combination with simultaneous, sequential or separate dosing of an anti-tumor agent or class selected from the list herein above.

In a further aspect of section 3 of the present invention there is provided the use of a compound of formula (I) or a pharmaceutically acceptable salt or an in vivo hydrolysable ester thereof in combination with simultaneous, sequential or separate dosing of an anti-tumor agent or class selected from the list herein above for use in the manufacture of a medicament for use in the treatment of cancer.

In a further aspect of section 3 of the present invention there is provided the use of a compound of formula (I) or a pharmaceutically acceptable salt or an in vivo hydrolysable ester thereof in combination with simultaneous, sequential or separate dosing of an anti-tumor agent or class selected from the list herein above for use in the treatment of cancer.

The anti-cancer treatment defined in section 3 may also include one or more of the following categories of pharmaceutical agents:
i) an agent useful in the treatment of anemia, for example, a continuous eythropoiesis receptor activator (such as epoetin alfa);
ii) an agent useful in the treatment of neutropenia, for example, a hematopoietic growth factor which regulates the production and function of neutrophils such as a human granulocyte colony stimulating factor, (G-CSF), for example filgrastim; and
iii) an anti-emetic agent to treat nausea or emesis, including acute, delayed, late-phase, and anticipatory emesis, which may result from the use of a compound of the present invention, alone or with radiation therapy, suitable examples of such anti emetic agents include neurokinin-1 receptor antagonists, 5H13 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid or Benecorten, an antidopaminergic, such as the phenothiazines (for example prochlorperazine, fluphenazine, thioridazine and mesoridazine), metoclopramide or dronabinol.

In section 3, such conjoint treatment may be achieved by way of the simultaneous, sequential or separate dosing of the individual components of the treatment. Such conjoint treatment employs the compounds of this invention within the dosage range described hereinbefore and the other pharmaceutically-active agent within its approved dosage range.

In a further aspect of section 3 of the present invention there is provided a compound of formula (I) or a pharmaceutically acceptable salt or an in vivo hydrolysable ester thereof in combination with simultaneous, sequential or separate dosing of another pharmaceutical agent or class selected from the list herein above.

Therefore in a further embodiment of section 3 the present invention provides a method for the treatment of cancer by administering to a human a compound of formula (I) or a pharmaceutically acceptable salt or an in vivo hydrolysable ester thereof in combination with simultaneous, sequential or separate dosing of another pharmaceutical agent or class selected from the list herein above.

In a further aspect of section 3 of the present invention there is provided the use of a compound of formula (I) or a pharmaceutically acceptable salt or an in vivo hydrolysable ester thereof in combination with simultaneous, sequential or separate dosing of another pharmaceutical agent or class selected from the list herein above for use in the manufacture of a medicament for use in the treatment of cancer.

In a further aspect of section 3 of the present invention there is provided the use of a compound of formula (I) or a pharmaceutically acceptable salt or an in vivo hydrolysable ester thereof in combination with simultaneous, sequential or separate dosing of another pharmaceutical agent or class selected from the list herein above for use in the treatment of cancer.

In addition to their use in therapeutic medicine, the compounds of formula (I) and their pharmaceutically acceptable salts of section 3 are also useful as pharmacological tools in the development and standardisation of in vitro and in vivo test systems for the evaluation of the effects of inhibitors of Eg5 in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described in section 3 also apply.

Section 3: Formulations

In section 3, compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

In section 3, the dosage will depend on the route of administration, the severity of the disease, age and weight of the patient and other factors normally considered by the attending physician, when determining the individual regimen and dosage level as the most appropriate for a particular patient.

In section 3, an effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

For preparing pharmaceutical compositions from the compounds of section 3 of this invention, inert, pharmaceutically acceptable carriers can be either solid or liquid. Solid form preparations include powders, tablets, dispersible granules, capsules, cachets, and suppositories.

In section 3, a solid carrier can be one or more substances, which may also act as diluents, flavoring agents, solubilizers, lubricants, suspending agents, binders, or tablet disintegrating agents; it can also be an encapsulating material.

In section 3, in powders, the carrier is a finely divided solid, which is in a mixture with the finely divided active component. In section 3, in tablets, the active component is mixed with the carrier having the necessary binding properties in suitable proportions and compacted in the shape and size desired.

In section 3, for preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

In section 3, suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In section 3, some of the compounds of the present invention are capable of forming salts with various inorganic and organic acids and bases and such salts are also within the scope of this invention. Examples of such acid addition salts in section 3 include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts in section 3 include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also in section 3, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred in section 3, although other salts are also useful, such as in isolating or purifying the product.

The salts of section 3 may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In section 3, in order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In section 3, in addition to the compounds of the present invention, the pharmaceutical composition of this invention may also contain, or be co-administered (simultaneously or sequentially) with, one or more pharmacological agents of value in treating one or more disease conditions referred to herein.

In section 3, the term composition is intended to include the formulation of the active component or a pharmaceutically acceptable salt with a pharmaceutically acceptable carrier. For example section 3 of this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

In section 3, liquid form compositions include solutions, suspensions, and emulsions. Sterile water or water-propylene glycol solutions of the active compounds of section 3 may be mentioned as an example of liquid preparations suitable for parenteral administration. In section 3, liquid compositions can also be formulated in solution in aqueous polyethylene glycol solution. In section 3, aqueous solutions for oral administration can be prepared by dissolving the active component in water and adding suitable colorants, flavoring agents, stabilizers, and thickening agents as desired. In section 3, aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

In section 3, the pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Section 3: Synthesis

The compounds of section 3 of the present invention can be prepared in a number of ways well known to one skilled in the art of organic synthesis. The compounds of section 3 of the present invention can be synthesized using the methods described below, together with synthetic methods known in the art of synthetic organic chemistry, or variations thereon as appreciated by those skilled in the art. Such methods include, but are not limited to, those described below in section 3. All references cited in section 3 are hereby incorporated in their entirety by reference.

The novel compounds of section 3 of this invention may be prepared using the reactions and techniques described herein. In section 3, the reactions are performed in solvents appropriate to the reagents and materials employed and are suitable for the transformations being effected. Also, in the description of the synthetic methods described below within section 3, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. In section 3, it is understood by one skilled in the art of organic synthesis that the functionality present on various portions of the molecule must be compatible with the reagents and reactions proposed. In section 3, such restrictions to the substituents, which are compatible with the reaction conditions, will be readily apparent to one skilled in the art and alternate methods must then be used.

In section 3, the starting materials for the Examples contained herein are either commercially available or are readily prepared by standard methods from known materials. For example the following reactions are illustrations but not limitations of the preparation of some of the starting materials and examples used in section 3.

In section 3, all chiral purifications to separate the respective enantiomers were carried out using a Chiralpak AD column (dimensions 250×20 mm, 10μ column) with a flow rate of 20 m/min unless otherwise stated. Approximate elution times may vary depending on the concentration of compound loaded. Chiral purification generally resulted in 99% purity of the (+) enantiomer.

In section 3, the signal refers to the direction of rotation of polarized light at 670 nm as measured by an Advanced Laser Polarimeter (PDR-Chiral, Inc., Lake Park, Fla.) at ambient temperature in the solvent composition indicated (reference Liu Y. S., Yu T., Armstrong D. W., LC-GC 17 (1999), 946-957).

### SECTION 3: EXAMPLES

Section 3 of the invention will now be illustrated by the following non limiting examples in which, unless stated otherwise:
(i) temperatures are given in degrees Celsius (° C.); operations were carried out at room or ambient temperature, that is, at a temperature in the range of 18-30° C.;
(ii) organic solutions were dried over anhydrous sodium sulphate; evaporation of solvent was carried out using a rotary evaporator under reduced pressure (6004000 Pascals; 4.5-30 mmHg) with a bath temperature of up to 60° C.;
(iii) in general, the course of reactions was followed by TLC or MS and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz using deuterated chloroform (CDCl₃) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (E1), fast atom bombardment (FAB); electrospray (ESP); or atmospheric pressure chemical ionization (APCI); values for m/z are given; generally, only ions which indicate the parent mass are reported;
(x) where a synthesis is described as being analogous to that described in a previous example the amounts used are the millimolar ratio equivalents to those used in the previous example;
(xi) the following abbreviations have been used:
   THF tetrahydrofuran;
   DMF N,N-dimethylformamide;
   EtOAc ethyl acetate;
   DCM dichloromethane; and
   DMSO dimethylsulphoxide; and
(xii) a Vigreux column is a glass tube with a series of indentations such that alternate sets of indentations point downward at an angle of 45 degree in order to promote the redistribution of liquid from the walls to the center of the column; The Vigreux column used herein is 150 mm long (between indents) with a 20 mm diameter and it was manufactured by Lab Glass.
   Section 3: Method 1

### 2-(1-Ethoxy-ethylidene)-malononitrile

Triethyl orthoacetate (97 g, 0.6 mol), malononitrile (33 g, 0.5 mol) and glacial acetic acid (1.5 g) were placed in a 1 L flask equipped with a stirrer, thermometer and a Vigreux column (20×1 in.) on top of which a distillation condenser was placed. The reaction mixture was heated and ethyl alcohol began to distill when the temperature of the reaction mixture was about 85-90° C. After about 40 min., the temperature of the reaction mixture reached 140° C. Then the reaction was concentrated in a rotary evaporator to remove the low-boiling materials and the residue was crystallized from absolute alcohol to yield the pure product (62.2 g, 91%) as a light yellow solid mp 91.6° C.

Section 3: Method 2

### (2E)-2-Cyano-3-ethoxybut-2-enethioamide

2-(1-Ethoxy-ethylidene)-malononitrile (Section 3: Method 1) (62 g, 0.45 mol) was dissolved in anhydrous benzene (800 mL) and 1 mL of triethylamine was added as catalyst. The mixture was stirred and hydrogen sulfide was bubbled into this solution for 40 min and a solid formed. The precipitated solid was filtered off and dried. The solid was recrystallized from absolute alcohol (100 mL) filtered and dried to isolate the pure (2E)-2-cyano-3-ethoxybut-2-enethioamide (19.3 g, 25%) as light brown crystals.

Section 3: Method 3

### (2E)-3-Amino-2-cyanobut-2-enethioamide

(2E)-2-Cyano-3-ethoxybut-2-enethioamide (Section 3: Method 2) (19.2 g, 0.136 mol) was dissolved in a saturated solution of ammonia in methanol (500 mL) and stirred at r.t. overnight. The reaction mixture was concentrated and the residue was dissolved in hot water (600 mL) and the undissoved solid was filtered and dried to recover 6 g of the starting thiocrotonamide. The aqueous solution on standing overnight provided the pure (2E)-3-amino-2-cyanobut-2-enethioamide (6.85 g, 63%) as off-white crystals. ¹H NMR (300 MHz, DMSO-d₆) δ 2.22 (s, 3H), 7.73 (bs, 1H), 8.53 (bs, 1H), 9.01 (bs, 1H), 11.60 (bs, 1H).

Section 3: Method 4

### 5-Amino-3-methylisothiazole-4-carbonitrile

To a stirred solution of (2E)-3-amino-2-cyanobut-2-enethioamide (Section 3: Method 3) (6.83 g, 48.4 mmol) in methanol (300 mL) was added dropwise 13.6 mL (124 mmol.) of 30% hydrogen peroxide. The mixture was stirred at 60° C. for 4 h and evaporated to 60 mL in a rotary evaporator and cooled in an ice-bath. The crystallized product was filtered off and recrystallized from EtOAc to provide the pure product 5-amino-3-methylisothiazole-4-carbonitrile (5.41 g, 80%) as a white crystalline solid. ¹H NMR (300 MHz, DMSO-d6) δ 2.24 (s, 3H), 8.00 (bs, 2H).

Section 3: Method 5

### N-(4-Cyano-3-methyl-isothiazol-5-yl)-butyramide

To a solution of 5-amino-3-methylisothiazole-4-carbonitrile (Section 3: Method 4) (5.31 g, 38.2 mmol) in DCM (200 mL) at 0° C., NEt₃ (5 g, 50 mmol) was added followed by the dropwise addition of a solution of the butyryl chloride (4.88 g, 45.8 mmol) in DCM (50 mL). After the completion of the addition the reaction mixture was allowed to warm to r.t. and stirred overnight. The reaction mixture was washed with water (100 mL), 1N HCl (100 mL), brine (200 mL) and dried over Na₂SO₄. Concentration of the DCM layer provided the crude product which was triturated from DCM/hexanes (1/10) and filtered off to isolate the pure N-(4-cyano-3-methyl-isothiazol-5-yl)-butyramide (7.57 g, 95%) as an orange solid.

Section 3: Method 6

### 5-Butyrylamino-3-methyl-isothiazole-4-carboxylic acid amide

To a solution of N-(4-cyano-3-methyl-isothiazol-5-yl)-butyramide (Section 3: Method 5) (4.18 g, 20 mmol) in 30% aqueous NH₄OH (250 mL), was added dropwise 100 mL of hydrogen peroxide at r.t. After the completion of the addition the reaction mixture was stirred at 60° C. overnight after which the TLC showed the complete disappearance of SM. The reaction mixture was cooled and extracted with chloroform (3×100 mL). The organic layer was dried (Na₂SO₄) and concentrated to get the pure 5-butyrylamino-3-methyl-isothiazole-4-carboxylic acid amide (2.9 g, 72%) as a white solid. ¹H NMR (300 MHz) δ 1.03 (t, 3H), 1.79 (m, 2H), 2.54 (t, 3H), 2.69 (s, 3H), 5.97 (bs, 2H), 11.78 (bs, 1H).

Section 3: Method 7

### 3-Methyl-6-propyl-5H-isothiazolo[5,4-d]pyrimidin-4-one

5-Butyrylamino-3-methyl-isothiazole-4-carboxylic acid amide (Section 3: Method 6) (1.9 g, 8.3 mmol) was suspended in 75 mL of 30% NH₃ and then was heated to 140° C. for 4 h in a pressure reactor. The mixture was cooled and neutralized to pH 8. The precipitated 3-methyl-6-propyl-5H-isothiazolo[5,4-d]pyrimidin-4-one was filtered off, washed with water (100 mL) and dried in vacuum oven at 40° C. overnight to get 800 mg (34%) of pure product. ¹H NMR (300 MHz) δ 1.03 (t, 3H), 1.74 (m, 2H), 2.67 (t, 3H), 2.78 (s, 3H).

Section 3: Method 8

### 5-Benzyl-3-methyl-6-propyl-5H-isothiazolo[5,4-d]pyrimidin-4-one

To a solution of 3-methyl-6-propyl-5H-isothiazolo[5,4-d]pyrimidin-4-one (Section 3: Method 7) (800 mg, 3.8 mmol) in 20 mL of anhydrous DMF was added 1.38 g (10 mmol) of anhydrous K₂CO₃ followed by benzyl bromide (655 mg, 3.8 mmol) and the mixture was stirred at room temperature overnight. The TLC of the reaction mixture showed the complete disappearance of the SM. The reaction mixture was poured into ice cold water and extracted with EtOAc (3×100 mL). The combined extracts were washed with water (100 mL), brine (100 mL), dried (Na₂SO₄) and concentrated. The TLC and the ¹H NMR showed the presence of two products N alkylated as well as O-alkylated products in a ratio of 1:1. The products were separated by column (silica gel, 116 g) chromatography using 10-20% EtOAc in hexanes. The desired N-alkylated product 5-benzyl-3-methyl-6-propyl-5H-isothiazolo[5,4-d]pyrimidin-4-one was isolated as white crystalline solid (369 mg, 32%). ¹H NMR (300 MHz) δ 0.96 (t, 3H), 1.71-1.84 (m, 2H), 2.73 (t, 3H), 2.81 (s, 3H), 5.38 (s, 2H), 7.14-7.38 (m, 5H).

Section 3: Methods 8a-8b

The following compounds were synthesized according to Section 3: Method 8:

| Section 3: | | | Alkylating |
|---|---|---|---|
| Method # | Compound Name | m/z | agent |
| 8a | 5-(4-Fluoro-benzyl)-3-methyl-6- | 318 | 4-fluorobenzyl |
| | propyl-5H-isothiazolo[5,4- | (MH⁺) | bromide |
| | d]pyrimidin-4-one | | |
| 8b | 5-(3-Fluoro-benzyl)-3-methyl-6- | 318 | 3-fluorobenzyl |
| | propyl-5H-isothiazolo[5,4- | (MH⁺) | bromide |
| | d]pyrimidin-4-one | | |

Section 3: Method 9

### 5-Benzyl-6-(1-bromo-propyl)-3-methyl-5H-isothiazolo[5,4-d]pyrimidin-4-one

To a solution of 5-benzyl-3-methyl-6-propyl-5H-isothiazolo[5,4-d]pyrimidin-4-one (Section 3: Method 8) (369 mg, 1.23 mmol) and sodium acetate (1 g) in acetic acid (5 mL) at 100° C., a solution of the bromine (318 mg, 2 mmol) in acetic acid (10 mL) was added dropwise over a period of 20 minutes. The reaction mixture was cooled after the addition and the TLC (eluent 10% EtOAc in hexanes) and MS showed the complete disappearance of the SM and only the product. The reaction mixture was poured into ice water and extracted with EtOAc (3×60 mL) and the organic layers were combined and washed with 2% sodium thiosulfate solution (60 mL), water (100 mL), brine (100 mL) and dried over Na₂SO₄. Concentration of the organic layer provided the pure 5-benzyl-6-(1-bromo-propyl)-3-methyl-5H-isothiazolo[5,4-d]pyrimidin-4-one, (460 mg, 100%) as white crystalline solid. ¹H NMR (300 MHz) δ 0.76 (t, 3H), 2:1-2.47 (m, 2H), 2.84 (s, 3H), 4.62 (t, 1H), 4.88 (d, 1H), 6.20 (d, 1H), 7.10-7.40 (m, 5H).

Section 3: Methods 9a-9b

The following compounds were synthesized according to Section 3: Method 9:

| Section 3: | | | |
|---|---|---|---|
| Method # | Compound Name | m/z | SM |
| 9a | 6-(1-bromopropyl)-5-[(4- | 396, 398 | Section 3: |
| | fluorophenyl)methyl]-3-methyl- | (MH⁺) | Method 8a |
| | isothiazolo[5,4-d]pyrimidin- | | |
| | 4(5H)-one | | |
| 9b | 6-(1-bromopropyl)-5-[(3- | 396, 398 | Section 3: |
| | fluorophenyl)methyl]-3-methyl- | (MH⁺) | Method 8b |
| | isothiazolo[5,4-d]pyrimidin- | | |
| | 4(5H)-one | | |

Section 3: Method 10

### {3-[1-(5-Benzyl-3-methyl-4-oxo-4,5-dihydro-isothiazolo[5,4-d]pyrimidin-6-yl)-propylamino]propyl}-carbamic acid tert-butyl ester

To a solution of 5-benzyl-6-(1-bromo-propyl)-3-methyl-5H-isothiazolo[5,4-d]pyrimidin-4-one (Section 3: Method 9) (0.46 g, 1.22 mmol) in anhydrous ethanol (20 mL), was added tert-butyl 3-aminopropyl-carbamate (0.211 g, 1.22 mmol) followed by the addition of anhydrous diisopropylethylamine (0.258 g, 2 mmol) and the mixture was stirred at reflux for 16 hours. The TLC of the RM showed the complete disappearance of the starting bromide. The reaction mixture was poured into ice water (200 mL) and extracted with EtOAc (3×100 mL). The organic layer was washed with water (100 mL), brine (100 mL) and dried (Na₂SO₄). Concentration of the organic layer provided the crude product which was purified by column (silica gel) chromatography using 30-50% EtOAc in hexanes to isolate the pure amine {3-[1-(5-benzyl-3-methyl-4-oxo-4,5-dihydro-isothiazolo[5,4-d]pyrimidin-6-yl)-propylamino]-propyl}-carbamic acid tert-butyl ester (0.1 g, 17%) as a white foam. ¹H NMR (300 MHz) δ 0.95 (t, 3H), 1.33 (t, 2H), 1.42 (s, 9H), 1.49-1.51 (m, 2H), 1.87-1.99 (m, 1H), 2.35-2.45 (m, 1H), 2.83 (s, 3H), 2.92-3.20 (m, 2H), 3.64-3.70 (m, 1H), 4.98 (d, 1H), 5.17 (bs, 1H), 5.85 (d, 1H), 7.10-7.40 (m, 5H).

Section 3: Methods 10a-10d

The following compounds were synthesized according to Section 3: Method 10:

| Section 3: | | | | |
|---|---|---|---|---|
| Method # | Compound Name | m/z | SM | Amine |
| 10a | {3-({1-[5-(4-fluorobenzyl)-3-methyl-4- | 490 | Section 3: | tert-butyl 3- |
| | oxo-4,5-dihydro-isothiazolo[5,4- | (MH⁺) | Method 9a | aminopropyl- |
| | d]pyrimidin-6-yl]-propyl}amino)- | | | carbamate |
| | propyl}-carbamic acid tert-butyl ester | | | |
| 10b | {3-({1-[5-(3-fluorobenzyl)-3-methyl-4- | 490 | Section 3: | tert-butyl 3- |
| | oxo-4,5-dihydro-isothiazolo[5,4- | (MH⁺) | Method 9b | aminopropyl- |
| | d]pyrimidin-6-yl]-propyl}amino)- | | | carbamate |
| | propyl}-carbamic acid tert-butyl ester | | | |
| 10c | 5-Benzyl-6-[1-(3-dimethylamino- | 400 | Section 3: | N,N- |
| | propylamino)-propyl]-3-methyl-5H- | (MH⁺) | Method 9 | Dimethylpropane- |
| | isothiazolo[5,4-d]pyrimidin-4-one | | | 1,3-diamine |
| 10d | {2-[1-(5-Benzyl-3-methyl-4-oxo-4,5- | 458 | Section 3: | (2-Amino- |
| | dihydro-isothiazolo[5,4-d]pyrimidin-6- | (MH⁺) | Method 9 | ethyl)-carbamic |
| | yl)-propylamino]-ethyl}-carbamic acid | | | acid tert-butyl |
| | tert-butyl ester | | | ester |

Section 3: Method 11

### {3-[[1-(5-Benzyl-3-methyl-4-oxo-4,5-dihydro-isothiazolo[5,4-d]pyrimidin-6-yl)-propyl]-(4-methyl-benzoyl)-amino]-propyl}-carbamic acid tert-butyl ester

To a solution of {3-[1-(5-benzyl-3-methyl-4-oxo-4,5-dihydro-isothiazolo[5,4-d]pyrimidin-6-yl)-propylamino]-propyl}-carbamic acid tert-butyl ester (Section 3: Method 10) (0.1 g, 0.21 mmol) and triethylamine (0.303 g, 3 mmol) in DCM (20 mL) at r.t. was added dropwise a solution of p-toluoyl chloride (0.1 g, 0.6 mmol) in DCM (10 mL). The resulting solution was stirred at r.t. for 30 min. after which the TLC showed the disappearance of the SM. The reaction mixture was diluted with DCM (60 mL) washed with satd. NaHCO₃ (100 mL), water (100 mL), brine (100 mL) and dried (Na₂SO₄). Concentration of the organic layer provided the crude product which was purified by column (silica gel) chromatography using 20-30% EtOAc in hexanes as eluent. Yield was 0.117 g (94%). m/z 590 (MH⁺).

Section 3: Methods 11a-11i

The following compounds were synthesized according to Section 3: Method 11:

| Section 3: | | | | |
|---|---|---|---|---|
| Method # | Compound Name | m/z | SM | Acylating agent |
| 11a | {3-[{1-[5-(4-Fluoro-benzyl)-3-methyl-4- | 608 | Section | 4-methyl-benzoyl |
| | oxo-4,5-dihydro-isothiazolo[5,4- | (MH⁺) | 3: | chloride |
| | d]pyrimidin-6-yl]-propyl}-(4-methyl- | | Method | |
| | benzoyl)-amino]-propyl}-carbamic acid | | 10a | |
| | tert-butyl ester | | | |
| 11b | {3-[{1-[5-(3-Fluoro-benzyl)-3-methyl-4- | 608 | Section | 4-methyl-benzoyl |
| | oxo-4,5-dihydro-isothiazolo[5,4- | (MH⁺) | 3: | chloride |
| | d]pyrimidin-6-yl]-propyl}-(4-methyl- | | Method | |
| | benzoyl)-amino]-propyl}-carbamic acid | | 10b | |
| | tert-butyl ester | | | |
| 11c | {3-[[1-(5-Benzyl-3-methyl-4-oxo-4,5- | 610 | Section | 4-chloro-benzoyl |
| | dihydro-isothiazolo[5,4-d]pyrimidin-6- | (MH⁺) | 3: | chloride |
| | yl)-propyl]-(4-chloro-benzoyl)-amino]- | | Method | |
| | propyl}-carbamic acid tert-butyl ester | | 10 | |
| 11d | {3-[[1-(5-Benzyl-3-methyl-4-oxo-4,5- | 608 | Section | 3-fluoro-4- |
| | dihydro-isothiazolo[5,4-d]pyrimidin-6- | (MH⁺) | 3: | methyl-benzoyl |
| | yl)-propyl]-(3-fluoro-4-methyl-benzoyl)- | | Method | chloride |
| | amino]-propyl}-carbamic acid tert-butyl | | 10 | |
| | ester | | | |
| 11e | {3-[[1-(5-Benzyl-3-methyl-4-oxo-4,5- | 644, | Section | 2,3-dichloro- |
| | dihydro-isothiazolo[5,4-d]pyrimidin-6- | 645, | 3: | benzoyl chloride |
| | yl)-propyl]-(2,3-dichloro-benzoyl)- | 646 | Method | |
| | amino]-propyl}-carbamic acid tert-butyl | (MH⁺) | 10 | |
| | ester | | | |
| 11f | (3-{(Benzo[b]thiophene-2-carbonyl)-[1- | 632 | Section | 1- |
| | (5-benzyl-3-methyl-4-oxo-4,5-dihydro- | (MH⁺) | 3: | benzothiophene- |
| | isothiazolo[5,4-d]pyrimidin-6-yl)-propyl]- | | Method | 2-carbonyl |
| | amino}-propyl)-carbamic acid tert-butyl | | 10 | chloride |
| | ester | | | |
| 11g | {3-[[1-(5-Benzyl-3-methyl-4-oxo-4,5- | 654, | Section | 4-bromo-benzoyl |
| | dihydro-isothiazolo[5,4-d]pyrimidin-6- | 656 | 3: | chloride |
| | yl)-propyl]-(4-bromo-benzoyl)-amino]- | (MH⁺) | Method | |
| | propyl}-carbamic acid tert-butyl ester | | 10 | |
| 11h | {2-[[1-(5-Benzyl-3-methyl-4-oxo-4,5- | 576 | Section | 4-methyl-benzoyl |
| | dihydro-isothiazolo[5,4-d]pyrimidin-6- | (MH⁺) | 3: | chloride |
| | yl)-propyl]-(4-methyl-benzoyl)-amino]- | | Method | |
| | ethyl}-carbamic acid tert-butyl ester | | 10d | |
| 11i | N-[1-(5-Benzyl-3-methyl-4-oxo-4,5- | 518 | Section | 4-methyl-benzoyl |
| | dihydro-isothiazolo[5,4-d]pyrimidin-6- | (MH⁺) | 3: | chloride |
| | yl)-propyl]-N-(3-dimethylamino-propyl)- | | Method | |
| | 4-methyl-benzamide | | 10c | |

Section 3: Method 12

### Chiral purification of (+) (3-[[1-(5-Benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester

100 mg of (+/−) {3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 11) were dissolved in 2:1 IPA:hexanes and the compound was purified using a Chiralpak AD, 250×20 mm, 10μ column with a flow rate of 20 ml/min with 80% hexane, 20% isopropanol (0.1% diethylamine) as eluent. Elution time:—10.42 min. Chiral purification generally resulted in 99% purity of the (+) enantiomer.

Section 3: Methods 12a-12I

The following compounds were chirally purified in same manner as (+) (3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 12):

| Section 3: | | Column | Solvent | (+) Enantiomer | |
|---|---|---|---|---|---|
| Method # | Compound Name | Type | composition | retention time | SM |
| 12a | (+) {3-[{1-[5-(4- | Chiralpak | 85% hexanes | 10.7 min | Section |
| | Fluoro-benzyl)-3- | AD | 15% isopropanol | | 3: |
| | methyl-4-oxo-4,5- | | 0.1% diethylamine | | Method |
| | dihydro- | | | | 11a |
| | isothiazolo[5,4- | | | | |
| | d]pyrimidin-6-yl]- | | | | |
| | propyl}-(4-methyl- | | | | |
| | benzoyl)-amino]- | | | | |
| | propyl}-carbamic | | | | |
| | acid tert-butyl ester | | | | |
| 12b | (+) {3-[{1-[5-(3- | Chiralpak | 75% hexanes | 7.6 min | Section |
| | Fluoro-benzyl)-3- | AD | 25% isopropanol | | 3: |
| | methyl-4-oxo-4,5- | | 0.1% diethylamine | | Method |
| | dihydro- | | | | 11b |
| | isothiazolo[5,4- | | | | |
| | d]pyrimidin-6-yl]- | | | | |
| | propyl}-(4-methyl- | | | | |
| | benzoyl)-amino]- | | | | |
| | propyl}-carbamic | | | | |
| | acid tert-butyl ester | | | | |
| 12c | (+) {3-[[1-(5- | Chiralpak | 80% hexanes | 10.8 min | Section |
| | Benzyl-3-methyl-4- | AD | 20% isopropanol | | 3: |
| | oxo-4,5-dihydro- | | 0.1% diethylamine | | Method |
| | isothiazolo[5,4- | | | | 11c |
| | d]pyrimidin-6-yl)- | | | | |
| | propyl]-(4-chloro- | | | | |
| | benzoyl)-amino]- | | | | |
| | propyl}-carbamic | | | | |
| | acid tert-butyl ester | | | | |
| 12d | (+) {3-[[1-(5- | Chiralpak | 80% hexanes | 8.6 min | Section |
| | Benzyl-3-methyl-4- | AD | 20% isopropanol | | 3: |
| | oxo-4,5-dihydro- | | 0.1% diethylamine | | Method |
| | isothiazolo[5,4- | | | | 11d |
| | d]pyrimidin-6-yl)- | | | | |
| | propyl]-(3-fluoro-4- | | | | |
| | methyl-benzoyl)- | | | | |
| | amino]-propyl}- | | | | |
| | carbamic acid tert- | | | | |
| | butyl ester | | | | |
| 12e | (+) {3-[[1-(5- | Chiralpak | 90% hexanes | 7.5 min | Section |
| | Benzyl-3-methyl-4- | OD | 5% methanol | | 3: |
| | oxo-4,5-dihydro- | | 5% ethanol | | Method |
| | isothiazolo[5,4- | | 0.1% diethylamine | | 11e |
| | d]pyrimidin-6-yl)- | | | | |
| | propyl]-(2,3- | | | | |
| | dichloro-benzoyl)- | | | | |
| | amino]-propyl}- | | | | |
| | carbamic acid tert- | | | | |
| | butyl ester | | | | |
| 12f | (+) (3- | Chiralpak | 50% hexanes | 7.2 min | Section |
| | {(Benzo[b]thiophene- | AD | 50% isopropanol | | 3: |
| | 2-carbonyl)-[1-(5- | | 0.1% diethylamine | | Method |
| | benzyl-3-methyl-4- | | | | 11f |
| | oxo-4,5-dihydro- | | | | |
| | isothiazolo[5,4- | | | | |
| | d]pyrimidin-6-yl)- | | | | |
| | propyl]-amino}- | | | | |
| | propyl)-carbamic | | | | |
| | acid tert-butyl ester | | | | |
| 12g | (+) {3-[[1-(5- | Chiralpak | 75% hexanes | 10.5 min | Section |
| | Benzyl-3-methyl-4- | AD | 25% isopropanol | | 3: |
| | oxo-4,5-dihydro- | | 0.1% diethylamine | | Method |
| | isothiazolo[5,4- | | | | 11g |
| | d]pyrimidin-6-yl)- | | | | |
| | propyl]-(4-bromo- | | | | |
| | benzoyl)-amino]- | | | | |
| | propyl}-carbamic | | | | |
| | acid tert-butyl ester | | | | |
| 12h | (+) {2-[[1-(5- | Chiralpak | 80% hexanes | 11.8 min | Section |
| | Benzyl-3-methyl-4- | AD | 20% isopropanol | | 3: |
| | oxo-4,5-dihydro- | | 0.1% diethylamine | | Method |
| | isothiazolo[5,4- | | | | 11h |
| | d]pyrimidin-6-yl)- | | | | |
| | propyl]-(4-methyl- | | | | |
| | benzoyl)-amino]- | | | | |
| | ethyl}-carbamic | | | | |
| | acid tert-butyl ester | | | | |
| 12i | (+) N-[1-(5-Benzyl- | Chiralpak | 90% hexanes | 9.5 min | Section |
| | 3-methyl-4-oxo-4,5- | AD | 10% isopropanol | | 3: |
| | dihydro- | | 0.1% diethylamine | | Method |
| | isothiazolo[5,4- | | | | 11i |
| | d]pyrimidin-6-yl)- | | | | |
| | propyl]-N-(3- | | | | |
| | dimethylamino- | | | | |
| | propyl)-4-methyl- | | | | |
| | benzamide | | | | |
| | Section 3: Example | | | | |
| | A-10 | | | | |

Chiral purification generally resulted in 99% purity of the (+) enantiomer.

Section 3: Method 13 and Section 3: Example A-1

### (+) N-(3-Amino-propyl)-N-[1-(5-benzyl-3-methyl-4-oxo-4,5-dihydro-isothiazolo[5,4-d]pyrimidin-6-yl)-propyl]-4-methyl-benzamide hydrogen chloride

(+) {3-[[1-(5-Benzyl-3-methyl-4-oxo-4,5-dihydro-isothiazolo[5,4-d]pyrimidin-6-yl)-propyl]-(4-methyl-benzoyl)-amino]-propyl}-carbamic acid tert-butyl ester (Section 3: Method 12) (0.117 g, 0.19 mmol) was dissolved in 2M HCl in ether and the mixture was stirred at r.t. for 20 h. The precipitated product was filtered off and washed with ether and dried in vacuo to yield the pure (+) N-(3-amino-propyl)-N-[1-(5-benzyl-3-methyl-4-oxo-4,5-dihydro-isothiazolo[5,4-d]pyrimidin-6-yl)-propyl]-4-methyl-benzamide chloride salt (91 mg, 87%). White powder, mp. 127.8-129.2° C. m/z 490 (MH⁺), ¹H NMR (DMSO-d₆, 500 MHz, 96° C.) δ: 0.63 (t, 3H), 1.40-1.74 (m, 2H), 1.75-1.96 (m, 1H), 2.05-2.20 (m, 1H), 2.39 (s, 3H), 2.46 (t, 2H), 2.72 (s, 3H), 3.36 (t, 2H), 4.83 (d, 1H), 5.50 (bs, 1H), 5.77 (d, 1H), 6.95-7.37 (m, 9H), 7.79 (bs, 3H).

Section 3: Methods 13a-13h

The following compounds were synthesized according to Section 3: Method 13:

| Section 3: | | | |
|---|---|---|---|
| Method # | Compound Name | m/z | SM |
| 13a | (+) N-(3-Amino-propyl)-N-[1-(5-{4-fluorobenzyl}-3- | 508 | Section |
| | methyl-4-oxo-4,5-dihydro-isothiazolo[5,4-d]pyrimidin-6-yl)- | (MH⁺) | 3: |
| | propyl]-4-methyl-benzamide hydrogen chloride | | Method |
| | Section 3: Example A-2 | | 12a |
| 13b | (+) N-(3-Amino-propyl)-N-[1-(5-{3-fluorobenzyl}-3- | 508 | Section |
| | methyl-4-oxo-4,5-dihydro-isothiazolo[5,4-d]pyrimidin-6-yl)- | (MH⁺) | 3: |
| | propyl]-4-methyl-benzamide hydrogen chloride | | Method |
| | Section 3: Example A-3 | | 12b |
| 13c | (+) N-(3-Amino-propyl)-N-[1-(5-benzyl-3-methyl-4-oxo- | 510 | Section |
| | 4,5-dihydro-isothiazolo[5,4-d]pyrimidin-6-yl)-propyl]-4- | (MH⁺) | 3: |
| | chloro-benzamide hydrogen chloride | | Method |
| | Section 3: Example A-5 | | 12c |
| 13d | (+) N-(3-Amino-propyl)-N-[1-(5-benzyl-3-methyl-4-oxo- | 508 | Section |
| | 4,5-dihydro-isothiazolo[5,4-d]pyrimidin-6-yl)-propyl]-3- | (MH⁺) | 3: |
| | fluoro-4-methyl-benzamide hydrogen chloride | | Method |
| | Section 3: Example A-6 | | 12d |
| 13e | (+) N-(3-Amino-propyl)-N-[1-(5-benzyl-3-methyl-4-oxo- | 544, | Section |
| | 4,5-dihydro-isothiazolo[5,4-d]pyrimidin-6-yl)-propyl]-2,3- | 545, | 3: |
| | dichloro-benzamide hydrogen chloride | 546 | Method |
| | Section 3: Example A-7 | (MH⁺) | 12e |
| 13f | (+) Benzo[b]thiophene-2-carboxylic acid (3-amino-propyl)- | 532 | Section |
| | [1-(5-benzyl-3-methyl-4-oxo-4,5-dihydro-isothiazolo[5,4- | (MH⁺) | 3: |
| | d]pyrimidin-6-yl)-propyl]amide hydrogen chloride | | Method |
| | Section 3: Example A-8 | | 12f |
| 13g | (+) N-(3-Amino-propyl)-N-[1-(5-benzyl-3-methyl-4-oxo- | 554, | Section |
| | 4,5-dihydro-isothiazolo[5,4-d]pyrimidin-6-yl)-propyl]-4- | 556 | 3: |
| | bromo-benzamide hydrogen chloride | (MH⁺) | Method |
| | Section 3: Example A-4 | | 12g |
| 13h | (+) N-(2-Amino-ethyl)-N-[1-(5-benzyl-3-methyl-4-oxo-4,5- | 476 | Section |
| | dihydro-isothiazolo[5,4-d]pyrimidin-6-yl)-propyl]-4-methyl- | (MH⁺) | 3: |
| | benzamide hydrogen chloride | | Method |
| | Section 3: Example A-9 | | 12h |
| 13g | N-(3-Amino-propyl)-N-[1-(5-benzyl-3-methyl-4-oxo-4,5- | 490 | Section |
| | dihydro-isothiazolo[5,4-d]pyrimidin-6-yl)-propyl]-4-methyl- | (MH⁺) | 3: |
| | benzamide hydrogen chloride | | Method |
| | | | 11 |

Section 3: Method 14

### N-[1-(5-Benzyl-3-methyl-4-oxo-4,5-dihydro-isothiazolo[5,4-d]pyrimidin-6-yl)-propyl]-N-(3-isopropylamino-propyl)-4-methyl-benzamide

To a solution of N-(3-amino-propyl)-N-[1-(5-benzyl-3-methyl-4-oxo-4,5-dihydro-isothiazolo[5,4-d]pyrimidin-6-yl)-propyl]-4-methyl-benzamide hydrogen chloride (Section 3: Method 13 g) (1.24 g, 2.54 mmol), in the presence of molecular sieves (2 g) was added acetone (1 mL) and the mixture was stirred at room temperature for 2 h. Analysis of the reaction mixture by MS showed the completion of the schiff's base formation. To this mixture was added two drops of acetic acid followed by sodium triacetoxyborohydride (220 mg) and the mixture was stirred overnight. The reaction mixture was filtered and the filtrate was washed with water, dried (Na₂SO₄) and concentrated to get the crude product which was purified by column chromatography (silica gel) using 0-30% EtOAc in hexanes. N-[1-(5-Benzyl-3-methyl-4-oxo-4,5-dihydro-isothiazolo[5,4-d]pyrimidin-6-yl)-propyl]-N-(3-isopropylamino-propyl)-4-methyl-benzamide was isolated as a white foam. Yield 0.206 g (15%). m/z 532 (MH⁺); ¹H NMR (DMSO-d₆, 96° C.) δ: 0.65 (t, 3H), 1.05 (d, 6H), 1.26-1.48 (m, 1H), 1.65-1.70 (m, 1H), 1.80-1.98 (m, 1H), 2.00-2.17 (m, 1H), 2.35 (s, 3H), 2.63 (b, 2H), 2.80 (s, 3H), 3.05 (b, 1H), 3.40 (t, 2H), 4.90 (d, 1H), 5.50 (bs, 1H), 5.80 (d, 1H), 7.35-7.00 (m, 9H).

Section 3: Method 15 and Section 3: Example B-1

### Chiral purification of (+) N-[α-(5-Benzyl-3-methyl-4-oxo-4,5-dihydro-isothiazolo[5,4-d]pyrimidin-6-yl)-propyl]-N-(3-isopropylamino-propyl)-4-methyl-benzamide

The following compound was chirally purified in same manner as (+) (3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 12). Chiral purification generally resulted in 99% purity of the (+) enantiomer.

| Section | | | | | |
|---|---|---|---|---|---|
| 3: | | Column | Solvent | (+) Enantiomer | |
| Method # | Compound Name | Type | composition | retention time | SM |
| 15 | (+) N-[1-(5-Benzyl-3- | Chiralpak | 85% hexanes | 8.0 min | Section |
| | methyl-4-oxo-4,5- | AD | 15% | | 3: |
| | dihydro- | | isopropanol | | Method |
| | isothiazolo[5,4- | | 0.1% | | 14 |
| | d]pyrimidin-6-yl)- | | diethylamine | | |
| | propyl]-N-(3- | | | | |
| | isopropylamino- | | | | |
| | propyl)-4-methyl- | | | | |
| | benzamide | | | | |
| | Section 3: Example | | | | |
| | B-1 | | | | |

Section 3: Method 16

### 5-Butyrylamino-3-methyl-isoxazole-4-carboxylic acid amide

A mixture of 5-amino-3-methyl-isoxazole-4-carboxylic acid amide (2 g, 14.18 mmol) in 10 ml of butyric anhydride was stirred at 150° C. for 0.5˜1 h. The brown solution was diluted with hexane (100 ml) and cooled to room temperature. The solid crushed out from the mixture was filtered and washed with hexane, dried in vacuo. The title amide (2.6 g) was obtained as white solid.

Section 3: Method 17

### 3-Methyl-6-propyl-5H-isoxazolo[5,4-d]pyrimidin-4-one

A suspension of 5-butyrylamino-3-methyl-isoxazole-4-carboxylic acid amide (Section 3: Method 16) (2.6 g, split into 20 vials) in 3.5 ml of 2N NaOH aq was subjected to microwave irradiation under the temperature of 140° C. for 20 min. The resulting solution was cooled with an ice bath, and the pH was adjusted to 1˜3 with concentrated HCl. The crushed out solid was filtered, washed with water, dried over vacuum at 40° C. overnight. The title pyrimidinone (1.749 g) was obtained as white solid. ¹H NMR (DMSO-d₆): 0.91 (t, 3H), 1.71 (m, 2H), 2.44 (s, 3H), 2.64 (t, 2H), 12.78 (s, 1H).

Section 3: Method 18

### 5-Benzyl-3-methyl-6-propyl-5H-isoxazolo[5,4-d]pyrimidin-4-one

A suspension of 3-methyl-6-propyl-5H-isoxazolo[5,4-d]pyrimidin-4-one (Section 3: Method 17) (1.698 g, 8.8 mmol), benzylbromide (1.5 g, 8.8 mmol), potassium carbonate (2.43 g, 17.6 mmol) in 10 ml DMF was stirred at room temperature overnight. The mixture was diluted with water, extracted with EtOAc (50 ml×3), the combined organic phases were dried, concentrated, purified by flash column chromatography (elute: hexane-EtOAc=5:1). 1.69 g (68%) of the title compound was obtained as white solid. ¹H NMR (DMSO-d₆): 0.80 (t, 3H), 1.61 (m, 2H), 2.43 (s, 3H), 2.73 (t, 2H), 5.35 (s, 2H), 7.12-7.35 (m, 5H).

Section 3: Method 19

### 5-Benzyl-6-(1-bromo-propyl)-3-methyl-5H-isoxazolo[5,4-d]pyrimidin-4-one

A solution of 5-benzyl-3-methyl-6-propyl-5H-isoxazolo[5,4-d]pyrimidin-4-one (Section 3: Method 18) (3.167 g, 11.2 mmol) and sodium acetate (4.59 g, 56 mmol, 5 eq) in glacial acetic acid (26 ml) was treated with a preformed bromine solution (0.7 ml bromine in 10 ml of glacial acetic acid) (8.64 ml, 22.4 mmol, 2 eq). The mixture was stirred at 100° C. for 24 hrs. Excess bromine (8.64 ml, 22.4 mmol, 2 eq) was added to the mixture. The mixture was then stirred at 100° C. for another 24 hrs. Water was added to the reaction mixture, followed by aq. potassium carbonate. The mixture was extracted with DCM (50 ml×3), the combined organic phases were washed with water and dried, then concentrated to give the crude product which was purified by flash chromatography (elute: hexane-EtOAc). 2.5 g product was furnished as a white solid. ¹H NMR (DMSO-d₆): 0.79 (t, 3H), 2.18 (m, 1H), 2.35 (m, 1H), 2.58 (s, 3H), 5.12 (t, 1H), 5.25 (d, 1H), 5.80 (d, 1H), 7.27-7.42 (m, 5H).

Section 3: Method 20

### {3-[1-(5-Benzyl-3-methyl-4-oxo-4,5-dihydro-isoxazolo[5,4-d]pyrimidin-6-yl)-propylamino]-propyl}-carbamic acid tert-butyl ester

To a suspension of 5-benzyl-6-(1-bromo-propyl)-3-methyl-5H-isoxazolo[5,4-d]pyrimidin-4-one (Section 3: Method 19) (2.8 g, 7.73 mmol) and potassium carbonate (2.67 g, 19.38 mmol) in acetonitrile (100 ml) was added tert-butyl-N-(3-aminopropyl)-carbamate (1.345 g, 7.73 mmol). The mixture was stirred at 100° C. overnight. Water (30 ml) was added to the mixture, which was extracted with EtOAc (3×50 ml). The combined organic phases were washed with brine (10 ml), dried, concentrated to obtain the crude title amine which was purified by flash chromatography column (elute: EtOAc-hexane=1-4˜1-1) to give 2.6 g (74%) of product as white solid. ¹H NMR (DMSO-d₆): 0.85 (t, 3H), 1.32 (m, 2H), 1.41 (s, 9H), 1.58 (m, 1H), 1.65 (m, 1H), 2.09 (m, 1H), 2.40 (m, 1H), 2.60 (s, 3H), 2.81 (m, 2H), 3.29 (m, 1H), 3.75 (m, 1H), 5.42 (d, 1H), 5.63 (d, 1H), 6.72 (br, 1H), 7.25-7.45 (m, 5H).

Section 3: Method 21

### (3-[[1-(5-Benzyl-3-methyl-4-oxo-4,5-dihydro-isoxazolo[5,4-d]pyrimidin-6-yl)-propyl]-(4-methyl-benzoyl)-amino]-propyl)-carbamic acid tert-butyl ester

A solution of {3-[1-(5-benzyl-3-methyl-4-oxo-4,5-dihydro-isoxazolo[5,4-d]pyrimidin-6-yl)-propylamino]-propyl}-carbamic acid tert-butyl ester (Section 3: Method 20) (135 mg, 0.297 mmol) in DCM (4 ml) was added to 4-methyl-benzoyl chloride (46 mg, 0.297 mmol) followed by triethylamine (60 mg, 0.594 mmol). The mixture was stirred at room temperature for 1 hr. Then diluted with DCM, washed with saturated aq. sodium bicarbonate. The organic phase was dried, filtered, and concentrated. The crude oil was purified by flash column chromatography (solvent: EtOAc-hexane) to furnish (3-[[1-(5-benzyl-3-methyl-4-oxo-4,5-dihydro-isoxazolo[5,4-d]pyrimidin-6-yl)-propyl]-(4-methyl-benzoyl)-amino]-propyl)-carbamic acid tert-butyl ester (130 mg) (76% yield) as a white solid. ¹H NMR (500 MHz, 100° C., DMSO-d₆): 0.71 (t, 3H), 1.12 (m, 1H), 1.35 (s, 9H), 1.47 (m, 1H), 1.92 (m, 1H), 2.14 (m, 1H), 2.37 (s, 3H), 2.56 (s, 3H), 2.57 (m, 2H), 3.29 (m, 2H), 5.01 (d, 1H), 5.68 (m, br, 1H), 5.79 (d, 1H), 6.06 (br, 1H), 7.14-7.36 (m, 9H).

Section 3: Method 22

### Chiral purification of (+) (3-[[1-(5-Benzyl-3-methyl-4-oxo-4,5-dihydro-isoxazolo[5,4-d]pyrimidin-6-yl)-propyl]-(4-methyl-benzoyl)-amino]-propyl)-carbamic acid tert-butyl ester

The following compound was chirally purified in same manner as (+) (3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 12). Chiral purification generally resulted in 99% purity of the (+) enantiomer.

| Section | | | | | |
|---|---|---|---|---|---|
| 3: | | Column | Solvent | (+) Enantiomer | |
| Method # | Compound Name | Type | composition | retention time | SM |
| 22 | (+) (3-[[1-(5-Benzyl-3- | Chiralpak | 70% hexanes | 12.1 min | Section |
| | methyl-4-oxo-4,5- | AD | 30% | | 3: |
| | dihydro-isoxazolo[5,4- | | isopropanol | | Method |
| | d]pyrimidin-6-yl)- | | 0.1% | | 21 |
| | propyl]-(4-methyl- | | diethylamine | | |
| | benzoyl)-amino]- | | | | |
| | propyl)-carbamic acid | | | | |
| | tert-butyl ester | | | | |

### Section 3: Method 23 and Section 3: Example C-1

### (+) N-(3-Amino-propyl)-N-[1-(5-benzyl-3-methyl-4-oxo-4,5-dihydro-isoxazolo[5,4-d]-pyrimidin-6-yl)-propyl]-4-methyl-benzamide hydrogen chloride

A solution of (+) (3-[[1-(5-benzyl-3-methyl-4-oxo-4,5-dihydro-isoxazolo[5,4-d]pyrimidin-6-yl)-propyl]-(4-methyl-benzoyl)-amino]-propyl)-carbamic acid tert-butyl ester (Section 3: Method 22) (23 mg, 0.04 mmol) in 3 ml of 4 M HCl in dioxane was stirred at room temperature for 2 hr. The solvent was distilled off by vacuo, the residue was dried at 40˜50° C. for overnight under vacuum. The corresponding amine chloride salt was obtained. Yield was 19 mg (93%). m/z 474 (MH⁺) ¹H NMR (500 MHz, 100° C., DMSO-d₆): 0.68 (t, 3H), 1.52 (m, 1H), 1.72 (m, 1H), 1.92 (m, 1H), 2.10 (m, 1H), 2.39 (s, 3H), 2.51 (m, 2H), 2.57 (s, 3H), 3.41 (m, 2H), 4.85 (br, 1H), 5.50 (br, 1H), 5.77 (d, 1H), 7.07 (br, 2H), 7.24-7.35 (m, 7H), 7.73 (br, 3H).

Section 3: Method 24

### N-(4-Cyano-3-methyl-isothiazol-5-yl)-3-methyl-butyramide

To a solution of 5-amino-3-methylisothiazole-4-carbonitrile (Section 3: Method 4) (6.38 g, 45.9 mmol) in pyridine (20 mL) at 0° C., isovaleryl chloride (6.65 g, 55 mmol) was added dropwise. After the completion of the addition the reaction mixture was allowed to warm to r.t. and stirred overnight. The TLC and the MS showed the complete disappearance of the starting material and the reaction mixture was diluted with CHCl₃ (200 mL), washed with water (200 mL), 2N HCl (225 mL), satd. NaHCO₃ (200 mL), brine (200 mL) and dried over Na₂SO₄. Concentration of the CHCl₃ layer provided the crude product which was triturated from DCM/hexanes (1/10) and filtered off to isolate N-(4-cyano-3-methyl-isothiazol-5-yl)-3-methyl-butyramide (8.1 g, 79%) as an off-white crystalline solid. ¹H NMR (300 MHz) δ 1.04 (d, 6H), 2.18-2.32 (m, 1H), 2.46 (d, 2H), 2.53 (s, 3H), 9.87 (bs, 1H).

Section 3: Method 25

### 3-Methyl-5-(3-methyl-butyrylamino)-isothiazole-4-carboxylic acid amide

To a solution of N-(4-cyano-3-methyl-isothiazol-5-yl)-3-methyl-butyramide (Section 3: Method 24) (8 g, 35.8 mmol) in 30% aqueous NH₄OH (200 mL), was added dropwise 100 mL of hydrogen peroxide at r.t. After the completion of the addition the reaction mixture was stirred at 60° C. overnight after which the TLC showed the complete disappearance of SM. The reaction mixture was concentrated to 40 mL and extracted with chloroform (3×100 mL). The organic layer was dried (Na₂SO₄) and concentrated to obtain 3-methyl-5-(3-methyl-butyrylamino)-isothiazole-4-carboxylic acid amide (6.1 g, 71%) as a light yellow solid. ¹H NMR (300 MHz) δ 1.03 (d, 6H), 2.24 (m, 1H), 2.43 (d, 2H), 2.69 (s, 3H), 5.98 (bs, 2H), 11.77 (bs, 1H).

Section 3: Method 26

### 6-Isobutyl-3-methyl-5H-isothiazolo[5,4-d]pyrimidin-4-one

3-Methyl-5-(3-methyl-butyrylamino)-isothiazole-4-carboxylic acid amide (Section 3: Method 25) (6 g, 25 mmol) was suspended in 150 mL of 30% NH₃ and then was heated to 140° C. for 5 h in a pressure reactor. The mixture was cooled and neutralized to pH 7. The reaction mixture was extracted with EtOAc (3×100 mL) and the combined organic layers were washed with water (100 mL), brine (100 mL) and concentrated to get the crude product which was further purified by column (silica gel) chromatography using 30% EtOAc in hexanes as eluent. Concentration of the pure product fractions provided 6-isobutyl-3-methyl-5H-isothiazolo[5,4-d]pyrimidin-4-one (2.2 g, 38%) as an off-white powder. ¹H NMR (300 MHz) δ 1.05 (d, 6H), 2.32 (m, 1H), 2.69 (d, 2H), 2.82 (s, 3H).

Section 3: Method 27

### 5-Benzyl-6-isobutyl-3-methyl-5H-isothiazolo[5,4-d]pyrimidin-4-one

To a solution of 6-isobutyl-3-methyl-5H-isothiazolo[5,4-d]pyrimidin-4-one (Section 3: Method 26) (1.31 g, 5.8 mmol) in 20 mL of anhydrous DMF was added 1.38 g (10 mmol) of anhydrous K₂CO₃ followed by benzyl bromide (1.18 g, 6.9 mmol) and the mixture was stirred at room temperature overnight. The TLC of the reaction mixture showed the complete disappearance of the SM. The reaction mixture was poured into ice-cold water and extracted with EtOAc (3×100 mL). The combined extracts were washed with water (100 mL), brine (100 mL), dried (Na₂SO₄) and concentrated. The TLC and the ¹H NMR showed the presence of two products N alkylated as well as O-alkylated products in a ratio of 7:3. The products were separated by column (silica gel, 116 g) chromatography using 10% EtOAc in hexanes. 5-Benzyl-6-isobutyl-3-methyl-5H-isothiazolo[5,4-d]pyrimidin-4-one was isolated as white crystalline solid (1.3 g, 70%). m/z 314 (MH⁺), ¹H NMR (300 MHz) δ 0.94 (d, 6H), 2.23-2.37 (m, 1H), 2.64 (d, 2H), 2.82 (s, 3H), 5.38 (s, 2H), 7.10-7.38 (m, 5H).

Section 3: Methods 27a-b

The following compounds were synthesized according to Section 3: Method 27:

| Section 3: | | | Alkylating |
|---|---|---|---|
| Method # | Compound Name | m/z | agent |
| 27a | 5-(4-Fluoro-benzyl)-6-isobutyl-3- | 332 | 4-fluorobenzyl |
| | methyl-5H-isothiazolo[5,4- | (MH⁺) | bromide |
| | d]pyrimidin-4-one | | |
| 27b | 5-(3-Fluoro-benzyl)-6-isobutyl-3- | 332 | 3-fluorobenzyl |
| | methyl-5H-isothiazolo[5,4- | (MH⁺) | bromide |
| | d]pyrimidin-4-one | | |

Section 3: Method 28

### 5-Benzyl-6-(1-bromo-2-methyl-propyl)-3-methyl-5H-isothiazolo[5,4-d]pyrimidin-4-one

To a solution of 5-benzyl-6-isobutyl-3-methyl-5H-isothiazolo[5,4-d]pyrimidin-4-one (Section 3: Method 27) (1.3 g, 4.2 mmol) and sodium acetate (2 g) in acetic acid (10 mL) at 100° C., a solution of the bromine (1.32 g, 8.4 mmol) in acetic acid (10 mL) was added dropwise over a period of 20 minutes. The reaction mixture was stirred at that temperature for 30 min and cooled and the TLC (eluent 10% EtOAc in hexanes) and MS showed the complete disappearance of the SM and only the product. The reaction mixture was poured into ice water and extracted with EtOAc (3×60 mL) and the organic layers were combined and washed with 2% sodium thiosulfate solution (60 mL), water (100 mL), brine (100 mL) and dried over Na₂SO₄. Concentration of the organic layer provided 5-benzyl-6-(1-bromo-2-methyl-propyl)-3-methyl-5H-isothiazolo[5,4-d]pyrimidin-4-one (1.61 g, 99%) as white crystalline solid. m/z 392, 394 (MH⁺), ¹H NMR (300 MHz) δ 0.54 (d, 3H), 1.11 (d, 3H), 2.62-2.76 (m, 1H), 2.83 (s, 3H), 4.42 (d, 1H), 4.80 (d, 1H), 6.22 (d, 1H), 7.12-7.42 (m, 5H).

Section 3: Methods 28a-b

The following compounds were synthesized according to Section 3: Method 28:

| Section 3: | | | |
|---|---|---|---|
| Method # | Compound Name | m/z | SM |
| 28a | 6-(1-Bromo-2-methyl-propyl)-5-(4-fluoro- | 410, | Section |
| | benzyl)-3-methyl-5H-isothiazolo[5,4- | 412 | 3: |
| | d]pyrimidin-4-one | (MH⁺) | Method |
| | | | 27a |
| 28b | 6-(1-Bromo-2-methyl-propyl)-5-(3-fluoro- | 410, | Section |
| | benzyl)-3-methyl-5H-isothiazolo[5,4- | 412 | 3: |
| | d]pyrimidin-4-one | (MH⁺) | Method |
| | | | 27b |

Section 3: Method 29

### 6-(1-Azido-2-methyl-propyl)-5-benzyl-3-methyl-5H-isothiazolo[5,4-d]pyrimidin-4-one

To a solution of 5-benzyl-6-(1-bromo-2-methyl-propyl)-3-methyl-5H-isothiazolo[5,4-d]pyrimidin-4-one (Section 3: Method 28) (0.6 g, 1.52 mmol) in anhydrous DMF (20 mL), sodium azide (0.65 g, 10 mmol) was added and the mixture was stirred at room temperature for 1 hour. The TLC of the RM showed the complete disappearance of the starting bromide. The reaction mixture was poured into ice water (300 mL) and extracted with EtOAc (3×100 mL). The organic layer was washed with water (100 mL), brine (100 mL) and dried (Na₂SO₄). Concentration of the organic layer provided the crude product which was purified by column (silica gel) chromatography using 30% EtOAc in hexanes as eluent to isolate 6-(1-azido-2-methyl-propyl)-5-benzyl-3-methyl-5H-isothiazolo[5,4-d]pyrimidin-4-one (0.506 g, 94%) as a low melting solid. m/z 355 (MH⁺), ¹H NMR (300 MHz) δ 0.57 (d, 3H), 1.07 (d, 3H), 2.50-2.74 (m, 1H), 2.98 (s, 3H), 3.71 (d, 1H), 5.05 (d, 1H), 5.78 (d, 1H), 7.12-7.40 (m, 5H).

Section 3: Methods 29a-b

The following compounds were synthesized according to Section 3: Method 29:

| Section 3: | | | |
|---|---|---|---|
| Method # | Compound Name | m/z | SM |
| 29a | 6-(1-Azido-2-methyl-propyl)-5-(4-fluoro- | 373 | Section |
| | benzyl)-3-methyl-5H-isothiazolo[5,4- | (MH⁺) | 3: |
| | d]pyrimidin-4-one | | Method |
| | | | 28a |
| 29b | 6-(1-Azido-2-methyl-propyl)-5-(3-fluoro- | 373 | Section |
| | benzyl)-3-methyl-5H-isothiazolo[5,4- | (MH⁺) | 3: |
| | d]pyrimidin-4-one | | Method |
| | | | 28b |

Section 3: Method 30

### 6-(1-Amino-2-methyl-propyl)-5-benzyl-3-methyl-5H-isothiazolo[5,4-d]pyrimidin-4-one

To a solution of 6-(1-azido-2-methyl-propyl)-5-benzyl-3-methyl-5H-isothiazolo[5,4-d]pyrimidin-4-one (Section 3: Method 29) (0.5 g, 1.41 mmol) in methanol (20 mL) was added 5% Pd/C (20% by wt.) and the resulting mixture was stirred at r.t. in an atmosphere of H₂ and the progress of the reaction was monitored by MS. After the disappearance of the starting material the reaction mixture was filtered through celite and washed with EtOAc. Concentration of the filtrate provided 6-(1-amino-2-methyl-propyl)-5-benzyl-3-methyl-5H-isothiazolo[5,4-d]pyrimidin-4-one as a thick oil. The product was used as such in the next reaction with out further purification. m/z 349 (MH⁺).

Section 3: Methods 30a-b

The following compounds were synthesized according to Section 3: Method 30:

| Section 3: | | | |
|---|---|---|---|
| Method # | Compound Name | m/z | SM |
| 30a | 6-(1-Amino-2-methyl-propyl)-5- | 367 | Section |
| | (4-fluoro-benzyl)-3-methyl- | (MH⁺) | 3: |
| | 5H-isothiazolo[5,4-d]pyrimidin-4-one | | Method |
| | | | 29a |
| 30b | 6-(1-Amino-2-methyl-propyl)-5-(3- | 367 | Section |
| | fluoro-benzyl)-3-methyl- | (MH⁺) | 3: |
| | 5H-isothiazolo[5,4-d]pyrimidin-4-one | | Method |
| | | | 29b |

Section 3: Method 31

### {3-[1-(5-Benzyl-3-methyl-4-oxo-4,5-dihydro-isothiazolo[5,4-d]pyrimidin-6-yl)-2-methyl-propylamino]-propyl}-carbamic acid tert-butyl ester

To a solution of 6-(1-amino-2-methyl-propyl)-5-benzyl-3-methyl-5H-isothiazolo[5,4-d]pyrimidin-4-one (Section 3: Method 30) in DCM (30 mL), 4 Å molecular sieves (5 g) was added followed by (3-oxo-propyl)-carbamic acid tert-butyl ester (1.2 eq) and the reaction mixture was stirred at r.t. for 3 h and the progress of the reaction was monitored by MS. After the complete disappearance of the starting amine, a catalytic amount of acetic acid was added to the reaction followed by sodium triacetoxyborohydride (1.2 eq) and the reaction mixture was stirred at r.t. overnight. After the completion of the reaction (MS), the reaction mixture was filtered and the residue was washed with DCM and the filtrate was washed with water (100 mL), brine (100 mL) and concentrated to get the crude product which was used as such for the next reaction. m/z 486 (MH⁺).

Section 3: Methods 31a-c

The following compounds were synthesized according to Section 3: Method 31:

| Section 3: | | | |
|---|---|---|---|
| Method # | Compound Name | m/z | SM |
| 31a | (3-{1-[5-(4-Fluoro-benzyl)-3-methyl-4-oxo-4,5-dihydro- | 504 | Section |
| | isothiazolo[5,4-d]pyrimidin-6-yl]-2-methyl-propylamino}- | (MH⁺) | 3: |
| | propyl)-carbamic acid tert-butyl ester | | Method |
| | | | 30a |
| 31b | (3-{1-[5-(3-Fluoro-benzyl)-3-methyl-4-oxo-4,5-dihydro- | 504 | Section |
| | isothiazolo[5,4-d]pyrimidin-6-yl]-2-methyl-propylamino}- | (MH⁺) | 3: |
| | propyl)-carbamic acid tert-butyl ester | | Method |
| | | | 30b |
| 31c | {2-[1-(5-Benzyl-3-methyl-4-oxo-4,5-dihydro-isothiazolo[5,4- | 472 | Section |
| | d]pyrimidin-6-yl)-2-methyl-propylamino]-ethyl}-carbamic | (MH⁺) | 3: |
| | acid tert-butyl ester | | Method |
| | | | 30 |

Section 3: Method 32

### 5-Benzyl-6-[1-(2-[1,3]dioxolan-2-yl-ethylamino)-2-methyl-propyl]-3-methyl-5H-isothiazolo[5,4-d]pyrimidin-4-one

To a solution of 6-(1-amino-2-methyl-propyl)-5-benzyl-3-methyl-5H-isothiazolo[5,4-d]pyrimidin-4-one (Section 3: Method 30) (1.6 g, 4.88 mmol) in anhydrous DMF (20 mL), 2-(2-bromo-ethyl)-[1,3]dioxolane (0.88 g, 4.88 mmol) was added and the resulting solution was heated at 70° C. for 2 h. The reaction mixture was cooled, diluted with water and extracted with EtOAc (3×60 mL). The combined organic extracts were dried (Na₂SO₄) and concentrated to provide the crude product (2 g), which was used as such in the next reaction. m/z 429 (MH⁺); ¹H-NMR (300 MHz) δ 0.88 (d, 3H), 0.96 (d, 3H), 1.54-1.62 (m, 2H), 1.86-2.05 (m, 2H), 2.18 (bs, 1H), 2.38-2.46 (m, 1H), 2.84 (s, 3H), 3.57 (d, 1H), 3.74-3.94 (m, 4H), 4.78 (t, 1H), 4.99 (d, 1H), 5.85 (d, 1H), 7.15-7.38 (m, 5H).

Section 3: Method 33

### {3-[[1-(5-Benzyl-3-methyl-4-oxo-4,5-dihydro-isothiazolo[5,4-d]pyrimidin-6-yl)-2-methyl-propyl]-(4-methyl-benzoyl)-amino]-propyl}-carbamic acid tert-butyl ester

To a solution of the crude {3-[1-(5-benzyl-3-methyl-4-oxo-4,5-dihydro-isothiazolo[5,4-d]pyrimidin-6-yl)-2-methyl-propylamino]-propyl}-carbamic acid tert-butyl ester (Section 3: Method 31) in pyridine (10 mL) at r.t., a solution of the p-toluoyl chloride (0.616 g, 4 mmol) in DCM (10 mL) was added dropwise and the resulting solution was stirred at r.t. for 2 days. The reaction mixture was diluted with DCM (100 mL) washed with water (2×100 mL), brine (100 mL) and dried (Na₂SO₄). Concentration of the organic layer provided the crude product which was purified by column (silica gel) chromatography using 20-30% EtOAc in hexanes as eluent. Product isolated was 0.276 g. m/z 604 (MH⁺).

Section 3: Methods 33a-g

The following compounds were synthesized according to Section 3: Method 33:

| Section 3: | | | | |
|---|---|---|---|---|
| Method # | Compound Name | m/z | SM | Acylating agent |
| 33a | {3-[{1-[5-(4-Fluoro-benzyl)-3-methyl-4- | 622 | Section | 4-methyl-benzoyl |
| | oxo-4,5-dihydro-isothiazolo[5,4- | (MH⁺) | 3: | chloride |
| | d]pyrimidin-6-yl]-2-methyl-propyl}-(4- | | Method | |
| | methyl-benzoyl)-amino]-propyl}- | | 31a | |
| | carbamic acid tert-butyl ester | | | |
| 33b | {3-[{1-[5-(3-Fluoro-benzyl)-3-methyl-4- | 622 | Section | 4-methyl-benzoyl |
| | oxo-4,5-dihydro-isothiazolo[5,4- | (MH⁺) | 3: | chloride |
| | d]pyrimidin-6-yl]-2-methyl-propyl}-(4- | | Method | |
| | methyl-benzoyl)-amino]-propyl}- | | 31b | |
| | carbamic acid tert-butyl ester | | | |
| 33c | {2-[[1-(5-Benzyl-3-methyl-4-oxo-4,5- | 590 | Section | 4-methyl-benzoyl |
| | dihydro-isothiazolo[5,4-d]pyrimidin-6- | (MH⁺) | 3: | chloride |
| | yl)-2-methyl-propyl]-(4-methyl- | | Method | |
| | benzoyl)-amino]-ethyl}-carbamic acid | | 31c | |
| | tert-butyl ester | | | |
| 33d | {2-[[1-(5-Benzyl-3-methyl-4-oxo-4,5- | 654, | Section | 4-bromo-benzoyl |
| | dihydro-isothiazolo[5,4-d]pyrimidin-6- | 656 | 3: | chloride |
| | yl)-2-methyl-propyl]-(4-bromo-benzoyl)- | (MH⁺) | Method | |
| | amino]-ethyl}-carbamic acid tert-butyl | | 31c | |
| | ester | | | |
| 33e | {2-[[1-(5-Benzyl-3-methyl-4-oxo-4,5- | 608 | Section | 3-fluoro-4- |
| | dihydro-isothiazolo[5,4-d]pyrimidin-6- | (MH⁺) | 3: | methyl-benzoyl |
| | yl)-2-methyl-propyl]-(3-fluoro-4-methyl- | | Method | chloride |
| | benzoyl)-amino]-ethyl}-carbamic acid | | 31c | |
| | tert-butyl ester | | | |
| 33f | {3-[[1-(5-Benzyl-3-methyl-4-oxo-4,5- | 622 | Section | 3-fluoro-4- |
| | dihydro-isothiazolo[5,4-d]pyrimidin-6- | (MH⁺) | 3: | methyl-benzoyl |
| | yl)-2-methyl-propyl]-(3-fluoro-4-methyl- | | Method | chloride |
| | benzoyl)-amino]-propyl}-carbamic acid | | 31 | |
| | tert-butyl ester | | | |
| 33g | {3-[[1-(5-Benzyl-3-methyl-4-oxo-4,5- | 668, | Section | 4-bromo-benzoyl |
| | dihydro-isothiazolo[5,4-d]pyrimidin-6- | 670 | 3: | chloride |
| | yl)-2-methyl-propyl]-(4-bromo-benzoyl)- | (MH⁺) | Method | |
| | amino]-propyl}-carbamic acid tert-butyl | | 31 | |
| | ester | | | |

Section 3: Methods 34a-g

The following compounds were chirally purified in same manner as (+) (3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 12). Chiral purification generally resulted in 99% purity of the (+) enantiomer.

| Section | | | | | |
|---|---|---|---|---|---|
| 3: | | Column | Solvent | (+) Enantiomer | |
| Method # | Compound Name | Type | composition | retention time | SM |
| 34a | (+) {3-[{1-[5-(4-Fluoro- | Chiralpak | 85% hexanes | 7.1 min | Section |
| | benzyl)-3-methyl-4-oxo- | AD | 15% | | 3: |
| | 4,5-dihydro- | | isopropanol | | Method |
| | isothiazolo[5,4- | | 0.1% | | 33a |
| | d]pyrimidin-6-yl]-2- | | diethylamine | | |
| | methyl-propyl}-(4- | | | | |
| | methyl-benzoyl)-amino]- | | | | |
| | propyl}-carbamic acid | | | | |
| | tert-butyl ester | | | | |
| 34b | (+) {3-[{1-[5-(3-Fluoro- | Chiralpak | 85% hexanes | 8.0 min | Section |
| | benzyl)-3-methyl-4-oxo- | AD | 15% | | 3: |
| | 4,5-dihydro- | | isopropanol | | Method |
| | isothiazolo[5,4- | | 0.1% | | 33b |
| | d]pyrimidin-6-yl]-2- | | diethylamine | | |
| | methyl-propyl}-(4- | | | | |
| | methyl-benzoyl)-amino]- | | | | |
| | propyl}-carbamic acid | | | | |
| | tert-butyl ester | | | | |
| 34c | (+) {2-[[1-(5-Benzyl-3- | Chiralpak | 80% hexanes | 7.7 min | Section |
| | methyl-4-oxo-4,5- | AD | 20% | | 3: |
| | dihydro-isothiazolo[5,4- | | isopropanol | | Method |
| | d]pyrimidin-6-yl)-2- | | 0.1% | | 33c |
| | methyl-propyl]-(4- | | diethylamine | | |
| | methyl-benzoyl)-amino]- | | | | |
| | ethyl}-carbamic acid tert- | | | | |
| | butyl ester | | | | |
| 34d | (+) {2-[[1-(5-Benzyl-3- | Chiralpak | 75% hexanes | 7.9 min | Section |
| | methyl-4-oxo-4,5- | AD | 25% | | 3: |
| | dihydro-isothiazolo[5,4- | | isopropanol | | Method |
| | d]pyrimidin-6-yl)-2- | | 0.1% | | 33d |
| | methyl-propyl]-(4- | | diethylamine | | |
| | bromo-benzoyl)-amino]- | | | | |
| | ethyl}-carbamic acid tert- | | | | |
| | butyl ester | | | | |
| 34e | (+) {2-[[1-(5-Benzyl-3- | Chiralpak | 75% hexanes | 6.3 min | Section |
| | methyl-4-oxo-4,5- | AD | 25% | | 3: |
| | dihydro-isothiazolo[5,4- | | isopropanol | | Method |
| | d]pyrimidin-6-yl)-2- | | 0.1% | | 33e |
| | methyl-propyl]-(3-fluoro- | | diethylamine | | |
| | 4-methyl-benzoyl)- | | | | |
| | amino]-ethyl}-carbamic | | | | |
| | acid tert-butyl ester | | | | |
| 34f | (+) {3-[[1-(5-Benzyl-3- | Chiralpak | 80% hexanes | 8.6 min | Section |
| | methyl-4-oxo-4,5- | AD | 20% | | 3: |
| | dihydro-isothiazolo[5,4- | | isopropanol | | Method |
| | d]pyrimidin-6-yl)-2- | | 0.1% | | 33f |
| | methyl-propyl]-(3-fluoro- | | diethylamine | | |
| | 4-methyl-benzoyl)- | | | | |
| | amino]-propyl}-carbamic | | | | |
| | acid tert-butyl ester | | | | |
| 34g | (+) {3-[[1-(5-Benzyl-3- | Chiralpak | 80% hexanes | 7.0 min | Section |
| | methyl-4-oxo-4,5- | AD | 20% | | 3: |
| | dihydro-isothiazolo[5,4- | | isopropanol | | Method |
| | d]pyrimidin-6-yl)-2- | | 0.1% | | 33g |
| | methyl-propyl]-(4- | | diethylamine | | |
| | bromo-benzoyl)-amino]- | | | | |
| | propyl}-carbamic acid | | | | |
| | tert-butyl ester | | | | |

Chiral purification generally resulted in 99% purity of the (+) enantiomer.

Section 3: Method 35

### N-(3-Amino-propyl)-N-[1-(5-benzyl-3-methyl-4-oxo-4,5-dihydro-isothiazolo[5,4-d]pyrimidin-6-yl)-2-methyl-propyl]-4-methyl-benzamide hydrogen chloride

{3-[[1-(5-Benzyl-3-methyl-4-oxo-4,5-dihydro-isothiazolo[5,4-d]pyrimidin-6-yl)-2-methyl-propyl]-(4-methyl-benzoyl)-amino]-propyl}-carbamic acid tert-butyl ester (Section 3: Method 33) (0.245 g, 0.40 mmol) was dissolved in 4M HCl in 1,4-dioxane and the mixture was stirred at r.t. for 20 min and the TLC showed the complete disappearance of the starting material. The reaction mixture was concentrated in a rotary evaporator and the residue was triturated with ether. The precipitated product was filtered off and washed with ether and dried under vacuo to yield N-(3-amino-propyl)-N-[1-(5-benzyl-3-methyl-4-oxo-4,5-dihydro-isothiazolo[5,4-d]pyrimidin-6-yl)-2-methyl-propyl]-4-methyl-benzamide as the hydrochloride salt (0.219 g, 100%). White powder, mp. 139-140° C. m/z 504 (MH⁺), ¹H NMR (DMSO-d₆, 300 MHz, 96° C.) δ: 0.45 (d, 3H), 0.90 (d, 3H), 1.12-1.30 (m, 1H), 1.46-1.63 (m, 1H), 2.25 (t, 2H), 2.36 (s, 3H), 2.64-2.7 (m, 1H), 2.68 (s, 3H), 3.34 (t, 2H), 5.06 (d, 1H), 5.59 (d, 1H), 5.90 (d, 1H), 7.20-7.40 (m, 9H), 7.71 (bs, 3H).

Section 3: Methods 35a-g

The following compounds were synthesized according to Section 3: Method 35:

| Section 3: | | | |
|---|---|---|---|
| Method # | Compound Name | m/z | SM |
| 35a | (+)N-(3-Amino-propyl)-N-{1-[5-(4-fluoro-benzyl)-3-methyl- | 522 | Section |
| | 4-oxo-4,5-dihydro-isothiazolo[5,4-d]pyrimidin-6-yl]-2-methyl- | (MH⁺) | 3: |
| | propyl}-4-methyl-benzamide hydrogen chloride | | Method |
| | Section 3: Example D-1 | | 34a |
| 35b | (+)N-(3-Amino-propyl)-N-{1-[5-(3-fluoro-benzyl)-3-methyl- | 522 | Section |
| | 4-oxo-4,5-dihydro-isothiazolo[5,4-d]pyrimidin-6-yl]-2-methyl- | (MH⁺) | 3: |
| | propyl}-4-methyl-benzamide hydrogen chloride | | Method |
| | Section 3: Example D-3 | | 34b |
| 35c | (+)N-(2-Amino-ethyl)-N-[1-(5-benzyl-3-methyl-4-oxo-4,5- | 490 | Section |
| | dihydro-isothiazolo[5,4-d]pyrimidin-6-yl)-2-methyl-propyl]-4- | (MH⁺) | 3: |
| | methyl-benzamide hydrogen chloride | | Method |
| | Section 3: Example D-5 | | 34c |
| 35d | (+)N-(2-Amino-ethyl)-N-[1-(5-benzyl-3-methyl-4-oxo-4,5- | 554, | Section |
| | dihydro-isothiazolo[5,4-d]pyrimidin-6-yl)-2-methyl-propyl]-4- | 556 | 3: |
| | bromo-benzamide hydrogen chloride | (MH⁺) | Method |
| | Section 3: Example D-4 | | 34d |
| 35e | (+)N-(2-Amino-ethyl)-N-[1-(5-benzyl-3-methyl-4-oxo-4,5- | 508 | Section |
| | dihydro-isothiazolo[5,4-d]pyrimidin-6-yl)-2-methyl-propyl]-3- | (MH⁺) | 3: |
| | fluoro-4-methyl-benzamide hydrogen chloride | | Method |
| | Section 3: Example D-6 | | 34e |
| 35f | (+)N-(3-Amino-propyl)-N-[1-(5-benzyl-3-methyl-4-oxo-4,5- | 522 | Section |
| | dihydro-isothiazolo[5,4-d]pyrimidin-6-yl)-2-methyl-propyl]-3- | (MH⁺) | 3: |
| | fluoro-4-methyl-benzamide hydrogen chloride | | Method |
| | Section 3: Example D-7 | | 34f |
| 35g | (+)N-(3-Amino-propyl)-N-[1-(5-benzyl-3-methyl-4-oxo-4,5- | 568, | Section |
| | dihydro-isothiazolo[5,4-d]pyrimidin-6-yl)-2-methyl-propyl]-4- | 570 | 3: |
| | bromo-benzamide hydrogen chloride | (MH⁺) | Method |
| | Section 3: Example D-8 | | 34g |

### Section 3: Method 36 and Section 3: Example D-2

### Chiral purification of (+) N-(3-Amino-propyl)-N-[1-(5-benzyl-3-methyl-4-oxo-4,5-dihydro-isothiazolo[5,4-d]pyrimidin-6-yl)-2-methyl-propyl]-4-methyl-benzamide

The following compound was chirally purified in same manner as (+) (3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 12). Chiral purification generally resulted in 99% purity of the (+) enantiomer.

| Section | | | | | |
|---|---|---|---|---|---|
| 3: | | Column | Solvent | (+) Enantiomer | |
| Method # | Compound Name | Type | composition | retention time | SM |
| 36 | (+) N-(3-Amino- | Chiralpak | 70% hexanes | 8.0 min | Section |
| | propyl)-N-[1-(5-benzyl- | AD | 30% | | 3: |
| | 3-methyl-4-oxo-4,5- | | isopropanol | | Method |
| | dihydro-isothiazolo[5,4- | | 0.1% | | 35 |
| | d]pyrimidin-6-yl)-2- | | diethylamine | | |
| | methyl-propyl]-4- | | | | |
| | methyl-benzamide | | | | |
| | Section 3: Example D-2 | | | | |

Section 3: Method 37

### N-[1-(5-Benzyl-3-methyl-4-oxo-45-dihydro-isothiazolo[5,4-d]pyrimidin-6-yl)-2-methyl-propyl]-4-bromo-N-(2-[1,3]dioxolan-2-yl-ethyl-benzamide

5-Benzyl-6-[1-(2-[1,3]dioxolan-2-yl-ethylamino)-2-methyl-propyl]-3-methyl-5H-isothiazolo[5,4-d]pyrimidin-4-one (Section 3: Method 32) (1 g, 2.33 mmol) was dissolved in chloroform (70 mL) and to the chloroform solution diisopropylethyl amine (0.9 g, 6.99 mmol) was added followed by the addition of 4-bromobenzoyl chloride (0.76 g, 3.49 mmol) and the mixture was refluxed overnight. The MS showed the disappearance of the starting material and only the product peak at 611 (MH⁺). The reaction mixture was concentrated and column purified (silica gel, 160 g) using 10-20% EtOAc in hexanes as eluent. The concentration of the product fractions provided the pure product as white foam (1.1 g, 77%). m/z 611, 613 (MH⁺); ¹H-NMR (300 MHz) δ 0.35 (d, 3H), 0.94 (d, 3H), 0.94-1.06 (m, 1H), 1.36-1.46 (m, 1H), 2.68-2.78 (m, 1H), 2.88 (s, 3H), 3.38-3.52 (m, 1H), 3.54-3.70 (m, 5H), 4.34 (t, 1H), 5.18 (d, 1H), 5.73 (d, 1H), 6.13 (d, 1H), 7.20 (d, 2H), 7.26-7.46 (m, 5H), 7.56 (d, 2H).

Section 3: Methods 37a-b

The following compounds were synthesized according to Section 3: Method 37:

| Section 3: | | | | Acylating |
|---|---|---|---|---|
| Method # | Compound Name | m/z | SM | agent |
| 37a | N-[1-(5-Benzyl-3-methyl-4-oxo-4,5- | 547 | Section 3: | 4-methyl- |
| | dihydro-isothiazolo[5,4-d]pyrimidin-6-yl)- | (MH⁺) | Method | benzoyl |
| | 2-methyl-propyl]-N-(2-[1,3]dioxolan-2-yl- | | 32 | chloride |
| | ethyl)-4-methyl-benzamide | | | |
| 37b | N-[1-(5-Benzyl-3-methyl-4-oxo-4,5- | 565 | Section 3: | 3-fluoro-4- |
| | dihydro-isothiazolo[5,4-d]pyrimidin-6-yl)- | (MH⁺) | Method | methyl- |
| | 2-methyl-propyl]-N-(2-[1,3]dioxolan-2-yl- | | 32 | benzoyl |
| | ethyl)-3-fluoro-4-methyl-benzamide | | | chloride |

Section 3: Method 38

### N-[1-(5-Benzyl-3-methyl-4-oxo-4,5-dihydro-isothiazolo[5,4-d]pyrimidin-6-yl)-2-methyl-propyl]-4-bromo-N-(3-oxo-propyl)-benzamide

N-[1-(5-Benzyl-3-methyl-4-oxo-4,5-dihydro-isothiazolo[5,4-d]pyrimidin-6-yl)-2-methyl-propyl]-4-bromo-N-(2-[1,3]dioxolan-2-yl-ethyl)-benzamide (Section 3: Method 37) (1.1 g, 1.8 mmol) was dissolved in 20 mL of 80% acetic acid and the solution was heated at 80° C. for 2 h. The reaction mixture was cooled in an ice bath and neutralized slowly by the addition of solid NaHCO₃ until pH 8. The thus obtained mixture was extracted with DCM (3×100 mL). The combined organic layers was washed with brine (100 mL) and dried (Na₂SO₄). Concentration of the DCM layer provided a yellow foam (1 g crude yield) and it was used as such in the next reaction. m/z 567, 569 (MH⁺).

Section 3: Methods 38a-b

The following compounds were synthesized according to Section 3: Method 38:

| Section 3: | | | |
|---|---|---|---|
| Method # | Compound Name | m/z | SM |
| 38a | N-[1-(5-Benzyl-3-methyl-4- | 503 | Section 3: |
| | oxo-4,5-dihydro-isothiazolo[5,4- | (MH⁺) | Method 37a |
| | d]pyrimidin-6-yl)-2-methyl- | | |
| | propyl]-4-methyl-N-(3-oxo- | | |
| | propyl)-benzamide | | |
| 38b | N-[1-(5-Benzyl-3-methyl-4-oxo- | 521 | Section 3: |
| | 4,5-dihydro-isothiazolo[5,4- | (MH⁺) | Method 37b |
| | d]pyrimidin-6-yl)-2-methyl-propyl]- | | |
| | 3-fluoro-4-methyl-N-(3-oxo- | | |
| | propyl)-benzamide | | |

Section 3: Method 39

### N-[1-(5-Benzyl-3-methyl-4-oxo-4,5-dihydro-isothiazolo[5,4-d]pyrimidin-6-yl]-2-methyl-pr 4-bromo-N-(3-dimethylamino-propyl)-benzamide

To a solution of N-[1-(5-benzyl-3-methyl-4-oxo-4,5-dihydro-isothiazolo[5,4-d]pyrimidin-6-yl)-2-methyl-propyl]-4-bromo-N-(3-oxo-propyl)-benzamide (Section 3: Method 38) (1 g, 1.76 mmol) in methanol (20 mL) two drops of acetic acid were added followed by the addition of dimethylamine (1 mL, 2M solution in THF) and sodium cyanoborohydride (0.314 g, 5 mmol) and the mixture was stirred at room temperature for 3 h. The reaction mixture was concentrated and the residue was dissolved in DCM (100 mL) and the organic layer was washed with satd. NaHCO₃ (3×100 mL). The organic layer was concentrated and the crude product was purified by column chromatography using 0-10% MeOH in EtOAc. The pure product fractions were concentrated and the thus obtained foam was crystallized from ether/hexanes to get the product as white crystalline solid. Yield was 0.366 g (35%). m/z 596, 598 (MH⁺); ¹H-NMR (300 MHz) δ 0.35 (d, 3H), 0.66-0.77 (m, 1H), 0.93 (d, 3H), 0.18-1.27 (m, 1H), 1.65-1.85 (m, 2H), 1.80 (s, 6H), 2.66-2.76 (m, 1H), 2.89 (s, 3H), 3.30-3.41 (m, 2H), 5.20 (d, 1H), 5.73 (d, 1H), 6.15 (d, 1H), 7.20 (d, 2H), 7.28-7.41 (m, 5H), 7.56b (d, 2H).

Section 3: Methods 39a-b

The following compounds were synthesized according to Section 3: Method 39:

| Section 3: | | | |
|---|---|---|---|
| Method # | Compound Name | m/z | SM |
| 39a | N-[1-(5-Benzyl-3-methyl-4-oxo- | 532 | Section 3: |
| | 4,5-dihydro-isothiazolo[5,4- | (MH⁺) | Method 38a |
| | d]pyrimidin-6-yl)-2-methyl-propyl]- | | |
| | N-(3-dimethylamino-propyl)- | | |
| | 4-methyl-benzamide | | |
| 39b | N-[1-(5-Benzyl-3-methyl-4-oxo- | 540 | Section 3: |
| | 4,5-dihydro-isothiazolo[5,4- | (MH⁺) | Method 38b |
| | d]pyrimidin-6-yl)-2-methyl-propyl]- | | |
| | N-(3-dimethylamino- | | |
| | propyl)-3-fluoro-4-methyl-benzamide | | |

Section 3: Methods 40-40b

The following compounds were chirally purified in same manner as (+) (3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 12). Chiral purification generally resulted in 99% purity of the (+) enantiomer.

| Section | | | | | |
|---|---|---|---|---|---|
| 3: | | Column | Solvent | (+) Enantiomer | |
| Method # | Compound Name | Type | composition | retention time | SM |
| 40 | (+) N-[1-(5-Benzyl-3- | Chiralpak | 85% hexanes | 7.6 min | Section |
| | methyl-4-oxo-4,5- | AD | 15% | | 3: |
| | dihydro-isothiazolo[5,4- | | isopropanol | | Method |
| | d]pyrimidin-6-yl)-2- | | 0.1% | | 39 |
| | methyl-propyl]-4-bromo- | | diethylamine | | |
| | N-(3-dimethylamino- | | | | |
| | propyl)-benzamide | | | | |
| | Section 3: Example E-2 | | | | |
| 40a | (+) N-[1-(5-Benzyl-3- | Chiralpak | 90% hexanes | 7.7 min | Section |
| | methyl-4-oxo-4,5- | AD | 10% | | 3: |
| | dihydro-isothiazolo[5,4- | | isopropanol | | Method |
| | d]pyrimidin-6-yl)-2- | | 0.1% | | 39a |
| | methyl-propyl]-N-(3- | | diethylamine | | |
| | dimethylamino-propyl)- | | | | |
| | 4-methyl-benzamide | | | | |
| | Section 3: Example E-1 | | | | |
| 40b | (+) N-[1-(5-Benzyl-3- | Chiralpak | 90% hexanes | 7.5 min | Section |
| | methyl-4-oxo-4,5- | AD | 10% | | 3: |
| | dihydro-isothiazolo[5,4- | | isopropanol | | Method |
| | d]pyrimidin-6-yl)-2- | | 0.1% | | 39b |
| | methyl-propyl]-N-(3- | | diethylamine | | |
| | dimethylamino-propyl)- | | | | |
| | 3-fluoro-4-methyl- | | | | |
| | benzamide | | | | |
| | Section 3: Example E-3 | | | | |

Section 3: Method 41

### 3-Methyl-5-(3-methyl-butyryl)-isoxazole-4-carboxylic acid amide

A mixture of 5-amino-3-methyl-isoxazole-4-carboxylic acid amide (10 g, 70 mmol) in 25 ml of isovaleric anhydride was stirred at 110-145° C. for 1 h. The brown solution was diluted with hexane (500 ml) and cooled down. The precipitated gum was separated from the mixture and washed with hexane, dried in vacuo. 3-Methyl-5-(3-methyl-butyryl)-isoxazole-4-carboxylic acid amide was obtained as a yellow gum. Further used without purification in Section 3: Method 42.

Section 3: Method 42

### 6-Isobutyl-3-methyl-5H-isoxazolo[5,4-d]pyrimidin-4-one

A suspension of 3-methyl-5-(3-methyl-butyryl)-isoxazole-4-carboxylic acid amide (Section 3: Method 41) (split into 40 vials) in 3.5 ml of 2N NaOH aq was subjected to microwave irradiation at 140° C. for 20 min. The resulting solution was cooled with an ice bath, and the pH was adjusted to 1˜3 with concentrated HCl. The solid was filtered, washed with water, dried over vacuum at 40° C. overnight. 6-Isobutyl-3-methyl-5H-isoxazolo[5,4-d]pyrimidin-4-one (8 g) was obtained as a white solid. 55% yield for two steps. m/z: 208 (MH⁺), ¹H NMR (DMSO-d₆): 0.76 (d, 6H), 1.95 (m, 1H), 2.25 (s, 3H), 2.32 (d, 2H), 12.55 (s, 1H).

Section 3: Method 43

### 5-Benzyl-6-isobutyl-3-methyl-5H-isoxazolo[5,4-d]pyrimidin-4-one

A suspension of 6-isobutyl-3-methyl-5H-isoxazolo[5,4-d]pyrimidin-4-one (Section 3: Method 42) (5 g, 24.4 mmol), benzylbromide (4.17 g, 24.4 mmol), potassium carbonate (6.7 g, 48.8 mmol) in 20 ml DMF was stirred at room temperature for 2 days. The mixture was diluted with water, extracted with EtOAc (100 ml×3), the combined organic phases were dried, concentrated, purified by flash column chromatography (elute: hexane-EtOAc=7:1). 5-benzyl-6-isobutyl-3-methyl-5H-isoxazolo[5,4-d]pyrimidin-4-one was obtained as white solid (3 g, 10.1 mmol) (41%). m/z: 298 (MH⁺), ¹H NMR (DMSO-d₆): 0.90 (d, 6H), 2.30 (m, 1H), 2.55 (s, 3H), 2.75 (d, 2H), 5.42 (s, 2H), 7.22-7.43 (m, 5H).

Section 3: Methods 43a-b

The following compounds were synthesized according to Section 3: Method 43:

| Section 3: | | |
|---|---|---|
| Method # | Compound Name | m/z |
| 43a | 5-(4-Fluoro-benzyl)-6-isobutyl-3-methyl- | 316 |
| | 5H-isoxazolo[5,4-d]pyrimidin-4-one | (MH⁺) |
| 43b | 5-(3-Fluoro-benzyl)-6-isobutyl-3-methyl- | 316 |
| | 5H-isoxazolo[5,4-d]pyrimidin-4-one | (MH⁺) |

Section 3: Method 44

### 5-Benzyl-6-(1-bromo-2-methyl-propyl)-3-methyl-5H-isoxazolo[5,4-d]pyrimidin-4-one

A solution of 5-benzyl-6-isobutyl-3-methyl-5H-isoxazolo[5,4-d]pyrimidin-4-one (Section 3: Method 43) (130 mg, 0.44 mmol) and sodium acetate (90 mg, 1.09 mmol, 2.5 eq) in glacial acetic acid (2 ml) was treated with a preformed bromine solution (0.7 ml bromine in 10 ml of glacial acetic acid) (1.54 ml, 2 mmol). The mixture was stirred at 110-120° C. for 1 day. Excess bromine (1.54 ml, 2 mmol) was added to the mixture every 4 hours for two times at 110-120° C. Water was added to the mixture to which was subsequently added potassium carbonate and extracted with DCM (20 ml×3), the combined organic phases were washed with water and dried, then concentrated to give the crude product which was purified by ISCO (elute: hexane-EtOAc). 100 mg (60%) of 5-benzyl-6-(1-bromo-2-methyl-propyl)-3-methyl-5H-isoxazolo[5,4-d]pyrimidin-4-one was obtained as a yellow gum. m/z: 376, 378 (MH⁺), ¹H NMR (DMSO-d₆): 0.55 (d, 3H), 1.02 (d, 3H), 2.48 (m, 4H), 4.75 (d, 1H), 5.60 (d, 1H), 5.70 (d, 1H), 7.16-7.30 (m, 5H).

Section 3: Methods 44a-b

The following compounds were synthesized according to Section 3: Method 44:

| Section 3: | | | |
|---|---|---|---|
| Method # | Compound Name | m/z | SM |
| 44a | 6-(1-Bromo-2-methyl-propyl)- | 394, 396 | Section 3: |
| | 5-(4-fluoro-benzyl)-3-methyl-5H- | (MH⁺) | Method 43a |
| | isoxazolo[5,4-d]pyrimidin-4-one | | |
| 44b | 6-(1-Bromo-2-methyl-propyl)-5- | 394, 396 | Section 3: |
| | (3-fluoro-benzyl)-3-methyl-5H- | (MH⁺) | Method 43b |
| | isoxazolo[5,4-d]pyrimidin-4-one | | |

Section 3: Method 45

### 6-(1-Azido-2-methyl-propyl)-5-benzyl-3-methyl-5H-isoxazolo[5,4-d]pyrimidin-4-one

A suspension of 5-benzyl-6-(1-bromo-2-methyl-propyl)-3-methyl-5H-isoxazolo[5,4-d]pyrimidin-4-one (Section 3: Method 44) (100 mg, 0.266 mmol) and sodium azide (34.5 mg, 0.53 mmol) in DMF (2 ml) was stirred at 60° C. for 1 h. Water (5 ml) was added to the mixture and then extracted with EtOAc (3×20 ml). The combined organic phases were washed with brine (10 ml), dried, concentrated to obtain 6-(1-azido-2-methyl-propyl)-5-benzyl-3-methyl-5H-isoxazolo[5,4-d]pyrimidin-4-one which was purified by ISCO (Hexane-EtOAc). 50 mg (56%) of a colorless oil was obtained. m/z: 339 (MH⁺), ¹H NMR (DMSO-d₆): 0.60 (d, 3H), 0.95 (d, 3H), 2.25 (m, 1H), 2.45 (s, 3H), 4.19 (d, 1H), 5.30 (d, 1H), 5.42 (d, 1H), 7.12-7.30 (m, 5H).

Section 3: Methods 45a-b

The following compounds were synthesized according to Section 3: Method 45:

| Section 3: | | | |
|---|---|---|---|
| Method # | Compound Name | m/z | SM |
| 45a | 6-(1-Azido-2-methyl-propyl)-5- | 357 | Section |
| | (4-fluoro-benzyl)-3-methyl- | (MH⁺) | 3: |
| | 5H-isoxazolo[5,4-d]pyrimidin-4-one | | Method |
| | | | 44a |
| 45b | 6-(1-Azido-2-methyl-propyl)-5- | 357 | Section |
| | (3-fluoro-benzyl)-3-methyl- | (MH⁺) | 3: |
| | 5H-isoxazolo[5,4-d]pyrimidin-4-one | | Method |
| | | | 44b |

Section 3: Method 46

### 6-(1-Amino-2-methyl-propyl)-5-benzyl-3-methyl-5H-isoxazolo[5,4-d]pyrimidin-4-one

A mixture of 6-(1-azido-2-methyl-propyl)-5-benzyl-3-methyl-5H-isoxazolo[5,4-d]pyrimidin-4-one (Section 3: Method 45) (40 mg, 1.118 mmol), triphenylphosphine (62 mg, 0.237 mmol) and water (4 μl) in THF was stirred at 60° C. for 5 hours. Excess amount of water (30 μl) was added to the mixture and stirred at 60° C. for another 10 hours. The volatile solvent was distilled out, the crude product was purified by ISCO (EtOAc:hexane=60%. 25 mg (68%) of 6-(1-amino-2-methyl-propyl)-5-benzyl-3-methyl-5H-isoxazolo[5,4-d]pyrimidin-4-one was obtained as colorless oil. m/z: 313 (MH⁺), ¹H NMR (DMSO-d₆): 0.55 (d, 3H), 0.95 (d, 3H), 2.02 (m, 1H), 2.15 (br, 2H), 2.55 (s, 3H), 3.59 (d, 1H), 5.38 (d, 1H), 5.65 (d, 1H), 7.25-7.42 (m, 5H).

Section 3: Methods 46a-b

The following compounds were synthesized according to Section 3: Method 46:

| Section 3: | | | |
|---|---|---|---|
| Method # | Compound Name | m/z | SM |
| 46a | 6-(1-Amino-2-methyl-propyl)-5-(4-fluoro- | 331 | Section |
| | benzyl)-3-methyl-5H-isoxazolo[5,4- | (MH⁺) | 3: |
| | d]pyrimidin-4-one | | Method |
| | | | 45a |
| 46b | 6-(1-Amino-2-methyl-propyl)-5-(3-fluoro- | 331 | Section |
| | benzyl)-3-methyl-5H-isoxazolo[5,4- | (MH⁺) | 3: |
| | d]pyrimidin-4-one | | Method |
| | | | 45b |

Section 3: Method 47

### {3-[1-(5-Benzyl-3-methyl-4-oxo-4,5-dihydro-isoxazolo[5,4-d]-pyrimidin-6-yl)-2-methyl-propylamino]-propyl}-carbamic acid tert-butyl ester

A mixture of 6-(1-amino-2-methyl-propyl)-5-benzyl-3-methyl-5H-isoxazolo[5,4-d]pyrimidin-4-one (Section 3: Method 46) (20 mg, 0.064 mmol) and (3-oxo-propyl)-carbamic acid tert-butyl ester (11 mg, 0.064 mmol) in DCM (5 ml) with dried 4AMS was stirred for 1 h at room temperature. Then sodium triacetoxyborohydride (2 eq) and 1 drop of acetic acid were added to the mixture. The mixture was stirred at room temperature for 1 day. The mixture was filtered through a 2μ cartridge, the filtrate was concentrated, the crude mixture was purified by ISCO (elute: EtOAc-hexane=30%˜60%) to give 18 mg (60%) of {3-[1-(5-benzyl-3-methyl-4-oxo-4,5-dihydro-isoxazolo[5,4-d]pyrimidin-6-yl)-2-methyl-propylamino]-propyl}-carbamic acid tert-butyl ester as a white solid. m/z: 470 (MH⁺), ¹H NMR (DMSO-d₆): 0.65 (d, 3H), 0.80 (d, 3H), 1.10 (m, 2H), 1.25 (s, 9H), 1.32 (d, 1H), 1.70-1.90 (m, 2H), 2.18 (m, 1H), 2.49 (s, 3H), 2.70 (m, 2H), 3.48 (d, 1H), 5.15 (d, 1H), 5.51 (d, 1H), 6.55 (br, 1H), 7.12-7.32 (m, 5H).

Section 3: Methods 47a-b

The following compounds were synthesized according to Section 3: Method 47:

| Section 3: | | | |
|---|---|---|---|
| Method # | Compound Name | m/z | SM |
| 47a | (3-{1-[5-(4-Fluoro-benzyl)- | 488 | Section |
| | 3-methyl-4-oxo-4,5-dihydro- | (MH⁺) | 3: |
| | isoxazolo[5,4-d]pyrimidin-6-yl]- | | Method |
| | 2-methyl-propylamino}-propyl)- | | 46a |
| | carbamic acid tert-butyl ester | | |
| 47b | (3-{1-[5-(3-Fluoro-benzyl)- | 488 | Section |
| | 3-methyl-4-oxo-4,5-dihydro- | (MH⁺) | 3: |
| | isoxazolo[5,4-d]pyrimidin- | | Method |
| | 6-yl]-2-methyl-propylamino}- | | 46b |
| | propyl)-carbamic acid tert- | | |
| | butyl ester | | |

Section 3: Method 48

### {3-[[1-(5-Benzyl-3-methyl-4-oxo-4,5-dihydro-isoxazolo[5,4-d]pyrimidin-6-yl)-2-methyl-propyl-(4-methyl-benzoyl)-amino]-propyl}-carbamic acid tert-butyl ester

A solution of {3-[1-(5-benzyl-3-methyl-4-oxo-4,5-dihydro-isoxazolo[5,4-d]pyrimidin-6-yl)-2-methyl-propylamino]-propyl}-carbamic acid tert-butyl ester (Section 3: Method 47) (100 mg, 0.213 mmol) in DCM (4 ml) was added p-toluoyl chloride (66 mg, 0.426 mmol) followed by triethylamine (65 mg, 0.639 mmol). The mixture was stirred at 30-40° C. for 2 days. The mixture was then diluted with DCM, washed with saturated sodium bicarbonate aq. The organic phase was dried, filtered, and concentrated. The crude oil was purified by ISCO (solvent: EtOAc-hexane) to give {3-[[1-(5-benzyl-3-methyl-4-oxo-4,5-dihydro-isoxazolo[5,4-d]pyrimidin-6-yl)-2-methyl-propyl]-(4-methyl-benzoyl)-amino]-propyl}-carbamic acid tert-butyl ester as white solid (115 mg, 0.196 mmol). m/z: 588 (MH⁺).

Section 3: Methods 48a-b

The following compounds were synthesized according to Section 3: Method 48:

| Section 3: | | | | Acylating |
|---|---|---|---|---|
| Method # | Compound Name | m/z | SM | agent |
| 48a | {3-[{1-[5-(4-Fluoro-benzyl)- | 606 | Section | 4-methyl- |
| | 3-methyl-4-oxo-4,5-dihydro- | (MH⁺) | 3: | benzoyl |
| | isoxazolo[5,4-d]pyrimidin-6- | | Method | chloride |
| | yl]-2-methyl-propyl}-(4- | | 47a | |
| | methyl-benzoyl)-amino]- | | | |
| | propyl}-carbamic acid | | | |
| | tert-butyl ester | | | |
| 48b | {3-[{1-[5-(3-Fluoro- | 606 | Section | 4-methyl- |
| | benzyl)-3-methyl-4-oxo-4,5- | (MH⁺) | 3: | benzoyl |
| | dihydro-isoxazolo[5,4- | | Method | chloride |
| | d]pyrimidin-6-yl]-2- | | 47b | |
| | methyl-propyl}-(4- | | | |
| | methyl-benzoyl)-amino]- | | | |
| | propyl}-carbamic acid | | | |
| | tert-butylester | | | |

Section 3: Method 49

### Chiral purification of (+) {3-[{1-[5-(3-Fluoro-benzyl)-3-methyl-4-oxo-4,5-dihydro-isoxazolo[5,4-d]pyrimidin-6-yl]-2-methyl-propyl}-(4-methyl-benzoyl)-amino]-propyl}-carbamic acid tert-butyl ester

The following compound was chirally purified in same manner as (+) (3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 12). Chiral purification generally resulted in 99% purity of the (+) enantiomer.

| Section 3: | | Column | Solvent | (+) Enantiomer | |
|---|---|---|---|---|---|
| Method # | Compound Name | Type | composition | retention time | SM |
| 49 | (+) {3-[{1-[5-(3- | Chiralpak | 80% hexane | 7.4 min | Section |
| | Fluoro-benzyl)-3- | AD | 20% | | 3: |
| | methyl-4-oxo-4,5- | | isopropanol | | Method |
| | dihydro-isoxazolo[5,4- | | 0.1% | | 48b |
| | d]pyrimidin-6-yl]-2- | | diethylamine | | |
| | methyl-propyl}-(4- | | | | |
| | methyl-benzoyl)- | | | | |
| | amino]-propyl}- | | | | |
| | carbamic acid tert- | | | | |
| | butyl ester | | | | |

Section 3: Method 50

### N-(3-Amino-propyl)-N-[1-(5-benzyl-3-methyl-4-oxo-4,5-dihydro-isoxazolo[5,4-d]pyrimidin-6-yl)-2-methyl-propyl]-4-methyl-benzamide hydrogen chloride

A solution of {3-[[1-(5-benzyl-3-methyl-4-oxo-4,5-dihydro-isoxazolo[5,4-d]pyrimidin-6-yl)-2-methyl-propyl]-(4-methyl-benzoyl)-amino]-propyl}-carbamic acid tert-butyl ester (Section 3: Method 48) (0.058 g, 0.1 mmol) in 3 ml of 4 M HCl in dioxane was stirred at room temperature for 2 hr. The solvent was distilled off by vacuo, the residue was dried at 40˜50° C. for overnight under vacuum. N-(3-Amino-propyl)-N-[1-(5-benzyl-3-methyl-4-oxo-4,5-dihydro-isoxazolo[5,4-d]pyrimidin-6-yl)-2-methyl-propyl]-4-methyl-benzamide was obtained as the HCl salt. Yield was 0.046 g (88%). m/z 488 (MH⁺), ¹H NMR (500 MHz, 100° C., DMSO-d₆): 0.48 (d, 3H), 0.94 (d, 3H), 1.30 (m, 1H), 1.60 (m, 1H), 2.35 (m, 2H), 2.38 (s, 3H), 2.58 (s, 3H), 2.70 (m, 1H), 3.37 (m, 2H), 5.11 (d, 1H), 5.64 (d, 1H), 5.90 (d, 1H), 7.23-7.39 (m, 9H), 7.63 (br, 3H).

Section 3: Methods 50a-b

The following compounds were synthesized according to Section 3: Method 50:

| Section 3: | | | |
|---|---|---|---|
| Method # | Compound Name | m/z | SM |
| 50a | N-(3-Amino-propyl)- | 506 | Section |
| | N-{1-[5-(4-fluoro-benzyl)- | (MH⁺) | 3: |
| | 3-methyl-4-oxo-4,5-dihydro- | | Method |
| | isoxazolo[5,4-d]pyrimidin- | | 48a |
| | 6-yl]-2-methyl-propyl}- | | |
| | 4-methyl-benzamide | | |
| | hydrogen chloride | | |
| 50b | (+) N-(3-Amino-propyl)- | 506 | Section |
| | N-{1-[5-(3-fluoro-benzyl)- | (MH⁺) | 3: |
| | 3-methyl-4-oxo-4,5-dihydro- | | Method |
| | isoxazolo[5,4-d]pyrimidin- | | 49 |
| | 6-yl]-2-methyl-propyl}-4- | | |
| | methyl-benzamide | | |
| | hydrogen chloride | | |
| | Section 3: Example F-3 | | |

Section 3: Methods 51 and 51a

The following compounds were chirally purified in same manner as (+) (3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 12). Chiral purification generally resulted in 99% purity of the (+) enantiomer.

| Section | | | | | |
|---|---|---|---|---|---|
| 3: | | Column | Solvent | (+) Enantiomer | |
| Method # | Compound Name | Type | composition | retention time | SM |
| 51 | (+) N-(3-Amino-propyl)- | Chiralpak | 60% hexanes | 7.9 min | Section |
| | N-[1-(5-benzyl-3-methyl- | AD | 40% | | 3: |
| | 4-oxo-4,5-dihydro- | | isopropanol | | Method |
| | isoxazolo[5,4- | | 0.1% | | 50 |
| | d]pyrimidin-6-yl)-2- | | diethylamine | | |
| | methyl-propyl]-4-methyl- | | | | |
| | benzamide | | | | |
| | Section 3: Example F-1 | | | | |
| 51a | (+) N-(3-Amino-propyl)- | Chiralpak | 60% hexanes | 7.5 min | Section |
| | N-{1-[5-(4-fluoro- | AD | 40% | | 3: |
| | benzyl)-3-methyl-4-oxo- | | isopropanol | | Method |
| | 4,5-dihydro- | | 0.1% | | 50a |
| | isoxazolo[5,4- | | diethylamine | | |
| | d]pyrimidin-6-yl]-2- | | | | |
| | methyl-propyl}-4- | | | | |
| | methyl-benzamide | | | | |
| | Section 3: Example F-2 | | | | |

Section 3: Method 52

### 3-Amino-2-thioformyl-but-2-enoic acid ethyl ester

To an ice cold solution of phosphoryl chloride (20 mL, 220 mmol), anhydrous DMF (60 mL) was added dropwise and the resulting solution was added dropwise during 30 min to a stirred solution of the ethyl crotonate (25.83 g, 200 mmol) in anhydrous THF (400 mL) with the temperature maintained at 0° C. The resulting mixture was allowed to warm to room temperature and stirred overnight and then for 4 h at 30° C.; it was then allowed to stand overnight in a refrigerator. Addition of ether (200 mL) resulted in a yellow oil from which the ether layer was decanted. The resulting oil was washed several times with ether until the ether layer became clear. The oily product was dissolved in DCM (800 mL) and was vigorously shaken with aqueous sodium hydrogen sulfide (2M; 500 mL). The organic layer was separated and the aqueous layer washed with DCM (100 mL). The combined organic layers were washed with water (600 mL), brine (400 mL), dried (Na₂SO₄) and concentrated to get orange crystals. The thus obtained product was triturated with DCM/hexanes to get pure product as orange crystals (25.6 g, 74%). ¹H NMR (300 MHz) δ: 1.33 (t, 3H), 2.57 (s, 3H), 4.23 (q, 2H), 6.83 (bs, 1H), 10.97 (s, 1H), 13.93 (s, 1H).

Section 3: Method 53

### 3-Methyl-isothiazole-4-carboxylic acid ethyl ester

To a solution of 3-amino-2-thioformyl-but-2-enoic acid ethyl ester (Section 3: Method 52) (25.6 g, 147 mmol) in ethanol (300 mL), was added m-chloroperbenzoic acid (33.3 g, 77%, 149 mmol) in ethanol (200 mL) dropwise with stirring at room temperature. After the completion of the addition the reaction mixture was heated at 75° C. for 2 h after which the MS showed the complete disappearance of the starting material. The reaction mixture was diluted with ether (500 mL) and the ethereal solution was washed with 0.1 M NaOH solution (3×500 mL) and once with water (400 mL) dried (Na₂SO₄) and concentrated to get the pure product as light brown oil. Yield 23.5 g (93%). ¹H NMR (300 MHz) δ: 1.40 (t, 3H), 2.73 (s, 3H), 5.07 (t, 1H), 4.36 (q, 2H), 9.24 (s, 1H).

Section 3: Method 54

### 3-Methyl-isothiazole-4-carboxylic acid

To a solution of 3-methyl-isothiazole-4-carboxylic acid ethyl ester (Section 3: Method 53) (23.3 g, 136 mmol) in THF (200 mL) aqueous NaOH (6.5 g, 162 mmol, in 100 ml of water) was added and the mixture was stirred at room temperature for 16 h. The TLC of the reaction mixture showed the complete disappearance of the starting material. The reaction mixture was cooled in an ice bath and acidified to pH 5 using 6M HCl and the resultant mixture was extracted with ether (3×100 mL). The ether layers were combined, washed with water (100 mL), brine (100 mL), dried (Na₂SO₄) and concentrated to about 10 mL. Addition of hexanes to the above mixture resulted in the precipitation of the product which was filtered off, washed with hexanes and dried to provide the pure product as a tan powder. Yield 15.3 g (79%). ¹H NMR (300 MHz) δ 2.39 (s, 3H), 8.98 (s, 1H).

Section 3: Method 55

### (3-Methyl-isothiazol-4-yl)-carbamic acid tert-butyl ester

To a solution of 3-methyl-isothiazole-4-carboxylic acid (Section 3: Method 54) (14.8 g, 103 mmol) in anhydrous t-BuOH (100 mL) triethyl amine (10.5 g, 104 mmol) was added followed by the dropwise addition of diphenylphosphoryl azide (28.6 g, 104 mmol) and the resulting mixture was heated at reflux overnight after which the TLC showed the complete disappearance of the starting material. The reaction mixture was cooled to room temperature and poured into ice cold water (500 mL). The aqueous layer was extracted with ether (3×100 mL) and the combined organic layers were washed with satd, NaHCO₃ (100 mL), brine (100 mL) and dried (Na₂SO₄). Concentration of the ether solution provided the crude product which was purified by column chromatography to get the pure product as light brown crystals. Yield 21.4 g (97%). ¹H NMR (300 MHz) δ 1.53 (s, 9H), 2.40 (s, 3H), 6.50 (s, 1H), 8.66 (s, 1H).

Section 3: Method 56

### 4-tert-Butoxycarbonylamino-3-methyl-isothiazole-5-carboxylic acid

To a solution of (3-methyl-isothiazol-4-yl)-carbamic acid tert-butyl ester (Section 3: Method 55) (21.4 g, 100 mmol) in anhydrous THF (200 mL) at −78° C., LDA (139 mL, 1.8 M solution, 250 mmol) was added dropwise over a period of 1 h. The reaction mixture was stirred at that temperature for a further 3 h after which powdered dry ice was added and the reaction slowly allowed to warm to room temperature overnight. The reaction mixture was quenched by adding saturated NH₄Cl solution and extracted with ether (3×100 mL) and the combined ether layers were back extracted with satd. NaHCO₃ (3×100 mL). The aqueous layers were combined and acidified to pH 5 using 6M HCl and extracted with ether (4×100 mL). The combined ether layers were dried (Na₂CO₃) and concentrated to give the pure acid as an off white powder. Yield 11 g (39%). ¹H NMR (300 MHz) δ 1.47 (s, 9H), 2.44 (s, 3H), 8.53 (bs, 1H), 9.68 (bs, 1H).

Section 3: Method 57

### 4-Amino-3-methyl-isothiazole-5-carboxylic acid

4-tert-Butoxycarbonylamino-3-methyl-isothiazole-5-carboxylic acid (Section 3: Method 56) (11 g, 45 mmol) was dissolved in 50 mL of 4M solution of HCl in 1,4-dioxane (200 mmol) and the resulting solution was stirred at room temperature overnight. The TLC showed the complete disappearance of the starting acid. The reaction was concentrated and the residue was triturated with ether and the precipitated hydrochloride salt was filtered off and washed with ether and dried to provide the product as a light brown powder. Yield 8.2 g (100%). ¹H NMR (300 MHz, DMSO-d₆) δ 2.30 (s, 3H), 8.85 (bs, 3H).

Section 3: Method 58

### 3-Methyl-5-propyl-isothiazolo[4,5-d][1,3]oxazin-7-one

To a solution of 4-amino-3-methyl-isothiazole-5-carboxylic acid (Section 3: Method 57) (2.91 g, 15 mmol) in pyridine (20 mL) at 0° C., was added dropwise a solution of butyryl chloride (3.18 g, 30 mmol) in chloroform (30 m]L). The reaction mixture was allowed to warm to room temperature and stirred overnight. Chloroform (200 mL) was added to the reaction mixture followed by 2M HCl (200 mL) and the mixture was stirred. The chloroform layer was further washed with 2M HCl (100 mL), water (100 mL), brine (100 mL) and concentrated. Column purification of the thus obtained crude product provided the pure product as light brown solid. Yield 2 g (64%). ¹H NMR (300 MHz) 61.03 (t, 3H), 1.80-1.92 (m, 2H), 2.65 (s, 3H), 2.76 (t, 2H).

Section 3: Method 59

### 6-Benzyl-3-methyl-5-propyl-6H-isothiazolo[4,5-d]pyrimidin-7-one

3-Methyl-5-propyl-isothiazolo[4,5-d][1,3]oxazin-7-one (Section 3: Method 58) (200 mg, 1.02 mmol) was taken in a 10 m]L microwavable pyrex tube and benzyl amine (1 g, 9.34 mmol) was added to it. The resulting mixture was heated in a microwave synthesizer (CEM's. Discoverer) at 200° C. for 20 min. The MS of the reaction mixture showed the complete disappearance of the starting material and the presence of the product peak at 286 (MH⁺). The reaction mixture was diluted with 1N HCl (10 mL) and extracted with EtOAc (2×30 mL). The combined EtOAc layers were washed with water, brine, dried and concentrated. The thus obtained crude product was purified by column chromatography to isolate the pure product as a white solid. Yield 208 mg (71%). ¹H NMR (300 MHz) δ 0.98 (t, 3H), 1.76-1.88 (m, 2H), 2.68 (s, 3H), 2.74 (t, 2H), 5.42 (s, 2H), 7.10-7.19 (m, 2H), 7.28-7.39 (m, 3H).

Section 3: Method 60

### 6-Benzyl-5-(1-bromo-propyl)-3-methyl-6H-isothiazolo[4,5-d]pyrimidin-7-one

To a solution of 6-benzyl-3-methyl-5-propyl-6H-isothiazolo[4,5-d]pyrimidin-7-one (Section 3: Method 59) (208 mg, 0.69 mmol) and sodium acetate (0.5 g, 5 mmol) in acetic acid (10 mL) at 100° C., a solution of the bromine (0.232 g, 1.46 mmol) in acetic acid (20 mL) was added dropwise [The next drop of Bromine was added only after the previous drop had reacted completely by monitoring the decolorization] over a period of 30 min. The reaction mixture was cooled after the addition and the TLC (eluent 10% EtOAc in hexanes) and MS showed the complete disappearance of the SM and only the product. The reaction mixture was poured into ice water and extracted with EtOAc (3×30 mL) and the organic layers were combined and washed with 2% sodium thiosulfate solution (30 mL), water (50 mL), brine (50 mL) and dried (Na₂SO₄). Concentration of the organic layer provided the product and it was pure enough to be used in the next step. Yield 260 mg (99%). ¹H NMR (300 MHz) δ 0.77 (t, 3H), 2.20-2.54 (m, 2H), 2.70 (s, 3H), 4.67 (t, 1H), 4.95 (d, 1H), 6.25 (d, 1H) 7.10-7.19 (m, 2H), 7.30-7.39 (m, 3H).

Section 3: Method 61

### N-(3-Amino-propyl)-N-[1-(6-benzyl-3-methyl-7-oxo-6,7-dihydro-isothiazolo[4,5-d]pyrimidin-5-yl)-propyl]-4-methyl-benzamide hydrogen chloride

To a solution of 6-benzyl-5-(1-bromo-propyl)-3-methyl-6H-isothiazolo[4,5-d]pyrimidin-7-one (Section 3: Method 60) (260 mg, 0.70 mmol) in anhydrous DMF (10 mL), ethyl diisopropylamine (387 mg, 3 mmol) and N-(3-aminopropyl)carbamic acid tert-butyl ester (174 mg, 1 mmol) were added at room temperature and the mixture was stirred at room temperature for 1 h after which the MS analysis showed the complete disappearance of the starting bromide and only the product peak at 472 (MH⁺) was observed. The reaction mixture was diluted with water (100 mL) and extracted with EtOAc (3×60 mL). The combined organic extracts were dried and concentrated to get the crude amine which was dissolved in chloroform (40 mL) and diisopropylethylamine (387 mg, 3 mmol) was added and the mixture was heated to 60° C. To the stirred hot solution p-toluoyl chloride (154 mg, 1 mmol) in chloroform (20 mL) was added dropwise and the mixture was refluxed for 12 h after which the MS showed the complete disappearance of the amine and only the product peak at 590 (MH⁺). The reaction mixture was concentrated and the crude product was purified by column chromatography to isolate the pure acylated product (80 mg, 20% overall from bromide) which was treated with 4M HCl in 1,4-dioxane (10 mL) for 30 min. The dioxane was evaporated in a rotary evaporator and the residue was dissolved in water and freeze dried to get the pure product as a white fluffy solid. Yield 60 mg (16% overall from bromide). m/z 490 (MH⁺); ¹H NMR (300 MHz, DMSO-d₆, 96° C.) δ 0.65 (t, 3H), 1.36-1.50 (m, 1H), 1.60-1.72 (m, 1H), 1.88-1.99 (m, 1H), 2.14-2.26 (m, 1H), 2.35 (s, 3H), 2.47 (t, 2H), 2.68 (s, 3H), 3.32-3.44 (m, 2H), 4.90 (d, 1H), 5.50 (bs, 1H), 5.76 (d, 1H), 6.96-7.34 (m, 9H), 7.68 (bs, 3H).

Section 3: Method 62

### Chiral purification of (+) N-(3-Amino-propyl)-N-[1-(6-benzyl-3-methyl-7-oxo-6,7-dihydro-isothiazolo[4,5-d]pyrimidin-5-yl)-propyl]-4-methyl-benzamide

The following compound was chirally purified in same manner as (+) (3-[[1-(5-benzyl-3-methyl-4-oxo-4,5-dihydro-isothiazolo[5,4-d]pyrimidin-6-yl)-propyl]-(4-methyl-benzoyl)-amino]-propyl)-carbamic acid tert-butyl ester (Section 3: Method 12). Chiral purification generally resulted in 99% purity of the (+) enantiomer.

| Section | | | | | |
|---|---|---|---|---|---|
| 3: | | Column | Solvent | (+) Enantiomer | |
| Method # | Compound Name | Type | composition | retention time | SM |
| 62 | (+) N-(3-Amino- | Chiralpak | 70% hexane | 11.7 min | Section |
| | propyl)-N-[1-(6-benzyl- | AD | 30% | | 3: |
| | 3-methyl-7-oxo-6,7- | | isopropanol | | Method |
| | dihydro-isothiazolo[4,5- | | 0.1% | | 61 |
| | d]pyrimidin-5-yl)- | | diethylamine | | |
| | propyl]-4-methyl- | | | | |
| | benzamide | | | | |
| | Section 3: Example G-1 | | | | |

Alternative Procedures to Prepare Certain Starting Materials
Section 3: Method 1

### 2-(1-Ethoxy-ethylidene)-malononitrile (alternative procedure)

Triethyl orthoacetate (1.6 L, 9 mol), malononitrile (500 g, 7.57 mol) and glacial acetic acid (25 ml) were placed in a 5 l RB flask equipped with a stirrer, thermometer and a Vigreux column (20×1 in.) on top of which a distillation condenser was placed. The reaction mixture was heated and ethyl alcohol began to distil when the temperature of the reaction mixture was about 85-90° C. After about 3 h., the temperature of the reaction mixture reached 140° C. Then the reaction was concentrated in a rotary evaporator to remove the low-boiling materials and the residue was stirred with isopropyl alcohol (1 l) and cooled in an ice bath. The crystallized product was filtered off washed with isopropyl alcohol (200 ml), hexanes (600 ml) and dried at 50° C. in a vacuum oven overnight to yield 2-(1-ethoxy-ethylidene)-malononitrile (974 g, 94%) as a golden yellow solid [mp 92. ° C. (lit. 90-92° C., MCCall. M. A. J. Org. Chem. 1962, 27, 2433-2439.)].

Section 3: Method 2

### (2E)-2-Cyano-3-ethoxybut-2-enethioamide (alternative procedure)

2-(1-Ethoxy-ethylidene)-malononitrile (Section 3: Method 1) (300 g, 2.2 mol) was dissolved in anhydrous benzene (3.1 l, slight warming required) and 20 ml of triethylamine was added. The mixture was mechanically stirred and hydrogen sulfide was bubbled into this solution for 2 h and a solid formed. Then N₂ was bubbled through the reaction mixture for 40 min. The precipitated solid was filtered off, washed with cold benzene (200 ml) and dried in a vacuum oven overnight to isolate (2E)-2-cyano-3-ethoxybut-2-enethioamide (332 g, 88%) as light brown crystals.

Section 3: Method 3

### (2E)-3-Amino-2-cyanobut-2-enethioamide (alternative procedure)

(2E)-2-Cyano-3-ethoxybut-2-enethioamide (Section 3: Method 2) (150 g, 0.88 mol) was dissolved in 7M solution of ammonia in methanol (2.9 L) and stirred at r.t. overnight. The reaction mixture was concentrated and the residue was crystallized from hot water (1. L) to provide (2E)-3-amino-2-cyanobut-2-enethioamide (111.6 g, 89%) as brown crystals. ¹H NMR (300 MHz, DMSO-d₆) δ 2.22 (s, 3H), 7.73 (bs, 1H), 8.53 (bs, 1H), 9.01 (bs, 1H), 11.60 (bs, 1H).

Section 3: Method 4

### 5-Amino-3-methylisothiazole-4-carbonitrile (alternative procedure)

To a stirred solution of (2E)-3-amino-2-cyanobut-2-enethioamide (Section 3: Method 3) (111 g, 0.78 mol) in methanol (2 L) was added dropwise 200 ml of 35% hydrogen peroxide over a period of 30 min. After the completion of the addition the mixture was stirred at 60° C. for 3 h after which the TLC showed the completion of the reaction. The reaction mixture was evaporated to 300 ml in a rotary evaporator and cooled in an ice-bath. The crystallized product was filtered off and washed with isopropyl alcohol (100 ml) and dried in vacuum at 50° C. overnight to provide 5-amino-3-methylisothiazole-4-carbonitrile (105.63 g, 96%) as a light yellow crystalline solid. ¹H NMR (300 MHz, DMSO-d₆) δ 2.24 (s, 3H), 8.00 (bs, 2H).

Section 3: Method 24

### N-(4-Cyano-3-methyl-isothiazol-5-yl)-3-methyl-butyramide (alternative procedure)

To a solution of 5-amino-3-methylisothiazole-4-carbonitrile (Section 3: Method 4) (105.6 g, 0.76 mol) in pyridine (250 ml) at 0° C., isovaleryl chloride (100 g, 0.83 mol) in chloroform (300 ml) was added dropwise. After the completion of the addition the reaction mixture was allowed to warm to r.t. and stirred overnight. The TLC and the MS showed the complete disappearance of the starting material and the reaction mixture was diluted with CHCl₃ (600 ml), washed with water (200 ml), 2N HCl (600 ml), satd. NaHCO₃ (200 ml), brine (200 ml) and dried over Na₂SO₄. Concentration of the CHCl₃ layer provided the crude product which was triturated from DCM/hexanes (1/10) and filtered off to isolate N-(4-cyano-3-methyl-isothiazol-5-yl)-3-methyl-butyramide (149.7 g, 88%) as an off-white crystalline solid. ¹H NMR (300 MHz) δ 1.04 (d, 6H), 2.18-2.32 (m, 1H), 2.46 (d, 2H), 2.53 (s, 3H), 9.87 (bs, 1H).

Section 3: Method 25

### 3-Methyl-5-(3-methyl-butyrylamino)-isothiazole-4-carboxylic acid amide (alternative procedure)

To a solution of N-(4-cyano-3-methyl-isothiazol-5-yl)-3-methyl-butyramide (Section 3: Method 24) (72 g, 322 mmol) in 30% aqueous NH₄OH (2.1 L), was added dropwise 1.3 L of hydrogen peroxide at 40° C. After 20 min the temperature of the reaction mixture rose to 60° C. The addition was completed in 1.5 h. After an additional 2 h the MS showed the completion of the reaction. The reaction mixture was cooled in ice and con HCl was slowly added with cooling till the pH of the reaction mixture turns 7.6. The precipitated product was filtered and dried in vacuum oven to get the pore amide (36 g, 46%). The filtrate was saturated with NaCl and extracted with super solvent (34:66, t-butanol: 1,2-dichloroethane) and the combined organic extracts were washed with water (500 ml), brine (600 ml) and dried (Na₂SO₄) and concentrated. The residue on trituration with EtOAc/hexanes (1/4) provided an additional 9.8 g of pure product. Total yield of 45.8 g (58%) 3-methyl-5-(3-methyl-butyrylamino)-isothiazole-4-carboxylic acid amide. ¹H NMR (300 MHz) δ 1.03 (d, 6H), 2.24 (m, 1H), 2.43 (d, 2H), 2.69 (s, 3H), 5.98 (bs, 2H), 11.77 (bs, 1H).

Section 3: Method 26

### 6-Isobutyl-3-methyl-5H-isothiazolo[5,4-d]pyrimidin-4-one (alternative procedure)

The 3-methyl-5-(3-methyl-butyrylamino)-isothiazole-4-carboxylic acid amide (Section 3: Method 25) (45.8 g, 190 mmol) was suspended in 700 ml of 30% NH₃ and then was heated to 140° C. for 5 h in a pressure reactor. The mixture was poured into a 4 L beaker and cooled in an ice bath. To the cold solution con HCl (560 ml) was added dropwise to pH 7.5 and a white precipitate was formed. The precipitated product was filtered off, washed with water (100 ml) and dried under vacuum overnight. 6-Isobutyl-3-methyl-5H-isothiazolo[5,4-d]pyrimidin-4-one (11 g, 26%) was isolated as an off-white powder. ¹H NMR (300 MHz) δ 1.05 (d, 6H), 2.32 (m, 1H), 2.69 (d, 2H), 2.82 (s, 3H).

Section 3: Method 27

### 5-Benzyl-6-isobutyl-3-methyl-5H-isothiazolo[5,4-d]pyrimidin-4-one (alternative procedure)

To a solution of the 6-isobutyl-3-methyl-5H-isothiazolo[5,4-d]pyrimidin-4-one (Section 3: Method 26) (11 g, 49 mmol) in 60 ml of anhydrous DMF at 0° C., was added 13.8 g (100 mmol) of anhydrous K₂CO₃ followed by benzyl bromide (9.3 g, 54 mmol) and the mixture was stirred at 0-20° C. overnight. The TLC of the reaction mixture showed the complete disappearance of the SM. The reaction mixture was poured into ice-cold water and extracted with EtOAc (3×100 ml). The combined extracts were washed with water (100 ml), brine (100 ml), dried (Na₂SO₄) and concentrated. The TLC and the ¹H NMR showed the presence of two products N alkylated as well as O-alkylated products in a ratio of 75:25. The products were separated by column (silica gel) chromatography using 10% EtOAc in hexanes. The major N-alkylated product 5-benzyl-6-isobutyl-3-methyl-5H-isothiazolo[5,4-d]pyrimidin-4-one was isolated as white crystalline solid (10.8 g, 70%). ¹H NMR (300 MHz) δ 0.94 (d, 6H), 2.23-2.37 (m, 1H), 2.64 (d, 2H), 2.82 (s, 3H), 5.38 (s, 2H), 7.10-7.38 (m, 5H).

Section 3: Method 28

### 5-Benzyl-6-(1-bromo-2-methyl-propyl)-3-methyl-5H-isothiazolo[5,4-d]pyrimidin-4-one (alternative procedure)

To a solution of 5-benzyl-6-isobutyl-3-methyl-5H-isothiazolo[5,4-d]pyrimidin-4-one (Section 3: Method 27) (5.81 g, 18.5 mmol) and sodium acetate (10 g) in acetic acid (100 ml) at 100° C., a solution of the bromine (6 g, 38 mmol) in acetic acid (60 ml) was added dropwise over a period of 20 minutes. The reaction mixture was stirred at that temperature for 30 min and cooled and the TLC (eluent 10% EtOAc in hexanes) and MS showed the complete disappearance of the SM and only the product. The reaction mixture was poured into ice water and extracted with EtOAc (3×60 ml) and the organic layers were combined and washed with 2% sodium thiosulfate solution (60 ml), water (100 ml), brine (100 ml) and dried over Na₂SO₄. Concentration of the organic layer provided 5-benzyl-6-(1-bromo-2-methyl-propyl)-3-methyl-5H-isothiazolo[5,4-d]pyrimidin-4-one (7.27 g, 99%) as white crystalline solid. ¹H NMR (300 MHz) δ 0.54 (d, 3H), 1.11 (d, 3H), 2.62-2.76 (m, 1H), 2.83 (s, 3H), 4.42 (d, 1H), 4.80 (d, 1H), 6.22 (d, 1H), 7.12-7.42 (m, 5H).

Section 3: Method 29

### 6-(1-Azido-2-methyl-propyl)-5-benzyl-3-methyl-5H-isothiazolo[5,4-d]pyrimidin-4-one (alternative procedure)

To a solution of 5-benzyl-6-(1-bromo-2-methyl-propyl)-3-methyl-5H-isothiazolo[5,4-d]pyrimidin-4-one (Section 3: Method 28) (7.27 g, 18.5 mmol) in anhydrous DMF (60 ml), sodium azide (2.33 g, 37 mmol) was added and the mixture was stirred at room temperature for 2 hour. The TLC of the RM showed the complete disappearance of the starting bromide. The reaction mixture was poured into ice water (300 ml) and extracted with EtOAc (3×100 ml). The organic layer was washed with water (100 ml), brine (100 ml) and dried (Na₂SO₄). Concentration of the organic layer provided the crude product which was purified by column (silica gel) chromatography using 30% EtOAc in hexanes as eluent to isolate 6-(1-azido-2-methyl-propyl)-5-benzyl-3-methyl-5H-isothiazolo[5,4-d]pyrimidin-4-one (6.16 g, 94%) as a low melting solid. ¹H NMR (300 MHz) δ 0.57 (d, 3H), 1.07 (d, 3H), 2.50-2.74 (m, 1H), 2.98 (s, 3H), 3.71 (d, 1H), 5.05 (d, 1H), 5.78 (d, 1H), 7.12-7.40 (m, 5H).

Section 3: Method 30

### 6-(1-Amino-2-methyl-propyl)-5-benzyl-3-methyl-5H-isothiazolo[5,4-d]pyrimidin-4-one (alternative procedure)

To a solution of 6-(1-azido-2-methyl-propyl)-5-benzyl-3-methyl-5H-isothiazolo[5,4-d]pyrimidin-4-one (Section 3: Method 29) (6.8 g, 19.2 mmol) in methanol (400 ml) was added 5% Pd/C (1 g, 20% by wt.) and the resulting mixture was stirred at r.t. in an atmosphere of H₂ and the progress of the reaction was monitored by MS. After the disappearance of the starting material the reaction mixture was filtered through celite and washed with EtOAc. Concentration of the filtrate provided 6-(1-amino-2-methyl-propyl)-5-benzyl-3-methyl-5H-isothiazolo[5,4-d]pyrimidin-4-one (5.42 g, 86%).

Section 3: Method 31

### {3-[1-(5-Benzyl-3-methyl-4-oxo-4,5-dihydro-isothiazolo[5,4-d]pyrimidin-6-yl]-2-methyl-propylamino]-propyl}-carbamic acid tert-butyl ester (alternative procedure)

To a solution of 6-(1-amino-2-methyl-propyl)-5-benzyl-3-methyl-5H-isothiazolo[5,4-d]pyrimidin-4-one (Section 3: Method 30) (5.4 g, 16.5 mmol) in DCM (100 ml), 4 Å molecular sieves (50 g) was added followed by N-boc protected 3-aminopropanal (2.84 g, 16.5 mmol)) and the reaction mixture was stirred at r.t. overnight and the progress of the reaction was monitored by MS. After the complete disappearance of the starting amine, a catalytic amount of acetic acid was added to the reaction followed by sodium triacetoxyborohydride (3.49 g, 16.5 mmol) and the reaction mixture was stirred at r.t. for 4 h. After the completion of the reaction (MS), the reaction mixture was filtered and the residue was washed with DCM and the filtrate was washed with water (100 mL), brine (100 mL) and concentrated to give {3-[1-(5-Benzyl-3-methyl-4-oxo-4,5-dihydro-isothiazolo[5,4-d]pyrimidin-6-yl)-2-methyl-propylamino]-propyl}-carbamic acid tert-butyl ester (8.3 g, theoretical yield=7.9 g) which was used as such for the next reaction.

Section 3: Method 33

### {3-[[1-(5-Benzyl-3-methyl-4-oxo-4,5-dihydro-isothiazolo[5,4-d]pyrimidin-6-yl)-2-methyl-propyl]-(4-methyl-benzoyl)-amino]-propyl}-carbamic acid tert-butyl ester (alternative procedure)

To a solution of {3-[1-(5-Benzyl-3-methyl-4-oxo-4,5-dihydro-isothiazolo[5,4-d]pyrimidin-6-yl)-2-methyl-propylamino]-propyl}-carbamic acid tert-butyl ester obtained from Section 3: Method 31 alternative procedure above in chloroform (300 ml), diisopropylethylamine (6 g, 46.5 mmol) was added and the reaction mixture was heated to 60° C. To the hot solution a solution of the p-toluoyl chloride (3.78 g, 24.4 mmol) in chloroform (150 ml) was added dropwise and the resulting solution was refluxed overnight. The TLC showed the disappearance of most of the SM. The reaction mixture was washed with water (2×100 ml), satd, NaHCO₃ (200 ml) brine (100 ml) and dried (Na₂SO₄). Concentration of the organic layer provided the crude product which was purified by column (silica gel) chromatography using 10-30% EtOAc in hexanes as eluent. Yield=6.14 g (62%) of {3-[[1-(5-benzyl-3-methyl-4-oxo-4,5-dihydro-isothiazolo[5,4-d]pyrimidin-6-yl)-2-methyl-propyl]-(4-methyl-benzoyl)-amino]-propyl}-carbamic acid tert-butyl ester. White foam, mp. 70-71° C. m/z 604 (MH⁺), ¹H NMR (DMSO-d₆, 300 MHz, 95° C.) δ: 0.48 (d, 3H), 0.90 (d, 3H), 1.26 m, 1H), 1.28 (s, 9H), 2.33 (s, 3H), 2.47 (d, 2H), 2.72-2.64 (m, 1H), 2.72 (s, 3H), 3.24 (t, 2H), 5.08 (d, 1H), 5.60 (d, 1H), 5.90 (d, 1H), 7.20-7.40 (m, 9H).

Section 3: Method 63

### 5-Amino-3-methylisothiazole-4-carboxamide

To a chilled solution of sulfuric acid (7.2 volumes, 12.9 equivs) was charged 5-amino-3-methylisothiazole-4-carbonitrile (Section 3: Method 4) (1.0 equiv). The temperature was maintained below 55° C. The reaction mixture was heated to 70° C. and held for 1 hour until TLC showed disappearance of starting material. The mixture was cooled to 60-65° C. before the ammonia (21 volumes) was charged to pH 10. The mixture was cooled to 20° C., aged overnight and filtered. The resulting solid was washed with dilute ammonia (3.6 volumes) and dried at 40° C. to give a pale brown solid (typical yield 80%). ¹H NMR (300 MHz, DMSO-d6) δ 2.46(s, 3H), 6.28 (s, 1H).

Section 3: Method 26

### 6-Isobutyl-3-methyl-5H-isothiazolo[5,4-d]pyrimidin-4-one (alternative procedure)

To a 2 L flask equipped with Dean Stark was charged 5-amino-3-methylisothiazole-4-carboxamide (Section 3: Method 63) (1 equiv), p-toluene sulphonic acid (0.049 equiv), DMF (9.75 volumes). The reaction was stirred until a solution was obtained and isovaleraldehyde (1.10 equiv) and toluene (4.9 volumes) were added. The resulting mixture was heated to 130° C. and held at reflux for 1 hour removing water via a Dean Stark apparatus. Once the reaction was complete toluene was removed under vacuum distillation. Sodium bisulfite (2.50 equiv) was charged and the mixture was held at 115° C. for 7 hours, then cooled to room temperature overnight. The solid was removed by filtration through harborlite and washed with DMF (1 volume). Analysis showed conversion to product and the reaction was heated to 50° C., water (15 volumes) was added and the resulting precipitate was cooled to room temperature and held for 1 h. The product was isolated by filtration and washed with water (2×0.5 volumes), dried to give a pale brown solid (typical yield 89%).

Section 3: Method 31

### {3-[1-(5-Benzyl-3-methyl-4-oxo-4,5-dihydro-isothiazolo[5,4-d]pyrimidin-6-yl]-2-meth propylamino]-propyl}-carbamic acid tert-butyl ester (alternative procedure)

To (3,3-diethoxypropyl)amine (1.00 equiv) in THF (2 volumes) was charged di-t-butyldicarbonate (1.05 equiv) in THF (3 volumes). The reaction was heated to 45° C. and held for ½ h. Analysis showed the disappearance of starting material, and the resulting solution was heated to 65° C. p-Toluene sulphonic acid (0.1 equiv) and water (5 volumes) were charged over 10 mins, heating continued at 65° C. and held for ½ hour. Analysis showed disappearance of tert-butyl (3,3-diethoxypropyl)carbamate. Toluene (15 volumes) charged, layers separated and washed with water (5 volumes). A fraction of the solution obtained (0.95 equivs) was charged to a solution containing 6-(1-amino-2-methyl-propyl)-5-benzyl-3-methyl-5H-isothiazolo[5,4-d]pyrimidin-4-one (Section 3: Method 30) (1 equiv), toluene (5 volumes) and molecular sieves (1 weight equivalent). The reaction mixture was stirred overnight at room temperature until the reaction was complete. THF (2.5 volumes) were charged followed by sodium acetoxyborohydride (2.0 equiv) and the resulting mixture held overnight until reaction was complete. Aqueous acetic acid (20% v/v, 2.5 volumes) were charged over 10 minutes, stirred at room temperature for 10 minutes, filtered and washed with water (2.5 volumes). The layers were separated and the organic layer was concentrated under vacuo at 50° C. Further toluene was charged (2.5 volumes) and the solvent removed. The product was obtained as an orange oil (typical yield 92%). m/z 486 (MH⁺).

### EXAMPLE A

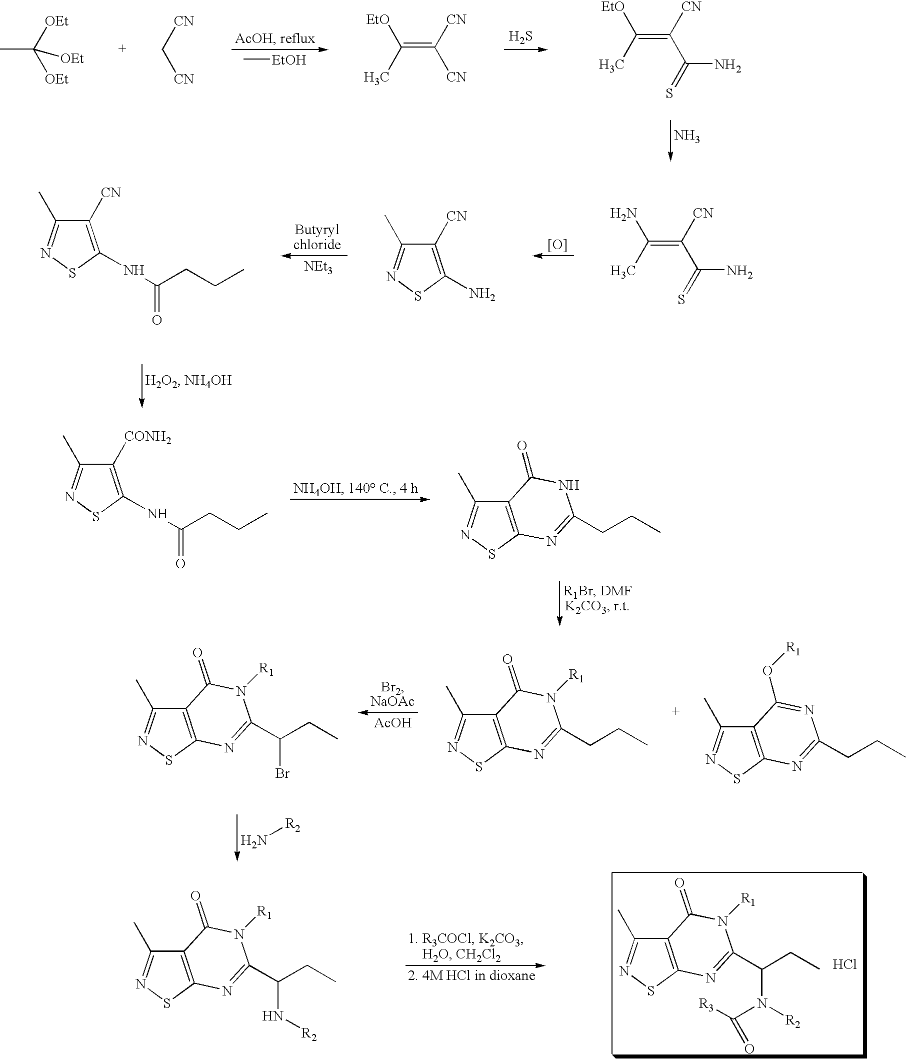

### SECTION 3: EXAMPLES A

The following compounds were synthesized according to synthetic scheme A above:

| Ex. | Compound | ¹H NMR | m/z | SM |
|---|---|---|---|---|
| A1 | (+) N-(3-Amino-propyl)- | (DMSO-d₆, 500MHz, 96° C.) δ: 0.63(t, | m/z | Section |
| | N-[1-(5-benzyl-3-methyl- | 3H), 1.40-1.74(m, 2H), 1.75-1.96(m, | 490(MH⁺) | 3: |
| | 4-oxo-4,5-dihydro- | 1H), 2.05-2.20(m, 1H), 2.39(s, | | Method |
| | isothiazolo[5,4- | 3H), 2.46(t, 2H), 2.72(s, 3H), 3.36(t, | | 13 |
| | d]pyrimidin-6-yl)-propyl]- | 2H), 4.83(d, 1H), 5.50(bs, 1H), | | |
| | 4-methyl-benzamide | 5.77(d, 1H), 6.95-7.37(m, 9H), 7.79(bs, | | |
| | hydrogen chloride | 3H) | | |
| A2 | (+) N-(3-Amino-propyl)- | (DMSO-d₆, 500MHz, 96° C.) δ: 0.66(t, | m/z | Section |
| | N-{1-[5-(4-fluoro-benzyl)- | 3H), 1.38-1.74(m, 2H), 1.82-1.98(m, | 508(MH⁺) | 3: |
| | 3-methyl-4-oxo-4,5- | 1H), 2.02-2.20(m, 1H), 2.34(s, | | Method |
| | dihydro-isothiazolo[5,4- | 3H), 2.42(t, 2H), 2.72(s, 3H), 3.36(t, | | 13a |
| | d]pyrimidin-6-yl]-propyl}- | 2H), 4.85(d, 1H), 5.49(bs, 1H), | | |
| | 4-methyl-benzamide | 5.70(d, 1H), 7.05-7.27(m, 8H), 7.76(bs, | | |
| | hydrogen chloride | 3H) | | |
| A3 | (+) N-(3-Amino-propyl)- | (500MHz, DMSO-d₆, 100° C.) δ | m/z | Section |
| | N-{1-[5-(3-fluoro-benzyl)- | ppm: 0.70(t, 3H), 1.40-1.54(m, | 508(MH⁺) | 3: |
| | 3-methyl-4-oxo-4,5- | 1H), 1.62-1.76(m, 1H), 1.85-2.01(m, | | Method |
| | dihydro-isothiazolo[5,4- | 1H), 2.14-2.27(m, 1H), 2.38(s, | | 13b |
| | d]pyrmidin-6-yl]-propyl}- | 3H), 2.44-2.49(m, 2H), 2.76(s, | | |
| | 4-methyl-benzamide | 3H), 3.35-3.46(m, 2H), 4.87(br s, | | |
| | hydrogen chloride | 1H), 5.48(br s, 1H), 5.75(d, 1H), | | |
| | | 6.84-6.96(m, 2H), 7.06-7.15(m, | | |
| | | 1H), 7.20-7.31(m, 4H), 7.33-7.41(m, | | |
| | | 1H), 7.52(br s, 3H) | | |
| A4 | (+) N-(3-Amino-propyl)- | (DMSO-d₆, 500MHz, 96° C.) δ: 0.68(t, | m/z | Section |
| | N-[1-(5-benzyl-3-methyl- | 3H), 1.50-1.72(m, 2H), 1.91-1.96(m, | 554, | 3: |
| | 4-oxo-4,5-dihydro- | 1H), 2.13-2.17(m, 1H), 2.47(t, | 556(MH⁺) | Method |
| | isothiazolo[5,4- | 2H), 2.77(s, 3H), 3.38(t, 2H), 4.95(d, | | 13g |
| | d]pyrimidin-6-yl)-propyl]- | 1H), 5.57(bs, 1H), 5.80(d, 1H), | | |
| | 4-bromo-benzamide | 7.13(m, 2H), 7.28-7.36(m, 5H), | | |
| | hydrogen chloride | 7.64(d, 2H), 7.80(br, 1H) | | |
| A5 | (+) N-(3-Amino-propyl)- | (DMSO-d₆, 500MHz, 96° C.) δ: 0.69(t, | m/z | Section |
| | N-[1-(5-benzyl-3-methyl- | 3H), 1.42-1.83(m, 2H), 1.89-2.01(m, | 510(MH⁺) | 3: |
| | 4-oxo-4,5-dihydro- | 1H), 2.10-2.20(m, 1H), | | Method |
| | isothiazolo[5,4- | 2.46(hidden by DMSO, 2H), 2.77(s, | | 13c |
| | d]pyrimidin-6-yl)-propyl]- | 3H), 3.39(bm, 2H), 4.94(d, 1H), | | |
| | 4-chloro-benzamide | 5.58(bs, 1H), 5.81(d, 1H), 7.12-7.56(m, | | |
| | hydrogen chloride | 9H) | | |
| A6 | (+) N-(3-Amino-propyl)- | (DMSO-d₆, 500MHz, 96° C.) δ: 0.67(t, | m/z | Section |
| | N-[1-(5-benzyl-3-methyl- | 3H), 1.45(m, 1H), 1.70(m, 1H), | 508(MH⁺) | 3: |
| | 4-oxo-4,5-dihydro- | 1.92(m, 1H), 2.16(m, 1H), 2.31(s, | | Method |
| | isothiazolo[5,4- | 3H), 2.46 (2H, hidden by DMSO), | | 13d |
| | d]pyrimidin-6-yl)-propyl]- | 2.76(s, 3H), 3.39(t, 2H), 4.93(d, | | |
| | 3-fluoro-4-methyl- | 1H), 5.54(bs, 1H), 5.81(d, 1H), | | |
| | benzamide hydrogen | 7.09-7.52(m, 8H), 7.74(br, 3H) | | |
| | chloride | | | |
| A7 | (+) N-(3-Amino-propyl)- | (DMSO-d₆, 500MHz, 96° C.) δ: 0.70(t, | m/z | Section |
| | N-[1-(5-benzyl-3-methyl- | 3H), 1.50-1.70(m, 2H), 1.94(m, | 544, | 3: |
| | 4-oxo-4,5-dihydro- | 1H), 2.20(m, 1H), 2.46(m, 2H), | 545, | Method |
| | isothiazolo[5,4- | 2.78(s, 3H), 3.30(m, 2H), 5.10(d, | 546(MH⁺) | 13e |
| | d]pyrimidin-6-yl)-propyl]- | 1H), 5.82(bs, 1H), 5.93(d, 1H), | | |
| | 2,3-dichloro-benzamide | 7.29-7.72(m, 8H), 7.72(br, 3H) | | |
| | hydrogen chloride | | | |
| A8 | (+) Benzo[b]thiophene-2- | (DMSO-d₆, 500MHz, 96° C.) δ: 0.71(t, | m/z | Section |
| | carboxylic acid (3-amino- | 3H), 1.46-1.61(m, 1H), 1.88-1.92(m, | 532(MH⁺) | 3: |
| | propyl)-[1-(5-benzyl-3- | 1H), 1.93-1.97(m, 1H), 2.21-2.26(m, | | Method |
| | methyl-4-oxo-4,5-dihydro- | 1H), 2.62(t, 2H), 2.76(s, | | 13f |
| | isothiazolo[5,4- | 3H), 3.65(t, 2H), 4.96(d, 1H), 5.66(bs, | | |
| | d]pyrimidin-6-yl)- | 1H), 5.78(d, 1H), 7.05(bm, | | |
| | propyl]amide hydrogen | 2H), 7.25(bm, 3H), 7.30-7.50(m, | | |
| | chloride | 2H), 7.60(s, 1H), 7.85-7.99(m, 2H) | | |
| A9 | (+) N-(2-Amino-ethyl)-N- | (DMSO-d₆, 500MHz, 96° C.) δ: 0.60(t, | m/z | Section |
| | [1-(5-benzyl-3-methyl-4- | 3H), 1.85-2.05(m, 2H), 2.45(s, | 476(MH⁺) | 3: |
| | oxo-4,5-dihydro- | 3H), 2.76(s, 3H), 2.90(m, 2H), 3.80(m, | | Method |
| | isothiazolo[5,4- | 2H), 4.70(d, 1H), 5.35(bs, 1H), | | 13h |
| | d]pyrimidin-6-yl)-propyl]- | 5.80(d, 1H), 6.88-7.35(m, 9H), | | |
| | 4-methyl-benzamide | 7.75-7.85(br, 3H) | | |
| | hydrogen chloride | | | |
| A10 | (+) N-[1-(5-Benzyl-3- | (DMSO-d₆, 90° C.) δ: 0.66(t, 3H), | m/z | Section |
| | methyl-4-oxo-4,5-dihydro- | 0.90-1.10(m, 1H), 1.30-1.49(m, | 518(MH⁺) | 3: |
| | isothiazolo[5,4- | 1H), 1.81(m, 8H), 1.85-1.95(m, | | Method |
| | d]pyrimidin-6-yl)-propyl]- | 1H), 2.05-2.15(m, 1H), 2.35(s, 3H), | | 12i |
| | N-(3-dimethylamino- | 2.75(s, 3H), 3.41(t, 2H), 4.96(d, | | |
| | propyl)-4-methyl- | 1H), 5.71(bs, 1H), 5.92(d, 1H), | | |
| | benzamide | 7.10-7.44(m, 9H) | | |

### EXAMPLE B

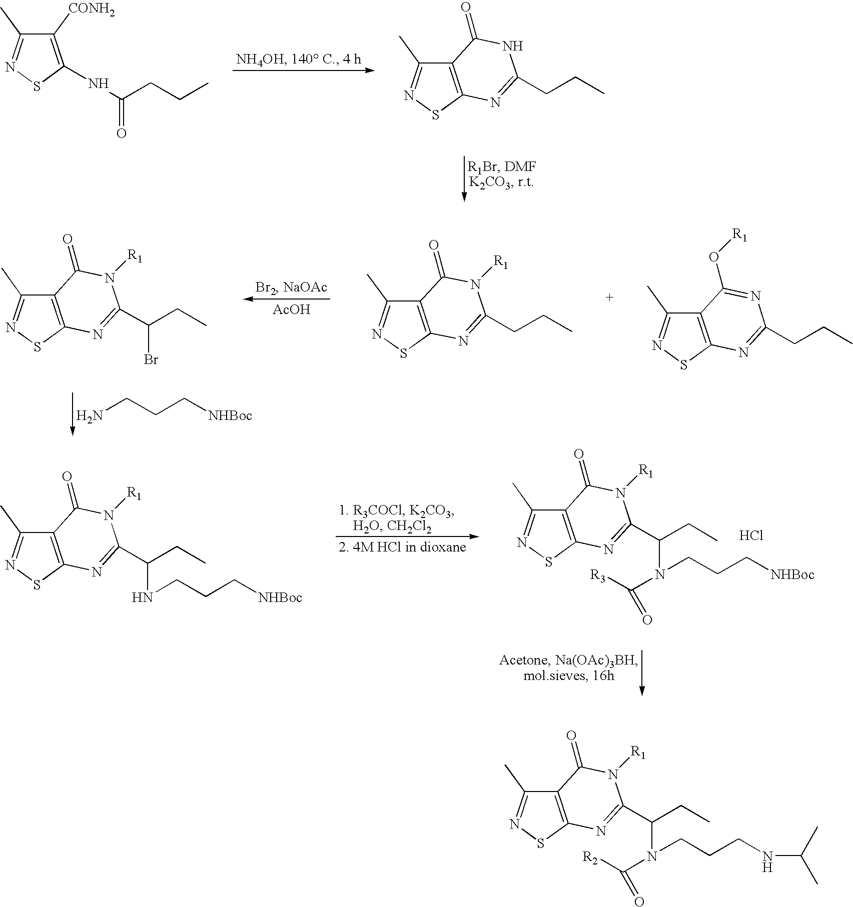

### SECTION 3: EXAMPLE B

The following compounds were synthesized according to synthetic scheme B above:

| Ex. | Compound | ¹H NMR | m/z | SM |
|---|---|---|---|---|
| B1 | (+) N-[1-(5-Benzyl-3-methyl- | (DMSO-d₆, 90° C.) δ: 0.65(t, 3H), | m/z | Section |
| | 4-oxo-4,5-dihydro- | 0.75-0.85(d, 6H), 1.01-1.11(m, | 532(MH⁺) | 3: |
| | isothiazolo[5,4-d]pyrimidin- | 1H), 1.35-1.50(m, 1H), 1.80-1.98(m, | | Method |
| | 6-yl)-propyl]-N-(3- | 1H), 2.00-2.19(m, 3H), 2.35(s, | | 15 |
| | isopropylamino-propyl)-4- | 3H), 2.80(s, 3H), 3.00-3.05(b, 2H), | | |
| | methyl-benzamide | 3.40(m, 2H), 4.90(d, 1H), 5.70(bs, | | |
| | | 1H), 5.80(d, 1H), 7.00-7.40(m, 9H) | | |

### EXAMPLE C

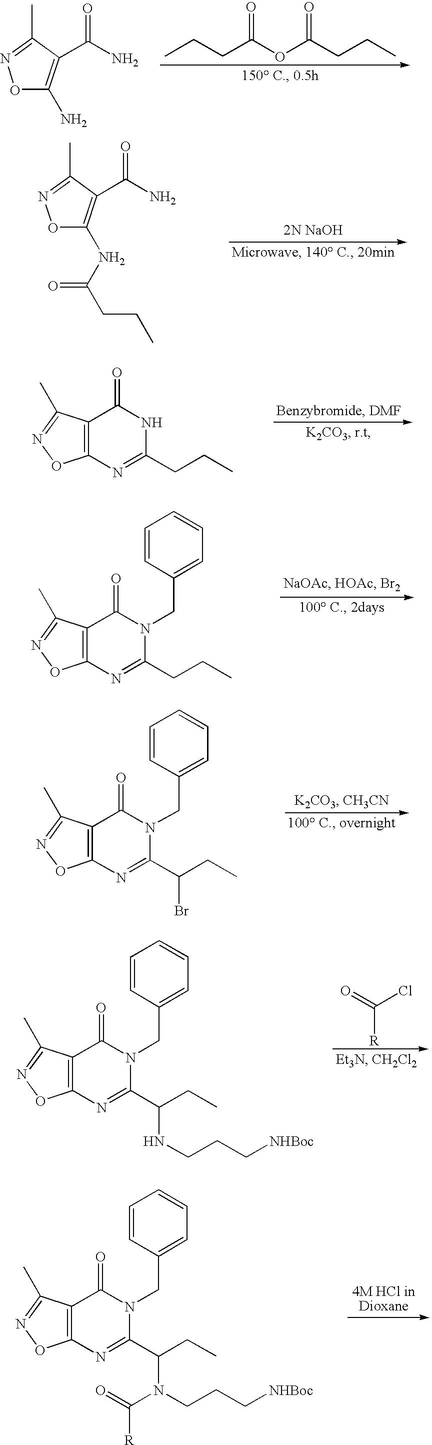

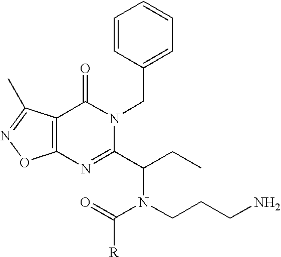

### SECTION 3: EXAMPLE C

The following compounds were synthesized according to synthetic scheme C above:

| Ex. | Compound | ¹H NMR | m/z | SM |
|---|---|---|---|---|
| C1 | (+) N-(3-Amino-propyl)-N-[1- | (500MHz, 100° C.), DMSO-d₆) | m/z | Section |
| | (5-benzyl-3-methyl-4-oxo-4,5- | δ: 0.68(t, 3H), 1.52(m, 1H), | 474(MH⁺) | 3: |
| | dihydro-isoxazolo[5,4- | 1.72(m, 1H), 1.92(m, 1H), | | Method |
| | d]pyrimidin-6-yl)-propyl]-4- | 2.10(m, 1H), 2.39(s, 3H), 2.51(m, | | 23 |
| | methyl-benzamide hydrogen | 2H), 2.57(s, 3H), 3.41(m, | | |
| | chloride | 2H), 4.85(br, 1H), 5.50(br, | | |
| | | 1H), 5.77(d, 1H), 7.07(br, 2H), | | |
| | | 7.24-7.35(m, 7H), 7.73(br, 3H) | | |

### EXAMPLE D

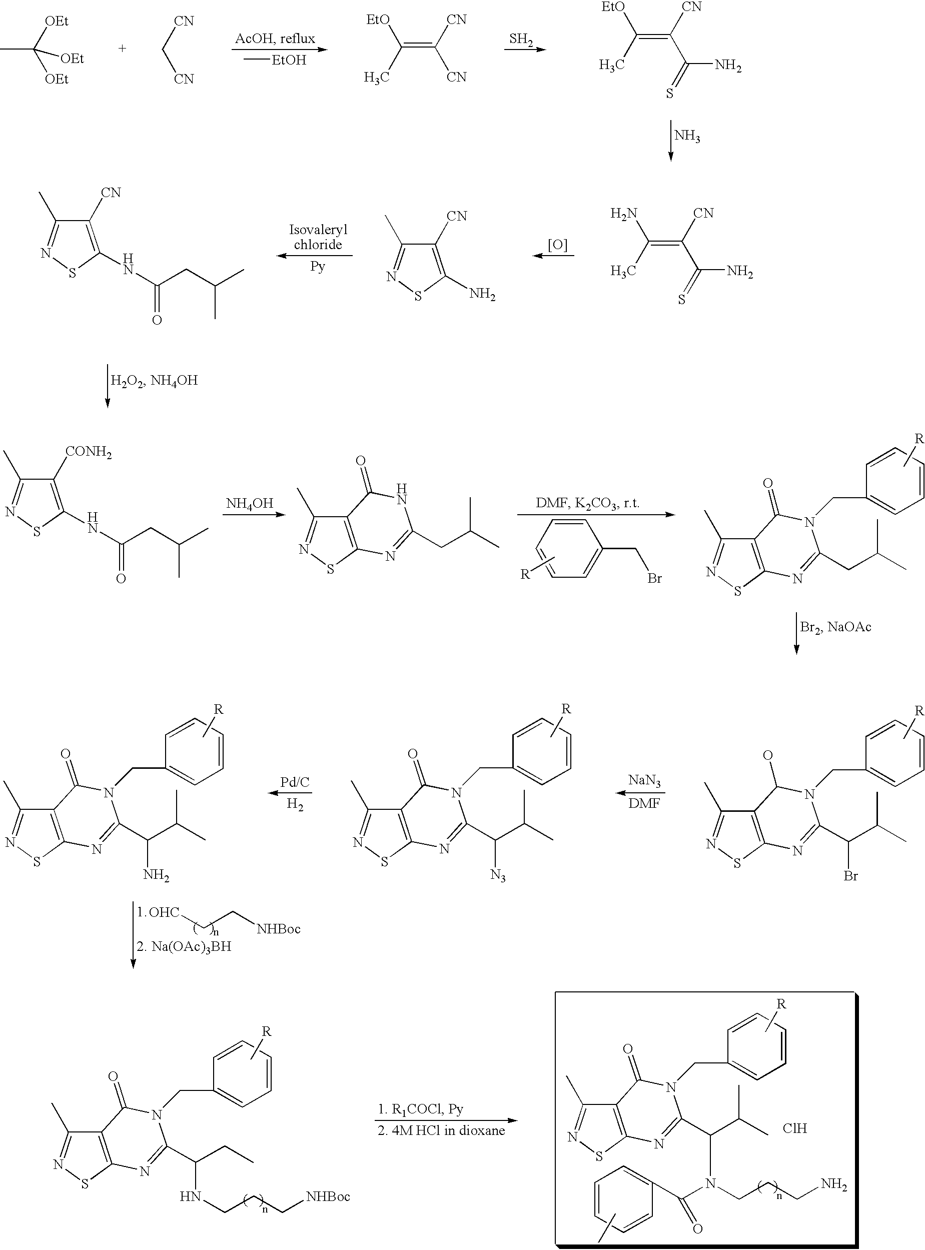

### SECTION 3: EXAMPLES D

The following compounds were synthesized according to synthetic scheme D above:

| Ex. | Compound | ¹H NMR | m/z | SM |
|---|---|---|---|---|
| D1 | (+) N-(3-Amino-propyl)-N- | (90° C., DMSO-d₆) δ: 0.47(d, 3H), | m/z | Section |
| | {1-[5-(4-fluoro-benzyl)-3- | 0.92(d, 3H), 1.10-1.28(m, 1H), | 522(MH⁺) | 3: |
| | methyl-4-oxo-4,5-dihydro- | 1.44-1.56(m, 1H), 2.27(t, 2H), 2.36(s, | | Method |
| | isothiazolo[5,4-d]pyrimidin- | 3H), 2.66-2.72(m, 1H), 2.75(s, | | 35a |
| | 6-yl]-2-methyl-propyl}-4- | 3H), 3.35(t, 2H), 5.04(d, 1H), 5.57(d, | | |
| | methyl-benzamide hydrogen | 1H), 5.86(d, 1H), 7.12-7.43(m, | | |
| | chloride | 8H), 7.71-7.81(m, 3H) | | |
| D2 | (+) N-(3-Amino-propyl)-N- | (500MHz, 96° C., DMSO-d₆) δ: 0.45(d, | m/z | Section |
| | [1-(5-benzyl-3-methyl-4-oxo- | 3H), 0.90(d, 3H), 1.12-1.30(m, | 504(MH⁺) | 3: |
| | 4,5-dihydro-isothiazolo[5,4- | 1H), 1.46-1.63(m, 1H), 2.25(t, 2H), | | Method |
| | d]pyrimidin-6-yl)-2-methyl- | 2.36(s, 3H), 2.64-2.7(m, 1H), 2.68(s, | | 36 |
| | propyl]-4-methyl-benzamide | 3H), 3.34(t, 2H), 5.06(d, 1H), | | |
| | | 5.59(d, 1H), 5.90(d, 1H), 7.20-7.40(m, | | |
| | | 9H), 7.71(bs, 3H) | | |
| D3 | (+) N-(3-Amino-propyl)-N- | (500MHz, DMSO-d₆, 90° C.) δ: 0.52(d, | m/z | Section |
| | {1-[5-(3-fluoro-benzyl)-3- | 3H), 0.94(d, 3H), 1.15-1.25(m, | 522(MH⁺) | 3: |
| | methyl-4-oxo-4,5-dihydro- | 1H), 1.26-1.33(m, 1H), 1.45-1.58(m, | | Method |
| | isothiazolo[5,4-d]pyrimidin- | 1H), 2.32(m, 2H), 2.38(s, 3H), | | 35b |
| | 6-yl]-2-methyl-propyl}-4- | 2.78(s, 3H), 3.32-3.40(m, 2H), 5.11(bd, | | |
| | methyl-benzamide | 1H), 5.56 (bd, 1H), 5.90-5.93(d, | | |
| | | 1H), 7.11-7.38(m, 8H), 7.58(b, | | |
| | | 2H) | | |
| D4 | (+) N-(2-Amino-ethyl)-N-[1- | (500MHz, DMSO-d₆, 96° C.) δ: 0.44(d, | m/z | Section |
| | (5-benzyl-3-methyl-4-oxo- | 3H), 0.90(d, 3H), 1.09-1.12(m, | 554, | 3: |
| | 4,5-dihydro-isothiazolo[5,4- | 1H), 2.55-2.75(m, 2H), 2.79(s, 3H), | 556(MH⁺) | Method |
| | d]pyrimidin-6-yl)-2-methyl- | 3.62-3.75(m, 2H), 5.05(m, 1H), | | 35d |
| | propyl]-4-bromo-benzamide | 5.60(d, 1H), 5.93(d, 1H), 7.21-7.40(m, | | |
| | hydrogen chloride | 9H), 7.61(m, 4H) | | |
| D5 | (+) N-(2-Amino-ethyl)-N-[1- | (DMSO-d₆, 500MHz, 90° C.) δ: 0.39(d, | m/z | Section |
| | (5-benzyl-3-methyl-4-oxo- | 3H), 0.93(d, 3H), 2.40(bm, 4H), | 490(MH⁺) | 3: |
| | 4,5-dihydro-isothiazolo[5,4- | 2.55-2.70(m, 2H), 2.79(s, 3H), | | Method |
| | d]pyrimidin-6-yl)-2-methyl- | 3.68-3.75(m, 2H), 5.00(b, 1H), 5.55(b, | | 35c |
| | propyl]-4-methyl-benzamide | 1H), 5.91-5.95(d, 1H), 7.15-7.43(m, | | |
| | hydrogen chloride | 9H), 7.60-7.71(bs, 2H). | | |
| D6 | (+) N-(2-Amino-ethyl)-N-[1- | (500MHz, DMSO-d₆, 90° C.) δ: 0.39(d, | m/z | Section |
| | (5-benzyl-3-methyl-4-oxo- | 3H), 0.93(d, 3H), 2.20-2.39(m, | 508(MH⁺) | 3: |
| | 4,5-dihydro-isothiazolo[5,4- | 4H), 2.60-2.70(m, 2H), 2.79(s, 3H), | | Method |
| | d]pyrimidin-6-yl)-2-methyl- | 3.63-3.74(m, 2H), 5.00(b, 1H), 5.55(b, | | 35e |
| | propyl]-3-fluoro-4-methyl- | 1H), 5.91-5.95(d, 1H), 7.15-7.48(m, | | |
| | benzamide hydrogen chloride | 8H), 7.68(bs, 2H) | | |
| D7 | (+) N-(3-Amino-propyl)-N- | (500MHz, DMSO-d₆, 90° C.) δ: 0.48(d, | m/z | Section |
| | [1-(5-benzyl-3-methyl-4-oxo- | 3H), 0.93(d, 3H), 1.18(m, 1H), | 522(MH⁺) | 3: |
| | 4,5-dihydro-isothiazolo[5,4- | 1.53(m, 1H), 2.32-2.51(s, m, 5H), | | Method |
| | d]pyrimidin-6-yl)-2-methyl- | 2.82(s, 4H), 3.35-3.43(m, 2H), 5.10(m, | | 35f |
| | propyl]-3-fluoro-4-methyl- | 1H), 5.62(m, 1H), 5.94(d, 1H), | | |
| | benzamide hydrogen chloride | 7.11-7.38(m, 8H), 7.51(b, 2H) | | |
| D8 | (+) N-(3-Amino-propyl)-N- | (DMSO-d₆, 90° C.) δ: 0.48(d, 3H), | m/z | Section |
| | [1-(5-benzyl-3-methyl-4-oxo- | 0.93(m, 3H), 1.10-1.20(m, 1H), | 568, | 3: |
| | 4,5-dihydro-isothiazolo[5,4- | 1.45-1.60(m, 1H), 2.28-2.41(t, 2H), | 570(MH⁺) | Method |
| | d]pyrimidin-6-yl)-2-methyl- | 2.63-2.79(m, s, 4H), 3.35-3.43(m, | | 35g |
| | propyl]-4-bromo-benzamide | 2H), 5.08(m, 1H), 5.62(m, 1H), | | |
| | hydrogen chloride | 5.96(d, 1H), 7.30-7.50(m, 7H), | | |
| | | 7.52-7.80(br, m, 4H) | | |

### EXAMPLE E

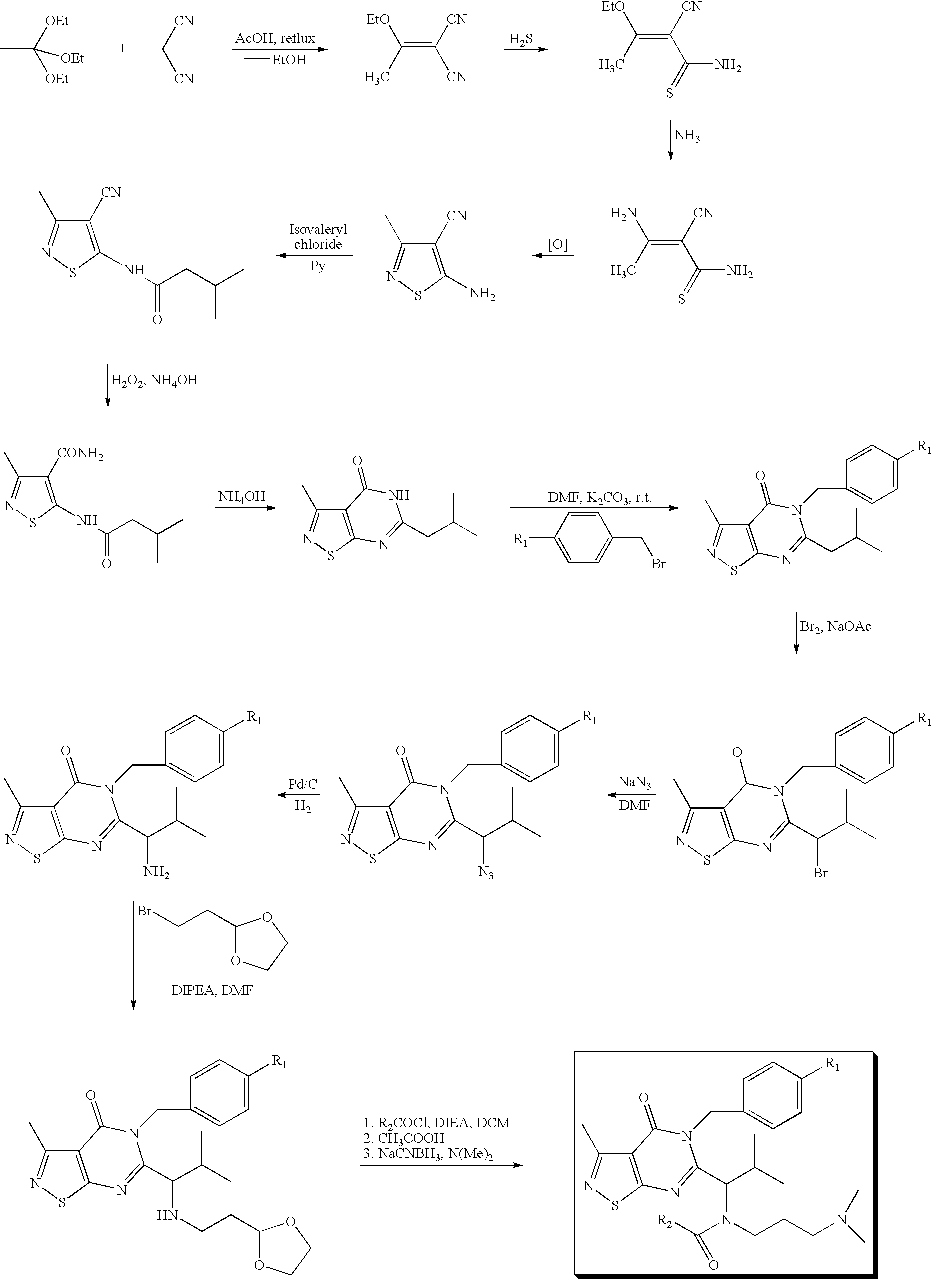

### SECTION 3: EXAMPLES E

The following compounds were synthesized according to synthetic scheme E above:

| Ex. | Compound | ¹H NMR | m/z | SM |
|---|---|---|---|---|
| E1 | (+) N-[1-(5-Benzyl-3- | (DMSO-d₆, 90° C.) δ: 0.36(d, 3H), | m/z | Section |
| | methyl-4-oxo-4,5- | 0.73(m, 1H), 0.96(d, 3H), 1.26-1.27(m, | 532(MH⁺) | 3: |
| | dihydro-isothiazolo[5,4- | 1H), 1.65-1.87(br m, s, 8H), 2.37(s, | | Method |
| | d]pyrimidin-6-yl)-2- | 3H), 2.72(m, 1H), 2.87(s, 3H), | | 40a |
| | methyl-propyl]-N-(3- | 3.35-3.41(m, 2H), 5.22-5.27(d, 1H), | | |
| | dimethylamino-propyl)-4- | 5.73-5.76(d, 1H), 6.12-6.17(d, 1H), | | |
| | methyl-benzamide | 7.22-7.41(m, 9H) | | |
| E2 | (+) N-[1-(5-Benzyl-3- | (DMSO-d₆, 90° C.) δ: 0.36(d, 3H), | m/z | Section |
| | methyl-4-oxo-4,5- | 0.73(m, 1H), 0.95(d, 3H), 1.20-1.23(m, | 596, | 3: |
| | dihydro-isothiazolo[5,4- | 1H), 1.64-1.82(br m, s, 8H), 2.69(m, | 598(MH⁺) | Method |
| | d]pyrimidin-6-yl)-2- | 1H), 2.87(s, 3H), 3.35-3.37(m, | | 40 |
| | methyl-propyl]-N-(3- | 2H), 5.17-5.22(d, 1H), 5.71-5.75(d, | | |
| | dimethylamino-propyl)-4- | 1H), 6.12-6.17(d, 1H), 7.21-7.57(m, | | |
| | bromo-benzamide | 9H) | | |
| E3 | (+) N-[1-(5-Benzyl-3- | (DMSO-d₆, 90° C.) δ: 0.36(d, 3H), | m/z | Section |
| | methyl-4-oxo-4,5- | 0.73(m, 1H), 0.94(d, 3H), 1.20-1.23(m, | 540(MH⁺) | 3: |
| | dihydro-isothiazolo[5,4- | 1H), 1.65-1.83(br m, s, 8H), 2.30(s, | | Method |
| | d]pyrimidin-6-yl)-2- | 3H), 2.69(m, 1H), 2.87(s, 3H), | | 40b |
| | methyl-propyl]-N-(3- | 3.35-3.41(t, 2H), 5.17-5.23(d, 1H), | | |
| | dimethylamino-propyl)-3- | 5.71-5.74(d, 1H), 6.11-6.16(d, 1H), | | |
| | fluoro-4-methyl- | 6.99-7.39(m, 8H) | | |
| | benzamide | | | |

### SECTION 3: EXAMPLE F

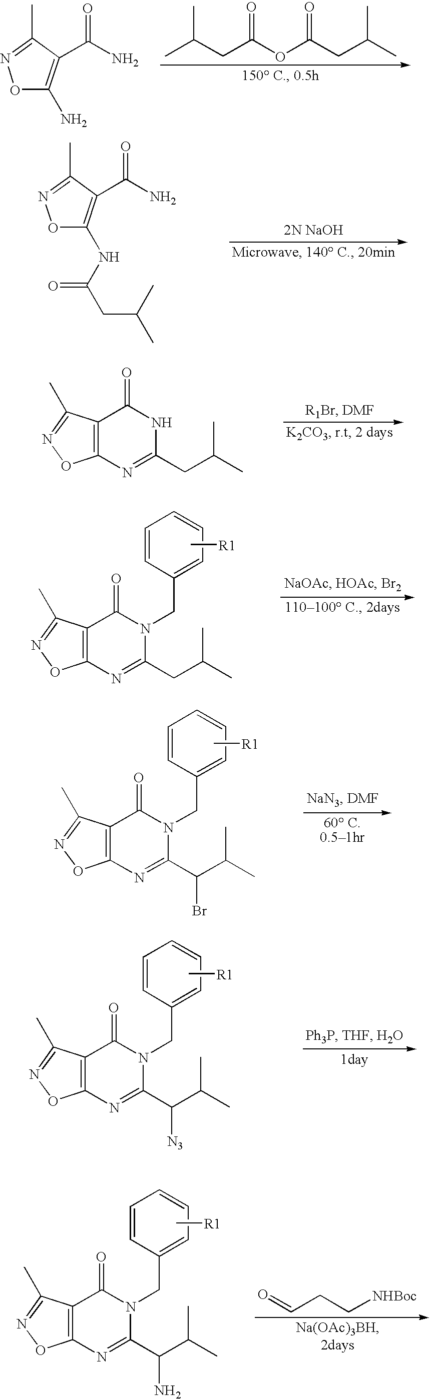

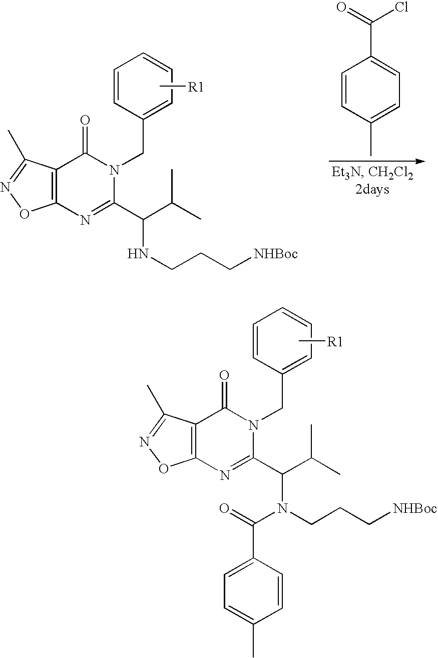

### SECTION 3: EXAMPLE F

The following compounds were synthesized according to synthetic scheme F above:

| Ex. | Compound | ¹H NMR | m/z | SM |
|---|---|---|---|---|
| F1 | (+) N-(3-Amino-propyl)-N- | (500MHz, 100° C., DMSO-d₆): δ: 0.48(d, | m/z | Section |
| | [1-(5-benzyl-3-methyl-4- | 3H), 0.94(d, 3H), 1.20-1.45(m, | 488(MH⁺) | 3: |
| | oxo-4,5-dihydro- | 2H), 2.15(m, 2H), 2.38(s, 3H), 2.58(s, | | Method |
| | isoxazolo[5,4-d]pyrimidin- | 3H), 2.70(m, 1H), 3.37(m, 2H), | | 51 |
| | 6-yl)-2-methyl-propyl]-4- | 5.11(d, 1H), 5.64(d, 1H), 5.90(d, | | |
| | methyl-benzamide | 1H), 7.23-7.39(m, 9H) | | |
| F2 | (+) N-(3-Amino-propyl)-N- | (500MHz, 100° C., DMSO-d₆) δ: 0.50(d, | m/z | Section |
| | {1-[5-(4-fluoro-benzyl)-3- | 3H), 0.95-1.10(d, m, 4H), 1.55(m, | 506(MH⁺) | 3: |
| | methyl-4-oxo-4,5-dihydro- | 1H), 2.32(m, 2H), 2.40(s, 3H), 2.60(s, | | Method |
| | isoxazolo[5,4-d]pyrimidin- | 3H), 2.75(m, 1H), 3.40(m, 2H), | | 51a |
| | 6-yl]-2-methyl-propyl}-4- | 5.10(d, 1H), 5.60(d, 1H), 5.88(d, | | |
| | methyl-benzamide | 1H), 7.17-7.34(m, 8H) | | |
| F3 | (+) N-(3-Amino-propyl)-N- | (90° C., DMSO-d₆) δ: 0.44(d, 3H), | m/z | Section |
| | {1-[5-(3-fluoro-benzyl)-3- | 0.96(d, 3H), 1.15-1.35(m, 1H), 1.50-1.71(m, | 506(MH⁺) | 3: |
| | methyl-4-oxo-4,5-dihydro- | 1H), 2.36(m, s, 4H), 2.60(s, | | Method |
| | isoxazolo[5,4-d]pyrimidin- | 3H), 2.60-2.80(m, 2H), 3.43-3.54(m, | | 50b |
| | 6-yl]-2-methyl-propyl}-4- | 2H), 5.10(m, 1H), 5.62(d, 1H), 5.82(d, | | |
| | methyl-benzamide | 1H), 7.12-7.37(m, 8H), 7.60(br, | | |
| | hydrogen chloride | 3H) | | |

### EXAMPLE G

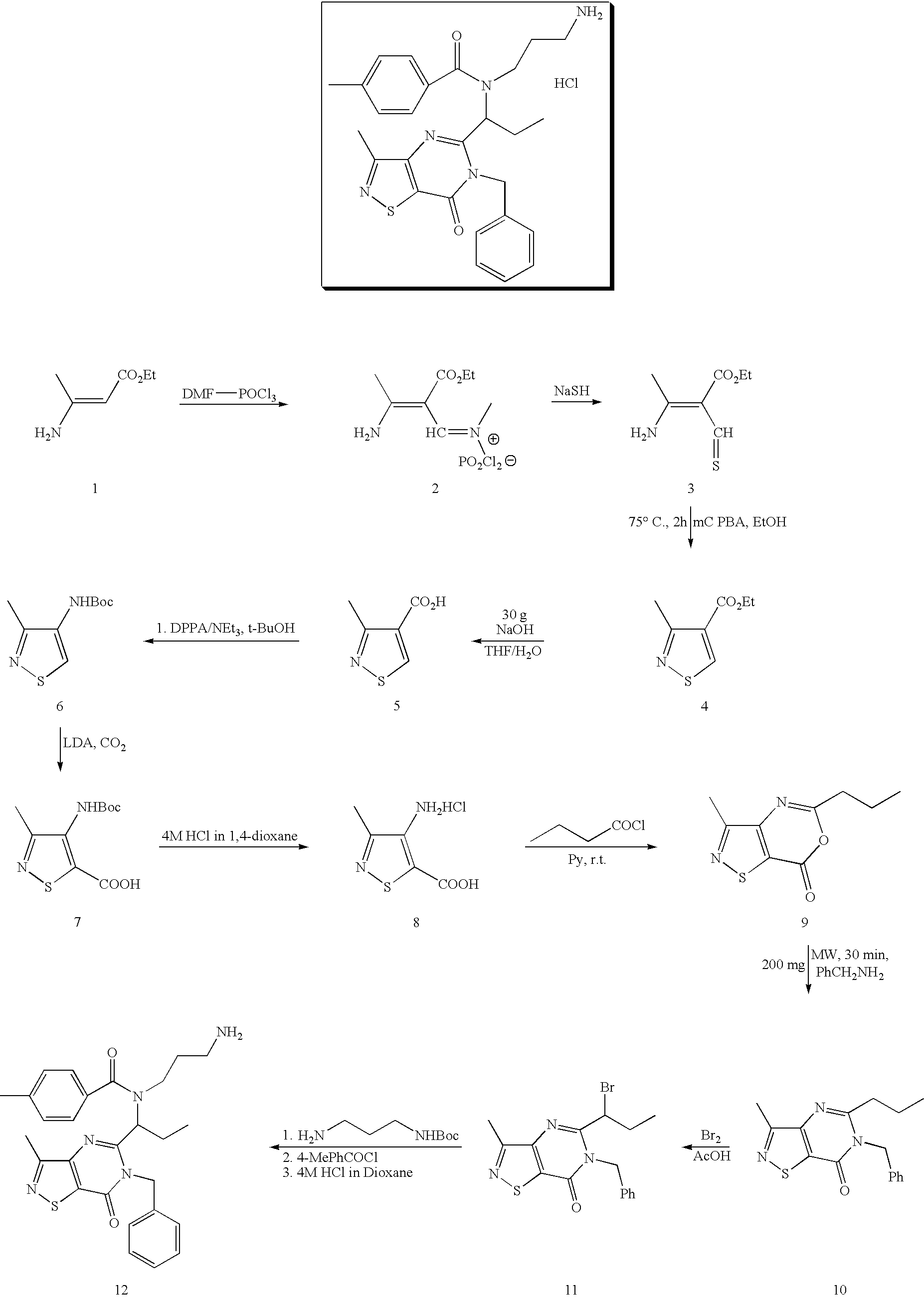

### SECTION 3: EXAMPLE G

The following compounds were synthesized according to synthetic scheme G above:

| Ex. | Compound | ¹H NMR | m/z | SM |
|---|---|---|---|---|
| G1 | (+)N-(3-Amino-propyl)-N-[1- | (DMSO-d₆, 90° C.) δ 0.65(t, | m/z | Section |
| | (6-benzyl-3-methyl-7-oxo-6,7- | 3H), 1.36-1.50(m, 1H), 1.60-1.72(m, | 490(MH⁺) | 3: |
| | dihydro-isothiazolo[4,5- | 1H), 1.88-1.99(m, 1H), | | Method |
| | d]pyrimidin-5-yl)-propyl]-4- | 2.14-2.26(m, 1H), 2.35(s, 3H), | | 62 |
| | methyl-benzamide | 2.47(t, 2H), 2.68(s, 3H), 3.32-3.44(m, | | |
| | | 2H), 4.90(d, 1H), 5.50(b, | | |
| | | 1H), 5.76(d, 1H), 6.96-7.34(m, | | |
| | | 9H), 7.68(bs, 3H). | | |

Chiral Rotations of the Section 3: Examples

Rotations were measured on a Perkin Elmer Model 341 polarimeter. The compounds were dissolved to a concentration of 1 mg/ml in methanol and the measurements were made at 20.0° C., at 589 nM. 1 ml of solution was used.

| Section 3: | |
|---|---|
| Example | Rotation |
| A1 | + |
| C1 | + |
| D2 | + |
| A8 | + |
| D5 | + |
| A5 | + |
| A4 | + |
| D4 | + |
| D6 | + |
| A6 | + |
| A7 | + |
| F2 | + |
| F1 | + |
| A3 | + |
| A10 | + |
| A2 | + |
| A9 | + |
| D7 | + |
| D8 | + |
| D3 | + |
| D1 | + |
| B1 | + |
| E2 | + |
| E1 | + |
| E3 | + |
| G1 | + |
| F3 | + |

Section 3: Utility

Compounds of formula (I) of section 3 have been shown to inhibit the microtubule motor protein HsEg5 in vitro. Inhibitors of Eg5 have been shown to inhibit the formation of a mitotic spindle and therefore for cell division. Inhibitors of Eg5 have been shown to block cells in the metaphase of mitosis leading to apoptosis of effected cells, and to therefore have anti-proliferative effects. It is believed that Eg5 inhibitors act as modulators of cell division and are expected to be active against neoplastic disease such as carcinomas of the brain, breast, ovary, lung, colon, prostate or other tissues, as well as multiple myeloma leukemias, for example myeloid leukemia, acute lymphoblastic leukemia, chronic myeloid leukemia, chronic lymphocytic leukemia, and lymphomas for example Hodgkins disease and non-Hodgkins lymphoma, tumors of the central and peripheral nervous system, and other tumor types such as melanoma, fibrosarcoma, Ewing's sarcoma and osteosarcoma. Therefore it is believed that the compounds of formula (I) of section 3 may be used for the treatment of neoplastic disease. Hence the compounds of formula (I) and their salts and their in vivo hydrolysable esters of section 3 are expected to be active against carcinomas of the brain, breast, ovary, lung, colon, prostate or other tissues, as well as leukemias and lymphomas, tumors of the central and peripheral nervous system, and other tumor types such as melanoma, fibrosarcoma and osteosarcoma. The compounds of formula (I) and their salts and their in vivo hydrolysable esters of section 3 are expected to be active against neoplastic disease such as carcinomas of the brain, breast, ovary, lung, colon, prostate or other tissues, as well as multiple myeloma leukemias, for example myeloid leukemia, acute lymphoblastic leukemia, chronic myeloid leukemia, chronic lymphocytic leukemia, and lymphomas for example Hodgkins disease and non-Hodgkins lymphoma, tumors of the central and peripheral nervous system, and other tumor types such as melanoma, fibrosarcoma, Ewing's sarcoma and osteosarcoma. It is expected that the compounds of formula (I) of section 3 would most likely be used in combination with a broad range of agents but could also be used as a single agent.

Generally, the compounds of formula (I) of section 3 have been identified in the Malachite Green Assay described herein as having an IC₅₀ value of 100 micromolar or less. For example compound A7 ((+) N-(3-Amino-propyl)-N-[1-(5-benzyl-3-methyl-4-oxo-4,5-dihydro-isothiazolo[5,4-d]pyrimidin-6-yl)-propyl]-2,3-dichloro-benzamide hydrogen chloride) has an IC₅₀ value of 136 nM.

Compounds provided by section 3 of this invention should also be useful as standards and reagents in determining the ability of a potential pharmaceutical to inhibit Eg5. These would be provided in commercial kits comprising a compound of this invention.

Section 3: Malachite Green Assay

Enzymatic activity of the Eg5 motor and effects of inhibitors was measured using a malachite green assay, which measures phosphate liberated from ATP, and has been used previously to measure the activity of kinesin motors (Hackney and Jiang, 2001). Enzyme was recombinant HsEg5 motor domain (amino acids 1-369-8His) and was added at a final concentration of 6 nM to 100 μl reactions. Buffer consisted of 25 mM PIPES/KOH, pH 6.8, 2 mM MgCl₂, 1 mM EGTA, 1 mM dtt, 0.01% Triton X-100 and 5 μM paclitaxel. Malachite green/ammonium molybdate reagent was prepared as follows: for 800 ml final volume, 0.27 g of Malachite Green (J. T. Baker) was dissolved in 600 ml of H₂O in a polypropylene bottle. 8.4 g ammonium molybdate (Sigma) was dissolved in 200 ml 4N HCl. The solutions were mixed for 20 min and filtered through 0.02 μm filter directly into a polypropylene container. 5 μl of compound diluted in 12% DMSO was added to the wells of 96 well plates. 80 μl of enzyme diluted in buffer solution above was added per well and incubated with compound for 20 min. After this pre-incubation, substrate solution containing 2 mM ATP (final concentration: 300 μM) and 6.053 μM polymerized tubulin (final concentration: 908 nM) in 15 μl of buffer were then added to each well to start reaction. Reaction was mixed and incubated for an additional 20 min at room temperature. The reactions were then quenched by the addition of 150 μl malachite green/ammonium molybdate reagent, and absorbance read at 650 nanometers exactly 5 min after quench using a Spectramax Plus plate reader (Molecular Devices). Data was graphed and IC₅₀s calculated using ExCel Fit (Microsoft).

1. A compound having the structural formula (I): 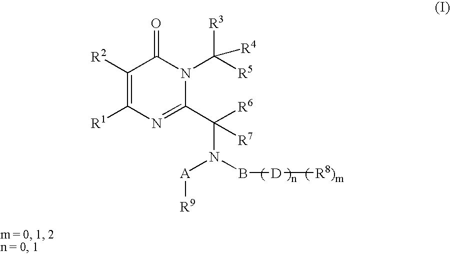
wherein,
A is C═O, CH₂, or SO₂;
B represents optionally substituted alkyl, optionally substituted alkenyl, optionally substituted alkynyl, optionally substituted aryl, optionally substituted cycloalkyl, or optionally substituted heterocycle;
D is O or N wherein O is optionally substituted with one R⁸, wherein N is optionally substituted with one or more R⁸, and when n is 0 and m is not 0, R⁸ is attached directly to B;
R¹ and R² in combination form a fused 5-membered heteroaromatic ring that is optionally substituted with 1 or 2 substituents, said ring having at least one nitrogen, oxygen or sulfur atoms, but no more than 2 oxygen atoms or 2 sulfur atoms or 1 oxygen and 1 sulfur atom;
R³ is independently selected from H, optionally substituted alkyl, optionally substituted alkenyl, optionally substituted alkynyl, optionally substituted cycloalkyl, optionally substituted cycloalkenyl, optionally substituted cycloalkynyl, optionally substituted aryl or optionally substituted heterocycle;
R⁴ and R⁵ are independently selected from H or optionally substituted alkyl, or R⁴ and R⁵ in combination form a 3-, 4-, 5- or 6-membered ring, which may also be optionally substituted;
R⁶ and R⁷ are independently selected from H, optionally substituted alkyl, optionally substituted alkenyl, optionally substituted alkynyl, optionally substituted cycloalkyl, optionally substituted cycloalkenyl, optionally substituted cycloalkynyl, optionally substituted heterocycle, optionally substituted aryl, or R⁶ and R⁷ in combination form a 3-, 4-, 5- or 6-membered ring, which may also be substituted;
R⁸ is independently selected from H, optionally substituted alkyl, optionally substituted alkenyl, optionally substituted alkynyl, optionally substituted cycloalkyl, optionally substituted cycloalkenyl, optionally substituted cycloalkynyl, optionally substituted aryl, or optionally substituted heterocycle;
R⁹ is independently selected from H, optionally substituted alkyl, optionally substituted alkenyl, optionally substituted alkynyl, optionally substituted cycloalkyl, optionally substituted cycloalkenyl, optionally substituted cycloalkynyl, optionally substituted aryl, or optionally substituted heterocycle.

2. A compound as recited in claim 1 wherein A is C═O.

3. A compound as recited in claim 1 wherein B is optionally substituted C₁₋₄alkyl.

4. A compound as recited in claim 1 wherein B is an optionally substituted C₁₋₄alkyl wherein such substituent is independently selected from —NH₂, —OH, —NCH₃, —N(CH₃)₂, —N-cyclopropane, —N cyclobutane, azetidine, pyrrolidine, or piperidine.

5. A compound as recited in claim 1 wherein D is N optionally substituted with one or more R⁸.

6. A compound as recited in claim 1 wherein R¹ and R² in combination form a fused 5-membered heteroaromatic ring that is optionally substituted with 1 or 2 substituents, said ring having one nitrogen atom and one sulfur atom, or one nitrogen atom and one oxygen atom.

7. A compound as recited in claim 1 wherein R¹ and R² in combination form an optionally substituted fused isothiazole, or an optionally substituted fused isoxazole.

8. A compound as recited in claim 1 wherein R¹ and R² in combination form a fused 5-membered heteroaromatic ring that is optionally substituted with 1 or 2 substituents, said ring having one nitrogen atom and one sulfur atom, or one nitrogen atom and one oxygen atom and wherein said substituent is selected from C₁₋₆alkyl, or halogen.

9. A compound as recited in claim 1 wherein R³ is optionally substituted C₅₋₇aryl.

10. A compound as recited in claim 1 wherein R³ is optionally substituted C₅₋₇aryl wherein said substituent is independently selected from C₁₋₆alkyl, F, Cl, Br, or I.

11. A compound as recited in claim 1 wherein R⁴ and R⁵ are H.

12. A compound as recited in claim 1 wherein R⁶ and R⁷ are independently selected from H, or C₁₋₆alkyl.

13. A compound as recited in claim 1 wherein R⁸ is independently selected from H, optionally substituted alkyl, or optionally substituted heterocycle.

14. A compound as recited in claim 1 wherein R⁹ is C₅₋₇aryl optionally substituted with 1 or 2 substituents wherein said substituent is independently selected from —C₁₋₆alkyl, —OC₁₋₆alkyl, F, Cl, Br, I.

15. A compound as recited in claim 1 wherein:
n is 1;
A is CO or CH₂;
B is optionally substituted C₁₋₆alkyl;
D is N or O;
R¹ and R² in combination form a fused isothiazole, isoxazole;
R³ is optionally substituted phenyl;
R⁴ and R⁵ are H;
R⁶ and R⁷ are H or optionally substituted alkyl;
R⁸ is H or optionally substituted C₁₋₆alkyl;
R⁹ is optionally substituted phenyl.

16. A compound of formula (I) selected from:
N-(3-Amino-propyl)-N-{1-[5-(4-fluoro-benzyl)-3-methyl-4-oxo-4,5-dihydro-isothiazolo[5,4-d]pyrimidin-6-yl]-propyl}-4-methyl-benzamide
N-(3-Amino-propyl)-N-[1-(5-benzyl-3-methyl-4-oxo-4,5-dihydro-isothiazolo[5,4-d]pyrimidin-6-yl)-propyl]-4-bromo-benzamide;
N-(3-Amino-propyl)-N-[1-(5-benzyl-3-methyl-4-oxo-4,5-dihydro-isothiazolo[5,4-d]pyrimidin-6-yl)-propyl]-4-chloro-benzamide;
N-(3-Amino-propyl)-N-[1-(5-benzyl-3-methyl-4-oxo-4,5-dihydro-isothiazolo[5,4-d]pyrimidin-6-yl)-propyl]-3-fluoro-4-methyl-benzamide;
N-(3-Amino-propyl)-N-[1-(5-benzyl-3-methyl-4-oxo-4,5-dihydro-isothiazolo[5,4-d]pyrimidin-6-yl)-propyl]-2,3-dichloro-benzamide;
Naphthalene-2-carboxylic acid (3-amino-propyl)-[1-(5-benzyl-3-methyl-4-oxo-4,5-dihydro-isothiazolo[5,4-d]pyrimidin-6-yl)-propyl]-amide;
Benzo[b]thiophene-2-carboxylic acid (3-amino-propyl)-[1-(5-benzyl-3-methyl-4-oxo-4,5-dihydro-isothiazolo[5,4-d]pyrimidin-6-yl)-propyl]-amide;
N-Azetidin-3-ylmethyl-N-[1-(5-benzyl-3-methyl-4-oxo-4,5-dihydro-isothiazolo[5,4-d]pyrimidin-6-yl)-propyl]-4-methyl-benzamide;
N-[1-(5-Benzyl-3-methyl-4-oxo-4,5-dihydro-isothiazolo[5,4-d]pyrimidin-6-yl)-propyl]-4-methyl-N-piperidin-3-ylmethyl-benzamide;
N-(2-Amino-ethyl)-N-[1-(5-benzyl-3-methyl-4-oxo-4,5-dihydro-isothiazolo[5,4-d]pyrimidin-6-yl)-propyl]-4-methyl-benzamide;
N-[1-(5-Benzyl-3-methyl-4-oxo-4,5-dihydro-isothiazolo[5,4-d]pyrimidin-6-yl)-propyl]-N-(2-dimethylamino-ethyl)-4-methyl-benzamide;
N-[1-(5-Benzyl-3-methyl-4-oxo-4,5-dihydro-isothiazolo[5,4-d]pyrimidin-6-yl)-propyl]-N-(3-dimethylamino-propyl)-4-methyl-benzamide;
N-[1-(5-Benzyl-3-methyl-4-oxo-4,5-dihydro-isothiazolo[5,4-d]pyrimidin-6-yl)propyl]-N-[3-(isopropylamino)propyl]-4-methylbenzamide;
N-[1-(5-benzyl-3-methyl-4-oxo-4,5-dihydro-isothiazolo[5,4-d]pyrimidin-6-yl)propyl]-N-[3-(cyclopropylamino)propyl]4-methylbenzamide;
N-(3-azetidin-1-ylpropyl)-N-[1-(5-benzyl-3-methyl-4-oxo-4,5-dihydro-isothiazolo[5,4-d]pyrimidin-6-yl)propyl]-4-methylbenzamide;
N-[1-(5-benzyl-3-methyl-4-oxo-4,5-dihydro-isothiazolo[5,4-d]pyrimidin-6-yl)propyl]-4-methyl-N-[3-(3-pyrrolidin-1-ylpropyl) benzamide;
N-[1-(5-benzyl-3-methyl-4-oxo-4,5-dihydro-isothiazolo[5,4-d]pyrimidin-6-yl)propyl]-4-methyl-N-[3-(methylamino) propyl] benzamide;
N-[1-(5-Benzyl-3-methyl-4-oxo-4,5-dihydro-isothiazolo[5,4-d]pyrimidin-6-yl)-propyl]-N-(3-hydroxy-propyl)-4-methyl-benzamide;
N-(3-Amino-propyl)-N-[1-(5-benzyl-3-methyl-4-oxo-4,5-dihydro-isothiazolo[5,4-d]pyrimidin-6-yl)-propyl]-4-methyl-benzamide;
N-(3-Amino-propyl)-N-[1-(5-benzyl-3-methyl-4-oxo-4,5-dihydro-isothiazolo[5,4-d]pyrimidin-6-yl)-2-methyl-propyl]-4-methyl-benzamide;
N-(3-Amino-propyl)-N-{1-[5-(4-fluoro-benzyl)-3-methyl-4-oxo-4,5-dihydro-isothiazolo[5,4-d]pyrimidin-6-yl]-2-methyl-propyl}-4-methyl-benzamide;
5-Benzyl-6-{1-[(3-hydroxy-propyl)-(4-methyl-benzyl)-amino]-propyl}-3-methyl-5H-isothiazolo[5,4-d]pyrimidin-4-one;
N-(3-Amino-propyl)-N-{1-[5-(4-fluoro-benzyl)-3-methyl-4-oxo-4,5-dihydro-isoxazolo[5,4-d]pyrimidin-6-yl]-2-methyl-propyl}-4-methyl-benzamide;
N-(3-Amino-propyl)-3-fluoro-N-{1-[5-(4-fluoro-benzyl) 3-methyl-4-oxo-4,5-dihydro-isoxazolo[5,4-d]pyrimidin-6-yl]-2-methyl-propyl}-4-methyl-benzamide;
N-(3-Amino-propyl)-N-[1-(5-benzyl-3-methyl-4-oxo-4,5-dihydro-isoxazolo[5,4-d]pyrimidin-6-yl)-2-methyl-propyl]-3-fluoro-4-methyl-benzamide;
N-(3-Amino-propyl)-N-[1-(5-benzyl-3-methyl-4-oxo-4,5-dihydro-isoxazolo[5,4-d]pyrimidin-6-yl)-2-methyl-propyl]-4-methyl-benzamide;
N-(3-Amino-propyl)-N-[1-(5-benzyl-3-methyl-4-oxo-4,5-dihydro-isoxazolo[5,4-d]pyrimidin-6-yl)-propyl]-4-methyl-benzamide;
N-(3-Amino-propyl)-N-[1-(5-benzyl-3-methyl-4-oxo-4,5-dihydro-isoxazolo[5,4-d]pyrimidin-6-yl)-propyl]-4-fluoro-benzamide;
N-(3-Amino-propyl)-N-[1-(5-benzyl-3-methyl-4-oxo-4,5-dihydro-isoxazolo[5,4-d]pyrimidin-6-yl)-propyl]-2,3-dichloro-benzamide;
N-(3-Amino-propyl)-N-[1-(5-benzyl-3-methyl-4-oxo-4,5-dihydro-isoxazolo[5,4-d]pyrimidin-6-yl)-propyl]-3-fluoro-4-methyl-benzamide;
N-(3-Amino-propyl)-N-[1-(5-benzyl-3-methyl-4-oxo-4,5-dihydro-isoxazolo[5,4-d]pyrimidin-6-yl)-propyl]-4-methoxy-benzamide.

17. A method for the treatment of cancer associated with comprising administering to a host in need of such treatment a therapeutically effective amount of a compound as defined in claim 1.

18. A method of producing a cell cycle inhibitory (anti-cell-proliferation) effect in a warm-blooded animal, such as man, in need of such treatment with comprises administering to said animal an effective amount of a compound as claimed in claim 1.

19. A pharmaceutical composition comprising a compound as defined in claim 1, or a pharmaceutically acceptable salt or in vivo hydrolysable ester thereof, together with at least one pharmaceutically acceptable carrier, diluent or excipient.

20. A compound of formula (I): 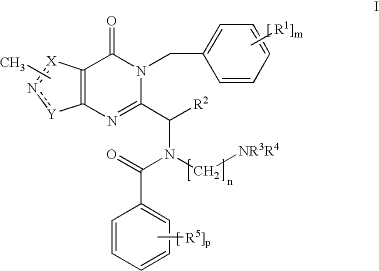
including a pharmaceutically acceptable salt thereof,
wherein:
X is selected from C or S provided that when X is S then Y is C;
Y is selected from C or O or S provided that when Y is C then X is not C;
m is 0, or 1;
R¹ is F, when m is 1;
R² is selected from C₁₋₃alkyl;
n is 2 or 3;
R³ and R⁴ are independently selected from H or C₁₋₂alkyl;
R⁵ is selected from F, Cl, Br, or C₁₋₂alkyl;
p is 1 or 2;
selected from:
N-(3-amino-propyl)-N-{1-[5-(3-fluoro-benzyl)-3-methyl-4-oxo-4,5-dihydro-isothiazolo[5,4-d]pyrimidin-6-yl]-propyl}-4-methyl-benzamide;
N-(3-amino-propyl)-N-{1-[5-(3-fluoro-benzyl)-3-methyl-4-oxo-4,5-dihydro-isothiazolo[5,4-d]pyrimidin-6-yl]-2-methyl-propyl}-4-methyl-benzamide;
N-(2-amino-ethyl)-N-[1-(5-benzyl-3-methyl-4-oxo-4,5-dihydro-isothiazolo[5,4-d]pyrimidin-6-yl)-2-methyl-propyl]-4-bromo-benzamide;
N-(2-amino-ethyl)-N-[1-(5-benzyl-3-methyl-4-oxo-4,5-dihydro-isothiazolo[5,4-d]pyrimidin-6-yl)-2-methyl-propyl]-4-methyl-benzamide;
N-(2-amino-ethyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
N-(3-amino-propyl)-N-[1-(5-benzyl-3-methyl-4-oxo-4,5-dihydro-isothiazolo[5,4-d]pyrimidin-6-yl)-2-methyl-propyl]-3-fluoro-4-methyl-benzamide;
N-(3-amino-propyl)-N-[1-(5-benzyl-3-methyl-4-oxo-4,5-dihydro-isothiazolo[5,4-d]pyrimidin-6-yl)-2-methyl-propyl]-4-bromo-benzamide;
N-[1-(5-benzyl-3-methyl-4-oxo-4,5-dihydro-isothiazolo[5,4-d]pyrimidin-6-yl)-2-methyl-propyl]-N-(3-dimethylamino-propyl)-4-methyl-benzamide;
N-[1-(5-benzyl-3-methyl-4-oxo-4,5-dihydro-isothiazolo[5,4-d]pyrimidin-6-yl)-2-methyl-propyl]-N-(3-dimethylamino-propyl)-4-bromo-benzamide;
N-[1-(5-benzyl-3-methyl-4-oxo-4,5-dihydro-isothiazolo[5,4-d]pyrimidin-6-yl)-2-methyl-propyl]-N-(3-dimethylamino-propyl)-3-fluoro-4-methyl-benzamide;
N-(3-amino-propyl)-N-{1-[5-(3-fluoro-benzyl)-3-methyl-4-oxo-4,5-dihydro-isoxazolo[5,4-d]pyrimidin-6-yl]-2-methyl-propyl}-4-methyl-benzamide;
N-(3-amino-propyl)-N-[1-(6-benzyl-3-methyl-7-oxo-6,7-dihydro-isothiazolo[4,5-d]pyrimidin-5-yl)-propyl]-4-methyl-benzamide.
